(19) United States
(12) Patent Application Publication
(10) Pub. No.: US 2023/0002777 A1
(43) Pub. Date: Jan. 5, 2023

(54) Production System

(71) Applicant: Oxford BioMedica (UK) Limited, Oxford (GB)

(72) Inventor: Daniel Farley, Oxford (GB)

(21) Appl. No.: 17/775,922

(22) PCT Filed: Nov. 11, 2020
(86) PCT No.: PCT/GB2020/052873

§ 371 Date: May 11, 2022

(30) Foreign Application Priority Data
Nov. 12, 2019 (GB) 1916452.4
Feb. 13, 2020 (GB) 2001998.0

(51) Int. Cl. C12N 15/67; A61K 48/00; C12N 15/86

(52) U.S. Cl. CPC C12N 15/67; A61K 48/00; C12N 15/86; C12N 2740/16043; C12N 2740/16051; C12N 2750/14143; C12N 2750/14151; C12N 2840/102; C12N 2840/20; C12N 2840/203

(57) ABSTRACT

The present invention relates to a nucleic acid sequence comprising a nucleotide of interest and a tryptophan RNA-binding attenuation protein (TRAP) binding site, and optionally a Kozak sequence, wherein said TRAP binding site overlaps the Kozak sequence and/or the ATG start codon of the nucleotide of interest. The present invention further relates to a nucleic acid sequence comprising a nucleotide of interest and a Kozak sequence, wherein said Kozak sequence comprises a portion of a tryptophan RNA-binding attenuation protein (TRAP) binding site. The present invention further relates to a nucleic acid sequence comprising a nucleotide of interest and TRAP binding site wherein the TRAP binding site comprises a portion of the start codon ATG of said nucleotide of interest or wherein the ATG start codon comprises a portion of the TRAP binding site. The present invention further relates to a nucleic acid sequence comprising a nucleotide of interest, a binding site for tryptophan RNA-binding attenuation protein (TRAP), a multiple cloning site and a Kozak sequence, wherein said multiple cloning site is overlapping with or located downstream to the 3′ KAGN2-3 repeat of the TRAP binding site and upstream of the Kozak sequence.

### FIELD OF THE INVENTION

The invention relates to the production of viral vectors. More specifically, the present invention relates to modification of the translation of a nucleotide of interest which is encoded by a viral vector, in a viral vector production cell.

### BACKGROUND TO THE INVENTION

Gene therapy broadly involves the use of genetic material to treat disease. It includes the supplementation in cells with defective genes (e.g. those harbouring mutations) with functional copies of those genes, the inactivation of improperly functioning genes and the introduction of new therapeutic genes.

Therapeutic genetic material may be incorporated into the target cells of a host using vectors to enable the transfer of nucleic acids. Such vectors can be generally divided into viral and non-viral categories.

Viruses naturally introduce their genetic material into target cells of a host as part of their replication cycle. Engineered viral vectors harness this ability to enable the delivery of a nucleotide of interest (NOI) or transgene to a target cell. To date, a number of viruses have been engineered as vectors for gene therapy. These include retroviruses, adenoviruses (AdV), adeno-associated viruses (AAV), herpes simplex viruses (HSV) and vaccinia viruses.

In addition to modification to carry the NOI, viral vectors are typically further engineered to be replication defective. As such, the recombinant vectors can directly infect a target cell, but are incapable of producing further generations of infective virions. Other types of viral vectors may be conditionally replication competent within cancer cells only, and may additionally encode a toxic transgene or pro-enzyme.

Retroviral vectors have been developed as therapies for various genetic disorders and are now showing increasing promise in clinical trials (e.g. Galy, A. and A. J. Thrasher (2010) Curr Opin Allergy Clin Immunol 11(6): 545-550; Porter, D. L., B. L. Levine, M. Kalos, A. Bagg and C. H. June (2011) N Engl J Med 365(8): 725-733; Campochiaro, P. A. (2012) Gene Ther 19(2): 121-126; Cartier, N., S. Hacein-Bey-Abina, C. C. Bartholomae, P. Bougneres, M. Schmidt, C. V. Kalle, A. Fischer, M. Cavazzana-Calvo and P. Aubourg (2012) Methods Enzymol 507: 187-198; Sadelain, M., I. Riviere, X. Wang, F. Boulad, S. Prockop, P. Giardina, A. Maggio, R. Galanello, F. Locatelli and E. Yannaki (2010) Ann N Y Acad Sci 1202: 52-58; DiGiusto, D. L., A. Krishnan, L. Li, H. Li, S. Li, A. Rao, S. Mi, P. Yam, S. Stinson, M. Kalos, J. Alvarnas, S. F. Lacey, J. K. Yee, M. Li, L. Couture, D. Hsu, S. J. Forman, J. J. Rossi and J. A. Zaia (2010) Sci Transl Med 2(36): 36ra43 and Segura M M, M. M., Gaillet B, Gamier A. (2013) Expert opinion in biological therapy).

Important examples of such vectors include the gamma-retrovirus vector system (based on MMLV), the primate lentivirus vector system (based on HIV-1) and the non-primate lentivirus vector system (based on EIAV).

Reverse genetics has allowed these virus-based vectors to be heavily engineered such that vectors encoding large heterologous sequences (circa 10 kb) can be produced by transfection of mammalian cells with appropriate DNA sequences (reviewed in Bannert, K. (2010) Caister Academic Press: 347-370).

Engineering and use of retroviral vectors at the research stage typically involves the production of reporter-gene vectors encoding, for example, GFP or lacZ. The titres of these clinically irrelevant vectors are usually in the region of 1×10⁶ to 1×10⁷ transducing units per mL (TU/mL) of crude harvest material. Further concentration and purification of this material can achieve working stocks in excess of 1×10⁻¹⁰ TU/mL. However, the production of vectors encoding therapeutically relevant NOIs often results in substantially reduced titres compared to these reporter vectors.

There are several factors that are potentially responsible for this effect:
1. The size of the therapeutic genome. Very large genomes can be packaged by retroviruses, but it is thought that reverse transcription and/or integration steps become less efficient as size increases.
2. The stability of vector genome RNA. This may be reduced by the presence of unpredicted instability elements in the NOI.
3. Suboptimal nucleotide usage within the vector genome RNA. Wild-type virus genomes often have a certain nucleotide bias (e.g. HIV-1 is AT rich). Vector genomes tend to be less AT rich, which may affect packaging and/or post-maturation steps.
4. Expression of the NOI in viral vector production cells. (Over-)expressed protein may have an indirect or direct effect on vector virion assembly and/or infectivity.

It has been empirically shown that expression of the protein encoded by the NOI within viral vector production cells can adversely affect therapeutic vector titres (described in WO2015/092440).

Incorporation of a protein encoded by the NOI (the protein of interest, POI) into vector virions may also impact downstream processing of vector particles; for example, an NOI encoding a transmembrane POI may lead to high surface expression of the transmembrane protein in the viral vector virion, potentially altering the physical properties of the virions. Furthermore, this incorporation may present the POI to the patient's immune system at the site of delivery, which may negatively impact transduction and/or the long-term expression of the therapeutic gene in vivo. The NOI could also induce the production of undesirable secondary proteins or metabolites which could impact production, purification, recovery and immunogenicity and it is therefore desirable to minimise this.

The capability to repress the expression of a NOI in viral vector production cells while maintaining effective expression of the NOI in target cells is also desirable. Whatever mechanism is employed, the ‘natural’ pathway of assembly and resulting functionality of the viral vector particles must not be impeded. This is not straightforward because the viral vector genome molecule that will be packaged into virions must necessarily encode the NOI expression cassette. In other words, because the vector genome molecule and NOI expression cassettes are operably linked, modification of the NOI expression cassette may have adverse consequences on the ability to produce the vector genome molecule in the cell. For example, if a physical transcription block (e.g. TetR repressor system) is used to repress the NOI expression cassette it is likely that production of the vector genome molecule would also be inhibited through steric hindrance. In addition, control mechanism modifications must also not adversely affect the functionality of the vector genome molecule after virion maturation and release (i.e. with regards to directing transduction of the target cell). For example, a retroviral vector genome RNA molecule must be capable of the processes of reverse transcription and integration—any modification to the NOI expression cassette must not impede these steps in the transduction process.

Repression of NOI expression within viral vector production cells may present further advantages. If NOI expression leads to a reduction in the viability of vector production cells, its repression may benefit manufacturing at large scale which requires large cell numbers. The reduction in cell debris due to cell death would also reduce impurities within the crude vector harvest material. Processing, purification and concentration of the vector platform (i.e. different therapeutic genes encoded within the same vector system) could be standardised; if the only heterologous genes expressed within viral vector production cells are those required for vector production, downstream processing could be more easily optimised for an entire platform of therapeutic vectors, resulting in very similar physical specifications of vector preparations. Variability of immune response to, and toxicity of, resulting vectors in vivo may be minimised, which may lead to more persistent therapeutic NOI expression in the target cells.

Tissue-specific promoters which limit expression of the NOI in production cells are a possible solution to this problem, although leakiness of these promoters might lead to adverse levels of transgene protein. However, greater and more robust expression of the NOI in target cells can be achieved using constitutive promoters. Indeed, such robust expression may be required for efficacy in vivo. In addition, tissue-specific promoters may be less predictable when following a therapeutic vector product through animal models and into humans during pre-clinical and clinical development.

WO2015/092440 (incorporated herein by reference) discloses the use of a heterologous translation control system in eukaryotic cell cultures to repress the translation of the NOI (repress transgene expression) during viral vector production and thus repress or prevent expression of the protein encoded by the NOI. This system is referred to as the Transgene Repression In vector Production cell system or TRIP system. In one form, the TRIP system utilises the bacterial trp operon regulation protein, tryptophan RNA-binding attenuation protein (TRAP), and the TRAP binding site/sequence (tbs) to mediate transgene repression. Surprisingly, the use of this system does not impede the production of packageable vector genome molecules nor the activity of vector virions, and does not interfere with the long-term expression of the NOI in the target cell.

### SUMMARY OF THE INVENTION

The present invention relates to modifications made to the transgene mRNA that enable improved levels of translation repression by TRAP, which may be used to improve the TRIP system. Improved nucleic acid sequences of the present invention may, for example, have the following features:
1. Improved 5′UTR leader sequences (upstream of tbs) composed of nucleotides derived from the first (non-coding) exon of the EF1α gene are surprisingly shown to be able to allow consistently lower ‘repressed’ levels of transgene expression mediated by the TRAP-tbs complex compared to 5′UTR leader sequences from a variety of constitutive promoters.
2. Improved UTR or ‘spacer’ sequences inserted between an internal ribosome entry site (IRES) and the tbs are surprisingly shown to improve both fold-repression and non-repressed levels (i.e. without TRAP)
3. Variant Kozak sequences that overlap with the 3′ end of the tbs are surprisingly shown to lead to improved occlusion of the transgene initiation codon by the TRAP-tbs complex.
4. Sequences comprising compressed, overlapping multi-cloning sites between the tbs and the transgene Kozak sequence (transgene start codon (ATG)) are surprisingly shown to enable ease of cloning whilst retaining low levels of transgene expression when repressed by TRAP.
5. Overlap of the 3′ end of the tbs with the transgene start codon ATG is surprisingly shown to lead to improved occlusion of the transgene initiation codon by the TRAP-tbs complex.

In one aspect, the present invention provides a nucleic acid sequence comprising a nucleotide of interest, a tryptophan RNA-binding attenuation protein (TRAP) binding site and a Kozak sequence, wherein said TRAP binding site overlaps the Kozak sequence.

In another aspect, the present invention provides a nucleic acid sequence comprising a nucleotide of interest and a Kozak sequence, wherein said Kozak sequence comprises a portion of a tryptophan RNA-binding attenuation protein (TRAP) binding site.

In one aspect the present invention provides a nucleic acid sequence comprising a nucleotide of interest (transgene) and a TRAP binding site, wherein the TRAP binding site comprises a portion of the transgene start codon ATG or vice versa.

In some embodiments, the nucleotide of interest is operably linked to the TRAP binding site or the portion thereof.

In some embodiments, the TRAP binding site or the portion thereof is capable of interacting with tryptophan RNA-binding attenuation protein such that translation of the nucleotide of interest is repressed in a viral vector production cell.

In some embodiments, the nucleotide of interest is translated in a target cell which lacks the tryptophan RNA-binding attenuation protein.

In some embodiments, the TRAP binding site or the portion thereof comprises multiple repeats of the sequence KAGN2-3.

In some embodiments, the TRAP binding site or the portion thereof comprises multiple repeats of the sequence KAGN2.

In some embodiments, the TRAP binding site or the portion thereof comprises at least 6 repeats of the sequence KAGN2.

In some embodiments, the TRAP binding site or the portion thereof comprises at least 8 repeats of the sequence KAGN2-3. The number of KAGNNN repeats may be 1 or less.

In some embodiments, the TRAP binding site or the portion thereof comprises at least 8-11 repeats of the sequence KAGN2.

In some embodiments, the TRAP binding site or the portion thereof comprises 11 repeats of the sequence KAGN2-3, wherein the number of KAGNNN repeats is 3 or less.

In some embodiments, the Kozak sequence overlaps the 3′ terminal of the TRAP binding site or of the portion thereof. The Kozak sequence may overlap the 3′ terminal KAGNN repeat of the TRAP binding site or of the portion thereof.

In some embodiments, said Kozak sequence comprises the sequence RNNATG.

In some embodiments, said Kozak sequence comprises the sequence RVVATG.

In some embodiments, said overlapping Kozak sequence and TRAP binding site or portion thereof comprises one of the following sequences:

| (a) | GAGATG; |
|---|---|
| (b) | KAGVATG; |
| (c) | KAGVVATG; |
| (d) | KAGRVVATG; |
| or | |
| (e) | KAGNRVVATG. |

In some embodiments, said nucleic acid sequence comprises one of the following sequences:

| (a) | KAGCCGAGATG; |
|---|---|
| (b) | KAGGCGAGCATG; |
| (c) | KAGNGGAGCCATG; |
| or | |
| (d) | KAGNNGAGACCATG. |

In some embodiments, said nucleic acid sequence comprises one of the following sequences:

| (a) | KAGCCGAGATG; |
|---|---|
| or | |
| (b) | KAGNGGAGCCATG |

In some embodiments, said nucleic acid sequence comprises a sequence as set forth in SEQ ID NOs: 69-92 or 108-112.

In some embodiments, the distance between the transcription start site/end of promoter to start of the TRAP binding site or of the portion thereof is less than 34 nucleotides.

In some embodiments, the distance between the transcription start site/end of promoter to start of the TRAP binding site or of the portion thereof is less than 13 nucleotides.

In some embodiments, the TRAP binding site or the portion thereof lacks a type II restriction enzyme site, preferably a SapI restriction enzyme site.

In some embodiments, said nucleic acid sequence comprises a 5′ leader sequence upstream of the TRAP binding site or the portion thereof. The leader sequence may comprise a sequence derived from the non-coding EF1α exon 1 region. The leader sequence may comprise a sequence as defined in SEQ ID NO:25 or SEQ ID NO:26.

In some embodiments, said nucleic acid sequence comprises an internal ribosome entry site (IRES). Said nucleic acid sequence may comprise a spacer sequence between an internal ribosome entry site (IRES) and the TRAP binding site or the portion thereof. The spacer may be between 0 and 30 nucleotides in length. The spacer may be 15 nucleotides in length.

In some embodiments, the spacer is 3 or 9 nucleotides from the 3′ end of the TRAP binding site or the portion thereof and the downstream initiation codon of the nucleotide of interest.

In some embodiments, the spacer comprises a sequence as defined in any one of SEQ ID NOs:38-44, preferably the spacer comprises a sequence as defined in SEQ ID NO:38.

In some embodiments, the nucleotide of interest gives rise to a therapeutic effect.

In some embodiments, the nucleic acid sequence further comprises an RRE sequence or functional substitute thereof.

In some embodiments, said nucleic acid sequence is a vector transgene expression cassette.

In some embodiments, the 3′ terminal KAGNN repeat of the TRAP binding site or of the portion thereof overlaps at least the first nucleotide of the ATG start codon.

In some embodiments, the 3′ terminal KAGNN repeat of the TRAP binding site or of the portion thereof overlaps the first two nucleotides of the ATG start codon.

In some embodiments, the 3′ terminal KAGNN repeat of the TRAP binding site or of the portion thereof overlaps the first nucleotide of the ATG start codon within a core Kozak sequence as defined herein.

In some embodiments, wherein the nucleic acid sequence comprises a sequence as defined in SEQ ID NO: 114 or SEQ ID NO: 116.

In a further aspect, the invention provides a viral vector comprising the nucleic acid sequence of the invention.

In some embodiments, the viral vector comprises more than one nucleotide of interest and wherein at least one nucleotide of interest is operably linked to a TRAP binding site or a portion thereof as defined herein.

In some embodiments, the viral vector is derived from a retrovirus, adenovirus, adeno-associated virus, herpes simplex virus, vaccinia virus or baculovirus. The viral vector may be derived from a lentivirus. The viral vector may be derived from HIV-1, HIV-2, SIV, FIV, BIV, EIAV, CAEV or visna lentivirus.

In a further aspect, the present invention provides a viral vector production system comprising a set of nucleic acid sequences encoding the components required for production of the viral vector, wherein the vector genome comprises the nucleic acid sequence of the invention. The viral vector may be derived from a retrovirus, adenovirus or adeno-associated virus.

In some embodiments, the viral vector is a retroviral vector and the viral vector production system further comprises nucleic acid sequences encoding Gag and Pol proteins, the tryptophan RNA-binding attenuation protein, and Env protein, or functional substitutes thereof.

In some embodiments, the viral vector production system further comprises a nucleic acid sequence encoding rev or a functional substitute thereof.

In some embodiments, the viral vector is derived from a lentivirus. The viral vector may be derived from HIV-1, HIV-2, SIV, FIV, BIV, EIAV, CAEV or visna lentivirus.

In a further aspect, the invention provides a DNA construct for use in the viral vector production system of the invention comprising the nucleic acid sequence of the invention.

In a further aspect, the invention provides a DNA construct for use in the viral vector production system of the invention comprising a nucleic acid sequence encoding the tryptophan-RNA binding attenuation protein.

In a further aspect, the invention provides a set of DNA constructs for use in the viral vector production system of the invention comprising the DNA construct of the invention, a DNA construct encoding Gag and Pol proteins, and a DNA construct encoding Env protein, or functional substitutes thereof.

In some embodiments, the set of DNA constructs further comprise a DNA construct encoding a rev sequence or a functional substitute thereof.

In a further aspect, the invention provides a viral vector production cell comprising the nucleic acid sequence of the invention, the viral vector production system of the invention or the DNA constructs of the invention.

In some embodiments, the cell is transiently transfected with a vector encoding a tryptophan-RNA binding attenuation protein. The cell may stably express a tryptophan-RNA binding attenuation protein.

In a further aspect, the invention provides a process for producing viral vectors comprising introducing the nucleic acid sequence of the invention, the viral vector production system of the invention or the DNA constructs of the invention into a viral vector production cell and culturing the production cell under conditions suitable for the production of the viral vectors.

In a further aspect, the invention provides a viral vector produced by the viral vector production system of the invention, using the viral vector production cell of the invention or by the process of the invention.

In some embodiments, the viral vector comprises the nucleic acid sequence of the invention.

In some embodiments, the viral vector is derived from a retrovirus, adenovirus or adeno-associated virus. The viral vector may be derived from a lentivirus. The viral vector may be derived from HIV-1, HIV-2, SIV, FIV, BIV, EIAV, CAEV or visna lentivirus.

In a further aspect, the invention provides a cell transduced by the viral vector of the invention.

In a further aspect, the invention provides the viral vector of the invention or the cell of the invention for use in medicine.

In a further aspect, the invention provides the use of the viral vector of the invention or the cell of the invention for the preparation of a medicament to deliver a nucleotide of interest to a target site in need of the same.

In a further aspect, the invention provides a method of treatment comprising administering the viral vector of the invention or the cell of the invention to a subject in need of the same.

In a further aspect, the invention provides a pharmaceutical composition comprising the viral vector of the invention or the cell of the invention in combination with a pharmaceutically acceptable carrier, diluent or excipient.

In a further aspect, the invention provides a method of identifying nucleic acid binding sites and/or nucleic acid binding proteins which are capable of interacting such that the translation of a nucleotide of interest is repressed in a viral vector production cell when operably linked to the nucleic acid binding site, wherein the method comprises analysing the expression of a reporter gene in a cell comprising both the nucleic acid binding site operably linked to the reporter gene and the nucleic acid binding protein.

In some embodiments, the reporter gene encodes a fluorescent protein.

In a further aspect, the invention provides a method of repressing translation of a nucleotide of interest (NOI) in a viral vector production cell, the method comprising introducing into the viral vector production cell the nucleic acid sequence of the invention, and a nucleic acid sequence encoding a tryptophan-RNA binding attenuation protein (TRAP), wherein the TRAP binds to the TRAP binding site, or the portion thereof, thereby repressing translation of the NOI.

In a further aspect, the invention provides a method of increasing viral vector titers in a eukaryotic vector production cell, the method comprising introducing into the eukaryotic vector production cell the viral vector production system of the invention and a nucleic acid sequence encoding a tryptophan-RNA binding attenuation protein (TRAP), wherein the TRAP binds to the TRAP binding site, or the portion thereof, and represses translation of the NOI, thereby increasing viral vector titres relative to a viral vector having no TRAP binding site.

In a further aspect, the invention provides a nucleic acid sequence comprising a nucleotide of interest, a binding site for tryptophan RNA-binding attenuation protein (TRAP), a multiple cloning site and a Kozak sequence, wherein said multiple cloning site is located downstream of the TRAP binding site and upstream of the Kozak sequence.

In some embodiments, the nucleic acid sequence of the invention further comprises a promoter. The TRAP binding site or portion thereof and Kozak sequence or the TRAP binding site, multiple cloning site and Kozak sequence may be located within the 5′ UTR of the promoter.

In some embodiments, the promoter further comprises an intron, preferably wherein the intron is upstream of the TRAP binding site or portion thereof. The promoter may be an engineered promoter comprising a heterologous intron within the 5′ UTR.

In some embodiments, the nucleic acid sequence of the invention comprises a sequence as set forth in any of SEQ ID NO: 117, 118 or 120 to 124.

In a further aspect, the invention provides a nucleic acid sequence encoding the RNA genome of a viral vector, wherein the RNA genome of the viral vector comprises a nucleic acid sequence as described herein.

In some embodiments, the nucleic acid sequence of the invention as described herein is comprised within an RNA genome of a viral vector.

In some embodiments, the nucleic acid sequence of the invention as described herein is operably linked to a nucleotide sequence encoding the RNA genome of a viral vector.

In some embodiments of the nucleic acid sequence of the invention as described herein or the viral vector production system of the invention as described herein wherein the major splice donor site in the RNA genome of the viral vector is inactivated.

In some embodiments, the major splice donor site and the cryptic splice donor site 3′ to the major splice donor site in the RNA genome of the viral vector are inactivated.

In some embodiments, the cryptic splice donor site is the first cryptic splice donor site 3′ to the major splice donor site.

In some embodiments, said cryptic splice donor site or sequence is within 6 nucleotides of the major splice donor site or sequence.

In some embodiments, the major splice donor site and cryptic splice donor site are mutated or deleted.

In some embodiments, the nucleotide sequence encoding the RNA genome of the viral vector prior to inactivation of the splice sites comprises a sequence as set forth in any of SEQ ID NOs: 94, 96, 97, 102, 103 and/or 106.

In some embodiments, the nucleotide sequence encoding the RNA genome of the viral vector comprises a sequence with a mutation or deletion relative to the sequence as set forth in any of SEQ ID NOs: 94, 96, 97, 102, 103 and/or 106.

In some embodiments, the nucleotide sequence encoding the RNA genome of the viral vector comprises an inactivated major splice donor site which would otherwise have a cleavage site between nucleotides corresponding to nucleotides 13 and 14 of SEQ ID NO:94.

In some embodiments, the nucleotide sequence of the major splice donor site prior to inactivation comprises the sequence as set forth in SEQ ID NO: 97.

In some embodiments, the nucleotide sequence of the cryptic splice donor site prior to inactivation comprises the sequence as set forth in SEQ ID NO: 103.

In some embodiments, the nucleotide sequence encoding the RNA genome of the viral vector comprises an inactivated cryptic splice donor site which would otherwise have a cleavage site between nucleotides corresponding to nucleotides 17 and 18 of SEQ ID NO:94.

In some embodiments, the nucleotide sequence encoding the RNA genome of the viral vector comprises a sequence as set forth in any of SEQ ID NOs: 95, 98, 99, 100, 101,104, 105 and/or 107.

In some embodiments, the nucleotide sequence encoding the RNA genome of the viral vector does not comprise a sequence as set forth in SEQ ID NO:102.

In some embodiments, the splicing activity from the major splice donor site and cryptic splice donor site of the RNA genome of the viral vector is suppressed or ablated.

In some embodiments, the splicing activity from the major splice donor site and cryptic splice donor site of the RNA genome of the viral vector is suppressed or ablated in transfected cells or in transduced cells.

In some embodiments, the viral vector is derived from a lentivirus.

### DESCRIPTION OF THE DRAWINGS

FIG. 1. Overview of improvements to 5′UTR sequences upstream of the tbs. A schematic to indicate the position of the 5′leader sequence derived from EF1a gene exon 1, placed upstream of a tbs sequence ([KAGNN]×11) within a transgene 5′UTR. Surprisingly, it is found that when compared to leader sequences from other promoters, use of such a leader sequence leads to improved levels of repression of the transgene by TRAP-tbs (TRAP denoted by doughnut shape). Without wishing to be bound by theory, it is presumed that such a leader sequence enables a more stable TRAP-tbs complex such that inhibition of ribosome scanning is maximised.

FIG. 2. Overview of improvements to 5′UTR sequences downstream of the tbs.

A. A schematic to show the DNA expression cassette of the 5′UTR encoding region of a TRAP-tbs repressible transgene cassette wherein a multicloning site (MCS) is inserted between the tbs and the initiation codon of the transgene (TRAP denoted by doughnut shape). The invention describes preferred, overlapping restriction enzymes sites that begin at/on the terminal KAGNN repeat of the tbs and contains up to five cloning sites upstream of the transgene initiation codon.

B. A schematic to show how the Kozak sequence of the transgene can be positioned such that it mostly or partially overlaps with the 3′ KAGNN repeat of the tbs; doing so can effectively ‘hide’ the main initiation codon within the TRAP-tbs complex making it even less accessible to translation machinery, leading to even lower ‘repressed’ levels of transgene expression.

C. A table to summarise preferred overlapping tbs and Kozak consensus sequences. The 3′ KAGNN repeat of the tbs is shown boxed and the core Kozak sequence is shown in bold.

FIG. 3. Enhanced repression by TRAP-tbs when using the L33 Improved leader sequence compared to a variety of constitutive promoters containing native UTR sequences.

A. A schematic indicating the organization of the GFP test reporter plasmids. The 5′UTR comprised the identical tbs sequence and other elements, except for the different promoters utilized, and the different leader sequences upstream of the tbs (these are shown in Panel I of Table I). Note (as per Table I), the intron-containing EF1a (EF1a) and UBC promoters considered directly comparable to their ‘short’, intron-less counterparts EFS and UBCs respectively because the intron sequences will not be present in mRNA. The 34nt leader was present within the CMV promoter-containing reporter as a control (this was previously been shown to enable >100-fold repression by TRAP-tbs). The other constitutive promoters contained leaders that comprised native leader sequence as well as some synthetic sequence, or were engineered in this study to harbor the L33 Improved leader sequence, which is derived from Exon 1 of the EF1a promoter.

B. The reporters were tested for non-repressed or repressed levels of GFP expression by co-transfection of the reporter plasmids with either pBlueScript (No TRAP) or pEF1α-TRAP (TRAP), respectively. Transfected HEK293T cells (suspension, serum-free) were analysed by flow cytometry two days post-transfection, and GFP Expression scores (% GFP×median fluorescence intensity) generated and log-10 transformed. The data in the chart is displayed by general promoter strength (No TRAP levels) from left to right, and compared to Mock transfected (pBlueScript only) or untransfected cells (UNT). In each case where the native leader is compared to the L33 Improved leader, the L33 Improved leader enables substantially lower levels of GFP expression in the presence of TRAP—enabling >10-fold improvement in repression by TRAP-tbs. In most cases, the non-repressed levels of GFP expressed were also slightly improved when using the L33 Improved leader. (Standard deviation bars, n=3).

FIG. 4. Design and evaluation of multicloning sites (MCS) inserted between the tbs and the Kozak sequence of the transgene cassette within an AAV vector genome, and the impact on transgene repression by TRAP-tbs.

A. A schematic indicating the MCS variants tested with the 5′UTR-tbs sequences; the seven variants contained 2-4 cloning sites not including the NcoI site, which is dependent on the presence of a certain Kozak sequence and the first nucleotide of the second codon of the transgene (and may therefore not necessarily be present in all transgene cassettes). The MCS variant reporter constructs were driven by the EFS promoter and contained the L33 Improved leader, whereas the ‘No MCS’ control reporter construct was driven by the CMV promoter and harbored the original 34nt leader (shown in FIG. 4 to perform similarly to the L33 Improved leader). The transgene cassettes were cloned into a scAAV2 vector genome plasmid (ITRs not shown).

B. The reporter AAV genome plasmids were tested for non-repressed or repressed levels of GFP expression by co-transfection of the reporter plasmids with either pBlueScript (No TRAP) or pEF1α-TRAP (TRAP), respectively. Transfected HEK293T cells (suspension, serum-free) were analysed by flow cytometry two days post-transfection, and GFP Expression scores (% GFP×median fluorescence intensity) generated and log-10 transformed. All the MCS variant reporters were repressed by TRAP-tbs by ˜1000-fold or greater and six of the seven variants were at least 10 fold better at reducing transgene levels. Furthermore, variants MCS2.1, MCS4.1 and MCS4.4 allowed TRAP-tbs to repress GFP levels to the limit of detection (compared to untransfected [UNT]). (Standard deviation bars, n=3).

FIG. 5. Demonstration of consistently potent transgene repression by TRAP-tbs using transgene cassettes driven by different constitutive promoters with 5′UTRs harboring the L33 Improved leader, tbs and optimized multicloning sites. A variety of constitutive promoters were cloned into the MCS2.1-GFP and MCS4.1-GFP scAAV2 reporter genome plasmids, containing the L33-tbs sequence. The reporters were tested for non-repressed or repressed levels of GFP expression by co-transfection of the reporter plasmids with either pBlueScript (No TRAP) or pEF1α-TRAP (TRAP), respectively. Transfected HEK293T cells (suspension, serum-free) were analysed by flow cytometry two days post-transfection, and GFP Expression scores (% GFP×median fluorescence intensity) generated and log-10 transformed. The data demonstrates that the L33 Improved leader and optimized MCS sites can be incorporated into the same cassette, and allow a high degree of transgene repression by TRAP-tbs in the context of a wide range of constitutive promoters. Average repression level across the experiment was ˜5000-fold. (Standard deviation bars, n=3).

FIG. 6. Identification of optimal Kozak sequences that overlap with the 3′end of the tbs within transgene 5′UTR in order to position the tbs closer to the ATG initiation codon.

A. A schematic indicating the position and sequence of Kozak sequences in engineered variants conforming to the core consensus ‘RVVATG’ and the broader consensus ‘GNNRVVATG’ positioned in such a manner that the Kozak overlaps with the 3′end of the tbs so that KAGNN repeat(s) are maintained. This allows the tbs to be positioned closer to the ATG initiation codon to identify tbs-Kozak junction variants that enable improved transgene repression levels (+TRAP) by ‘hiding’ the ATG initiation codon within the TRAP-tbs complex. The maintenance of the consensus Kozak sequence enables transgene non-repressed levels (No TRAP) to be retained at high levels (i.e. modelling expression in vector-transduced cells).

B. The reporters were tested for non-repressed or repressed levels of GFP expression by co-transfection of the reporter plasmids with either pBlueScript (No TRAP) or pEF1α-TRAP (TRAP), respectively into HEK293T cells. Transfected cells were analysed by flow cytometry two days post-transfection, and GFP Expression scores (% GFP×median fluorescence intensity) generated and log-10 transformed. All the tbs-Kozak junction variant reporters maintained the same non-repressed GFP levels compared to the original configuration. Variants ‘0’, ‘2’ and ‘3’ displayed improved repressed levels compared to the original configuration (Standard deviation bars, n=3).

FIG. 7. Identification of an improved spacer sequence between an IRES and tbs sequence to impart better repression of IRES-dependent transgenes by TRAP-tbs.

A. A schematic to indicate the transgene cassette configuration in testing spacer sequences. pCMV-Luciferase-IRES-(spacer)-tbs-GFP reporter constructs were designed (see Table III) and tested.

B. Reporters containing the Original [26nt] spacer or a Variant [26nt] or truncations of the two spacers were tested. The reporters were tested for non-repressed or repressed levels of GFP expression by co-transfection of the reporter plasmids with either pBlueScript (No TRAP) or pEF1α-TRAP (TRAP), respectively into HEK293T cells. Transfected cells were analysed by flow cytometry two days post-transfection, and GFP Expression scores (% GFP×median fluorescence intensity) generated and log-10 transformed. The study revealed the ‘Original [trunc-15nt]’ spacer as a variant with improved ‘ON’ and reduced ‘OFF’ levels compared to the Original spacer.

C The ‘Original [trunc-15nt]’ spacer was then incorporated with reporters harbouring either an 11×KAGNN repeat tbs or an 8×KAGNN repeat tbs and with either a 9nt or 3nt distance from the 3′end of the tbs and the downstream transgene ATG initiation codon. GFP expression was measured as before. The data indicate that improved ‘Original [trunc-15nt]’ spacer could be used with different tbs configuration and proximity to the main transgene ATG initiation codon (Standard deviation bars, n=3).

FIG. 8. Comparison of two Improved leaders derived from the EF1a Exon 1 sequence. A truncated leader 112′ was derived from the L33 Improved leader sequence, which comprises Exon 1 from the human EF1a gene (see Table I). The L12 Improved leader was cloned into six constitutive promoter-containing GFP reporter cassettes within scAAV2 vector genome plasmids, harboring either the MCS2.1 or MCS4.1 sequences between the tbs and the Kozak sequence. The reporters were tested for non-repressed or repressed levels of GFP expression by co-transfection of the reporter plasmids with either pBlueScript (No TRAP) or pEF1α-TRAP (TRAP), respectively. Transfected HEK293T cells (suspension, serum-free) were analysed by flow cytometry two days post-transfection, and GFP Expression scores (% GFP×median fluorescence intensity) generated and log-10 transformed. The data demonstrate that the L12 and L33 Improved leaders allow full repression by TRAP-tbs, with GFP levels repressed to background levels. Interestingly the ‘ON’ (no repressed) levels of GFP were slightly higher for L12 or L33 in different promoters; this allows flexibility in being able to choose between either L12 or L33 when considering utilization of the TRIP system with different promoters, so that gene expression levels in the absence of TRAP (i.e. in vector transduced cells) can be maximized, without losing the substantial levels of repression achieved by TRAP-tbs during vector production. (Standard deviation bars, n=3).

FIG. 9. Improvement in transgene repression in AAV vector genome plasmids by employing overlapping tbs-Kozak variants. Two ‘tbs-Kozak’ variants (0 and 3) were cloned into either EFS or huPGK promoter GFP reporter cassettes, additionally containing either the L33 or L12 Improved leader sequences. Non-overlapping tbs/Kozak variants were also cloned into EFS/huPGK-L33 cassettes; these differed only in the tbs-Kozak region (Original=[tbs]-ACAGCCACCATG; HpaI variant=[tbs-GAGTT]AACGCCACCATG). The reporters were tested for non-repressed or repressed levels of GFP expression by co-transfection of the reporter plasmids with either pBlueScript (No TRAP) or pEF1α-TRAP (TRAP), respectively. Transfected HEK293T cells (suspension, serum-free) were analysed by flow cytometry two days post-transfection, and GFP Expression scores (% GFP×median fluorescence intensity) generated and log-10 transformed. The data demonstrates that overlapping the tbs with the Kozak sequence allows improved repression of transgene expression by TRAP compared to the non-overlapping tbs/Kozak variants. (Standard deviation bars, n=3).

FIG. 10. Improvement in TRAP-mediated transgene repression in the context of the full length EF1a promoter.

A. Three ‘tbs-Kozak’ variants (0, 2 and 3) were cloned into an EF1a promoter GFP reporter cassette. After splicing the leader sequence comprises the L33 sequence (exon1) and a short 12nt sequence from exon2 immediately upstream of the tbs.

B. The reporters were tested for non-repressed or repressed levels of GFP expression by co-transfection of the reporter plasmids with either pBlueScript (No TRAP) or pEF1α-TRAP

(TRAP), respectively. Transfected HEK293T cells (suspension, serum-free) were analysed by flow cytometry two days post-transfection, and GFP Expression scores (% GFP×median fluorescence intensity) generated and log-10 transformed.

C. The GFP transgene cassettes were cloned into an HIV-1 lentiviral vector genome, and tested for non-repressed or repressed levels of GFP expression as described in B. The data demonstrates that overlapping the tbs with the Kozak sequence allows improved repression of transgene expression by TRAP. (Standard deviation bars, n=3).

FIG. 11: Aberrantly spliced mRNA expressing transgene during lentiviral vector production is abolished in MSD-2KO lentiviral vectors, reducing the amount of transgene mRNA required to be targeted by TRAP when utilising the TRiP system. A A schematic of a TRiP′ lentiviral vector genome encoding an EF1a-GFP transgene cassette, wherein the TRAP binding site (tbs) is positioned within the 5′UTR of the cassette (supply of TRAP during vector production reduces transgene expression levels). During production of MSD-2KO lentiviral vectors, the full length, unspliced packagable vRNA and transgene mRNA are the main forms of cytoplasmic RNA produced from the lentiviral vector cassette (i) (when the transgene promoter is active during production). However, the promiscuous activity of the MSD in standard lentiviral vector genomes leads to additional ‘aberrant’ splice products that may encode the transgene (ii); this could occur independently of the internal transgene promoter i.e. a tissue specific promoter. (Key: Pro, promoter; region from 5′R to gag contains the packaging element {ψ}; msd, major splice donor; cppt, central polypurine tract; Int, intron; sd/sa, splice donor/acceptor; GOI, gene of interest; grey arrow indicate position of forward {f} and reverse {r} primers to assess the proportion of Unspliced vRNA produced during 3rd generation lentiviral vector production. Post-transcriptional regulatory element {PRE} not shown for clarity). B Standard or MSD-2KO lentiviral vector genome plasmids containing an EF1a-GFP cassette were used to produce lentiviral vectors in HEK293T cells, and GFP expression scores generated (% GFP×MFI). Relative to the total amount of GFP produced in cultures during standard lentiviral vector production, the MSD-2KO modification had the substantial effect of reducing the amount of GFP produced even in the absence of TRAP (˜5-fold). Accordingly, the repressive effects of TRAP were augmented by use of the MSD-2KO lentiviral vector genome, leading to much lower levels of GFP in cultures. C The sequence of the stem loop 2 (SL2) region of ‘wild type’ HIV-1 (NL4-3; the ‘Standard’ sequence within current lentiviral vector genomes) is shown at the top. The sequence comprises the major splice donor site (MSD: consensus=CTGGT) and a cryptic splice donor site (that is utilized when the MSD site is mutated on its own (crSD: consensus=TGAGT). The nucleotides at the position of splicing when the splice donor site is used are identified in bold and by arrows. Four functional MSD mutations that ablate both the MSD and the crSD site splicing activities are described: MSD-2KO, which mutates the two ‘GT’ motifs from the MSD and the crSD sites (and is used widely in most Examples); MSD-2KOv2, which also comprises mutations that ablate both the MSD and crSD sites; MSD-2KOm5, which introduces an entirely new stem-loop structure lacking any splice donor sites; and ΔSL2, which deletes the SL2 sequence entirely. The substitutions introduced to the SL2 sequence in the MSD-2KO, MSD-2KOv2 and MSD-2KOm5 mutations are shown in lowercase italics.

FIG. 12: Implications of aberrant splicing from the major splice donor site (MSD) within HIV-1 based lentiviral vectors. Standard 3^{1d} generation lentiviral vector production was performed +/−rev in HEK293T cells and total RNA extracted from post-production cells. Total RNA was subjected to qPCR (SYBR green) using two primer sets: f+rT amplified total transcripts generated from the lentiviral vector expression cassette, and f+rUS amplified Unspliced transcripts; therefore the proportion of Unspliced-to-Total vRNA transcripts were calculated and plotted. The data indicates that the proportion of Unspliced vRNA relative to total during standard 3^{1d} generation lentiviral vector production is modest and varies according to the internal transgene cassette (in this case containing different promoters and the GFP gene); moreover, this proportion is only minimally increased by the action of rev.

FIG. 13. Testing TRAP-mediated transgene repression of overlapping tbs-Kozak variants in suspension (serum-free) HEK293T cells. The overlapping tbs-Kozak variants in Table IV were cloned into a pEF1α-GFP reporter plasmid and transfected into HEK293T cells+/−pTRAP, and flow cytometry performed 2 days post-transfection. A. GFP Expression scores (% GFP positive×MFI) were generated +/−TRAP and fold repression values generated and plotted. The variants are indicated along the x-axis, grouped according to the relative overlap of the 3′ tbs KAGNN repeat and the core Kozak sequence (overlap groups'-KAGatg, KAGNatg), KAGNNatg); the KAGNN is denoted by the black bracket and the core Kozak nucleotides by the grey line. Statistical analysis was performed comparing the following overlap groups (equal variance within overlap groups was confirmed by F-Test): fold-repression was statistically greater for KAGatg over KAGNatg (*p=0.0293); for KAGNatg over KAGNNatg (**p=0.00000482); and for KAGNNatg over the non-overlapping tbs (***p=0.000259), using two-tail T-Test. B. The non-repressed GFP Expression scores were plotted highest to lowest (left to right), and the two KAGatg overlap group variants tbskzkV0.G and tbskzkV0.T (showing greatest repression of all the variants in A) highlighted to show that the ‘G’ variant is preferred over the ‘T’ variant due to the former having the better ‘ON’ (non-repressed) levels.

FIG. 14. Improved repression of intron-containing promoters using an optimal overlapping tbs-Kozak variant. A. A schematic of expression cassettes used to exemplify the use of an overlapping tbs-Kozak variant compared to a non-overlapping tbs-Kozak variant. The widely used EF1a promoter sequence contains its own intron (see FIG. 10 and Example 5), as does the widely used CAG promoter. The CAG promoter is a very strong artificial promoter containing the CMV enhancer, the core promoter and exon1/intron sequence from the Chicken β-actin gene and the splice acceptor/exonic sequence from the Rabbit β-globin gene. In this work, the ‘EF1a-INT’ sequence from the EF1a promoter (containing exon 1 [L33]), all of the EF1a intron and splice acceptor, and 12 nucleotides from EF1a exon 2, was cloned into the CAG promoter, replacing the CAG exon/intron sequences. The ‘EF1a-INT’ sequence was also cloned into a CMV promoter construct. B. The constructs were evaluated for GFP expression and repression by TRAP in suspension (serum-free) HEK293T cells to model transgene expression during viral vector production. GFP Expression scores (% GFP×MFI) were generated and plotted, as well as fold-repression scores denoted in the presence of TRAP.

### DETAILED DESCRIPTION OF THE INVENTION

Various preferred features and embodiments of the present invention will now be described by way of non-limiting examples.

The practice of the present invention will employ, unless otherwise indicated, conventional techniques of chemistry, molecular biology, microbiology and immunology, which are within the capabilities of a person of ordinary skill in the art. Such techniques are explained in the literature. See, for example, J. Sambrook, E. F. Fritsch, and T. Maniatis (1989) Molecular Cloning: A Laboratory Manual, Second Edition, Books 1-3, Cold Spring Harbor Laboratory Press; Ausubel, F. M. et al. (1995 and periodic supplements) Current Protocols in Molecular Biology, Ch. 9, 13, and 16, John Wiley & Sons, New York, N.Y.; B. Roe, J. Crabtree, and A. Kahn (1996) DNA Isolation and Sequencing: Essential Techniques, John Wiley & Sons; J. M. Polak and James O'D. McGee (1990) In Situ Hybridization: Principles and Practice; Oxford University Press; M. J. Gait (ed.) (1984) Oligonucleotide Synthesis: A Practical Approach, IRL Press; and, D. M. J. Lilley and J. E. Dahlberg (1992) Methods of Enzymology: DNA Structure Part A: Synthesis and Physical Analysis of DNA Methods in Enzymology, Academic Press. Each of these general texts is herein incorporated by reference.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

Nucleic Acid Sequence

In one aspect, the present invention provides a nucleic acid sequence comprising a nucleotide of interest, a tryptophan RNA-binding attenuation protein (TRAP) binding site and a Kozak sequence, wherein said TRAP binding site (tbs) overlaps the Kozak sequence.

In one aspect the present invention provides a nucleic acid sequence comprising a nucleotide of interest (transgene) and a tryptophan RNA-binding attenuation protein (TRAP) binding site (tbs), wherein the TRAP binding site overlaps with the transgene start codon ATG.

In another aspect, the present invention provides a nucleic acid sequence comprising a nucleotide of interest and a Kozak sequence, wherein said Kozak sequence comprises a portion of a tryptophan RNA-binding attenuation protein (TRAP) binding site (tbs).

In one aspect the present invention provides a nucleic acid sequence comprising a nucleotide of interest (transgene) and a TRAP binding site, wherein the TRAP binding site (tbs) comprises a portion of the transgene start codon ATG or vice versa.

In some embodiments of the present invention, the nucleotide of interest is operably linked to the tbs or the portion thereof. In some embodiments, the nucleotide of interest is translated in a target cell which lacks TRAP.

The tbs or the portion thereof may be capable of interacting with TRAP such that translation of the nucleotide of interest is repressed or prevented in a viral vector production cell.

Thus, in another aspect, the present invention provides a method of repressing translation of a NOI in a viral vector production cell, the method comprising introducing into the viral vector production cell the nucleic acid sequence of the invention and a nucleic acid sequence encoding a TRAP, wherein the TRAP binds to the TRAP binding site, or the portion thereof, thereby repressing translation of the NOI.

Tryptophan RNA-Binding Attenuation Protein (TRAP)

Tryptophan RNA-binding attenuation protein (TRAP) is a bacterial protein that has been extensively characterised in Bacillus subtilis. It regulates tryptophan biosynthesis directed from the trpEDCFBA operon by participating in either transcription attenuation or translational control mechanisms (reviewed in Gollnick, B., Antson, and Yanofsky (2005) Annual Review of Genetics 39: 47-68).

In its natural context TRAP regulates tryptophan biosynthesis and transport by three distinct mechanisms:
1. Attenuation of transcription of the trpEDCFBA operon (Shimotsu H, K. M., Yanofsky C, Henner D J. (1986) Journal of Bacteriology 166: 461-471).
2. Promotion of formation of the trpE and trpD Shine-Dalgarno blocking hairpin (Yakhnin H, B. J., Yakhnin A V, Babitzke P. (2001) Journal of Bacteriology 183(20): 5918-5926).
3. Blocking ribosome access to the trpG and yhaG ribosome binding sites (Yang M, d. S. A., van Loon A P G M, Gollnick P. (1995) Journal of Bacteriology 177: 4272-4278).

In Bacillus subtilis TRAP is encoded by a single gene (mtrB) and the functional protein is composed of 11 identical subunits arranged as a toroid ring (Antson A A, D. E., Dodson G, Greaves R B, Chen X, Gollnick P. (1999) Nature 401(6750): 235-242). It is activated to interact with RNA by binding up to 11 molecules of tryptophan in pockets between neighbouring subunits. The target RNA is wound around the outside of this quaternary ring structure (Babitzke P, S. J., Shire S J, Yanofsky C. (1994) Journal of Biological Chemistry 269: 16597-16604).

Without wishing to be bound by theory, in the natural mechanism of sensing and controlling tryptophan synthesis, TRAP is understood to act at the level of transcription termination by binding to a binding site in the newly synthesised RNA leader. This destabilises an overlapping anti-terminator sequence such that a downstream rho-independent terminator is active, leading to the production of only short RNAs. When tryptophan is limiting within the bacterium, the TRAP ring can no longer bind to its RNA binding site. Accordingly, the anti-terminator is activated and transcription continues into the tryptophan synthesis gene operon. TRAP can also act at the translational level: tryptophan-dependent binding of TRAP to its binding site in the 5′-UTR of the RNA transcript liberates an anti-Shine-Dalgarno sequence, this forms a stable stem with the Shine-Dalgarno sequence so that ribosome initiation of translation is repressed. Finally, in other contexts when TRAP is bound to its tbs it is capable of repressing translation initiation by physically blocking the 40S scanning ribosome complex before it can reach the initiation codon, whereupon the more stable and higher-affinity translation machinery would otherwise form.

The TRAP open-reading frame may be codon-optimised for expression in mammalian (e.g. Homo sapiens) cells, since the bacterial gene sequence is likely to be non-optimal for expression in mammalian cells. The sequence may also be optimised by removing potential unstable sequences and splicing sites. The use of a HIS-tag C-terminally expressed on the TRAP protein appears to offer a benefit in terms of translation repression and may optionally be used. This C-terminal HIS-tag may improve solubility or stability of the TRAP within eukaryotic cells, although an improved functional benefit cannot be excluded. Nevertheless, both HIS-tagged and untagged TRAP allowed robust repression of transgene expression.

Certain cis-acting sequences within the TRAP transcription unit may also be optimised; for example, EF1a promoter-driven constructs enable better repression with low inputs of TRAP plasmid compared to CMV promoter-driven constructs in the context of transient transfection.

In one embodiment, the TRAP is derived from a bacteria.

In one embodiment of the present invention, TRAP is derived from a Bacillus species, for example Bacillus subtilis. For example, TRAP may comprise the sequence:

(SEQ ID NO: 1)
MNQKHSSDFVVIKAVEDGVNVIGLTRGTDTKFHHSEKLDKGEVIIAQFT
EHTSAIKVRGEALIQTAYGEMKSEKK

In a preferred embodiment of the present invention, SEQ ID NO: 1 is C-terminally tagged with six histidine amino acids (HIS×6 tag).

In an alternative embodiment, TRAP is derived from Aminomonas paucivorans. For example, TRAP may comprise the sequence:

(SEQ ID NO: 2)
MKEGEEAKTSVLSDYVVVKALENGVTVIGLTRGQETKFAHTEKLDDGEV
WIAQFTEHTSAIKVRGASEIHTKHGMLFSGRGRNEKG

In an alternative embodiment, TRAP is derived from Desulfotomaculum hydrothermale. For example, TRAP may comprise the sequence:

(SEQ ID NO: 3)
MNPMTDRSDITGDYVVVKALENGVTIIGLTRGGVTKFHHTEKLDKGEIM
IAQFTEHTSAIKIRGRAELLTKHGKIRTEVDS

In an alternative embodiment, TRAP is derived from B. stearothermophilus. For example, TRAP may comprise the sequence:

(SEQ ID NO: 4)
MYTNSDFVVIKALEDGVNVIGLTRGADTRFHHSEKLDKGEVLIAQFTEH
TSAIKVRGKAYIQTRHGVIESEGKK

In an alternative embodiment, TRAP is derived from B. stearothermophilus S72N. For example, TRAP may comprise the sequence:

(SEQ ID NO: 5)
MYTNSDFVVIKALEDGVNVIGLTRGADTRFHHSEKLDKGEVLIAQFTEHT
SAIKVRGKAYIQTRHGVIENEGKK

In an alternative embodiment, TRAP is derived from B. halodurans. For example, TRAP may comprise the sequence:

(SEQ ID NO: 6)
MNVGDNSNFFVIKAKENGVNVFGMTRGTDTRFHHSEKLDKGEVMIAQFT
EHTSAVKIRGKAIIQTSYGTLDTEKDE

In an alternative embodiment, TRAP is derived from Carboxydothermus hydrogenoformans. For example, TRAP may comprise the sequence:

(SEQ ID NO: 7)
MVCDNFAFSSAINAEYIVVKALENGVTIMGLTRGKDTKFHHTEKLDKGE
VMVAQFTEHTSAIKIRGKAEIYTKHGVIKNE

In one embodiment, TRAP is encoded by the tryptophan RNA-binding attenuation protein gene family mtrB (TrpBP superfamily e.g. with NCBI conserved domain database #cl03437).

In preferred embodiments, the TRAP is C-terminally tagged with six histidine amino acids (HIS×6 tag).

In a preferred embodiment, TRAP comprises an amino acid sequence that has 50%, 60%, 70%, 80%, 90%, 95%, 99% or 100% identity to any of SEQ ID NOs: 1 to 7 and is capable of interacting with an RNA-binding site such that expression of an operably linked NOI is modified, for example repressed or prevented, in a viral vector production cell.

In a preferred embodiment, TRAP comprises an amino acid sequence that has at least 50%, 55%, 60%, 65%, 70%, 75%, 80%, 85%, 90%, 95%, 97%, 99% or 100% identity to any of SEQ ID NOs: 1 to 7 and is capable of interacting with an RNA-binding site such that expression of an operably linked NOI is modified, for example repressed or prevented, in a viral vector production cell.

In another embodiment, TRAP may be encoded by a polynucleotide comprising a nucleotide sequence which encodes a protein which is capable of interacting with an RNA-binding site such that expression of an operably linked NOI is modified, for example repressed or prevented, in a viral vector production cell. For example, TRAP may be encoded by a polynucleotide comprising a nucleotide sequence which encodes a protein of SEQ ID NOs: 1 to 7.

All variants, fragments or homologues of TRAP for use in the invention will retain the ability to bind to the TRAP binding site as described herein such that translation of the NOI (which may be a marker gene) is repressed or prevented in a viral vector production cell.

TRAP Binding Site

The term “binding site” is to be understood as a nucleic acid sequence that is capable of interacting with a certain protein.

A consensus TRAP binding site sequence that is capable of binding TRAP is [KAGNN] repeated multiple times (e.g. 6, 7, 8, 9, 10, 11, 12 or more times); such sequence is found in the native trp operon. In the native context, occasionally AAGNN is tolerated and occasionally additional “spacing” N nucleotides result in a functional sequence. In vitro experiments have demonstrated that at least 6 or more consensus repeats are required for TRAP-RNA binding (Babitzke P, Y. J., Campanelli D. (1996) Journal of Bacteriology 178(17): 5159-5163). Therefore, preferably in one embodiment there are 6 or more continuous [KAGN_{≥2}] sequences present within the tbs, wherein K may be T or G in DNA and U or G in RNA.

For TRAP as the RNA-binding protein, preferably the TRIP system works maximally with a tbs sequence containing at least 8 KAGNN repeats, although 7 repeats may be used to still obtain robust transgene repression, and 6 repeats may be used to allow sufficient repression of the transgene to levels that could rescue vector titres. Whilst the KAGNN consensus sequence may be varied to maintain TRAP-mediated repression, preferably the precise sequence chosen may be optimised to ensure high levels of translation in the non-repressed state. For example, the tbs sequences may be optimised by removing splicing sites, unstable sequences or stem-loops that might hamper translation efficiency of the mRNA in the absence of TRAP (i.e. in target cells). Regarding the configuration of the KAGNN repeats of a given tbs, the number of N “spacing” nucleotides between the KAG repeats is preferably two. However, a tbs containing more than two N spacers between at least two KAG repeats may be tolerated (as many as 50% of the repeats containing three Ns may result in a functional tbs as judged by in vitro binding studies; Babitzke P, Y. J., Campanelli D. (1996) Journal of Bacteriology 178(17): 5159-5163). Indeed, it has been shown that an 11×KAGNN tbs sequence can tolerate up to three replacements with KAGNNN repeats and still retain some potentially useful translation-blocking activity in partnership with TRAP-binding.

In one embodiment of the present invention, the TRAP binding site or portion thereof comprises the sequence KAGN_{≥2} (e.g. KAGN₂₋₃). For the avoidance of doubt therefore, this tbs or portion thereof comprises, for example, any of the following repeat sequences: UAGNN, GAGNN, TAGNN, UAGNNN, GAGNNN, or TAGNNN.

“N” is to be understood as specifying any nucleotide at that position in the sequence. For example, this could be G, A, T, C or U. The number of such nucleotides is preferably 2 but up to three, for example 1, 2 or 3, KAG repeats of an 11× repeat tbs or portion thereof may be separated by 3 spacing nucleotides and still retain some TRAP-binding activity that leads to translation repression. Preferably not more than one N₃ spacer will be used in an 11× repeat tbs or portion thereof in order to retain maximal TRAP-binding activity that leads to translation repression.

In another embodiment, the tbs or portion thereof comprises multiple repeats of KAGN_{≥2} (e.g. multiple repeats of KAGN₂₋₃).

In another embodiment, the tbs or portion thereof comprises multiple repeats of the sequence KAGN₂.

In another embodiment, the tbs or portion thereof comprises at least 6 repeats of KAGN_{≥2} (e.g. at least 6 repeats of KAGN₂₋₃).

In another embodiment, the tbs or portion thereof comprises at least 6 repeats of KAGN₂. For example, the tbs or portion thereof may comprise 6, 7, 8, 9, 10, 11, 12 or more repeats of KAGN₂. For example, the tbs or portion thereof may comprise any one of SEQ ID NOs: 8-19 or 22.

In another embodiment, the tbs or portion thereof comprises at least 8 repeats of KAGN_{≥2} (e.g. at least 8 repeats of KAGN₂₋₃). For example, the tbs or portion thereof may comprise any one of SEQ ID NOs: 8, 9, 14-17, 20-24.

Preferably, the number of KAGNNN repeats present in the tbs or portion thereof is 1 or less. For example, the tbs or portion thereof may comprise any one of SEQ ID NOs: 8, 9, 14-17, 19-24.

In another embodiment, the tbs or portion thereof comprises 11 repeats of KAGN_{≥2} (e.g. 11 repeats of KAGN₂₋₃). Preferably, the number of KAGNNN repeats present in this tbs or portion thereof is 3 or less. For example, the tbs or portion thereof may comprise any one of SEQ ID NOs: 8, 9, 14, 20-22.

In another embodiment, the tbs or portion thereof comprises 12 repeats of KAGN_{≥2} (e.g. 12 repeats of KAGN₂₋₃).

In a preferred embodiment, the tbs or portion thereof comprises 8-11 repeats of KAGN₂ (e.g. 8, 9, 10 or 11 repeats of KAGN₂). For example, the tbs or portion thereof may comprise any one of SEQ ID NOs: 8, 9, 14-17, 20-24.

In one embodiment, the TRAP binding site or portion thereof may comprise any of SEQ ID NOs: 8-24.

For example, the TRAP binding site or portion thereof may comprise either of the following sequences:

(SEQ ID NO: 8)
GAGUUUAGCGGAGUGGAGAAGAGCGGAGCCGAGCCUAGCAGAGACGAG
UGGAGCU;
or
(SEQ ID NO: 9)
GAGUUUAGCGGAGUGGAGAAGAGCGGAGCCGAGCCUAGCAGAGACGAG
AAGAGCU

By “repeats of KAGN_{≥2}” it is to be understood that the general KAGN_{≥2} (e.g. KAGN₂₋₃) motif is repeated. Different KAGN_{≥2} sequences satisfying the criteria of this motif may be joined to make up the tbs or portion thereof. It is not intended that the resulting tbs or portion thereof is limited to repeats of only one sequence that satisfies the requirements of this motif, although this possibility is included in the definition. For example, “6 repeats of KAGN_{≥2}” includes, but is not limited to, the sequences:

(SEQ ID NO: 10)
UAGUU-UAGUU-UAGUU-UAGUU-UAGUU-UAGUU;
(SEQ ID NO: 11)
UAGUU-UAGUU-GAGUU-UAGUU-GAGUU-UAGUU;
(SEQ ID NO: 12)
GAGUUU-GAGUU-GAGUU-GAGUUU-GAGUU-GAGUU
and:
(SEQ ID NO: 13)
UAGUUU-GAGUU-UAGUU-GAGUUU-UAGUU-GAGUU

(the dashes are included here between the repeats for clarity only).

An 8-repeat tbs or portion thereof containing one KAGNNN repeat and seven KAGNN repeats retains TRAP-mediated repression activity. Less than 8-repeat tbs sequences or portions thereof (e.g. 7- or 6-repeat tbs sequences or portions thereof) containing one or more KAGNNN repeats may have lower TRAP-mediated repression activity. Accordingly, when fewer than 8-repeats are present, it is preferred that the tbs or portion thereof comprises only KAGNN repeats.

Preferred nucleotides for use in the KAGNN repeat consensus are:
a pyrimidine in at least one of the NN spacer positions;
a pyrimidine at the first of the NN spacer positions;
pyrimidines at both of the NN spacer positions;
G at the K position.

It is also preferred that G is used at the K position when the NN spacer positions are AA (i.e. it is preferred that TAGAA is not used as a repeat in the consensus sequence).

By “capable of interacting” it is to be understood that the nucleic acid binding site (e.g. tbs or portion thereof) is capable of binding to a protein, for example TRAP, under the conditions that are encountered in a cell, for example a eukaryotic viral vector production cell. Such an interaction with an RNA-binding protein such as TRAP results in the repression or prevention of translation of a NOI to which the nucleic acid binding site (e.g. the tbs or portion thereof) is operably linked.

By “operably linked” it is to be understood that the components described are in a relationship permitting them to function in their intended manner. Therefore a tbs or portion thereof for use in the invention operably linked to a NOI is positioned in such a way that translation of the NOI is modified when as TRAP binds to the tbs or portion thereof.

Placement of a tbs or portion thereof capable of interacting with an TRAP upstream of a NOI translation initiation codon of a given open reading frame (ORF) allows specific translation repression of mRNA derived from that ORF. The number of nucleotides separating the tbs or portion thereof and the translation initiation codon may be varied, for example from 0 to 34 nucleotides, without affecting the degree of repression. As a further example, 0 to 13 nucleotides may be used to separate the TRAP-binding site or portion thereof and the translation initiation codon.

The tbs or portion thereof may be placed downstream of an internal ribosome entry site (IRES) to repress translation of the NOI in a multicistronic mRNA. Indeed, this supplies further evidence that tbs-bound TRAP might block the passage of the 40S ribosome; IRES elements function to sequester the 40S ribosome subunit to an mRNA in a CAP-independent manner before the full translation complex is formed (see Thompson, S. (2012) Trends in Microbiology 20(11): 558-566) for a review on IRES translation initiation). Thus, it is possible for the TRIP system to repress multiple open-reading frames from a single mRNA expressed from viral vector genomes. This will be a useful feature of the TRIP system when producing vectors encoding multiple therapeutic genes, especially when all the transgene products might negatively affect vector titres to some degree.

In one embodiment, the nucleic acid sequence comprises a spacer sequence between an IRES and the tbs or the portion thereof. The IRES may be an IRES as described herein under the subheading “Internal ribosome entry site”. The spacer sequence may be between 0 and 30 nucleotides in length, preferably 15 nucleotides in length. The spacer may comprise the sequence as defined in any one of SEQ ID NOs:38-44, preferably the spacer comprises a sequence as defined in SEQ ID NO:38.

In one embodiment, the spacer sequence between an IRES and the tbs or portion thereof is 3 or 9 nucleotides from the 3′ end of the tbs or portion thereof and the downstream initiation codon of the NOI.

In one embodiment, the tbs or portion thereof lacks a type II restriction enzyme site. In a preferred embodiment, the tbs or portion thereof lacks a SapI restriction enzyme site.

In some embodiments, the nucleic acid sequence further comprises an RRE sequence or functional substitute thereof.

In some embodiments, the nucleic acid sequence is a vector transgene expression cassette.

Overlapping Kozak Sequence and TRAP Binding Site

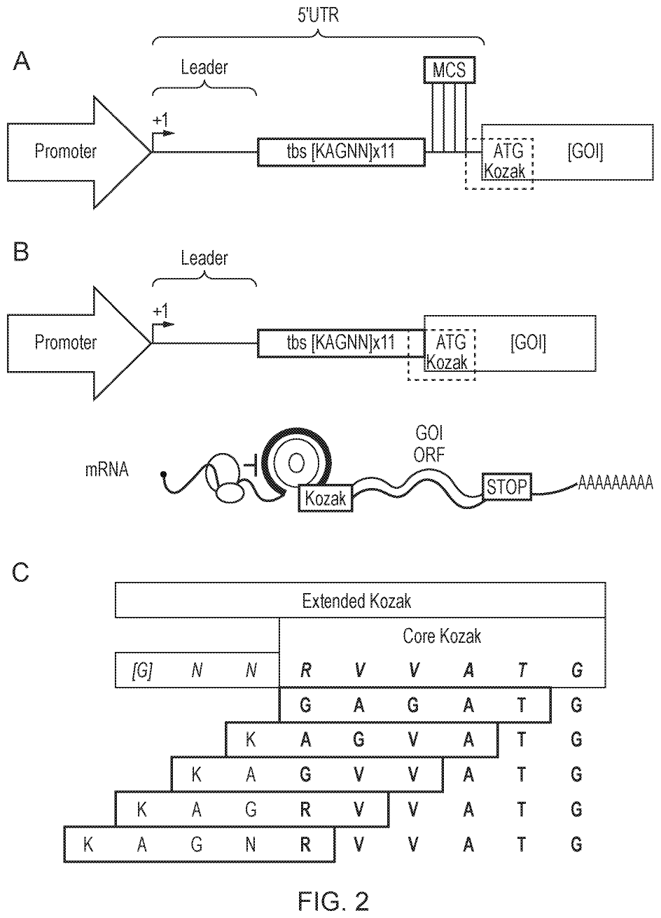

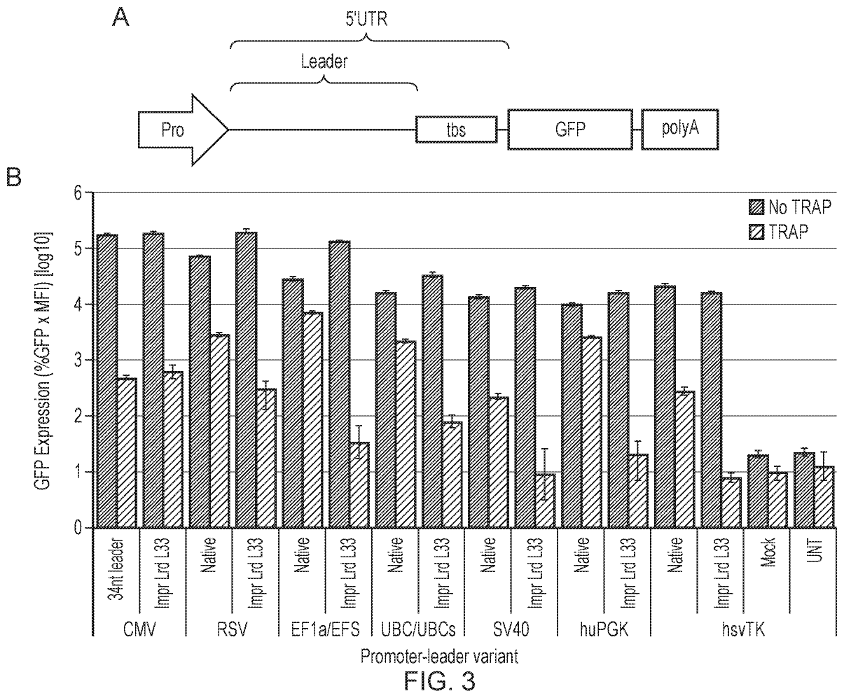

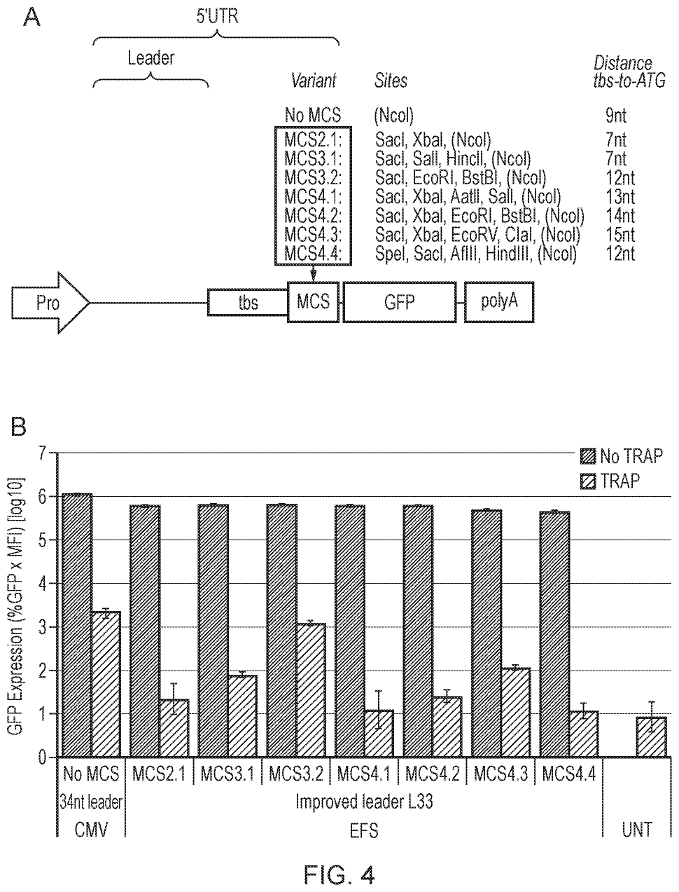

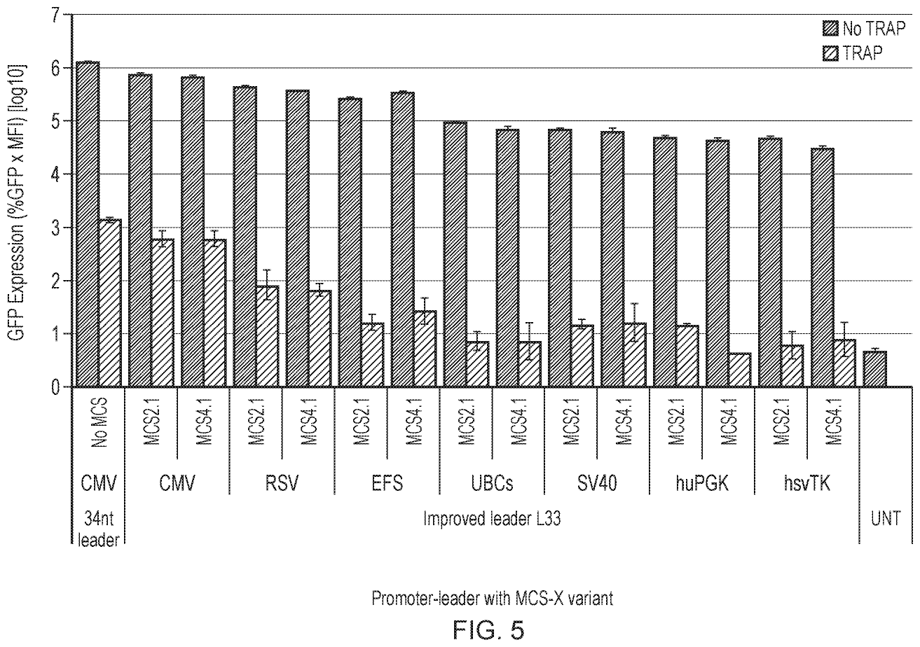

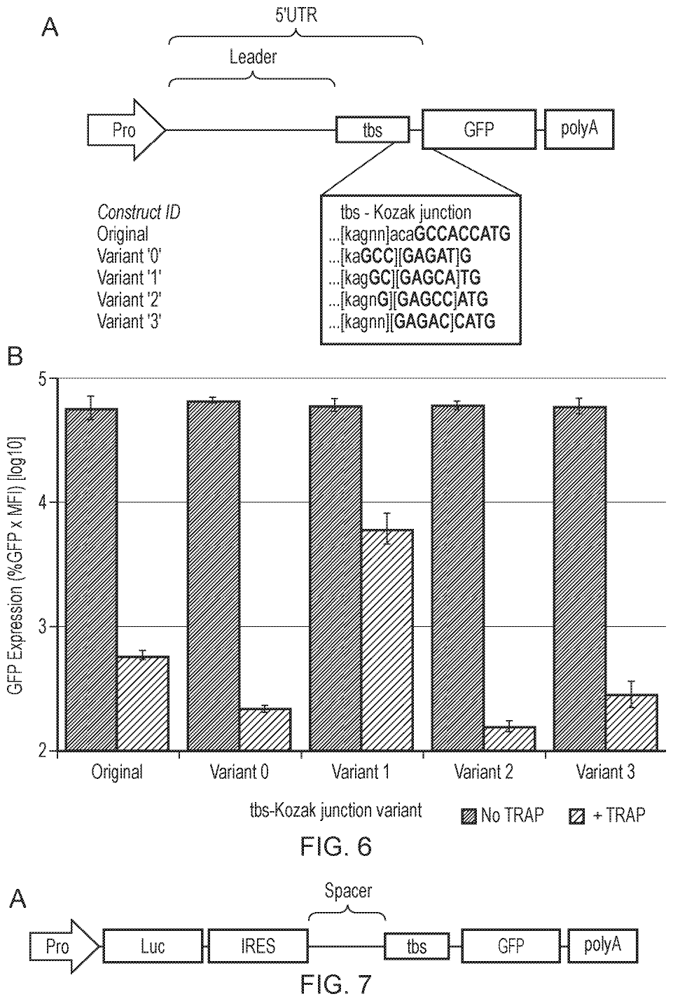

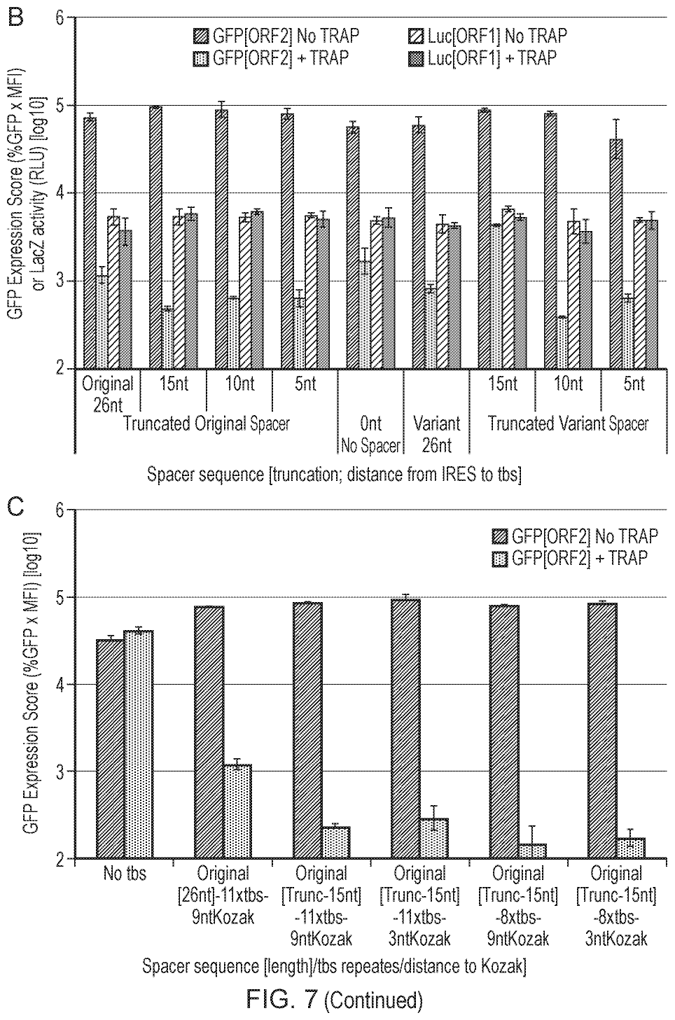

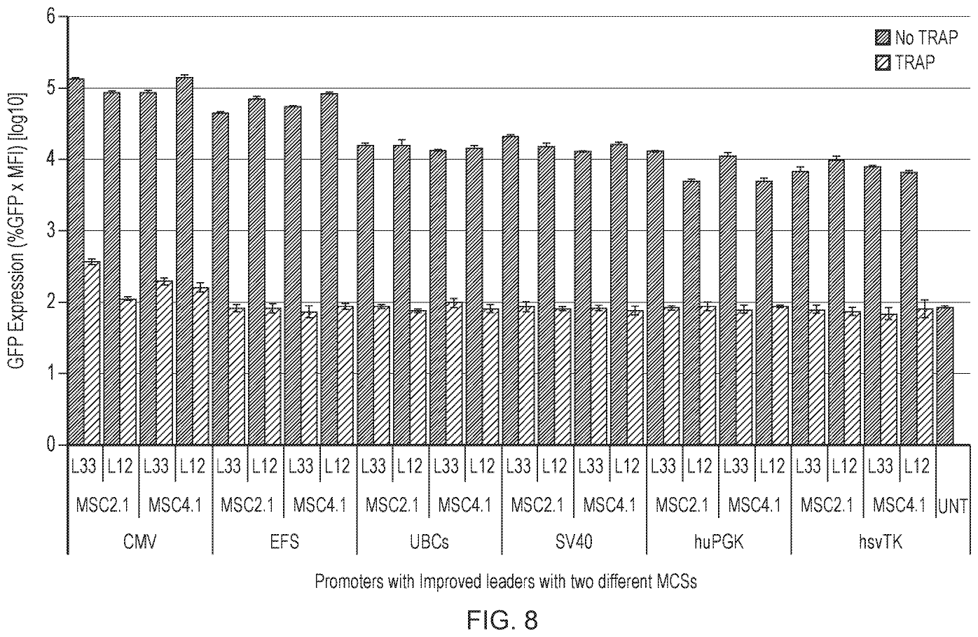

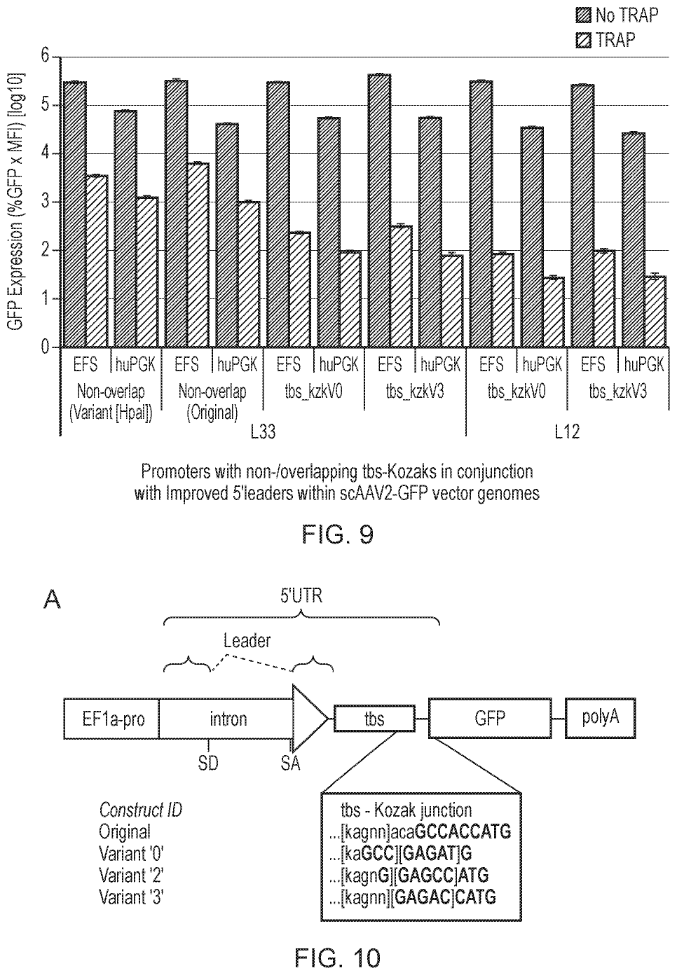

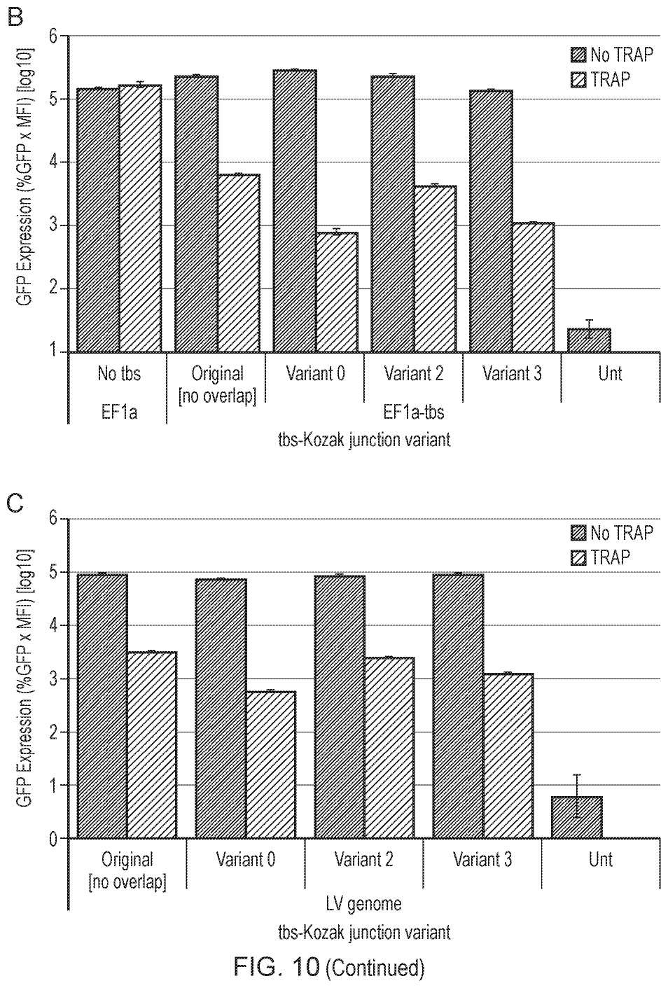

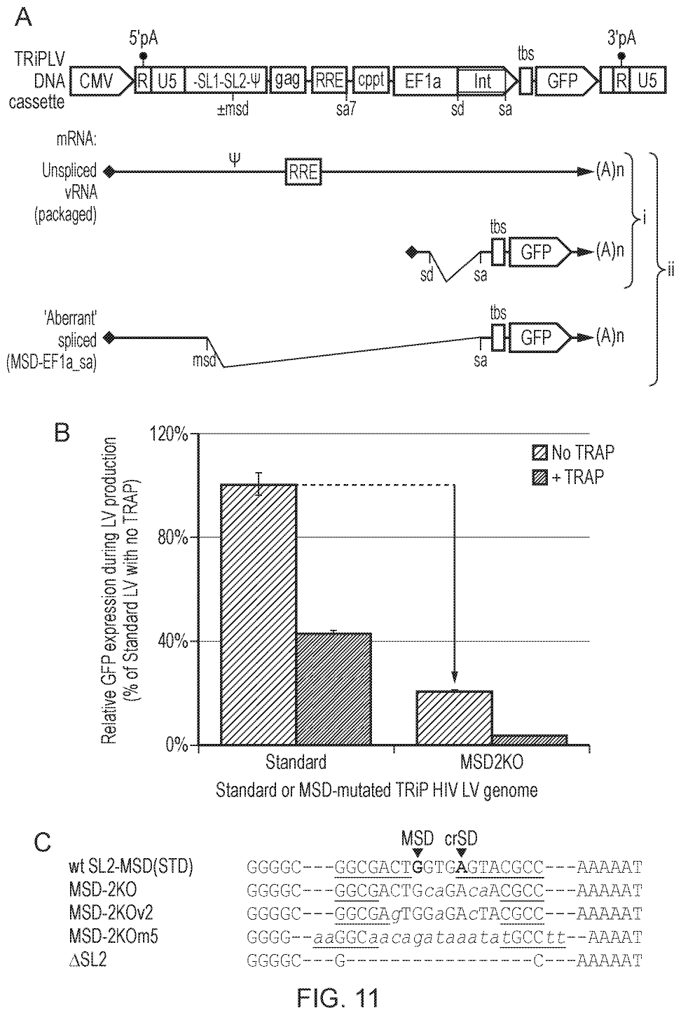

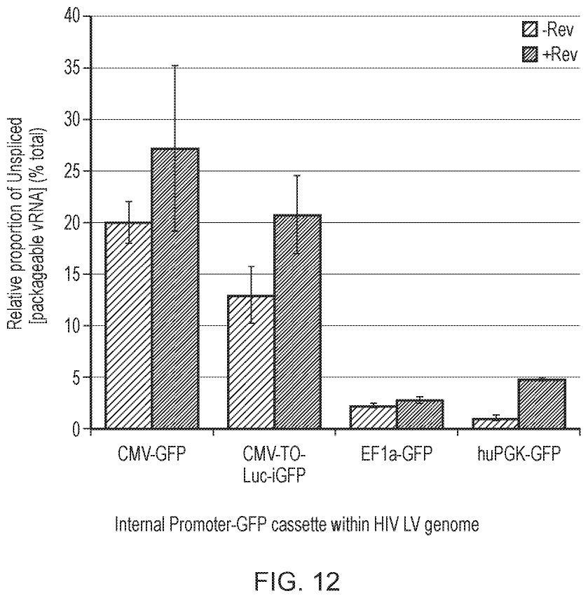

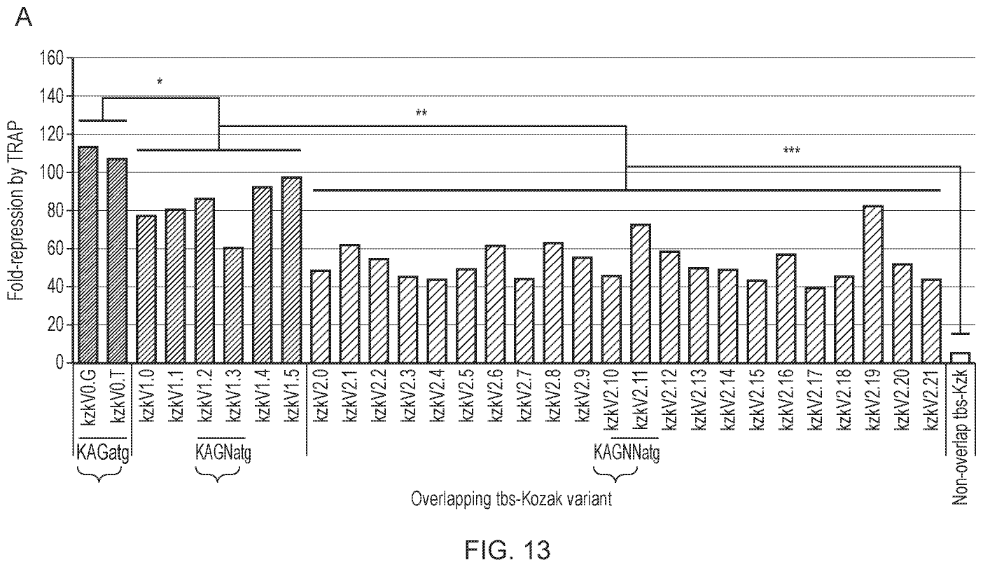

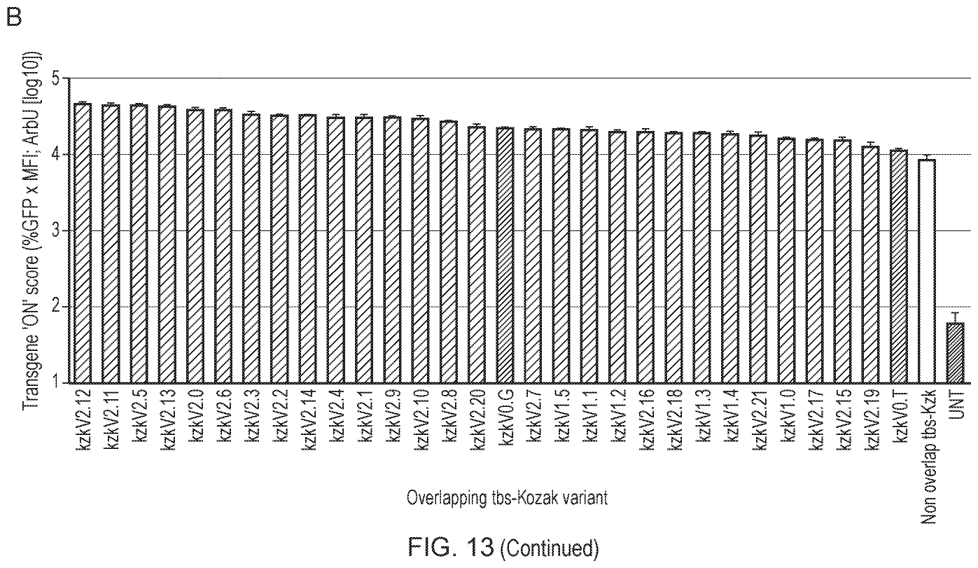

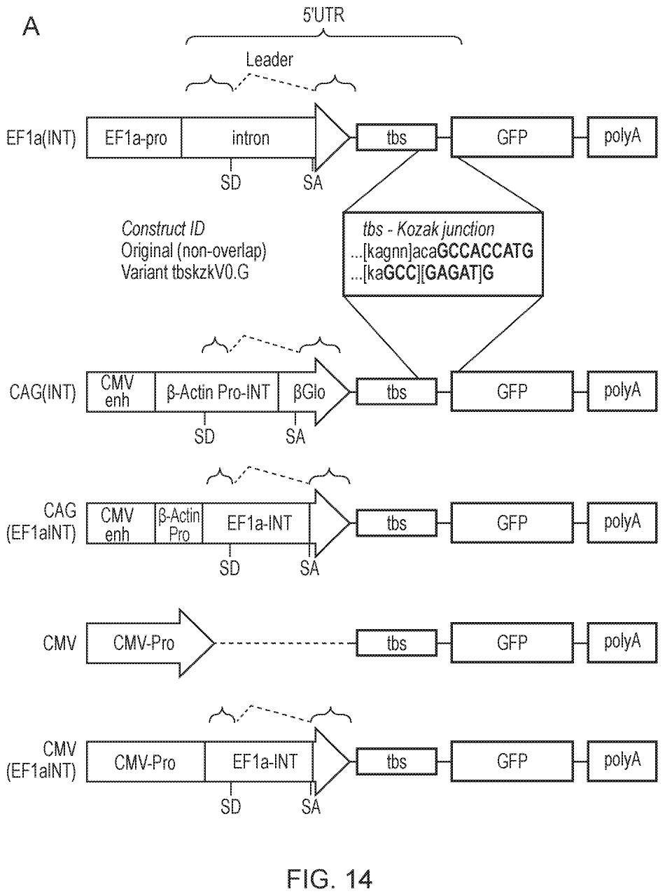

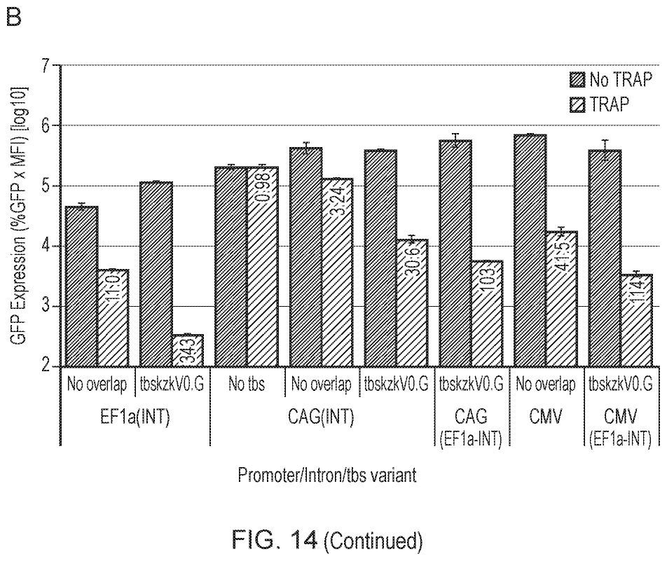

The present inventors have surprisingly found that improved levels of repression can be achieved by ‘hiding’ the Kozak sequence within the 3′ terminus of the tbs or portion thereof (using overlapping tbs and Kozak sequences; see FIGS. 2B and 2C), compared to the use of non-overlapping tbs and Kozak sequences. In addition, all of the tested overlapping Kozak and tbs sequences unexpectedly directed efficient levels of translation initiation, i.e. the tested overlapping sequences provided similar levels of transgene expression to the non-overlapping Kozak and tbs sequences in the absence of TRAP. Without wishing to be bound by theory, the improved levels of repression can be attributed to improved occlusion of the transgene initiation codon by the TRAP-tbs complex when the tbs or portion thereof overlaps the Kozak sequence.

The term “Kozak sequence” is to be understood as a consensus sequence in eukaryotic mRNA which is recognised by the ribosome as the translational start site. The Kozak sequence includes the ATG initiation (start) codon in DNA (AUG in mRNA). The exact Kozak sequence present in eukaryotic mRNA determines the efficiency of translation initiation, i.e. certain Kozak sequences will not lead to efficient translation initiation.

The full Kozak sequence is typically understood to have the consensus sequence (gcc)gccRccATGG for DNA and (gcc)gccRccAUGG for RNA, wherein: a lowercase letter denotes the most common base at a position where the base at this position can vary; an uppercase letter denotes a highly conserved base at this position; “R” denotes that a purine (i.e. A or G) is typically optimal at this position; and the sequence in parentheses (gcc) is of uncertain significance. T/U is generally the least preferred nucleotide at all of the positions of the Kozak sequence consensus that are upstream of the initiation codon.

As the first three bases of the full Kozak sequence are of uncertain significance, the Kozak sequence can also be understood to have the consensus sequence, referred to herein as the “extended Kozak sequence”, GNNRVVATGG for DNA (SEQ ID NO: 27) and GNNRVVAUGG for RNA, wherein “R” is to be understood as specifying a purine (i.e. A or G) at that position in the sequence, “V” is to be understood as specifying any nucleotide from G, A, or C and “N” is to be understood as specifying any nucleotide at that position in the sequence. For example, “N” could be G, A, T, C or U. It should be noted that the “R” at position −1 and “G” and position +3 (relative to the “A” of the ATG being position 0) are considered the most important positions in terms of Kozak strength. However, the presence of the “G” at position +3 in transgene sequences is dependent on the ORF being encoded, and so for the purposes of the specification the +3 position has not been considered as part of the ‘core’ Kozak sequence.

The bases found at the first six positions of the full Kozak sequence vary such that any base can be found at those positons (denoted (gcc)gcc above). Thus, the full Kozak consensus sequence can be considered to contain a ‘core’ Kozak sequence which consists of the portion of the full Kozak sequence having reduced variability, denoted RccAUG above. The ‘core’ Kozak consensus sequence is defined herein as: RVVAUG for mRNA and RVVATG for DNA, wherein “R” is to be understood as specifying a purine (i.e. A or G) at that position in the sequence and “V” is to be understood as specifying any nucleotide from G, A, or C.

In one preferred embodiment of the present invention, the Kozak sequence comprises the sequence RVVATG (SEQ ID NO: 28); wherein “R” is to be understood as specifying a purine (i.e. A or G) at that position in the sequence and “V” is to be understood as specifying any nucleotide from G, A, or C.

In one embodiment of the present invention, the Kozak sequence comprises the sequence RNNATG (SEQ ID NO: 125); wherein “R” is to be understood as specifying a purine (i.e. A or G) at that position in the sequence and “N” is to be understood as specifying any nucleotide from G, A, T/U or C, recognising that use of a “T/U” may give rise to reduced levels of expression in the absence of TRAP.

In some embodiments, the Kozak sequence overlaps the 3′ terminal KAGNN repeat of the TRAP binding site or of the portion thereof. Thus, the core Kozak sequence may overlap the the 3′ terminal KAGNN repeat of the TRAP binding site or of the portion thereof.

A summary of preferred overlapping tbs and Kozak consensus sequences is provided in FIG. 2C.

In a preferred embodiment, the 3′ terminal KAGNN repeat of the TRAP binding site or of the portion thereof overlaps at least the first, or first two, nucleotides of the ATG triplet within the core Kozak sequence.

As described herein, in one aspect of the present invention, the 3′ terminal KAGNN repeat of the TRAP binding site or of the portion thereof overlaps the ATG start codon of the nucleotide of interest (transgene ORF). In one aspect the 3′ terminal KAGNN repeat of the TRAP binding site or of the portion thereof overlaps the first one or two of the ATG start codon of the nucleotide of interest (transgene ORF).

In one aspect the overlapping tbs-Kozak sequence may have the consensus sequence KAGNNG (SEQ ID NO: 113), wherein “NN” is the first two nucleotides within the ATG triplet of the Kozak sequence.

The consensus sequence may be KAGATG (SEQ ID NO: 114); wherein “K” is either G or T/U.

In one aspect, the overlapping tbs-Kozak sequence may be GAGATG (SEQ ID NO: 29) as shown in FIG. 2C.

In one embodiment, the 3′ terminal KAGNN repeat of the TRAP binding site or of the portion thereof overlaps the first nucleotide of the ATG triplet within the nucleotide of interest.

In one aspect the sequence may comprise the sequence KAGNNTG (SEQ ID NO: 115), wherein the second “N” is the first nucleotide within the ATG triplet. The consensus sequence may be KAGNATG (SEQ ID NO: 116); wherein “K” is to be understood as specifying a G or T/U at that position in the sequence, “N” is to be understood as specifying any nucleotide from G, A, T, U or C but preferably “V”, i.e. from G, A or C. For example, the overlapping sequence may be KAGVATG (SEQ ID NO: 30) as shown in FIG. 2C.

In one embodiment, the overlapping Kozak sequence and TRAP binding site or portion thereof for use in the nucleic acid of the invention comprises one of the following sequences:

(a)
(SEQ ID NO: 29)
GAGATG;
(b)
(SEQ ID NO: 30)
KAGVATG;
(c)
(SEQ ID NO: 31)
KAGVVATG;
(d)
(SEQ ID NO: 32)
KAGRVVATG;
or
(e)
(SEQ ID NO: 33)
KAGNRVVATG;

wherein “K” may be T or G, “R” is to be understood as specifying a purine (i.e. A or G) at that position in the sequence, “V” is to be understood as specifying any nucleotide from G, A, or C, and “N” is to be understood as specifying any nucleotide at that position in the sequence. For example, “N” could be G, A, T, C or U.

In one embodiment, the nucleic acid sequence of the invention comprises one of the following sequences:

(a)
(SEQ ID NO: 29)
GAGATG
or
(SEQ ID NO: 114)
KAGATG;
(b)
(SEQ ID NO: 30)
KAGVATG;
(c)
(SEQ ID NO: 31)
KAGVVATG;
(d)
(SEQ ID NO: 32)
KAGRVVATG;
or
(e)
(SEQ ID NO: 33)
KAGNRVVATG;

wherein “K” may be T or G, “R” is to be understood as specifying a purine (i.e. A or G) at that position in the sequence, “V” is to be understood as specifying any nucleotide from G, A, or C, and “N” is to be understood as specifying any nucleotide at that position in the sequence. For example, “N” could be G, A, T, C or U.

Preferred overlapping tbs or portion thereof and core Kozak sequences corresponding to the consensus sequences GAGATG (SEQ ID NO: 29), KAGVATG (SEQ ID NO: 30) and KAGVVATG (SEQ ID NO: 31) for use in the nucleic acid of the invention (based upon the consensus tbs repeat sequence of KAGNN as defined herein and the consensus ‘core’ Kozak sequence RVVATG as defined herein) encompass:

(a)
(SEQ ID NO: 29)
GAGATG;
(b)
(SEQ ID NO: 69)
GAGAATG;
(c)
(SEQ ID NO: 70)
GAGCATG;
(d)
(SEQ ID NO: 71)
GAGGATG;
(e)
(SEQ ID NO: 72)
TAGAATG;
(f)
(SEQ ID NO: 73)
TAGCATG;
(g)
(SEQ ID NO: 74)
TAGGATG;
(h)
(SEQ ID NO: 75)
GAGAAATG;
(i)
(SEQ ID NO: 76)
GAGACATG;
(j)
(SEQ ID NO: 77)
GAGAGATG;
(k)
(SEQ ID NO: 78)
GAGCAATG;
(l)
(SEQ ID NO: 79)
GAGCCATG;
(m)
(SEQ ID NO: 80)
GAGCGATG;
(n)
(SEQ ID NO: 81)
GAGGAATG;
(o)
(SEQ ID NO: 82)
GAGGCATG;
(p)
(SEQ ID NO: 83)
GAGGGATG;
(q)
(SEQ ID NO: 84)
TAGAAATG;
(r)
(SEQ ID NO: 85)
TAGACATG;
(s)
(SEQ ID NO: 86)
TAGAGATG;
(t)
(SEQ ID NO: 87)
TAGCAATG;
(u)
(SEQ ID NO: 88)
TAGCCATG;
(v)
(SEQ ID NO: 89)
TAGCGATG;
(w)
(SEQ ID NO: 90)
TAGGAATG;
(x)
(SEQ ID NO: 91)
TAGGCATG;
(y)
(SEQ ID NO: 92)
TAGGGATG.

In some embodiments, the nucleic acid sequence comprises one of the following sequences:

(a)
(SEQ ID NO: 34)
KAGCCGAGATG;
(b)
(SEQ ID NO: 35)
KAGNGGAGCCATG;
or
(c)
(SEQ ID NO: 36)
KAGNNGAGACCATG;
(d)
(SEQ ID NO: 37)
KAGGCGAGCATG;

wherein “K” may be T or G and “N” is to be understood as specifying any nucleotide at that position in the sequence. For example, this could be G, A, T, C or U.

Preferably, the nucleic acid sequence comprises one of the following sequences:

(a)
(SEQ ID NO: 34)
KAGCCGAGATG;
or
(b)
(SEQ ID NO: 35)
KAGNGGAGCCATG;

wherein “K” may be T or G and “N” is to be understood as specifying any nucleotide at that position in the sequence. For example, this could be G, A, T, C or U.

In a preferred embodiment, the nucleic acid sequence of the invention comprises the overlapping tbs and Kozak sequence:

(SEQ ID NO: 60)
GAGTTTAGCGGAGTGGAGAAGAGCGGAGCCGAGCCTAGCAGAGACGAGC
CGAGATG.

Repression or prevention of the translation of the NOI is to be understood as alteration of the amount of the product (e.g. protein) of the NOI that is translated during viral vector production in comparison to the amount expressed in the absence of the nucleic acid sequence of the invention at the equivalent time point. Such alteration of translation results in a consequential repression or prevention of the expression of the protein encoded by the NOI.

In one embodiment, the nucleic acid sequence of the invention is capable of interacting TRAP, such that translation of the nucleotide of interest is repressed or prevented in a viral vector production cell.

The translation of the NOI at any given time during vector production may be reduced to 90%, 80%, 70%, 60%, 50%, 40%, 30%, 20%, 10%, 5%, 4%, 3%, 2%, 1%, 0.5% or 0.1% of the amount translated in the absence of the nucleic acid sequence of the invention at the same time-point in vector production.

The translation of the NOI at any given time during vector production may be reduced to less than 90%, 80%, 70%, 60%, 50%, 40%, 30%, 20%, 10%, 5%, 4%, 3%, 2%, 1%, 0.5% or 0.1% of the amount translated in the absence of the nucleic acid sequence of the invention at the same time-point in vector production.

In the context of the present invention, the translation of the NOI at any given time during vector production may be reduced to 90%, 80%, 70%, 60%, 50%, 40%, 30%, 20%, 10%, 5%, 4%, 3%, 2%, 1%, 0.5% or 0.1% of the amount translated in the presence of a nucleic acid sequence comprising non-overlapping Kozak and tbs sequences (as opposed to the nucleic acid sequence of the invention) at the same time-point in vector production.

In the context of the present invention the translation of the NOI at any given time during vector production may be reduced to less than 90%, 80%, 70%, 60%, 50%, 40%, 30%, 20%, 10%, 5%, 4%, 3%, 2%, 1%, 0.5% or 0.1% of the amount translated in the presence of a nucleic acid sequence comprising non-overlapping Kozak and tbs sequences (as opposed to the nucleic acid sequence of the invention) at the same time-point in vector production.

Preventing the translation of the NOI is to be understood as reducing the amount of translation to substantially zero.

The expression of the protein from the NOI at any given time during vector production may be reduced to 90%, 80%, 70%, 60%, 50%, 40%, 30%, 20%, 10%, 5%, 4%, 3%, 2%, 1%, 0.5% or 0.1% of the amount expressed in the absence of the nucleic acid sequence of the invention at the same time-point in vector production.

The expression of the protein from the NOI at any given time during vector production may be reduced to less than 90%, 80%, 70%, 60%, 50%, 40%, 30%, 20%, 10%, 5%, 4%, 3%, 2%, 1%, 0.5% or 0.1% of the amount expressed in the absence of the nucleic acid sequence of the invention at the same time-point in vector production.

In the context of the present invention the expression of the protein from the NOI at any given time during vector production may be reduced to 90%, 80%, 70%, 60%, 50%, 40%, 30%, 20%, 10%, 5%, 4%, 3%, 2%, 1%, 0.5% or 0.1% of the amount expressed in the presence of a nucleic acid sequence comprising non-overlapping Kozak and tbs sequences (as opposed to the nucleic acid sequence of the invention) at the same time-point in vector production.

In the context of the present invention the expression of the protein from the NOI at any given time during vector production may be reduced to less than 90%, 80%, 70%, 60%, 50%, 40%, 30%, 20%, 10%, 5%, 4%, 3%, 2%, 1%, 0.5% or 0.1% of the amount expressed in the presence of a nucleic acid sequence comprising non-overlapping Kozak and tbs sequences (as opposed to the nucleic acid sequence of the invention) at the same time-point in vector production.

Preventing the expression of the protein from the NOI is to be understood as reducing the amount of the protein that is expressed to substantially zero.

Methods for the analysis and/or quantification of the translation of an NOI are well known in the art.

A protein product from lysed cells may be analysed using methods such as SDS-PAGE analysis with visualisation by Coomassie or silver staining. Alternatively a protein product may be analysed using Western blotting or enzyme-linked immunosorbent assays (ELISA) with antibody probes which bind the protein product. A protein product in intact cells may be analysed by immunofluorescence.

Improved Leader Sequence

In application of the TRIP system to different promoters (containing different native 5′UTRs of different lengths and composition) it is desirable to be able to be able to simply apply the tbs sequence within a promoter-UTR context to afford efficient repression by TRAP, whilst also maintaining good levels of expression without TRAP. From the outset of this work, it was not known what would be the achievable level of repression mediated by TRAP-tbs, when the tbs is inserted into native UTRs of a variety of constitutive promoters. Ideally, it would be advantageous to be able to supply a single conserved 5′UTR leader sequence together with the tbs when modifying the promoter of choice in order to avoid any of the potential variability in repression levels that might be directed by native 5′UTR sequences. Surprisingly, it was found that the first exon of the EF1a promoter (SEQ ID NO: 25) provides consistently good levels of transgene repression by TRAP compared to 5′UTR leaders comprising native leader sequences, and this leader also provides good levels of transgene expression in the absence of TRAP.

In some embodiments, the nucleic acid sequence comprises a 5′ leader sequence upstream of the tbs or the portion thereof. The leader sequence may be immediately upstream of the TRAP binding site or the portion thereof, i.e. there may be no further sequences separating the leader sequence and the TRAP binding site or portion thereof. If the 5′ leader is derived from a splicing event, then the sequences from the exon/exon junction to the tbs should be kept to a minimal length (preferably 12nt). The leader sequence may comprise a sequence derived from the non-coding EF1a exon 1 region. In a preferred embodiment, the leader sequence comprises a sequence as defined in SEQ ID NO:25, SEQ ID NO:26 or SEQ ID NO: 93.

Multiple Cloning Site

To improve the tractability of the TRIP system it is desirable to be able to have the ability to clone a NOI directly into the expression cassette containing the promoter-5′UTR-tbs sequence via the option of several different restriction enzymes (RE), i.e. to incorporate a multiple cloning site (MCS) between the tbs and the Kozak sequence (see FIG. 2A). The present inventors have demonstrated that several different MCS can be tolerated by the TRIP system, i.e. transgene repression is still obtained when an MCS is used. This was unexpected given that 5′UTR leader sequences can modulate the degree of TRAP-mediated repression, that the close proximity of the tbs to the ATG initiation codon is important, and that an efficient Kozak sequence must be maintained with or between the MCS and the initiation codon of the NOI to ensure efficient translation initiation. In addition, the number and/or combinations of RE sites that could be used whilst maintaining TRAP-mediated repression could not be predicted.

As such, sequence ‘compression’ was required such that several (overlapping) RE sites could be incorporated in as short a distance as possible from the tbs to the ATG initiation codon (to retain proximity of tbs to ATG), whilst also maintaining an efficient core Kozak sequence of RVVATG.

In a further aspect, the invention provides a nucleic acid sequence comprising a nucleotide of interest, a tbs or a portion thereof as described herein, a multiple cloning site (MCS) and a Kozak sequence as described herein, wherein said MCS is located downstream of the tbs or portion thereof and upstream of the Kozak sequence. Suitably, the tbs or portion thereof and the Kozak sequence do not overlap.

As used herein, a “multiple cloning site” is to be understood as a DNA region which contains several restriction enzyme recognition sites (restriction enzyme sites) very close to each other. In one embodiment, the RE sites may be overlapping in the MCS for use in the invention.

As used herein, a “restriction enzyme site” or “restriction enzyme recognition site” is a location on a DNA molecule containing specific sequences of nucleotides, 4-8 nucleotides in length, which are recognised by restriction enzymes. A restriction enzyme recognises a specific RE site (i.e. a specific sequence) and cleaves the DNA molecule within, or nearby, the RE site.

A consensus TRAP binding site sequence that is capable of binding TRAP is [KAGNN] repeated multiple times (e.g. 6, 7, 8, 9, 10, 11, 12 or more times); wherein K may be T or G in DNA and U or G in RNA. In one embodiment of the present invention, the TRAP binding site or portion thereof comprises the sequence KAGN_{≥2} (e.g. KAGN₂₋₃). For the avoidance of doubt therefore, this tbs or portion thereof comprises, for example, any of the following repeat sequences: UAGNN, GAGNN, TAGNN, UAGNNN, GAGNNN, or TAGNNN. “N” is to be understood as specifying any nucleotide at that position in the sequence. For example, this could be G, A, T, C or U. The number of such nucleotides is preferably 2 but up to three, for example 1, 2 or 3, KAG repeats of an 11× repeat tbs or portion thereof may be separated by 3 spacing nucleotides and still retain some TRAP-binding activity that leads to translation repression. Preferably not more than one N₃ spacer will be used in an 11× repeat tbs or portion thereof in order to retain maximal TRAP-binding activity that leads to translation repression.

In one embodiment, the nucleic acid sequence comprises one of the following sequences:

(a)
(SEQ ID NO: 45)
GAGCTCTAGAVVATG;
(b)
(SEQ ID NO: 46)
GAGCTCGTCGACVATG;
(c)
(SEQ ID NO: 47)
GAGCTCGAATTCGAAVVATG;
(d)
(SEQ ID NO: 48)
GAGCTCTAGACGTCGACVATG;
(e)
(SEQ ID NO: 49)
GAGCTCTAGAATTCGAAVVATG;
(f)
(SEQ ID NO: 50)
GAGCTCTAGATATCGATRVVATG;
(g)
(SEQ ID NO: 51)
KAGACTAGTACTTAAGCTTRVVATG;
(h)
(SEQ ID NO: 52)
GAGCTCTAGACCATG;
(i)
(SEQ ID NO: 53)
GAGCTCGTCGACCATG;
(j)
(SEQ ID NO: 54)
GAGCTCGAATTCGAACCATG;
(k)
(SEQ ID NO: 55)
GAGCTCTAGACGTCGACCATG;
(l)
(SEQ ID NO: 56)
GAGCTCTAGAATTCGAACCATG;
(m)
(SEQ ID NO: 57)
GAGCTCTAGATATCGATACCATG;
or
(n)
(SEQ ID NO: 58)
KAGACTAGTACTTAAGCTTACCATG;

wherein “K” may be T or G, “R” is to be understood as specifying a purine (i.e. A or G) at that position in the sequence, and “V” is to be understood as specifying any nucleotide from G, A, or C.

In one embodiment, the nucleic acid sequence comprises one of the following sequences:

(a)
(SEQ ID NO: 52)
GAGCTCTAGACCATG;
(b)
(SEQ ID NO: 53)
GAGCTCGTCGACCATG;
(c)
(SEQ ID NO: 54)
GAGCTCGAATTCGAACCATG;
(d)
(SEQ ID NO: 55)
GAGCTCTAGACGTCGACCATG;
(e)
(SEQ ID NO: 56)
GAGCTCTAGAATTCGAACCATG;
(f)
(SEQ ID NO: 57)
GAGCTCTAGATATCGATACCATG;
or
(g)
(SEQ ID NO: 58)
KAGACTAGTACTTAAGCTTACCATG;

wherein “K” may be T or G.

In one embodiment, the nucleic acid sequence comprises one of the following sequences:

(a)
(SEQ ID NO: 52)
GAGCTCTAGACCATG;
(b)
(SEQ ID NO: 55)
GAGCTCTAGACGTCGACCATG;
or
(c)
(SEQ ID NO: 58)
KAGACTAGTACTTAAGCTTACCATG;

wherein “K” may be T or G.

In a preferred embodiment, the nucleic acid sequence of the invention comprises the overlapping tbs-MCS-Kozak sequence:

(SEQ ID NO: 61)
GAGTTTAGCGGAGTGGAGAAGAGCGGAGCCGAGCCTAGCAGAGACGAGA
AGAGCTCTAGACCATG.

In one embodiment, the NOI is operably linked to the tbs or the portion thereof.

In one embodiment, the tbs or portion thereof is capable of interacting with TRAP such that translation of the NOI is repressed in a viral vector production cell.

In one embodiment, the NOI is translated in a target cell which lacks TRAP.

In one embodiment, the tbs or the portion thereof comprises multiple repeats of the sequence KAGN₂₋₃.

In one embodiment, the tbs or the portion thereof comprises multiple repeats of the sequence KAGN₂.

In one embodiment, the tbs or the portion thereof comprises at least 6 repeats of the sequence KAGN₂. For example, the tbs or portion thereof may comprise any one of SEQ ID NOs: 8-19 or 22.

In one embodiment, the tbs or the portion thereof comprises at least 8 repeats of the sequence KAGN₂₋₃. For example, the tbs or portion thereof may comprise any one of SEQ ID NOs: 8, 9, 14-17, 20-24. Preferably, the number of KAGNNN repeats is 1 or less. For example, the tbs or portion thereof may comprise any one of SEQ ID NOs: 8, 9, 14-17, 19-24.

In one embodiment, the tbs or the portion thereof comprises at least 8-11 repeats of the sequence KAGN₂.

In one embodiment, the tbs or the portion thereof comprises 11 repeats of the sequence KAGN₂₋₃. Suitably, the number of KAGNNN repeats is 3 or less. For example, the tbs or portion thereof may comprise any one of SEQ ID NOs: 8, 9, 14, 20-22.

In one embodiment, the Kozak sequence comprises the sequence RVVATG (SEQ ID NO: 28); wherein “R” is to be understood as specifying a purine (i.e. A or G) at that position in the sequence, and “V” is to be understood as specifying any nucleotide from G, A, or C.

In one embodiment, the Kozak sequence comprises the sequence RNNATG (SEQ ID NO: 125); wherein “R” is to be understood as specifying a purine (i.e. A or G) at that position in the sequence, and “N” is to be understood as specifying any nucleotide from G, A, T/U or C.

In one embodiment, the distance between the transcription start site/end of promoter to start of the tbs or of the portion thereof is less than 34 nucleotides.

In one embodiment, the distance between the transcription start site/end of promoter to start of the tbs or of the portion thereof is less than 13 nucleotides.

In one embodiment, the tbs or the portion thereof lacks a type II restriction enzyme site, preferably a SapI restriction enzyme site.

In one embodiment, the nucleic acid sequence comprises a 5′ leader sequence upstream of the tbs or the portion thereof. The leader sequence may be immediately upstream of the TRAP binding site or the portion thereof, i.e. there may be no further sequences separating the leader sequence and the TRAP binding site or portion thereof.

In one embodiment, the leader sequence comprises a sequence derived from the non-coding EF1a exon 1 region.

In one embodiment, the leader sequence comprises a sequence as defined in SEQ ID NO:

25 or SEQ ID NO: 26.

In one embodiment, the nucleic acid sequence comprises an IRES.

In one embodiment, the nucleic acid sequence comprises a spacer sequence between an IRES and the tbs or the portion thereof.

In one embodiment, the spacer is between 0 and 30 nucleotides in length.

In one embodiment, the spacer is 15 nucleotides in length.

In one embodiment, the spacer is 3 or 9 nucleotides from the 3′ end of the tbs or the portion thereof and the downstream initiation codon of the NOI.

In one embodiment, the spacer comprises a sequence as defined in any one of SEQ ID NOs:38-44, preferably the spacer comprises a sequence as defined in SEQ ID NO: 38.

In one embodiment, the NOI gives rise to a therapeutic effect.

In one embodiment, the nucleic acid sequence further comprises an RRE sequence or functional substitute thereof.

In one embodiment, the nucleic acid sequence is a vector transgene expression cassette.

In one embodiment, the nucleic acid sequence of the invention further comprises a promoter. Typically, transcription of the promoter results in a 5′ UTR encoded in the resulting mRNA transcript. The promoter may be any promoter which is known in the art and is suitable for controlling the expression of the nucleotide of interest. For example, the promoter may be EF1a, EFS, CMV or CAG.

In a preferred embodiment, the overlapping tbs and Kozak sequence as described herein is located within the 5′ UTR of the promoter, wherein the 5′ UTR may comprise native sequence from the associated promoter, or more preferably, the 5′ UTR is composed of 5′ UTR sequences described herein.

In a preferred embodiment, the sequence comprising a compressed/overlapping MCS between the tbs and the Kozak sequence as described herein is located within said 5′ UTR.

The overlapping tbs and Kozak sequence as described herein or the sequence comprising a compressed/overlapping MCS between the tbs and the Kozak sequence as described herein may be located at the 3′ end of said 5′ UTR.

Preferably, the 5′ UTR comprises one of the following sequences: SEQ ID NO: 29-37, 45-58, 69-92 and 108-116. More preferably, the 5′ UTR comprises SEQ ID NO: 29 or SEQ ID NO: 108. Even more preferably, the 5′ UTR comprises SEQ ID NO: 29.

The promoter-5′ UTR region may comprise an intron. The intron may be a native intron or a heterologous intron. For example, the promoter may be EF1a or CAG.

The promoter may be a promoter which is typically used in viral vector genomes without an intron, for example CMV.

In a preferred embodiment, the promoter-5′ UTR region has been engineered to comprise an artificial 5′ UTR comprising a heterologous intron. Thus, the promoter-5′ UTR region has been engineered to contain a heterologous exon-intron-exon sequence, wherein the mature 5′ UTR encoded within the mRNA transcript results from splicing-out of the intron. The promoter-5′ UTR sequence may be engineered using methods known in the art. For example, the promoter may be engineered as described herein (see Example 8).

Preferably, expression of the transgene protein from its mature mRNA—resulting from splicing-out of the intron or heterologous intron—is efficiently repressed by TRAP. Suitably, the intron or heterologous intron may be the EF1a intron sequence as per SEQ ID No:122.

The intron or heterologous intron may be located upstream, i.e. 5′, of the overlapping tbs and Kozak sequence as described herein or of the sequence comprising an MCS between the tbs and the Kozak sequence as described herein.

The 5′ UTR may comprise the following sequence (chicken β-Actin/Rabbit β-globin chimeric 5′UTR-intron, exonic sequence in bold (spliced together to become 5′UTR leader)):

(SEQ ID NO: 121)
CGGCGGGCGGGAACGTTGCCTTCGCCCCGTGCCCCGCTCCGCGCCGCCT
CGCGCCGCCCGCCCCGGCTCTGACTGACCGCGTTACTCCCACAGGTGAG
CGGGCGGGACGGCCCTTCTCCCTCCGGGCTGTAATTAGCGCTTGGTTTA
ATGACGGCTCGTTTCTTTTCTGTGGCTGCGTGAAAGCCTTAAAGGGCTC
CGGGAGGGCCTTTGTGCGGGGGGGAGCGGCTCGGGGGGTGCGTGCGTGT
GTGTGTGCGTGGGGAGCGCCGCGTGCGGCCCGCGCTGCCCGGCGGCTGT
GAGCGCTGCGGGCGCGGCGCGGGGCTTTGTGCGCTCCGCGTGTGCGCGA
GGGGAGCGCGGGCCGGGGGCGGTGCCCCGCGGTGCGGGGGGGCTGCGAG
GGGAACAAAGGCTGCGTGCGGGGTGTGTGCGTGGGGGGGTGAGCAGGGG
GTGTGGGCGCGGCGGTCGGGCTGTAACCCCCCCCTGGCACCCCCCTCCC
CGAGTTGCTGAGCACGGCCCGGCTTCGGGTGCGGGGCTCCGTGCGGGGC
GTGGCGCGGGGCTCGCCGTGCCGGGCGGGGGGTGGCGGCAGGTGGGGGT
GCCGGGCGGGGCGGGGCCGCCTCGGGCCGGGGAGGGCTCGGGGGAGGGG
CGCGGCGGCCCCGGAGCGCCGGCGGCTGTCGAGGCGCGGCGAGCCGCAG
CCATTGCCTTTTATGGTAATCGTGCGAGAGGGCGCAGGGACTTCCTTTG
TCCCAAATCTGGCGGAGCCGAAATCTGGGAGGCGCCGCCGCACCCCCTC
TAGCGGGCGCGGGCGAAGCGGTGCGGCGCCGGCAGGAAGGAAATGGGCG
GGGAGGGCCTTCGTGCGTCGCCGCGCCGCCGTCCCCTTCTCCATCTCCA
GCCTCGGGGCTGCCGCAGGGGGACGGCTGCCTTCGGGGGGGACGGGGCA
GGGCGGGGTTCGGCTTCTGGCGTGTGACCGGCGGCTTTAGAGCCTCTGC
TAACCATGTTCATGCCTTCTTCTTTTTCCTACAGCTCCTGGGCAAA

The 5′ UTR may comprise the following sequence (EF1a 5′UTR-intron, exonic sequence in bold (spliced together to become 5′UTR leader)):

(SEQ ID NO: 122)
CTTTTTCGCAACGGGTTTGCCGCCAGAACACAGGTAAGTGCCGTGTGTG
GTTCCCGCGGGCCTGGCCTCTTTACGGGTTATGGCCCTTGCGTGCCTTG
AATTACTTCCACCTGGCTGCAGTACGTGATTCTTGATCCCGAGCTTCGG
GTTGGAAGTGGGTGGGAGAGTTCGTGGCCTTGCGCTTAAGGAGCCCCTT
CGCCTCGTGCTTGAGTTGTGGCCTGGCCTGGGCGCTGGGGCCGCCGCGT
GCGAATCTGGTGGCACCTTCGCGCCTGTCTCGCTGCTTTCGATAAGTCT
CTAGCCATTTAAAATTTTTGATGACCTGCTGCGACGCTTTTTTTCTGGC
AAGATAGTCTTGTAAATGCGGGCCAAGATCAGCACACTGGTATTTCGGT
TTTTGGGGCCGCGGGCGGCGACGGGGCCCGTGCGTCCCAGCGCACATGT
TCGGCGAGGCGGGGCCTGCGAGCGCGGCCACCGAGAATCGGACGGGGGT
AGTCTCAAGCTGCCCGGCCTGCTCTGGTGCCTGGCCTCGCGCCGCCGTG
TATCGCCCCGCCCTGGGCGGCAAGGCTGGCCCGGTCGGCACCAGTTGCG
TGAGCGGAAAGATGGCCGCTTCCCGGCCCTGCTGCAGGGAGCACAAAAT
GGAGGACGCGGCGCTCGGGAGAGCGGGCGGGTGAGTCACCCACACAAAG
GAAAAGGGCCTTTCCGTCCTCAGCCGTCGCTTCATGTGACTCCACGGAG
TACCGGGCGCCGTCCAGGCACCTCGATTAGTTCTCCAGCTTTTGGAGTA
CGTCGTCTTTAGGTTGGGGGGAGGGGTTTTATGCGATGGAGTTTCCCCA
CACTGAGTGGGTGGAGACTGAAGTTAGGCCAGCTTGGCACTTGATGTAA
TTCTCCTTGGAATTTGCCCTTTTTGAGTTTGGATCTTGGTTCATTCTCA
AGCCTCAGACAGTGGTTCAAAGTTTTTTTCTTCCATTTCAGGTGTCGTG
AAAA

In one embodiment, the promoter comprises the sequence (exonic sequence in bold (spliced together to become 5′UTR leader), tbs consensus in italics):

(SEQ ID NO: 123)
CGGCGGGCGGGAACGTTGCCTTCGCCCCGTGCCCCGCTCCGCGCCGCCT
CGCGCCGCCCGCCCCGGCTCTGACTGACCGCGTTACTCCCACAGGTGAG
CGGGCGGGACGGCCCTTCTCCCTCCGGGCTGTAATTAGCGCTTGGTTTA
ATGACGGCTCGTTTCTTTTCTGTGGCTGCGTGAAAGCCTTAAAGGGCTC
CGGGAGGGCCTTTGTGCGGGGGGGAGCGGCTCGGGGGGTGCGTGCGTGT
GTGTGTGCGTGGGGAGCGCCGCGTGCGGCCCGCGCTGCCCGGCGGCTGT
GAGCGCTGCGGGCGCGGCGCGGGGCTTTGTGCGCTCCGCGTGTGCGCGA
GGGGAGCGCGGGCCGGGGGCGGTGCCCCGCGGTGCGGGGGGGCTGCGAG
GGGAACAAAGGCTGCGTGCGGGGTGTGTGCGTGGGGGGGTGAGCAGGGG
GTGTGGGCGCGGCGGTCGGGCTGTAACCCCCCCCTGGCACCCCCCTCCC
CGAGTTGCTGAGCACGGCCCGGCTTCGGGTGCGGGGCTCCGTGCGGGGC
GTGGCGCGGGGCTCGCCGTGCCGGGCGGGGGGTGGCGGCAGGTGGGGGT
GCCGGGCGGGGCGGGGCCGCCTCGGGCCGGGGAGGGCTCGGGGGAGGGG
CGCGGCGGCCCCGGAGCGCCGGCGGCTGTCGAGGCGCGGCGAGCCGCAG
CCATTGCCTTTTATGGTAATCGTGCGAGAGGGCGCAGGGACTTCCTTTG
TCCCAAATCTGGCGGAGCCGAAATCTGGGAGGCGCCGCCGCACCCCCTC
TAGCGGGCGCGGGCGAAGCGGTGCGGCGCCGGCAGGAAGGAAATGGGCG
GGGAGGGCCTTCGTGCGTCGCCGCGCCGCCGTCCCCTTCTCCATCTCCA
GCCTCGGGGCTGCCGCAGGGGGACGGCTGCCTTCGGGGGGGACGGGGCA
GGGCGGGGTTCGGCTTCTGGCGTGTGACCGGCGGCTTTAGAGCCTCTGC
TAACCATGTTCATGCCTTCTTCTTTTTCCTACAGCTCCTGGGCAAA

[symbol]

In one embodiment, the promoter comprises the sequence (exonic sequence in bold (spliced together to become 5′UTR leader), tbs consensus in italics):

(SEQ ID NO: 124)
CTTTTTCGCAACGGGTTTGCCGCCAGAACACAGGTAAGTGCCGTGTGTG
GTTCCCGCGGGCCTGGCCTCTTTACGGGTTATGGCCCTTGCGTGCCTTG
AATTACTTCCACCTGGCTGCAGTACGTGATTCTTGATCCCGAGCTTCGG
GTTGGAAGTGGGTGGGAGAGTTCGTGGCCTTGCGCTTAAGGAGCCCCTT
CGCCTCGTGCTTGAGTTGTGGCCTGGCCTGGGCGCTGGGGCCGCCGCGT
GCGAATCTGGTGGCACCTTCGCGCCTGTCTCGCTGCTTTCGATAAGTCT
CTAGCCATTTAAAATTTTTGATGACCTGCTGCGACGCTTTTTTTCTGGC
AAGATAGTCTTGTAAATGCGGGCCAAGATCAGCACACTGGTATTTCGGT
TTTTGGGGCCGCGGGCGGCGACGGGGCCCGTGCGTCCCAGCGCACATGT
TCGGCGAGGCGGGGCCTGCGAGCGCGGCCACCGAGAATCGGACGGGGGT
AGTCTCAAGCTGCCCGGCCTGCTCTGGTGCCTGGCCTCGCGCCGCCGTG
TATCGCCCCGCCCTGGGCGGCAAGGCTGGCCCGGTCGGCACCAGTTGCG
TGAGCGGAAAGATGGCCGCTTCCCGGCCCTGCTGCAGGGAGCACAAAAT
GGAGGACGCGGCGCTCGGGAGAGCGGGCGGGTGAGTCACCCACACAAAG
GAAAAGGGCCTTTCCGTCCTCAGCCGTCGCTTCATGTGACTCCACGGAG
TACCGGGCGCCGTCCAGGCACCTCGATTAGTTCTCCAGCTTTTGGAGTA
CGTCGTCTTTAGGTTGGGGGGAGGGGTTTTATGCGATGGAGTTTCCCCA
CACTGAGTGGGTGGAGACTGAAGTTAGGCCAGCTTGGCACTTGATGTAA
TTCTCCTTGGAATTTGCCCTTTTTGAGTTTGGATCTTGGTTCATTCTCA
AGCCTCAGACAGTGGTTCAAAGTTTTTTTCTTCCATTTCAGGTGTCGTG
AAAA

[symbol]

In one embodiment, the promoter comprises the sequence (chicken β-Actin/Rabbit β-globin chimeric 5′UTR-intron with tbs-kzkV0.G variant, exonic sequence in bold (spliced together to become 5′UTR leader), tbskzkV0.G in italics):

(SEQ ID NO: 117)
CGGCGGGCGGGAACGTTGCCTTCGCCCCGTGCCCCGCTCCGCGCCGCCT
CGCGCCGCCCGCCCCGGCTCTGACTGACCGCGTTACTCCCACAGGTGAG
CGGGCGGGACGGCCCTTCTCCCTCCGGGCTGTAATTAGCGCTTGGTTTA
ATGACGGCTCGTTTCTTTTCTGTGGCTGCGTGAAAGCCTTAAAGGGCTC
CGGGAGGGCCTTTGTGCGGGGGGGAGCGGCTCGGGGGGTGCGTGCGTGT
GTGTGTGCGTGGGGAGCGCCGCGTGCGGCCCGCGCTGCCCGGCGGCTGT
GAGCGCTGCGGGCGCGGCGCGGGGCTTTGTGCGCTCCGCGTGTGCGCGA
GGGGAGCGCGGGCCGGGGGCGGTGCCCCGCGGTGCGGGGGGGCTGCGAG
GGGAACAAAGGCTGCGTGCGGGGTGTGTGCGTGGGGGGGTGAGCAGGGG
GTGTGGGCGCGGCGGTCGGGCTGTAACCCCCCCCTGGCACCCCCCTCCC
CGAGTTGCTGAGCACGGCCCGGCTTCGGGTGCGGGGCTCCGTGCGGGGC
GTGGCGCGGGGCTCGCCGTGCCGGGCGGGGGGTGGCGGCAGGTGGGGGT
GCCGGGCGGGGCGGGGCCGCCTCGGGCCGGGGAGGGCTCGGGGGAGGGG
CGCGGCGGCCCCGGAGCGCCGGCGGCTGTCGAGGCGCGGCGAGCCGCAG
CCATTGCCTTTTATGGTAATCGTGCGAGAGGGCGCAGGGACTTCCTTTG
TCCCAAATCTGGCGGAGCCGAAATCTGGGAGGCGCCGCCGCACCCCCTC
TAGCGGGCGCGGGCGAAGCGGTGCGGCGCCGGCAGGAAGGAAATGGGCG
GGGAGGGCCTTCGTGCGTCGCCGCGCCGCCGTCCCCTTCTCCATCTCCA
GCCTCGGGGCTGCCGCAGGGGGACGGCTGCCTTCGGGGGGGACGGGGCA
GGGCGGGGTTCGGCTTCTGGCGTGTGACCGGCGGCTTTAGAGCCTCTGC
TAACCATGTTCATGCCTTCTTCTTTTTCCTACAGCTCCTGGGCAAA

[symbol]

[symbol]

[symbol]

[symbol]

G

In one embodiment, the promoter comprises the sequence (EF1a 5′UTR-intron with overlapping tbs and Kozak sequence (tbskzkV0.G variant), exonic sequence in bold (spliced together to become 5′UTR leader), tbskzkV0.G in italics):

(SEQ ID NO: 118)
CTTTTTCGCAACGGGTTTGCCGCCAGAACACAGGTAAGTGCCGTGTGTG
GTTCCCGCGGGCCTGGCCTCTTTACGGGTTATGGCCCTTGCGTGCCTTG
AATTACTTCCACCTGGCTGCAGTACGTGATTCTTGATCCCGAGCTTCGG
GTTGGAAGTGGGTGGGAGAGTTCGTGGCCTTGCGCTTAAGGAGCCCCTT
CGCCTCGTGCTTGAGTTGTGGCCTGGCCTGGGCGCTGGGGCCGCCGCGT
GCGAATCTGGTGGCACCTTCGCGCCTGTCTCGCTGCTTTCGATAAGTCT
CTAGCCATTTAAAATTTTTGATGACCTGCTGCGACGCTTTTTTTCTGGC
AAGATAGTCTTGTAAATGCGGGCCAAGATCAGCACACTGGTATTTCGGT
TTTTGGGGCCGCGGGCGGCGACGGGGCCCGTGCGTCCCAGCGCACATGT
TCGGCGAGGCGGGGCCTGCGAGCGCGGCCACCGAGAATCGGACGGGGGT
AGTCTCAAGCTGCCCGGCCTGCTCTGGTGCCTGGCCTCGCGCCGCCGTG
TATCGCCCCGCCCTGGGCGGCAAGGCTGGCCCGGTCGGCACCAGTTGCG
TGAGCGGAAAGATGGCCGCTTCCCGGCCCTGCTGCAGGGAGCACAAAAT
GGAGGACGCGGCGCTCGGGAGAGCGGGCGGGTGAGTCACCCACACAAAG
GAAAAGGGCCTTTCCGTCCTCAGCCGTCGCTTCATGTGACTCCACGGAG
TACCGGGCGCCGTCCAGGCACCTCGATTAGTTCTCCAGCTTTTGGAGTA
CGTCGTCTTTAGGTTGGGGGGAGGGGTTTTATGCGATGGAGTTTCCCCA
CACTGAGTGGGTGGAGACTGAAGTTAGGCCAGCTTGGCACTTGATGTAA
TTCTCCTTGGAATTTGCCCTTTTTGAGTTTGGATCTTGGTTCATTCTCA
AGCCTCAGACAGTGGTTCAAAGTTTTTTTCTTCCATTTCAGGTGTCGTG
AAAA [symbol] [symbol] [symbol] [symbol]
G

Spliced sequence corresponding to SEQ ID NO: 117; 5′UTR leader sequence in bold, tbskzkV0.G in italics:

(SEQ ID NO: 119)
CGGCGGGCGGGAACGTTGCCTTCGCCCCGTGCCCCGCTCCGCGCCGCCT
CGCGCCGCCCGCCCCGGCTCTGACTGACCGCGTTACTCCCACAGCTCCT
GGGCAAA [symbol] [symbol] [symbol] [symbol]
G

Spliced sequence corresponding to SEQ ID NO: 118; 5′UTR leader sequence in bold, tbskzkV0.G in italics:

(SEQ ID NO: 120)
CTTTTTCGCAACGGGTTTGCCGCCAGAACACAGGTGTCGTGAAAA

[symbol]

[symbol]

[symbol]

[symbol]

G

Illustrative Nucleic Acid Sequences

Illustrative nucleic acid sequences of the invention are shown below.

SEQ ID NO: 62—Illustrative nucleic acid sequence 1 containing L33 Improved leader, optimal (overlapping) tbs ([KAGNNN]₈)-Kozak junction

CTTTTTCGCAACGGGTTGCCGCCAGAACACAGGAGTTTAGCGGAGTGGA
GAAGAGCGGAGCCGA

[symbol]

SEQ ID NO: 63—Illustrative nucleic acid sequence 2 containing L33 Improved leader, optimal (overlapping) tbs ([KAGNN]₁₁)-Kozak junction

CTTTTTCGCAACGGGTTGCCGCCAGAACACAGGAGTTTAGCGGAGTGGA
GAAGAGCGGAGCCGAGCCTAGCAGAGACGA

[symbol]

SEQ ID NO: 64—Illustrative nucleic acid sequence 3 containing L12 Improved leader, optimal (overlapping) tbs ([KAGNN]₁₁)-Kozak

CTTTTTCGCAACGAGTTTAGCGGAGTGGAGAAGAGCGGAGCCGAGCCTA
GCAGAGACGA

[symbol]

SEQ ID NO: 65—Illustrative nucleic acid sequence 4 for intron-containing 5′UTRs, resulting in a spliced leader comprising L33, optimal (overlapping) tbs ([KAGNN]₁₁)-Kozak junction

CTTTTTCGCAACGGGTTTGCCGCCAGAACACAGGTGTCGTGAAAAGAG
TTTAGCGGAGTGGAGAAGAGCGGAGCCGAGCCTAGCAGAGACGAGCCG
AGATG

SEQ ID NO: 66—Illustrative nucleic acid sequence 5 containing improved spacer, optimal (overlapping) tbs ([KAGNN]₈)-Kozak junction

ATAGCAGAGACGGCTGAGTTTAGCGGAGTGGAGAAGAGCGGAGCCGA

[symbol]

SEQ ID NO: 67—Illustrative nucleic acid sequence 6 containing L33 Improved leader tbs ([KAGNN]₁₁)-MCS-Kozak

CTTTTTCGCAACGGGTTGCCGCCAGAACACAGGAGTTTAGCGGAGAA
GAGCGGAGCCGAGCCTAGCAGAGACGAGAAGAGCTCTAGACCATG

SEQ ID NO: 68—Illustrative nucleic acid sequence 7 containing improved spacer, tbs ([KAGNN]₁₁)-MCS-Kozak

ATAGCAGAGACGGCTGAGTTTAGCGGAGTGGAGAAGAGCGGAGCCGAG
CCTAGCAGAGACGAGAAGAGCTCTAGACCATG

In one embodiment, the nucleic acid sequence comprises any one of SEQ ID NO: 62-68.

In one embodiment, the nucleic acid sequence comprises:
(a) (i) SEQ ID NO: 25 or 26; and/or
   (ii) any one of SEQ ID NOs: 38-44;
(b) any one of SEQ ID NOs: 10-13, 15-19, 23, 24; and
(c) (i) any one of SEQ ID NOs:29-36, preferably any one of SEQ ID NOs: 34-36;
   or
   (ii) any one of SEQ ID NOs: 45-58, preferably any one of SEQ ID NOs: 52-58.

In one embodiment, the nucleic acid sequence comprises:
(a) SEQ ID NO: 25 or 26;
(b) any one of SEQ ID NOs: 10-13, 16-19, 23, 24; and
(c) any one of SEQ ID NOs:29-36, preferably any one of SEQ ID NOs: 34-36.

In one embodiment, the nucleic acid sequence comprises:
(a) any one of SEQ ID NOs: 38-44;
(b) any one of SEQ ID NOs: 10-13, 16-19, 23, 24; and
(c) any one of SEQ ID NOs:29-36, preferably any one of SEQ ID NOs: 34-36.

In one embodiment, the nucleic acid sequence comprises:
(a) SEQ ID NO: 25 or 26;
(b) any one of SEQ ID NOs: 10-13, 15-19, 23, 24; and
(c) any one of SEQ ID NOs: 45-58, preferably any one of SEQ ID NOs: 52-58.

In one embodiment, the nucleic acid sequence comprises:
(a) any one of SEQ ID NOs: 38-44;
(b) any one of SEQ ID NOs: 10-13, 15-19, 23, 24; and
(c) any one of SEQ ID NOs: 45-58, preferably any one of SEQ ID NOs: 52-58.

Nucleotide of Interest

In one embodiment of the invention, the nucleotide of interest is translated in a target cell which lacks TRAP.

“Target cell” is to be understood as a cell in which it is desired to express the NOI. The NOI may be introduced into the target cell using a viral vector of the present invention. Delivery to the target cell may be performed in vivo, ex vivo or in vitro.

In a preferred embodiment, the nucleotide of interest gives rise to a therapeutic effect.

The NOI may have a therapeutic or diagnostic application. Suitable NOIs include, but are not limited to sequences encoding enzymes, co-factors, cytokines, chemokines, hormones, antibodies, anti-oxidant molecules, engineered immunoglobulin-like molecules, single chain antibodies, fusion proteins, immune co-stimulatory molecules, immunomodulatory molecules, chimeric antigen receptors a transdomain negative mutant of a target protein, toxins, conditional toxins, antigens, transcription factors, structural proteins, reporter proteins, subcellular localization signals, tumour suppressor proteins, growth factors, membrane proteins, receptors, vasoactive proteins and peptides, anti-viral proteins and ribozymes, and derivatives thereof (such as derivatives with an associated reporter group). The NOIs may also encode micro-RNA. Without wishing to be bound by theory, it is believed that the processing of micro-RNA will be inhibited by TRAP.

In one embodiment, the NOI may be useful in the treatment of a neurodegenerative disorder.

In another embodiment, the NOI may be useful in the treatment of Parkinson's disease.

In another embodiment, the NOI may encode an enzyme or enzymes involved in dopamine synthesis. For example, the enzyme may be one or more of the following: tyrosine hydroxylase, GTP-cyclohydrolase I and/or aromatic amino acid dopa decarboxylase. The sequences of all three genes are available (GenBank® Accession Nos. X05290, U19523 and M76180, respectively).

In another embodiment, the NOI may encode the vesicular monoamine transporter 2 (VMAT2). In an alternative embodiment the viral genome may comprise a NOI encoding aromatic amino acid dopa decarboxylase and a NOI encoding VMAT2. Such a genome may be used in the treatment of Parkinson's disease, in particular in conjunction with peripheral administration of L-DOPA.

In another embodiment the NOI may encode a therapeutic protein or combination of therapeutic proteins.

In another embodiment, the NOI may encode a protein or proteins selected from the group consisting of glial cell derived neurotophic factor (GDNF), brain derived neurotrophic factor (BDNF), ciliary neurotrophic factor (CNTF), neurotrophin-3 (NT-3), acidic fibroblast growth factor (aFGF), basic fibroblast growth factor (bFGF), interleukin-1 beta (IL-1β), tumor necrosis factor alpha (TNF-α), insulin growth factor-2, VEGF-A, VEGF-B, VEGF-C/VEGF-2, VEGF-D, VEGF-E, PDGF-A, PDGF-B, hetero- and homo-dimers of PDFG-A and PDFG-B.

In another embodiment, the NOI may encode an anti-angiogenic protein or anti-angiogenic proteins selected from the group consisting of angiostatin, endostatin, platelet factor 4, pigment epithelium derived factor (PEDF), placental growth factor, restin, interferon-α, interferon-inducible protein, gro-beta and tubedown-1, interleukin (IL)-1, IL-12, retinoic acid, anti-VEGF antibodies or fragments/variants thereof such as aflibercept, thrombospondin, VEGF receptor proteins such as those described in U.S. Pat. Nos. 5,952,199 and 6,100,071, and anti-VEGF receptor antibodies.

In another embodiment, the NOI may encode anti-inflammatory proteins, antibodies or fragment/variants of proteins or antibodies selected from the group consisting of NF-kB inhibitors, IL1beta inhibitors, TGFbeta inhibitors, IL-6 inhibitors, IL-23 inhibitors, IL-18 inhibitors, Tumour necrosis factor alpha and Tumour necrosis factor beta, Lymphotoxin alpha and Lymphotoxin beta, LIGHT inhibitors, alpha synuclein inhibitors, Tau inhibitors, beta amyloid inhibitors, IL-17 inhibitors,

In another embodiment the NOI may encode cystic fibrosis transmembrane conductance regulator (CFTR).

In another embodiment the NOI may encode a protein normally expressed in an ocular cell.

In another embodiment, the NOI may encode a protein normally expressed in a photoreceptor cell and/or retinal pigment epithelium cell.

In another embodiment, the NOI may encode a protein selected from the group comprising RPE65, arylhydrocarbon-interacting receptor protein like 1 (AIPL1), CRB1, lecithin retinal acetyltransferace (LRAT), photoreceptor-specific homeo box (CRX), retinal guanylate cyclise (GUCY2D), RPGR interacting protein 1 (RPGRIP1), LCA2, LCA3, LCA5, dystrophin, PRPH2, CNTF, ABCR/ABCA4, EMP1, TIMP3, MERTK, ELOVL4, MYO7A, USH2A, VMD2, RLBP1, COX-2, FPR, harmonin, Rab escort protein 1, CNGB2, CNGA3, CEP 290, RPGR, RS1, RP1, PRELP, glutathione pathway enzymes and opticin.

In other embodiments, the NOI may encode the human clotting Factor VIII or Factor IX.

In other embodiments, the NOI may encode protein or proteins involved in metabolism selected from the group comprising phenylalanine hydroxylase (PAH), Methylmalonyl CoA mutase, Propionyl CoA carboxylase, Isovaleryl CoA dehydrogenase, Branched chain ketoacid dehydrogenase complex, Glutaryl CoA dehydrogenase, Acetyl CoA carboxylase, propionyl CoA carboxylase, 3 methyl crotonyl CoA carboxylase, pyruvate carboxylase, carbamoyl-phophate synthase ammonia, ornithine transcarbamylase, glucosylceramidase beta, alpha galactosidase A, glucosylceramidase beta, cystinosin, glucosamine(N-acetyl)-6-sulfatase, N-acetyl-alpha-glucosaminidase, N-sulfoglucosamine sulfohydrolase, Galactosamine-6 sulfatase, arylsulfatase A, cytochrome B-245 beta, ABCD1, ornithine carbamoyltransferase, argininosuccinate synthase, argininosuccinate lysase, arginase 1, alanine glycoxhylate amino transferase, ATP-binding cassette, sub-family B members.

In other embodiments, the NOI may encode a chimeric antigen receptor (CAR) or a T cell receptor (TCR). In one embodiment, the CAR is an anti-5T4 CAR. In other embodiments, the NOI may encode B-cell maturation antigen (BCMA), CD19, CD22, CD20, CD138, CD30, CD33, CD123, CD70, prostate specific membrane antigen (PSMA), Lewis Y antigen (LeY), Tyrosine-protein kinase transmembrane receptor (ROR1), Mucin 1, cell surface associated (Muc1), Epithelial cell adhesion molecule (EpCAM), endothelial growth factor receptor (EGFR), insulin, protein tyrosine phosphatase, non-receptor type 22, interleukin 2 receptor, alpha, interferon induced with helicase C domain 1, human epidermal growth factor receptor (HER2), glypican 3 (GPC3), disialoganglioside (GD2), mesiothelin, vesicular endothelial growth factor receptor 2 (VEGFR2).

In other embodiments, the NOI may encode a chimeric antigen receptor (CAR) against NKG2D ligands selected from the group comprising ULBP1, 2 and 3, H60, Rae-1a, b, g, d, MICA, MICB.

In further embodiments the NOI may encode SGSH, SUMF1, GAA, the common gamma chain (CD132), adenosine deaminase, WAS protein, globins, alpha galactosidase A, O-aminolevulinate (ALA) synthase, δ-aminolevulinate dehydratase (ALAD), Hydroxymethylbilane (HMB) synthase, Uroporphyrinogen (URO) synthase, Uroporphyrinogen (URO) decarboxylase, Coproporphyrinogen (COPRO) oxidase, Protoporphyrinogen (PROTO) oxidase, Ferrochelatase, α-L-iduronidase, Iduronate sulfatase, Heparan sulfamidase, N-acetylglucosaminidase, Heparan-α-glucosaminide N-acetyltransferase, 3 N-acetylglucosamine 6-sulfatase, Galactose-6-sulfate sulfatase, β-galactosidase, N-acetylgalactosamine-4-sulfatase, β-glucuronidase and Hyaluronidase.

In addition to the NOI the vector may also comprise or encode a siRNA, shRNA, or regulated shRNA. (Dickins et al. (2005) Nature Genetics 37: 1289-1295, Silva et al. (2005) Nature Genetics 37:1281-1288).

Indications

The vectors, including retroviral and AAV vectors, according to the present invention may be used to deliver one or more NOI(s) useful in the treatment of the disorders listed in WO 1998/05635, WO 1998/07859, WO 1998/09985. The nucleotide of interest may be DNA or RNA. Examples of such diseases are given below:
A disorder which responds to cytokine and cell proliferation/differentiation activity; immunosuppressant or immunostimulant activity (e.g. for treating immune deficiency, including infection with human immunodeficiency virus, regulation of lymphocyte growth; treating cancer and many autoimmune diseases, and to prevent transplant rejection or induce tumour immunity); regulation of haematopoiesis (e.g. treatment of myeloid or lymphoid diseases); promoting growth of bone, cartilage, tendon, ligament and nerve tissue (e.g. for healing wounds, treatment of burns, ulcers and periodontal disease and neurodegeneration); inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating, for example, septic shock or Crohn's disease); macrophage inhibitory and/or T cell inhibitory activity and thus, anti-inflammatory activity; anti-immune activity (i.e. inhibitory effects against a cellular and/or humoral immune response, including a response not associated with inflammation); inhibition of the ability of macrophages and T cells to adhere to extracellular matrix components and fibronectin, as well as up-regulated fas receptor expression in T cells.
Malignancy disorders, including cancer, leukaemia, benign and malignant tumour growth, invasion and spread, angiogenesis, metastases, ascites and malignant pleural effusion.
Autoimmune diseases including arthritis, including rheumatoid arthritis, hypersensitivity, allergic reactions, asthma, systemic lupus erythematosus, collagen diseases and other diseases.
Vascular diseases including arteriosclerosis, atherosclerotic heart disease, reperfusion injury, cardiac arrest, myocardial infarction, vascular inflammatory disorders, respiratory distress syndrome, cardiovascular effects, peripheral vascular disease, migraine and aspirin-dependent anti-thrombosis, stroke, cerebral ischaemia, ischaemic heart disease or other diseases.
Diseases of the gastrointestinal tract including peptic ulcer, ulcerative colitis, Crohn's disease and other diseases.
Hepatic diseases including hepatic fibrosis, liver cirrhosis.
Inherited metabolic disorders including phenylketonuria PKU, Wilson disease, organic acidemias, urea cycle disorders, cholestasis, and other diseases.
Renal and urologic diseases including thyroiditis or other glandular diseases, glomerulonephritis or other diseases.
Ear, nose and throat disorders including otitis or other oto-rhino-laryngological diseases, dermatitis or other dermal diseases.
Dental and oral disorders including periodontal diseases, periodontitis, gingivitis or other dental/oral diseases.
Testicular diseases including orchitis or epididimo-orchitis, infertility, orchidal trauma or other testicular diseases.
Gynaecological diseases including placental dysfunction, placental insufficiency, habitual abortion, eclampsia, pre-eclampsia, endometriosis and other gynaecological diseases.
Ophthalmologic disorders such as Leber Congenital Amaurosis (LCA) including LCA10, posterior uveitis, intermediate uveitis, anterior uveitis, conjunctivitis, chorioretinitis, uveoretinitis, optic neuritis, glaucoma, including open angle glaucoma and juvenile congenital glaucoma, intraocular inflammation, e.g. retinitis or cystoid macular oedema, sympathetic ophthalmia, scleritis, retinitis pigmentosa, macular degeneration including age related macular degeneration (AMD) and juvenile macular degeneration including Best Disease, Best vitelliform macular degeneration, Stargardt's Disease, Usher's syndrome, Doyne's honeycomb retinal dystrophy, Sorby's Macular Dystrophy, Juvenile retinoschisis, Cone-Rod Dystrophy, Corneal Dystrophy, Fuch's Dystrophy, Leber's congenital amaurosis, Leber's hereditary optic neuropathy (LHON), Adie syndrome, Oguchi disease, degenerative fondus disease, ocular trauma, ocular inflammation caused by infection, proliferative vitreo-retinopathies, acute ischaemic optic neuropathy, excessive scarring, e.g. following glaucoma filtration operation, reaction against ocular implants, corneal transplant graft rejection, and other ophthalmic diseases, such as diabetic macular oedema, retinal vein occlusion, RLBP1-associated retinal dystrophy, choroideremia and achromatopsia.
Neurological and neurodegenerative disorders including Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, strokes, post-polio syndrome, psychiatric disorders, myelitis, encephalitis, subacute sclerosing pan-encephalitis, encephalomyelitis, acute neuropathy, subacute neuropathy, chronic neuropathy, Fabry disease, Gaucher disease, Cystinosis, Pompe disease, metachromatic leukodystrophy, Wiscott Aldrich Syndrome, adrenoleukodystrophy, beta-thalassemia, sickle cell disease, Guillaim-Barre syndrome, Sydenham chorea, myasthenia gravis, pseudo-tumour cerebri, Down's Syndrome, Huntington's disease, CNS compression or CNS trauma or infections of the CNS, muscular atrophies and dystrophies, diseases, conditions or disorders of the central and peripheral nervous systems, motor neuron disease including amyotropic lateral sclerosis, spinal muscular atropy, spinal cord and avulsion injury.
Other diseases and conditions such as cystic fibrosis, mucopolysaccharidosis including Sanfilipo syndrome A, Sanfilipo syndrome B, Sanfilipo syndrome C, Sanfilipo syndrome D, Hunter syndrome, Hurler-Scheie syndrome, Morquio syndrome, ADA-SCID, X-linked SCID, X-linked chronic granulomatous disease, porphyria, haemophilia A, haemophilia B, post-traumatic inflammation, haemorrhage, coagulation and acute phase response, cachexia, anorexia, acute infection, septic shock, infectious diseases, diabetes mellitus, complications or side effects of surgery, bone marrow transplantation or other transplantation complications and/or side effects, complications and side effects of gene therapy, e.g. due to infection with a viral carrier, or AIDS, to suppress or inhibit a humoral and/or cellular immune response, for the prevention and/or treatment of graft rejection in cases of transplantation of natural or artificial cells, tissue and organs such as cornea, bone marrow, organs, lenses, pacemakers, natural or artificial skin tissue.

siRNA, micro-RNA and shRNA

In certain other embodiments, the NOI comprises a micro-RNA. Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70nt precursor, which is post-transcriptionally processed into a mature ˜21nt form. Both let-7 and lin-4 are transcribed as hairpin RNA precursors which are processed to their mature forms by Dicer enzyme.

In addition to the NOI the vector may also comprise or encode a siRNA, shRNA, or regulated shRNA (Dickins et al. (2005) Nature Genetics 37: 1289-1295, Silva et al. (2005) Nature Genetics 37:1281-1288).

Post-transcriptional gene silencing (PTGS) mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nature Medicine 11:429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA>30 bp has been found to activate the interferon response leading to shut-down of protein synthesis and non-specific mRNA degradation (Stark et al., Annu Rev Biochem 67:227-64 (1998)). However this response can be bypassed by using 21nt siRNA duplexes (Elbashir et al., EMBO J. December 3; 20(23):6877-88 (2001), Hutvagner et al., Science. August 3, 293(5531):834-8. Eupub July 12 (2001)) allowing gene function to be analysed in cultured mammalian cells.

NOI and Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing PCR under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

Major Splice Donor

RNA splicing is catalysed by a large RNA-protein complex called the spliceosome, which is comprised of five small nuclear ribonucleoproteins (snRNPs). The borders between introns and exons are marked by specific nucleotide sequences within a pre-mRNA, which delineate where splicing will occur. Such boundaries are referred to as “splice sites.” The term “splice site” refers to polynucleotides that are capable of being recognized by the splicing machinery of a eukaryotic cell as suitable for being cut and/or ligated to another splice site.

Splice sites allow for the excision of introns present in a pre-mRNA transcript. Typically, the 5′ splice boundary is referred to as the “splice donor site” or the “5′ splice site,” and the 3′ splice boundary is referred to as the “splice acceptor site” or the “3′ splice site.” Splice sites include, for example, naturally occurring splice sites, engineered or synthetic splice sites, canonical or consensus splice sites, and/or non-canonical splice sites, for example, cryptic splice sites.

Splice acceptor sites generally consist of three separate sequence elements: the branch point or branch site, a polypyrimidine tract and the acceptor consensus sequence. The branch point consensus sequence in eukaryotes is YNYTRAC (where Y is a pyrimidine, N is any nucleotide, and R is a purine). The 3′ acceptor splice site consensus sequence is YAG (where Y is a pyrimidine) (see, e.g., Griffiths et al., eds., Modern Genetic Analysis, 2nd edition, W.H. Freeman and Company, New York (2002)). The 3′ splice acceptor site typically is located at the 3′ end of an intron.

As such, the major splice donor site may be inactivated in the nucleotide sequence encoding the RNA genome of the lentiviral vector for use in the present invention.

In one aspect the invention also provides a nucleic acid sequence according to the invention as described herein, wherein the nucleic acid sequence is comprised within the RNA genome of the lentiviral vector, and wherein major splice donor site in the RNA genome of the lentiviral vector is inactivated, for example is mutated or deleted.

In one aspect the invention also provides a nucleic acid sequence according to the invention as described herein, wherein the nucleic acid sequence is operably linked to the RNA genome of the lentiviral vector, and wherein major splice donor site in the RNA genome of the lentiviral vector is inactivated, for example is mutated or deleted.

In one aspect is provided a nucleotide sequence encoding the RNA genome of a lentiviral vector, wherein the major splice donor site in the RNA genome of said lentiviral vector is inactivated, for example is mutated or deleted.

The terms “canonical splice site” or “consensus splice site” may be used interchangeably and refer to splice sites that are conserved across species.

Consensus sequences for the 5′ donor splice site and the 3′ acceptor splice site used in eukaryotic RNA splicing are well known in the art. These consensus sequences include nearly invariant dinucleotides at each end of the intron: GT at the 5′ end of the intron, and AG at the 3′ end of an intron.

The canonical splice donor site consensus sequence may be (for DNA) AG/GTRAGT (where A is adenosine, T is thymine, G is guanine, C is cytosine, R is a purine and “I” indicates the cleavage site). It is well known in the art that a splice donor may deviate from this consensus, especially in viral genomes where other constraints bear on the same sequence, such as secondary structure for example within a vRNA packaging region. Non-canonical splice sites are also well known in the art, albeit they occur rarely compared to the canonical splice donor consensus sequence.

By “major splice donor site” is meant the first (dominant) splice donor site in the viral vector genome, encoded and embedded within the native viral RNA packaging sequence typically located in the 5′ region of the viral vector nucleotide sequence.

In one aspect the nucleotide sequence encoding the RNA genome of the lentiviral vector does not contain an active major splice donor site, that is splicing does not occur from the major splice donor site in said nucleotide sequence, and splicing activity from the major splice donor site is ablated.

The major splice donor site is located in the 5′ packaging region of a lentiviral genome.

In the case of the HIV-1 virus, the major splice donor consensus sequence is (for DNA) TG/GTRAGT (where A is adenosine, T is thymine, G is guanine, C is cytosine, R is a purine and “/” indicates the cleavage site).

In one aspect of the invention, the splice donor region, i.e. the region of the vector genome which comprises the major splice donor site prior to mutation may have the following sequence:

(SEQ ID NO: 94)
GGGGCGGCGACTGGTGAGTACGCCAAAAAT

In one aspect of the invention the mutated splice donor region may comprise the sequence:

(SEQ ID NO: 95-MSD-2KO)
GGGGCGGCGACTGCAGACAACGCCAAAAAT

In one aspect of the invention the mutated splice donor region may comprise the sequence:

(SEQ ID NO: 104-MSD-2KOv2)
GGGGCGGCGAGTGGAGACTACGCCAAAAAT

In one aspect of the invention the mutated splice donor region may comprise the sequence:

(SEQ ID NO: 105-MSD-2KOm5)
GGGGAAGGCAACAGATAAATATGCCTTAAAAT

In one aspect of the invention prior to modification the splice donor region may comprise the sequence:

(SEQ ID NO: 102)
GGCGACTGGTGAGTACGCC

This sequence is also referred to herein as the “stem loop 2” region (SL2). This sequence may form a stem loop structure in the splice donor region of the vector genome. In one aspect of the invention this sequence (SL2) may have been deleted from the nucleotide sequence according to the invention as described herein.

As such, the invention encompasses a nucleotide sequence that does not comprise SL2. The invention encompasses a nucleotide sequence that does not comprise a sequence according to SEQ ID NO:102.

In one aspect of the invention the major splice donor site may have the following consensus sequence, wherein R is a purine and “I” is the cleavage site:

(SEQ ID NO: 96)
TG/GTRAGT

In one aspect, R may be guanine (G).

In one aspect of the invention, the major splice donor and cryptic splice donor region may have the following core sequence, wherein “I” are the cleavage sites at the major splice donor and cryptic splice donor sites:

(SEQ ID NO: 106)
/GTGA/GTA.

In one aspect of the invention the MSD-mutated vector genome may have at least two mutations in the major splice donor and cryptic splice donor ‘region’ (SEQ ID NO:106), wherein the first and second ‘GT’ nucleotides are the immediately 3′ of the major splice donor and cryptic splice donor nucleotides respectively

In one aspect of the invention the major splice donor consensus sequence is CTGGT (SEQ ID NO:97). The major splice donor site may contain the sequence CTGGT.

In one aspect the nucleotide sequence, prior to inactivation of the splice sites, comprises the sequence as set forth in any of SEQ ID NOs: 94, 96, 97, 102, 103 and/or 106.

In one aspect the nucleotide sequence comprises an inactivated major splice donor site which would otherwise have a cleavage site between nucleotides corresponding to nucleotides 13 and 14 of SEQ ID NO:94.

According to the invention as described herein, the nucleotide sequence also contains an inactive cryptic splice donor site. In one aspect the nucleotide sequence does not contain an active cryptic splice donor site adjacent to (3′ of) the major splice donor site, that is to say that splicing does not occur from the adjacent cryptic splice donor site, and splicing from the cryptic splice donor site is ablated.

The term “cryptic splice donor site” refers to a nucleic acid sequence which does not normally function as a splice donor site or is utilised less efficiently as a splice donor site due to the adjacent sequence context (e.g. the presence of a nearby ‘preferred’ splice donor), but can be activated to become a more efficient functioning splice donor site by mutation of the adjacent sequence (e.g. mutation of the nearby ‘preferred’ splice donor).

In one aspect the cryptic splice donor site is the first cryptic splice donor site 3′ of the major splice donor.

In one aspect the cryptic splice donor site is within 6 nucleotides of the major splice donor site on the 3′ side of the major splice donor site. Preferably the cryptic splice donor site is within 4 or 5, preferably 4, nucleotides of the major splice donor cleavage site.

In one aspect of the invention the cryptic splice donor site has the consensus sequence

(SEQ ID NO: 103)
TGAGT.

In one aspect the nucleotide sequence comprises an inactivated cryptic splice donor site which would otherwise have a cleavage site between nucleotides corresponding to nucleotides 17 and 18 of SEQ ID NO:94.

In one aspect of the invention the major splice donor site and/or adjacent cryptic splice donor site contain a “GT” motif. In one aspect of the invention both the major splice donor site and adjacent cryptic splice donor site contain a “GT” motif which is mutated. The mutated GT motifs may inactivate splice activity from both the major splice donor site and adjacent cryptic splice donor site. An example of such a mutation is referred to herein as “MSD-2KO”.

In one aspect the splice donor region may comprise the following sequence:

(SEQ ID NO: 98)
CAGACA

For example, in one aspect the mutated splice donor region may comprise the following sequence:

(SEQ ID NO: 99)
GGCGACTGCAGACAACGCC

A further example of an inactivating mutation is referred to herein as “MSD-2KOv2”.

In one aspect the mutated splice donor region may comprise the following sequence:

(SEQ ID NO: 100)
GTGGAGACT

For example, in one aspect the mutated splice donor region may comprise the following sequence:

(SEQ ID NO: 101)
GGCGAGTGGAGACTACGCC

For example, in one aspect the mutated splice donor region may comprise the following sequence:

(SEQ ID NO: 107)
AAGGCAACAGATAAATATGCCTT

In one aspect the stem loop 2 region as described above may be deleted from the splice donor region, resulting in inactivation of both the major splice donor site and the adjacent cryptic splice donor site. Such a deletion is referred to herein as “ΔSL2”.

A variety of different types of mutations can be introduced into the nucleic acid sequence in order to inactivate the major and adjacent cryptic splice donor sites.

In one aspect the mutation is a functional mutation to ablate or suppress splicing activity in the splice region. The nucleotide sequence as described herein may contain a mutation or deletion in any of the nucleotides in any of SEQ ID NOs: 94, 96, 97, 102, 103 and/or 106. Suitable mutations will be known to one skilled in the art, and are described herein.

For example, a point mutation can be introduced into the nucleic acid sequence. The term “point mutation,” as used herein, refers to any change to a single nucleotide. Point mutations include, for example, deletions, transitions, and transversions; these can be classified as nonsense mutations, missense mutations, or silent mutations when present within protein coding sequence. A “nonsense” mutation produces a stop codon. A “missense” mutation produces a codon that encodes a different amino acid. A “silent” mutation produces a codon that encodes either the same amino acid or a different amino acid that does not alter the function of the protein. One or more point mutations can be introduced into the nucleic acid sequence comprising the cryptic splice donor site. For example, the nucleic acid sequence comprising the cryptic splice site can be mutated by introducing two or more point mutations therein.

At least two point mutations can be introduced in several locations within the nucleic acid sequence comprising the major splice donor and cryptic splice donor sites to achieve attenuation of splicing from the splice donor region. In one aspect the mutations may be within the four nucleotides at the splice donor cleavage site; in the canonical splice donor consensus sequence this is A¹G²/G³T⁴, wherein “I” is the cleavage site. It is well known in the art that a splice donor cleavage site may deviate from this consensus, especially in viral genomes where other constraints bear on the same sequence, such as secondary structure for example within a vRNA packaging region. It is well known that the G³T⁴ dinucleotide is generally the least variable sequence within the canonical splice donor consensus sequence, and mutations to the G³ and or T⁴ will most likely achieve the greatest attenuating effect. For example, for the major splice donor site in HIV-1 viral vector genomes this can be T¹G²/G³T⁴, wherein “/” is the cleavage site. For example, for the cryptic splice donor site in HIV-1 viral vector genomes this can be G¹A²/G³T⁴, wherein “/” is the cleavage site.

Additionally, the point mutation(s) can be introduced adjacent to a splice donor site. For example, the point mutation can be introduced upstream or downstream of a splice donor site. In embodiments where the nucleic acid sequence comprising a major and/or cryptic splice donor site is mutated by introducing multiple point mutations therein, the point mutations can be introduced upstream and/or downstream of the cryptic splice donor site.

Construction of Splice Site Mutants

Splice site mutants for use in the present invention may be constructed using a variety of techniques. For example, mutations may be introduced at particular loci by synthesising oligonucleotides containing a mutant sequence, flanked by restriction sites enabling ligation to fragments of the native sequence. Following ligation, the resulting reconstructed sequence comprises a derivative having the desired nucleotide insertion, substitution, or deletion.

Other known techniques allowing alterations of DNA sequence include recombination approaches such as Gibson assembly, Golden-gate cloning and In-fusion.

Alternatively, oligonucleotide-directed site-specific (or segment specific) mutagenesis procedures may be employed to provide an altered sequence having particular codon altered according to the substitution, deletion, or insertion required. Deletion or truncation derivatives of splice site mutants may also be constructed by utilising convenient restriction endonuclease sites adjacent to the desired deletion.

Subsequent to restriction, overhangs may be filled in, and the DNA religated.

Exemplary methods of making the alterations set forth above are disclosed by Sambrook et al. (Molecular cloning: A Laboratory Manual, 2d Ed., Cold Spring Harbor Laboratory Press, 1989).

Splice site mutants may also be constructed utilising techniques of PCR mutagenesis, chemical mutagenesis, chemical mutagenesis (Drinkwater and Klinedinst, 1986) by forced nucleotide misincorporation (e.g., Liao and Wise, 1990), or by use of randomly mutagenised oligonucleotides (Horwitz et al., 1989).

The present invention also provides a method for producing a lentiviral vector nucleotide sequence, comprising the steps of:
(i) providing a nucleotide sequence encoding the RNA genome of a lentiviral vector as described herein; and

mutating the major splice donor site and cryptic splice donor site as described herein in said nucleotide sequence.

Combination with Modified U1

MSD-mutated lentiviral vectors are preferable to current standard lentiviral vectors for use as gene therapy vectors due to their reduced capacity to partake in aberrant splicing events both during LV production and in target cells. However, until the present invention, the production of MSD-mutated vectors has either relied upon supply of the HIV-1 tat protein (1^{st} and 2^{nd} generation lentiviral vectors) or has been of lower efficiency due to the unstabilising effect of mutating the MSD on vector RNA levels (in 3^{rd} generation vectors). Due to safety reasons there is no desire or justification for ‘reintroducing’ tat back into contemporary 3rd generation LV systems, and consequently there is currently no solution to the reduction in production titres of MSD-mutated vectors intended for clinical use.

The present inventors show that MSD-mutated, 3^{rd} generation (i.e. tat-independent) LVs can be produced to high titre by co-expression of a modified U1 snRNA directed to bind to the 5′packaging region of the vector genome RNA during production. It is surprisingly shown that these modified U1 snRNAs can enhance the production titres of MSD-mutated LVs in a manner that is independent of the presence of the 5′polyA signal within the 5′R region, indicating a novel mechanism over others' use of modified U1 snRNAs to suppress polyadenylation (so called U1-interference, [Ui]). It is surprisingly shown that targeting the modified U1 snRNAs to critical sequences of the packaging region produce the greatest enhancement in MSD-mutated LV titres. The present inventors also disclose novel sequence mutation within the major splice donor region such that reduction in titres of MSD-mutated LV is less pronounced, and that enhancement in titres of such MSD-mutated LV variants by modified U1 snRNAs is greatest.

The present inventors have surprisingly found that the output titres of lentiviral vectors can be enhanced by co-expressing non-coding RNAs based on U1 snRNAs, which have been modified so that they no longer target the endogenous sequence (a splice donor site) but now target a sequence within the vRNA molecule. As demonstrated in the present Examples, the inventors show that the relative enhancement in output titres of lentiviral vectors harbouring attenuating mutations within the major splice donor region (containing the major splice donor and cryptic splice donor sites) by said modified U1 snRNAs are greater than standard lentiviral vectors containing a non-mutated major splice donor region.

As demonstrated in the present Examples, vector genomes harbouring a broad range of mutation types within the major splice donor region (point mutations, region deletion, and sequence replacement) that lead to reduced titres may be used in combination with a modified U1 snRNA. The approach may comprise co-expression of modified U1 snRNAs together with the other vector components during vector production. The modified U1 snRNAs are designed such that binding to the consensus splice donor site has been ablated by replacing it with a heterologous sequence that is complementary to a target sequence within the vector genome vRNA. The invention describes various modes of application and optimal characteristics of the modified U1 snRNAs, including target sequence and complementarity length, design and modes of expression.

In one aspect of the invention as described herein, the vector may be used in combination with a modified U1 snRNA. This is discussed further below.

Splicing and polyadenylation are key processes for mRNA maturation, particularly in higher eukaryotes where most protein-coding transcripts contain multiple introns. The elements within a pre-mRNA that are required for splicing include the 5′ splice donor signal, the sequence surrounding the branch point and the 3′ splice acceptor signal. Interacting with these three elements is the spliceosome, which is formed by five small nuclear RNAs (snRNAs), including U1 snRNA, and associated nuclear proteins (snRNP). U1 snRNA is expressed by a polymerase II promoter and is present in most eukaryotic cells (Lund et al., 1984, J. Biol. Chem., 259:2013-2021). Human U1 snRNA (small nuclear RNA) is 164 nt long with a well-defined structure consisting of four stem-loops (West, S., 2012, Biochemical Society Transactions, 40:846-849). U1 snRNA contains a short sequence at its 5′-end that is broadly complementary to the 5′ splice donor sites at exon-intron junctions. U1 snRNA participates in splice-site selection and spliceosome assembly by base pairing to the 5′ splice donor site. A known function for U1 snRNA outside splicing is in the regulation of 3′-end mRNA processing: it suppresses premature polyadenylation (polyA) at early polyA signals.

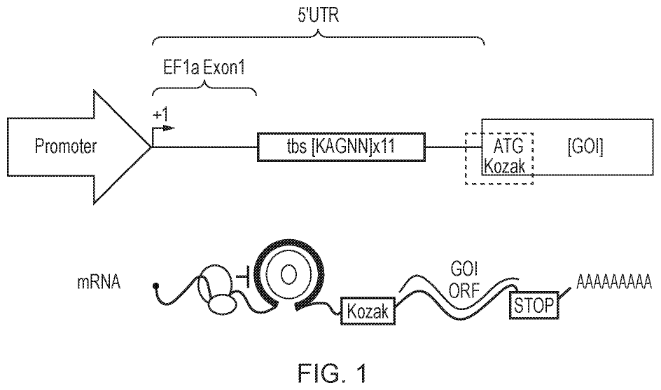

Human U1 snRNA (small nuclear RNA) is 164nt long with a well-defined structure consisting of four stem-loops (see FIG. 1). The endogenous non-coding RNA, U1 snRNA, binds to the consensus 5′ splice donor site (e.g. 5′-MAGGURR-3′, wherein M is A or C and R is A or G) via the native splice donor annealing sequence (e.g. 5′-ACUUACCUG-3′) during early steps of intron splicing. Stem loop I binds to U1A-70K protein that has been shown to be important for polyA suppression. Stem loop II binds to U1A protein, and the 5′-AUUUGUGG-3′ sequence binds to Sm proteins, which together with Stem loop IV, is important for U1 snRNA processing. As defined herein the modified U1 snRNA for use according to the present invention is modified to introduce a heterologous sequence that is complementary to a target sequence within the vector genome vRNA molecule at the site of the native splice donor targeting/annealing sequence (see FIG. 1).

As used herein, the terms “modified U1 snRNA”, “re-directed U1 snRNA”, “re-targeted U1 snRNA”, “re-purposed U1 snRNA” and “mutant U1 snRNA”, mean a U1 snRNA that has been modified so that it is no longer complementary to the consensus 5′ splice donor site sequence (e.g. 5′-MAGGURR-3′) that it uses to initiate the splicing process of a target gene. Thus, a modified U1 snRNA is a U1 snRNA which has been modified so that it is no longer complementary to the splice donor site sequence (e.g. 5′-MAGGURR-3′). Instead, the modified U1 snRNA is designed so that it targets or is complementary to a nucleotide sequence having a unique RNA sequence within the packaging region of a MSD-mutated lentiviral vector genome molecule (target site), i.e. a sequence that is unrelated to splicing of the vRNA. The nucleotide sequence within the packaging region of a MSD-mutated lentiviral vector genome molecule can be preselected. Thus, the modified U1 snRNA is a U1 snRNA which has been modified so that its 5′ end is complementary to a nucleotide sequence within the packaging region of a MSD-mutated lentiviral vector genome molecule. As a result, and not wishing to be bound by theory, the modified U1 snRNA is thought to bind to the target site sequence based on complementarity of the target site sequence with the short sequence at the 5′ end of the modified U1 snRNA, thus stabilising the vRNA leading to increased output vector titres of the MSD-mutated lentiviral vector.

As used herein, the terms “native splice donor annealing sequence” and “native splice donor targeting sequence” mean the short sequence at the 5′-end of the endogenous U1 snRNA that is broadly complementary to the consensus 5′ splice donor site of introns. The native splice donor annealing sequence may be 5′-ACUUACCUG-3′.

As used herein, the term “consensus 5′ splice donor site” means the consensus RNA sequence at the 5′ end of introns used in splice-site selection, e.g. having the sequence 5′-MAGGURR-3′.

As used herein, the terms “nucleotide sequence within the packaging region of a MSD-mutated lentiviral vector genome sequence”, “target sequence” and “target site” mean a site having a particular RNA sequence within the packaging region of a MSD-mutated lentiviral vector genome molecule which has been preselected as the target site for binding/annealing the modified U1 snRNA.

As used herein, the terms “packaging region of a MSD-mutated lentiviral vector genome molecule” and “packaging region of an MSD-mutated lentiviral vector genome sequence” means the region at the 5′ end of an MSD-mutated lentiviral vector genome from the beginning of the 5′ U5 domain to the terminus of the sequence derived from gag gene. Thus, the packaging region of a MSD-mutated lentiviral vector genome molecule includes the 5′ U5 domain, PBS element, stem loop (SL) 1 element, SL2 element, SL3ψ element, SL4 element and the sequence derived from the gag gene. It is common in the art to provide the complete gag gene in trans to the genome during lentiviral vector production to enable the production of replication-defective viral vector particle. The nucleotide sequence of the gag gene provided in trans need not be encoded by wild type nucleotides but may be codon-optimised; importantly the chief attribute of the gag gene provided in trans is that it encodes and directs expression of the gag and gagpol proteins. Accordingly, it will be understood by the person skilled in the art that, if the complete gag gene is to be provided in trans during lentiviral vector production, the term “packaging region of a lentiviral vector genome molecule” may mean the region at the 5′ end of the MSD-mutated lentiviral vector genome molecule from the beginning of the 5′ U5 domain through to the ‘core’ packaging signal at the SL3 ψ element, and the native gag nucleotide sequence from the ATG codon (present within SL4) to the end of the remaining gag nucleotide sequence present on the vector genome.

As used herein, the term “sequence derived from gag gene” means, any native sequence of the gag gene derived from the ATG codon to nucleotide 688 (Kharytonchyk, S. et. al., 2018, J. Mol. Biol., 430:2066-79) that may be present, e.g. remain, in the vector genome.

As used herein, the terms “to introduce within the first 11 nucleotides of the U1 snRNA, which encompasses the native splice donor annealing sequence, a heterologous sequence”, “to introduce within the nine nucleotides at positions 3-to-11 said heterologous sequence” and “to introduce within the first 11 nucleotides at the 5′ end of the U1 snRNA a heterologous sequence” include to replace the first 11 nucleotides, or the nine nucleotides at positons 3-to-11, of the U1 snRNA all or in part with said heterologous sequence or to modify the first 11 nucleotides, or the nine nucleotides at positons 3-to-11, of the U1 snRNA to have the same sequence as said heterologous sequence.

As used herein, the terms “to introduce within the native splice donor annealing sequence a heterologous sequence” and “to introduce within the native splice donor annealing sequence at the 5′ end of the U1 snRNA a heterologous sequence” include to replace the native splice donor annealing sequence all or in part with said heterologous sequence or to modify the native splice donor annealing sequence to have the same sequence as said heterologous sequence.

As used herein, the term “enhances lentiviral vector titres” includes “increases lentiviral vector titres”, “recovers lentiviral vector titres” and “improves lentiviral vector titres”.

Accordingly, in one embodiment, the modified U1 snRNA has been modified to bind to a nucleotide sequence within the packaging region of an MSD-mutated lentiviral vector genome sequence.

In some embodiments, the modified U1 snRNA is modified at the 5′ end relative to the endogenous U1 snRNA to introduce a heterologous sequence that is complementary to a nucleotide sequence within the packaging region of an MSD-mutated lentiviral vector genome.

In some embodiments, the modified U1 snRNA is modified at the 5′ end relative to the endogenous U1 snRNA to introduce within the native splice donor annealing sequence a heterologous sequence that is complementary to a nucleotide sequence within the packaging region of an MSD-mutated lentiviral vector genome.

The modified U1 snRNA may be modified at the 5′ end relative to the endogenous U1 snRNA to replace a sequence encompassing the native splice donor annealing sequence with a heterologous sequence that is complementary to said nucleotide sequence.

The modified U1 snRNA may be a modified U1 snRNA variant. The U1 snRNA variant which is modified in accordance with the invention may be a naturally occurring U1 snRNA variant, a U1 snRNA variant containing a mutation within the stem loop I region ablating U1-70K protein binding, or a U1 snRNA variant containing a mutation in the stem loop II region ablating U1A protein binding. The U1 snRNA variant containing a mutation within the stem loop I region ablating U1-70K protein binding may be U1_m1 or U1_m2, preferably U1A_m1 or U1A_m2.

In some embodiments, the modified U1 snRNA as described herein comprises a nucleotide sequence having at least 70% identity (suitable at least 75%, at least 80%, 85%, 90%, 91%, 92%, 93%, 94%, 95%, 96%, 97%, 98%, 99% identity) with the main U1 snRNA sequence [clover leaf] (nt 410-562) of the U1_256 sequence as described herein. In some embodiments, the modified U1 snRNA of the invention comprises the main U1 snRNA sequence [clover leaf] (nt 410-562) of the U1_256 sequence as described herein. The main U1 snRNA sequence [clover leaf] (nt 410-562) of the U1_256 sequence is as follows:

(SEQ ID NO: 131)
GCAGGGGAGATACCATGATCACGAAGGTGGTTTTCCCAGGGCGAGGC
TTATCCATTGCACTCCGGATGTGCTGACCCCTGCGATTTCCCCAAAT
GTGGGAAACTCGACTGCATAATTTGTGGTAGTGGGGGACTGCGTTCG
CGCTTTCCCCTG.

In some preferred embodiments, the first 11 nucleotides of the U1 snRNA, which encompasses the native splice donor annealing sequence, may be all or in part replaced with a heterologous sequence that is complementary to a nucleotide sequence within the packaging region of an MSD-mutated lentiviral vector genome. Suitably, 1-11 (suitably 2-11, 3-11, 1, 2, 3, 4, 5, 6, 7, 8, 9, 10 or 11), nucleic acids of the first 11 nucleotides of the U1 snRNA are replaced with a heterologous sequence that is complementary to a nucleotide sequence within the packaging region of an MSD-mutated lentiviral vector genome.

In some embodiments, the native splice donor annealing sequence, may be all or in part replaced with a heterologous sequence that is complementary to a nucleotide sequence within the packaging region of an MSD-mutated lentiviral vector genome. Suitably, 1-11 (suitably 2-11, 3-11, 5-11, 1, 2, 3, 4, 5, 6, 7, 8, 9, 10 or 11), nucleic acids of the native splice donor annealing sequence are replaced with a heterologous sequence that is complementary to a nucleotide sequence within the packaging region of an MSD-mutated lentiviral vector genome. In a preferred embodiment, the entire native splice donor annealing sequence is replaced with a heterologous sequence that is complementary to a nucleotide sequence within the packaging region of an MSD-mutated lentiviral vector genome, i.e. the native splice donor annealing sequence (e.g. 5′-ACUUACCUG-3′) is fully replaced with a heterologous sequence as described herein.

In some embodiments, a heterologous sequence that is complementary to a nucleotide sequence within the packaging region of an MSD-mutated lentiviral vector genome comprises at least 7 nucleotides of complementarity to said nucleotide sequence. In some embodiments, a heterologous sequence that is complementary to a nucleotide sequence within the packaging region of an MSD-mutated lentiviral vector genome comprises at least 9 nucleotides of complementarity to said nucleotide sequence. Preferably, a heterologous sequence for use in the present invention comprises 15 nucleotides of complementarity to said nucleotide sequence.

Suitably, a heterologous sequence for use in the present invention may comprise 7-25 (suitably 7-20, 7-15, 9-15, 7, 8, 9, 10, 11, 12, 13, 14, 15, 16, 17, 18, 19, 20, 21, 22, 23, 24 or 25) nucleotides.

Suitably, a heterologous sequence for use in the present invention may comprise 25 nucleotides.

In some embodiments, the nucleotide sequence within the packaging region of an MSD-mutated lentiviral vector genome is located within the 5′ U5 domain, PBS element, SL1 element, SL2 element, SL3ψ element, SL4 element and/or the sequence derived from gag gene. Suitably, the nucleotide sequence within the packaging region of an MSD-mutated lentiviral vector genome is located within the SL1, SL2 and/or SL3ψ element(s). In some preferred embodiments, the nucleotide sequence within the packaging region of an MSD-mutated lentiviral vector genome is located within the SL1 and/or SL2 element(s). In some particularly preferred embodiments, the nucleotide sequence within the packaging region of an MSD-mutated lentiviral vector genome is located within the SL1 element.

In some embodiments, a nucleotide sequence within the packaging region of an MSD-mutated lentiviral vector genome comprises at least 7 nucleotides. In some embodiments, a nucleotide sequence within the packaging region of an MSD-mutated lentiviral vector genome comprises at least 9 nucleotides. Suitably, a nucleotide sequence within the packaging region of an MSD-mutated lentiviral vector genome comprises 7-25 (suitably 7-20, 7-15, 9-15, 7, 8, 9, 10, 11, 12, 13, 14, or 15) nucleotides.

Preferably, a nucleotide sequence within the packaging region of an MSD-mutated lentiviral vector genome comprises 15 nucleotides.

The binding of a modified U1 snRNA as described herein to the nucleotide sequence within the packaging region of an MSD-mutated lentiviral vector genome may enhance lentiviral vector titre during lentiviral vector production relative to lentiviral vector production in the absence of a modified U1 snRNA as described herein. Thus, production of a lentiviral vector in the presence of a modified U1 snRNA as described herein enhances lentiviral vector titre relative to lentiviral vector production in the absence of a modified U1 snRNA as described herein. A suitable assay for the measurement of lentiviral vector titre is as described herein. Suitably, the lentiviral vector production involves co-expression of said modified U1 snRNA with vector components including gag, env, rev and the RNA genome of the lentiviral vector.

The RNA genome of the lentiviral vector may be an MSD-2KO RNA genome. In some embodiments, the enhancement of lentiviral vector titre occurs in the presence or absence of a functional 5′LTR polyA site. In some embodiments, the enhancement of lentiviral vector titres mediated by a modified U1 snRNA of the invention is independent of polyA site suppression in the 5′LTR of the vector genome.

In some embodiments, the binding of a modified U1 snRNA as described herein to the nucleotide sequence within the packaging region of an MSD-mutated lentiviral vector genome may increase lentiviral vector titre during lentiviral vector production by at least 30% relative to lentiviral vector production in the absence of a modified U1 snRNA as described herein. Suitably, the binding of a modified U1 snRNA as described herein to the nucleotide sequence within the packaging region of an MSD-mutated lentiviral vector genome may increase MSD-mutated lentiviral vector titre during production by at least 35% (suitably at least 40%, 45%, 50%, 60%, 70%, 100%, 150%, 200%, 250%, 300%, 350%, 400%, 450%, 500%, 550%, 600%, 650%, 700%, 750%, 800%, 850%, 900%, 950%, 1,000%, 2,000%, 5,000%, or 10,000%) relative to MSD-mutated lentiviral vector production in the absence of a modified U1 snRNA as described herein.

The modified U1 snRNAs as described herein may be designed by (a) selecting a target site in the packaging region of an MSD-mutated lentiviral vector genome for binding the modified U1 snRNA (the preselected nucleotide site); and (b) introducing within the native splice donor annealing sequence (e.g. 5′-ACUUACCUG-3′) at the 5′ end of the U1 snRNA a heterologous sequence that is complementary to the preselected nucleotide site selected in step (a).

The introduction of a heterologous sequence that is complementary to the target site within, or in place of, the native splice donor annealing sequence (e.g. 5′-ACUUACCUG-3′) at the 5′ end of the endogenous U1 snRNA using conventional techniques in molecular biology is within the capabilities of a person of ordinary skill in the art. Generally speaking, suitable routine methods include directed mutagenesis or replacement via homologous recombination.

The modification of the native splice donor annealing sequence (e.g. 5′-ACUUACCUG-3′) at the 5′ end of the endogenous U1 snRNA to have the same sequence as a heterologous sequence that is complementary to the target site using conventional techniques in molecular biology is within the capabilities of a person of ordinary skill in the art. For example, suitable methods include directed mutagenesis or random mutagenesis followed by selection for mutations which provide a modified U1 snRNA as described herein.

The modified U1 snRNAs as described herein can be manufactured according to methods generally known in the art. For example, the modified U1 snRNAs can be manufactured by chemical synthesis or recombinant DNA/RNA technology.

In one aspect the nucleotide sequence encoding a modified U1 snRNA may be on a different nucleotide sequence, for example on a different plasmid.

The introduction of a nucleotide sequence encoding a modified U1 snRNA as described herein into a cell using conventional molecular and cell biology techniques is within the capabilities of a person of ordinary skill in the art.

Vectors

Another aspect of the invention relates to a viral vector comprising the nucleic acid sequence of the invention.

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purposes of maintaining the heterologous nucleic acid (DNA or RNA) within the cell, or facilitating the replication of the vector comprising a segment of DNA or RNA or the expression of the protein encoded by a segment of nucleic acid.

The vectors of the invention may be, for example, viral vectors provided with an origin of replication, optionally a promoter for the expression of the said polynucleotide and optionally a regulator of the promoter. The vectors may contain one or more selectable marker genes (e.g. a neomycin resistance gene) and/or traceable marker genes (e.g. a gene encoding GFP). Vectors may be used, for example, to infect and/or transduce a target cell.

The vector of the invention may be used to replicate the NOI in a compatible target cell in vitro. Thus, the invention provides a method of making proteins in vitro by introducing a vector of the invention into a compatible target cell in vitro and growing the target cell under conditions which result in expression of the NOI. Protein may be recovered from the target cell by methods well known in the art. Suitable target cells include mammalian cell lines and other eukaryotic cell lines.

The vector may be an expression vector. Expression vectors as described herein comprise regions of nucleic acid containing sequences capable of being transcribed. Thus, sequences encoding mRNA, tRNA and rRNA are included within this definition. Preferably, an expression vector comprises a polynucleotide of the invention operably linked to a control sequence that is capable of providing for the expression of the coding sequence by the target cell.

Viral Vectors

In one embodiment of the invention, the vector is a viral vector. A viral vector may also be called a vector, vector virion or vector particle.

In one embodiment, the viral vector is produced by the viral vector production system as described herein.

In one embodiment, the viral vector comprises more than one NOI wherein at least one NOI is operably linked to a tbs or a portion thereof as described herein.

In another embodiment, the viral vector is derived from a retrovirus, adenovirus, adeno-associated virus, herpes simplex virus, vaccinia virus or baculovirus.

It is anticipated that the repression system of the invention will be of benefit to any viral vector system. The system will find particular use where the nucleotide of interest causes adverse effects, for example on the viral vector production cell or during virion assembly.

In another embodiment, the retrovirus is derived from a foamy virus.

In another embodiment, the retroviral vector is derived from a lentivirus.

In another embodiment, the lentiviral vector is derived from HIV-1, HIV-2, SIV, FIV, BIV, EIAV, CAEV or visna lentivirus.

Vector Titre

The skilled person will understand that there are a number of different methods of determining the titre of viral vectors. Titre is often described as transducing units/mL (TU/mL). Titre may be increased by increasing the number of infectious particles and by increasing the specific activity of a vector preparation.

Retroviral and Lentiviral Vectors

The retroviral vector of the present invention may be derived from or may be derivable from any suitable retrovirus. A large number of different retroviruses have been identified. Examples include: murine leukemia virus (MLV), human T-cell leukemia virus (HTLV), mouse mammary tumour virus (MMTV), Rous sarcoma virus (RSV), Fujinami sarcoma virus (FuSV), Moloney murine leukemia virus (Mo MLV), FBR murine osteosarcoma virus (FBR MSV), Moloney murine sarcoma virus (Mo-MSV), Abelson murine leukemia virus (A-MLV), Avian myelocytomatosis virus-29 (MC29) and Avian erythroblastosis virus (AEV). A detailed list of retroviruses may be found in Coffin et al. (1997) “Retroviruses”, Cold Spring Harbour Laboratory Press Eds: J M Coffin, S M Hughes, H E Varmus pp 758-763.

Retroviruses may be broadly divided into two categories, namely “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al (1997) ibid.

The basic structure of retrovirus and lentivirus genomes share many common features such as a 5′ LTR and a 3′ LTR, between or within which are located a packaging signal to enable the genome to be packaged, a primer binding site, integration sites to enable integration into a target cell genome and gag/pol and env genes encoding the packaging components—these are polypeptides required for the assembly of viral particles. Lentiviruses have additional features, such as the rev gene and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a typical retroviral vector of the present invention, at least part of one or more protein coding regions essential for replication may be removed from the virus; for example, gag/pol and env may be absent or not functional. This makes the viral vector replication-defective.

Portions of the viral genome may also be replaced by a library encoding candidate nucleic acid binding sequences as described herein operably linked to a regulatory control region and a reporter gene in the vector genome in order to generate a vector comprising candidate nucleic acid binding sequences as described herein which is capable of transducing a target non-dividing cell and/or integrating its genome into a host genome.

Lentiviruses are part of a larger group of retroviruses. A detailed list of lentiviruses may be found in Coffin et al (1997) “Retroviruses” Cold Spring Harbour Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV) and bovine immunodeficiency virus (BIV).

The lentivirus family differs from retroviruses in that lentiviruses have the capability to infect both dividing and non-dividing cells (Lewis et al (1992) EMBO J 11(8):3053-3058 and Lewis and Emerman (1994) J Virol 68 (1):510-516). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be derived from either a primate lentivirus (e.g. HIV-1) or a non-primate lentivirus.

Examples of non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV).

In general terms, a typical retroviral vector production system involves the separation of the viral genome from the essential viral packaging functions. These components are typically provided to the production cells on separate DNA expression cassettes (alternatively known as plasmids, expression plasmids, DNA constructs or expression constructs).

The vector genome comprises the NOI. Vector genomes typically require a packaging signal (ψ), a central polypurine tract (cppt), a rev-response element (RRE) the internal expression cassette harbouring the NOI, (optionally) a post-transcriptional element (PRE), typically a central polypurine tract (cppt), the 3′-ppu and a self-inactivating (SIN) LTR. The R-U5 regions are required for correct polyadenylation of both the vector genome RNA and NOI mRNA, as well as the process of reverse transcription. The vector genome may optionally include an open reading frame, as described in WO 2003/064665.

In one aspect nucleotide sequence may be suitable for use in a lentiviral vector in a tat-independent system for vector production. As described herein, 3^{1d} generation lentiviral vectors are tat-independent, and the nucleotide sequences according to the present invention may be used in the context of a 3^{1d} generation lentiviral vector. In one aspect of the invention tat is not provided in the lentiviral vector production system, for example tat is not provided in trans. In one aspect the cell or vector or vector production system as described herein does not comprise the tat protein.

The packaging functions include the gag/pol and env genes. These are required for the production of vector particles by the production cell. Providing these functions in trans to the genome facilitates the production of replication-defective virus.

Production systems for gamma-retroviral vectors are typically 3-component systems requiring genome, gag/pol and env expression constructs. Production systems for HIV-1-based lentiviral vectors additionally require the accessory gene rev to be provided in trans and for the vector genome to include the rev-responsive element (RRE). EIAV-based lentiviral vectors do not require rev if an open-reading frame (ORF) is present (see WO 2003/064665).

Usually both the “external” promoter (which drives the vector genome cassette) and “internal” promoter (which drives the NOI cassette) encoded within the vector genome cassette are strong eukaryotic or virus promoters, as are those driving the other vector system components. Examples of such promoters include CMV, EF1α, PGK, CAG, TK, SV40 and Ubiquitin promoters. Strong ‘synthetic’ promoters, such as those generated by DNA libraries (e.g. JeT promoter) may also be used to drive transcription. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human al-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAIb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Production of retroviral vectors involves either the transient transfection of the production cells with these DNA components or use of stable producer cell lines (PCLs) wherein the components are integrated within the production cell genome (e.g. Stewart, H. J., M. A. Leroux-Carlucci, C. J. Sion, K. A. Mitrophanous and P. A. Radcliffe (2009) Gene Ther. 16(6): 805-814 Epub 29 Mar. 2005). An alternative approach is to use a stable packaging cell (into which the packaging components are stably integrated) and then transiently transfect in the vector genome plasmid as required. In order to generate the viral vectors of the present invention the production cells must be capable of expressing TRAP. Thus, in one embodiment of the invention the production cells will stably express the TRAP construct. In another embodiment of the invention the production cells will transiently express the TRAP construct. In another embodiment of the invention the production cells will stably express a TRAP construct and also transiently express a TRAP construct.

It should be noted that, although the TRIP system has been mainly described for producing retroviral vectors, analogous strategies can be applied to other viral vectors.

In one embodiment of the present invention, the viral vector is derived from EIAV. EIAV has the simplest genomic structure of the lentiviruses and is particularly preferred for use in the present invention. In addition to the gag/pol and env genes, EIAV encodes three other genes: tat, rev, and S2. Tat acts as a transcriptional activator of the viral LTR (Derse and Newbold (1993) Virology 194(2):530-536 and Maury et al (1994) Virology 200(2):632-642) and rev regulates and coordinates the expression of viral genes through rev-response elements (RRE) (Martarano et al. (1994) J Virol 68(5):3102-3111). The mechanisms of action of these two proteins are thought to be broadly similar to the analogous mechanisms in the primate viruses (Martarano et al. (1994) J Virol 68(5):3102-3111). The function of S2 is unknown. In addition, an EIAV protein, Ttm, has been identified that is encoded by the first exon of tat spliced to the env coding sequence at the start of the transmembrane protein. In an alternative embodiment of the present invention the viral vector is derived from HIV: HIV differs from EIAV in that it does not encode S2 but unlike EIAV it encodes vif, vpr, vpu and nef.

The term “recombinant retroviral or lentiviral vector” (RRV) refers to a vector with sufficient retroviral genetic information to allow packaging of an RNA genome, in the presence of packaging components, into a viral particle capable of infecting a target cell. Infection of the target cell may include reverse transcription and integration into the target cell genome. The RRV carries non-viral coding sequences which are to be delivered by the vector to the target cell. A RRV is incapable of independent replication to produce infectious retroviral particles within the target cell. Usually the RRV lacks a functional gag/pol and/or env gene, and/or other genes essential for replication.

Preferably the RRV vector of the present invention has a minimal viral genome.

As used herein, the term “minimal viral genome” means that the viral vector has been manipulated so as to remove the non-essential elements whilst retaining the elements essential to provide the required functionality to infect, transduce and deliver a nucleotide sequence of interest to a target cell. Further details of this strategy can be found in WO 1998/17815 and WO 99/32646. A minimal EIAV vector lacks tat, rev and S2 genes and neither are these genes provided in trans in the production system. A minimal HIV vector lacks vif, vpr, vpu, tat and nef.

However, the expression plasmid used to produce the vector genome within a production cell will include transcriptional regulatory control sequences operably linked to the retroviral genome to direct transcription of the genome in a production cell/packaging cell. These regulatory sequences may be the natural sequences associated with the transcribed retroviral sequence, i.e. the 5′ U3 region, or they may be a heterologous promoter such as another viral promoter, for example the CMV promoter, as discussed below. Some lentiviral vector genomes require additional sequences for efficient virus production. For example, particularly in the case of HIV, RRE sequences may be included. However the requirement for RRE (and dependence on rev which is provided in trans) may be reduced or eliminated by codon optimisation. Further details of this strategy can be found in WO 2001/79518. Alternative sequences which perform the same function as the rev/RRE system are also known. For example, a functional analogue of the rev/RRE system is found in the Mason Pfizer monkey virus. This is known as the constitutive transport element (CTE) and comprises an RRE-type sequence in the genome which is believed to interact with a factor in the infected cell. The cellular factor can be thought of as a rev analogue. Thus, CTE may be used as an alternative to the rev/RRE system. Any other functional equivalents which are known or become available may be relevant to the invention. For example, it is also known that the Rex protein of HTLV-I can functionally replace the Rev protein of HIV-1. Rev and RRE may be absent or non-functional in the vector for use in the methods of the present invention; in the alternative rev and RRE, or functionally equivalent system, may be present.

SIN Vectors

The vectors for use in the methods of the present invention are preferably used in a self-inactivating (SIN) configuration in which the viral enhancer and promoter sequences have been deleted. SIN vectors can be generated and transduce non-dividing target cells in vivo, ex vivo or in vitro with an efficacy similar to that of wild-type vectors. The transcriptional inactivation of the long terminal repeat (LTR) in the SIN provirus should prevent mobilization of vRNA, and is a feature that further diminishes the likelihood of formation of replication-competent virus. This should also enable the regulated expression of genes from internal promoters by eliminating any cis-acting effects of the LTR.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive ‘provirus’. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it is important to prevent the adventitious activation of any endogenous oncogene. Yu et al., (1986) PNAS 83: 3194-98; Marty et al., (1990) Biochimie 72: 885-7; Naviaux et al., (1996) J. Virol. 70: 5701-5; Iwakuma et al., (1999) Virol. 261: 120-32; Deglon et al., (2000) Human Gene Therapy 11: 179-90. SIN lentiviral vectors are described in U.S. Pat. Nos. 6,924,123 and 7,056,699.

Non-Replicating Lentiviral Vectors

In the genome of a replication-defective lentiviral vector the sequences of gag/pol and/or env may be mutated, absent and/or not functional.

In a typical lentiviral vector of the present invention, at least part of one or more coding regions for proteins essential for virus replication may be removed from the vector. This makes the viral vector replication-defective. Portions of the viral genome may also be replaced by a nucleotide of interest (NOI) in order to generate a vector comprising an NOI which is capable of transducing a non-dividing target cell and/or integrating its genome into the target cell genome.

In one embodiment the lentiviral vectors are non-integrating vectors as described in WO 2006/010834 and WO 2007/071994.

In a further embodiment the vectors have the ability to deliver a sequence which is devoid of or lacking viral RNA. In a further embodiment a heterologous binding domain (heterologous to gag) located on the RNA to be delivered and a cognate binding domain on Gag or GagPol can be used to ensure packaging of the RNA to be delivered. Both of these vectors are described in WO 2007/072056.

Adenoviral Vectors

In another embodiment of the present invention, the vector may be an adenovirus vector. The adenovirus is a double-stranded, linear DNA virus that does not replicate through an RNA intermediate. There are over 50 different human serotypes of adenovirus divided into 6 subgroups based on their genetic sequence.

Adenoviruses are double-stranded DNA non-enveloped viruses that are capable of in vivo, ex vivo and in vitro transduction of a broad range of cell types of human and non-human origin. These cells include respiratory airway epithelial cells, hepatocytes, muscle cells, cardiac myocytes, synoviocytes, primary mammary epithelial cells and post-mitotically terminally differentiated cells such as neurons.

Adenoviral vectors are also capable of transducing non-dividing cells. This is very important for diseases, such as cystic fibrosis, in which the affected cells in the lung epithelium have a slow turnover rate. In fact, several trials are underway utilising adenovirus-mediated transfer of cystic fibrosis transporter (CFTR) into the lungs of afflicted adult cystic fibrosis patients.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹² transducing units per ml. Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus.

Adeno-Associated Virus Vectors

Adeno-associated virus (AAV) is an attractive vector system for use in the present invention as it has a high frequency of integration and it can infect non-dividing cells. This makes it useful for delivery of genes into mammalian cells. AAV has a broad host range for infectivity. Details concerning the generation and use of rAAV vectors are described in U.S. Pat. Nos. 5,139,941 and 4,797,368, each incorporated herein by reference.

Recombinant AAV vectors have been used successfully for in vitro, ex vivo and in vivo transduction of marker genes and genes involved in human diseases.

Certain AAV vectors have been developed which can efficiently incorporate large payloads (up to 8-9 kb). One such vector has an AAV5 capsid and an AAV2 ITR (Allocca M, et al J. Clin Invest (2008) 118: 1955-1964).

Herpes Simplex Virus Vectors

Herpes simplex virus (HSV) is an enveloped double-stranded DNA virus that naturally infects neurons. It can accommodate large sections of foreign DNA, which makes it attractive as a vector system, and has been employed as a vector for gene delivery to neurons (Manservigiet et al Open Virol J. (2010) 4:123-156).

The use of HSV in therapeutic procedures requires the strains to be attenuated so that they cannot establish a lytic cycle. In particular, if HSV vectors are used for gene therapy in humans, the polynucleotide should preferably be inserted into an essential gene. This is because if a viral vector encounters a wild-type virus, transfer of a heterologous gene to the wild-type virus could occur by recombination. However, as long as the polynucleotide is inserted into an essential gene, this recombinational transfer would also delete the essential gene in the recipient virus and prevent “escape” of the heterologous gene into the replication competent wild-type virus population.

Vaccinia Virus Vectors

The vector of the present invention may be a vaccinia virus vector such as MVA or NYVAC.

Alternatives to vaccinia vectors include avipox vectors such as fowlpox or canarypox known as ALVAC and strains derived therefrom which can infect and express recombinant proteins in human cells but are unable to replicate.

Baculovirus Vectors

The vector of the present invention may also be a baculovirus vector. The modification of baculovirus to enable the expression of encoded NOIs within mammalian cells is well known in the art. This can be achieved, for example, through the use of mammalian promoters upstream of the NOI.

Vectors Encoding Multiple NOIs

In one embodiment, the vector comprises more than one NOI wherein one or more NOI is operably linked to the tbs or portion thereof as described herein.

Internal Ribosome Entry Site (IRES)

As discussed above, the vectors of the invention may comprise more than one NOI. In order for these NOIs to be expressed, there may be two or more transcription units within the vector genome, one for each NOI. However, it is clear from the literature that retroviral vectors achieve the highest titres and most potent gene expression properties if they are kept genetically simple (WO 96/37623; Bowtell et al., 1988 J. Virol. 62, 2464; Correll et al., 1994 Blood 84, 1812; Emerman and Temin 1984 Cell 39, 459; Ghattas et al., 1991 Mol. Cell. Biol. 11, 5848; Hantzopoulos et al., 1989 PNAS 86, 3519; Hatzoglou et al., 1991 J. Biol. Chem 266, 8416; Hatzoglou et al., 1988 J. Biol. Chem 263, 17798; Li et al., 1992 Hum. Gen. Ther. 3, 381; McLachlin et al., 1993 Virol. 195, 1; Overell et al., 1988 Mol. Cell Biol. 8, 1803; Scharfman et al., 1991 PNAS 88, 4626; Vile et al., 1994 Gene Ther 1, 307; Xu et al., 1989 Virol. 171, 331; Yee et al., 1987 PNAS 84, 5197) and so it is preferable to use an internal ribosome entry site (IRES) to initiate translation of the second (and subsequent) coding sequence(s) in a polycistronic message (Adam et al 1991 J. Virol. 65, 4985).

Insertion of IRES elements into retroviral vectors is compatible with the retroviral replication cycle and allows expression of multiple coding regions from a single promoter (Adam et al (as above); Koo et al (1992) Virology 186:669-675; Chen et al 1993 J. Virol 67:2142-2148). IRES elements were first found in the non-translated 5′ ends of picornaviruses where they promote cap-independent translation of viral proteins (Jang et al (1990) Enzyme 44: 292-309). When located between open reading frames in an RNA, IRES elements allow efficient translation of the downstream open reading frame by promoting entry of the ribosome at the IRES element followed by downstream initiation of translation.

A review on IRES is presented by Mountford and Smith (TIG May 1995 vol 11, No 5:179-184). A number of different IRES sequences are known including those from encephalomyocarditis virus (EMCV) (Ghattas, I. R., et al., Mol. Cell. Biol., 11:5848-5859 (1991); BiP protein [Macejak and Sarnow, Nature 353:91 (1991)]; the Antennapedia gene of Drosophila (exons d and e) [Oh, et al., Genes & Development, 6:1643-1653 (1992)] as well as those in polio virus (PV) [Pelletier and Sonenberg, Nature 334: 320-325 (1988); see also Mountford and Smith, TIG 11, 179-184 (1985)].

IRES elements from PV, EMCV and swine vesicular disease virus have previously been used in retroviral vectors (Coffin et al, as above).

The term “IRES” includes any sequence or combination of sequences which work as or improve the function of an IRES.

The IRES(s) may be of viral origin (such as EMCV IRES (SEQ ID NO: 59), PV IRES, or FMDV 2A-like sequences) or cellular origin (such as FGF2 IRES, NRF IRES, Notch 2 IRES or EIF4 IRES).

In order for the IRES to be capable of initiating translation of each polynucleotide it should be located between or prior to the polynucleotides in the vector genome.

Promoter

Expression of a NOI may be controlled using control sequences, which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters.

Suitable promoting sequences are strong promoters including those derived from the genomes of viruses, such as polyoma virus, adenovirus, fowlpox virus, bovine papilloma virus, avian sarcoma virus, cytomegalovirus (CMV), retrovirus and Simian Virus 40 (SV40), or from heterologous mammalian promoters, such as the actin promoter, EF1α, CAG, TK, SV40, ubiquitin, PGK or ribosomal protein promoter. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human al-antitrypsin (hAAT) promoter, phosphoenolpyruvate carboxykinase (PEPCK), liver fatty acid binding protein promoter, Flt-1 promoter, INF-β promoter, Mb promoter, SP-B promoter, SYN1 promoter, WASP promoter, SV40/hAIb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Transcription of a gene may be increased further by inserting an enhancer sequence into the vector. Enhancers are relatively orientation- and position-independent; however, one may employ an enhancer from a eukaryotic cell virus, such as the SV40 enhancer on the late side of the replication origin (bp 100-270) and the CMV early promoter enhancer. The enhancer may be spliced into the vector at a position 5′ or 3′ to the promoter, but is preferably located at a site 5′ from the promoter.

The promoter can additionally include features to ensure or to increase expression in a suitable target cell. For example, the features can be conserved regions e.g. a Pribnow Box or a TATA box. The promoter may contain other sequences to affect (such as to maintain, enhance or decrease) the levels of expression of a nucleotide sequence. Suitable other sequences include the Sh1-intron or an ADH intron. Other sequences include inducible elements, such as temperature, chemical, light or stress inducible elements. Also, suitable elements to enhance transcription or translation may be present.

The TRAP-tbs interaction may be useful in forming the basis for a transgene protein repression system for the production of retroviral vectors, when a constitutive and/or strong promoter, including a tissue-specific promoter, driving the transgene is desirable and particularly when expression of the transgene protein in production cells leads to reduction in vector titres and/or elicits an immune response in vivo due to viral vector delivery of transgene-derived protein.

Regulators of NOIs

A complicating factor in the generation of retroviral packaging/producer cell lines and retroviral vector production is that constitutive expression of certain retroviral vector components and NOIs are cytotoxic leading to death of cells expressing these components and therefore inability to produce vector. Therefore, the expression of these components (e.g. gag-pol and envelope proteins such as VSV-G) can be regulated. The expression of other non-cytotoxic vector components, e.g. rev, can also be regulated to minimise the metabolic burden on the cell. Thus the modular constructs or nucleotide sequences encoding vector components and/or cells as described herein may comprise cytotoxic and/or non-cytotoxic vector components associated with at least one regulatory element. As used herein, the term “regulatory element” refers to any element capable of affecting, either increasing or decreasing, the expression of an associated gene or protein. A regulatory element includes a gene switch system, transcription regulation element and translation repression element

A number of prokaryotic regulator systems have been adapted to generate gene switches in mammalian cells. Many retroviral packaging and producer cell lines have been controlled using gene switch systems (e.g. tetracycline and cumate inducible switch systems) thus enabling expression of one or more of the retroviral vector components to be switched on at the time of vector production. Gene switch systems include those of the (TetR) protein group of transcription regulators (e.g. T-Rex, Tet-On, and Tet-Off), those of the cumate inducible switch system group of transcription regulators (e.g. CymR protein) and those involving an RNA-binding protein (e.g. TRAP).

One such tetracycline-inducible system is the tetracycline repressor (TetR) system based on the T-REx™ system. By way of example, in such a system tetracycline operators (TetO₂) are placed in a position such that the first nucleotide is 10 bp from the 3′ end of the last nucleotide of the TATATAA element of the human cytomegalovirus major immediate early promoter (hCMVp) then TetR alone is capable of acting as a repressor (Yao F, Svensjo T, Winkler T, Lu M, Eriksson C, Eriksson E., 1998, Hum Gene Ther, 9: 1939-1950). In such a system the expression of the NOI can be controlled by a CMV promoter into which two copies of the TetO₂ sequence have been inserted in tandem. TetR homodimers, in the absence of an inducing agent (tetracycline or its analogue doxycycline [dox]), bind to the TetO₂ sequences and physically block transcription from the upstream CMV promoter. When present, the inducing agent binds to the TetR homodimers, causing allosteric changes such that it can no longer bind to the TetO₂ sequences, resulting in gene expression. The TetR gene may be codon optimised as this was found to improve translation efficiency resulting in tighter control of TetO₂ controlled gene expression.

The TRiP system is described in WO 2015/092440 and provides another way of repressing expression of the NOI in the production cells during vector production. The TRAP-binding sequence (e.g. TRAP-tbs) interaction forms the basis for a transgene protein repression system for the production of retroviral vectors, when a constitutive and/or strong promoter, including a tissue-specific promoter, driving the transgene is desirable and particularly when expression of the transgene protein in production cells leads to reduction in vector titres and/or elicits an immune response in vivo due to viral vector delivery of transgene-derived protein (Maunder et al, Nat Commun. (2017) March 27; 8).

Briefly, the TRAP-tbs interaction forms a translational block, repressing translation of the transgene protein (Maunder et al, Nat Commun. (2017) March 27; 8). The translational block is only effective in production cells and as such does not impede the DNA- or RNA-based vector systems. The TRiP system is able to repress translation when the transgene protein is expressed from a constitutive and/or strong promoter, including a tissue-specific promoter from single- or multi cistronic mRNA. It has been demonstrated that unregulated expression of transgene protein can reduce vector titres and affect vector product quality. Repression of transgene protein for both transient and stable PaCL/PCL vector production systems is beneficial for production cells to prevent a reduction in vector titres: where toxicity or molecular burden issues may lead to cellular stress; where transgene protein elicits an immune response in vivo due to viral vector delivery of transgene-derived protein; where the use of gene-editing transgenes may result in on/off target affects; where the transgene protein may affect vector and/or envelope glycoprotein exclusion.

Packaging Sequence

As utilized within the context of the present invention the term “packaging signal”, which is referred to interchangeably as “packaging sequence” or “psi”, is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon. In EIAV the packaging signal comprises the R region into the 5′ coding region of Gag.

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Feline immunodeficiency virus (FIV) RNA encapsidation determinants have been shown to be discrete and non-continuous, comprising one region at the 5′ end of the genomic mRNA (R-U5) and another region that mapped within the proximal 311nt of gag (Kaye et al., J Virol. October; 69(10):6588-92 (1995).

Pseudotyping

In one preferred aspect, the viral vector of the present invention has been pseudotyped. In this regard, pseudotyping can confer one or more advantages. For example, the env gene product of the HIV based vectors would restrict these vectors to infecting only cells that express a protein called CD4. But if the env gene in these vectors has been substituted with env sequences from other enveloped viruses, then they may have a broader infectious spectrum (Verma and Somia (1997) Nature 389(6648):239-242). By way of example, workers have pseudotyped an HIV based vector with the glycoprotein from VSV (Verma and Somia (1997) Nature 389(6648):239-242).

In another alternative, the Env protein may be a modified Env protein such as a mutant or engineered Env protein. Modifications may be made or selected to introduce targeting ability or to reduce toxicity or for another purpose (Valsesia-Wittman et al 1996 J Virol 70: 2056-64; Nilson et al (1996) Gene Ther 3(4):280-286; and Fielding et al (1998) Blood 91(5):1802-1809 and references cited therein).

The vector may be pseudotyped with any molecule of choice.

VSV-G

The envelope glycoprotein (G) of Vesicular stomatitis virus (VSV), a rhabdovirus, is an envelope protein that has been shown to be capable of pseudotyping certain enveloped viruses and viral vector virions.

Its ability to pseudotype MoMLV-based retroviral vectors in the absence of any retroviral envelope proteins was first shown by Emi et al. (1991) Journal of Virology 65:1202-1207). WO 1994/294440 teaches that retroviral vectors may be successfully pseudotyped with VSV-G. These pseudotyped VSV-G vectors may be used to transduce a wide range of mammalian cells. More recently, Abe et al. (1998) J Virol 72(8) 6356-6361 teach that non-infectious retroviral particles can be made infectious by the addition of VSV-G.

Burns et al. (1993) Proc. Natl. Acad. Sci. USA 90:8033-7) successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al. (1993) ibid). They have also been shown to be more efficient than traditional amphotropic envelopes for a variety of cell lines (Yee et al., (1994) Proc. Natl. Acad. Sci. USA 91:9564-9568, Emi et al. (1991) Journal of Virology 65:1202-1207). VSV-G protein can be used to pseudotype certain retroviruses because its cytoplasmic tail is capable of interacting with the retroviral cores.

The provision of a non-retroviral pseudotyping envelope such as VSV-G protein gives the advantage that vector particles can be concentrated to a high titre without loss of infectivity (Akkina et al. (1996) J. Virol. 70:2581-5). Retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages.

WO 2000/52188 describes the generation of pseudotyped retroviral vectors, from stable producer cell lines, having vesicular stomatitis virus-G protein (VSV-G) as the membrane-associated viral envelope protein, and provides a gene sequence for the VSV-G protein.

Ross River Virus

The Ross River viral envelope has been used to pseudotype a non-primate lentiviral vector (FIV) and following systemic administration predominantly transduced the liver (Kang et al (2002) J Virol 76(18):9378-9388). Efficiency was reported to be 20-fold greater than obtained with VSV-G pseudotyped vector, and caused less cytotoxicity as measured by serum levels of liver enzymes suggestive of hepatotoxicity.

Baculovirus GP64

The baculovirus GP64 protein has been shown to be an alternative to VSV-G for viral vectors used in the large-scale production of high-titre virus required for clinical and commercial applications (Kumar M, Bradow B P, Zimmerberg J (2003) Hum Gene Ther. 14(1):67-77). Compared with VSV-G-pseudotyped vectors, GP64-pseudotyped vectors have a similar broad tropism and similar native titres. Because, GP64 expression does not kill cells, 293T-based cell lines constitutively expressing GP64 can be generated.

Alternative Envelopes

Other envelopes which give reasonable titre when used to pseudotype EIAV include Mokola, Rabies, Ebola and LCMV (lymphocytic choriomeningitis virus). Intravenous infusion into mice of lentivirus pseudotyped with 4070A led to maximal gene expression in the liver.

Viral Vector Production Systems and Cells

Another aspect of the invention relates to a viral vector production system comprising a set of nucleic acid sequences encoding the components required for production of the viral vector, wherein the vector genome sequence comprises the nucleic acid sequence of the invention.

“Viral vector production system” or “vector production system” or “production system” is to be understood as a system comprising the necessary components for viral vector production.

Accordingly, the vector production system comprises a set of nucleic acid sequences which encode the components necessary to generate viral vector particles. One such nucleic acid sequence may comprise the gene encoding a TRAP. In a preferred embodiment the RNA binding protein is the bacterial TRAP.

In one embodiment of the invention, the viral vector is a retroviral vector and the viral vector production system further comprises nucleic acid sequences encoding Gag and Gag/Pol proteins, and Env protein, or functional substitutes thereof and the vector genome sequence which comprises the nucleic acid sequence of the present invention. The production system may optionally comprise a nucleic acid sequence encoding the Rev protein and/or a nucleic acid sequence encoding TRAP.

In another embodiment of the viral vector production system of the invention, the viral vector is derived from a retrovirus, adenovirus or adeno-associated virus.

In another embodiment, the viral vector is derived from a lentivirus. In another embodiment, the viral vector is derived from HIV-1, HIV-2, SIV, FIV, BIV, EIAV, CAEV or visna lentivirus.

Another aspect of the invention relates to a method of increasing viral vector titers in a eukaryotic vector production cell, the method comprising introducing into the eukaryotic vector production cell the viral vector production system of the invention and a nucleic acid sequence encoding a TRAP, wherein the TRAP binds to the TRAP binding site, or the portion thereof, and represses translation of the NOI, thereby increasing viral vector titres relative to a viral vector having no TRAP binding site.

Another aspect of the invention relates to DNA constructs for use in the viral vector production system of the invention. Such DNA constructs (e.g. plasmids) may include the vector genome construct which comprises the nucleic acid sequence of the invention.

A further aspect of the invention relates to a DNA construct for use in the viral vector production system of the invention comprising a nucleic acid sequence encoding TRAP.

Another aspect of the invention relates to a set of DNA constructs for use in the viral vector production system of the invention comprising the DNA constructs of the invention and DNA constructs encoding Gag and Gag/Pol proteins and Env protein, or functional substitutes thereof.

In one embodiment of the invention, the set of DNA constructs additionally comprises a DNA construct encoding TRAP.

In one embodiment of the invention, the set of DNA constructs additionally comprises a DNA construct encoding Rev protein or a functional substitute thereof.

In one embodiment, the viral vector production system comprises modular nucleic acid constructs (modular constructs). A modular construct is a DNA expression construct comprising two or more nucleic acids used in the production of lentiviral vectors. A modular construct can be a DNA plasmid comprising two or more nucleic acids used in the production of lentiviral vectors. The plasmid may be a bacterial plasmid. The nucleic acids can encode for example, gag-pol, rev, env, vector genome. In addition, modular constructs designed for generation of packaging and producer cell lines may additionally need to encode transcriptional regulatory proteins (e.g. TetR, CymR) and/or translational repression proteins (e.g. TRAP) and selectable markers (e.g Zeocin™, hygromycin, blasticidin, puromycin, neomycin res/stance genes). Suitable modular constructs for use in the present invention are described in EP 3502260, which is hereby incorporated by reference in its entirety.

As the modular constructs for use in accordance with the present invention contain nucleic acid sequences encoding two or more of the retroviral components on one construct, the safety profile of these modular constructs has been considered and additional safety features directly engineered into the constructs. These features include the use of insulators for multiple open reading frames of retroviral vector components and/or the specific orientation and arrangement of the retroviral genes in the modular constructs. It is believed that by using these features the direct read-through to generate replication-competent viral particles will be prevented.

The nucleic acid sequences encoding the viral vector components may be in reverse and/or alternating transcriptional orientations in the modular construct. Thus, the nucleic acid sequences encoding the viral vector components are not presented in the same 5′ to 3′ orientation, such that the viral vector components cannot be produced from the same mRNA molecule. The reverse orientation may mean that at least two coding sequences for different vector components are presented in the ‘head-to-head’ and ‘tail-to-tail’ transcriptional orientations. This may be achieved by providing the coding sequence for one vector component, e.g. env, on one strand and the coding sequence for another vector component, e.g. rev, on the opposing strand of the modular construct. Preferably, when coding sequences for more than two vector components are present in the modular construct, at least two of the coding sequences are present in the reverse transcriptional orientation. Accordingly, when coding sequences for more than two vector components are present in the modular construct, each component may be orientated such that it is present in the opposite 5′ to 3′ orientation to all of the adjacent coding sequence(s) for other vector components to which it is adjacent, i.e. alternating 5′ to 3′ (or transcriptional) orientations for each coding sequence may be employed.

The modular construct for use according to the present invention may comprise nucleic acid sequences encoding two or more of the following vector components: gag-pol, rev, env, vector genome. The modular construct may comprise nucleic acid sequences encoding any combination of the vector components. In one embodiment, the modular construct may comprise nucleic acid sequences encoding:
i) the RNA genome of the retroviral vector and rev, or a functional substitute thereof;
ii) the RNA genome of the retroviral vector and gag-pol;
iii) the RNA genome of the retroviral vector and env;
iv) gag-pol and rev, or a functional substitute thereof;
v) gag-pol and env;
vi) env and rev, or a functional substitute thereof;
vii) the RNA genome of the retroviral vector, rev, or a functional substitute thereof, and gag-pol;
viii) the RNA genome of the retroviral vector, rev, or a functional substitute thereof, and env;
ix) the RNA genome of the retroviral vector, gag-pol and env; or
x) gag-pol, rev, or a functional substitute thereof, and env, wherein the nucleic acid sequences are in reverse and/or alternating orientations.

In one embodiment, a cell for producing retroviral vectors may comprise nucleic acid sequences encoding any one of the combinations i) to x) above, wherein the nucleic acid sequences are located at the same genetic locus and are in reverse and/or alternating orientations. The same genetic locus may refer to a single extrachromosomal locus in the cell, e.g. a single plasmid, or a single locus (i.e. a single insertion site) in the genome of the cell. The cell may be a stable or transient cell for producing retroviral vectors, e.g. lentiviral vectors.

Another aspect of the invention relates to a viral vector production cell comprising the nucleic acid sequence, the viral vector production system, or some or all of the DNA constructs of the invention.

A “viral vector production cell” is to be understood as a cell that is capable of producing a viral vector or viral vector particle. Viral vector production cells may be “producer cells” or “packaging cells”. One or more DNA constructs of the viral vector system may be stably integrated or episomally maintained within the viral vector production cell. Alternatively, all the DNA components of the viral vector system may be transiently transfected into the viral vector production cell. In yet another alternative, a production cell stably expressing some of the components may be transiently transfected with the remaining components.

The DNA expression cassette encoding the TRAP may be stably integrated or episomally maintained within the viral vector production cell. Alternatively, the DNA expression cassette encoding the TRAP may be transiently transfected into the viral vector production cell.

Thus, in one embodiment of the invention the production cells will stably express the TRAP construct. In another embodiment of the invention the production cells will transiently express the TRAP construct.

The level of repression required may vary in accordance with the NOI, thus the level of TRAP required in the production cell may also depend on the NOI. In some circumstances, a combination of stable and transient TRAP expression may therefore be desired. The stable expression may provide a continual level of TRAP expression in the production cell, while the transient expression may provide shorter term, increased levels of TRAP expression. For example, it is possible that repression of more problematic/toxic transgenes will benefit from both pre-existing (e.g. provided by stable expression) and high levels of TRAP during vector production.

Thus, in another embodiment of the invention the production cells will stably express a TRAP construct and also transiently express a TRAP construct. The transient expression may provide short term higher levels of TRAP expression than provided by the stable expression.

By “stable expression”, it is to be understood that the expression of TRAP from the construct providing the stable expression substantially does not vary over a prolonged period of time.

By “transient expression”, it is to be understood that the expression of TRAP from the construct providing the transient expression is not stable over a prolonged period of time. Preferably, the polynucleotide encoding TRAP which provides for the transient expression does not integrate into the production cell genome and is not episomally maintained in the production cell.

As used herein, the term “packaging cell” refers to a cell which contains the elements necessary for production of infectious vector particles but which are lack the vector genome. Typically, such packaging cells contain one or more expression cassettes which are capable of expressing viral structural proteins (such as gag, gag/pol and env).

Producer cells/packaging cells can be of any suitable cell type. Producer cells are generally mammalian cells but can be, for example, insect cells.

As used herein, the term “producer/production cell” or “vector producing/production cell” refers to a cell which contains all the elements necessary for production of retroviral vector particles, and expression of TRAP.

The producer cell may be either a stable producer cell line or derived transiently.

In one embodiment of the invention the envelope and nucleocapsid, TRAP and, if present, rev nucleotide sequences are all stably integrated in the producer and/or packaging cell. However, any one or more of these sequences could also exist in episomal form and gene expression could occur from the episome, or could be transfected transiently into the production cell.

The vector production cells may be cells cultured in vitro such as a tissue culture cell line. Suitable cell lines include, but are not limited to, mammalian cells such as murine fibroblast derived cell lines or human cell lines. Preferably the vector production cells are derived from a human cell line.

In one embodiment the vectors of the present invention use as their production system, four transcription units expressing a vector genome comprising the nucleic acid sequence of the invention operably linked to the NOI, the gag-pol components, an envelope and TRAP. The envelope expression cassette may include one of a number of heterologous envelopes such as VSV-G. Optionally the rev component may also be included.

Viral Vector Production Processes

Another aspect of the invention relates to a process for producing viral vectors comprising introducing the nucleic acid sequence, the viral vector production system, or some or all of the DNA constructs of the invention into a viral vector production cell and culturing the production cell under conditions suitable for the production of the viral vectors.

Suitable “production cells” are those cells which are capable of producing viral vectors or viral vector particles when cultured under appropriate conditions. They are generally mammalian or human cells, for example HEK293T, HEK293, CAP, CAP-T or CHO cells, but can be, for example, insect cells such as SF9 cells.

The production cells may also be avian cells, for example EB66® (Sigma) cells. Avian cells may be particularly useful for the production of human and veterinary virus-based vaccines, for example influenza and Newcastle Disease virus vaccines.

Methods for introducing nucleic acids into production cells are well known in the art and have been described previously.

In one embodiment, the production cell comprises TRAP.

Another aspect of the invention relates to a viral vector produced by the viral vector production system of the invention, using the viral vector production cell of the invention or by the process of the invention.

In one embodiment, the viral vector particle comprises the nucleic acid sequence of the invention. The viral vector particle may be derived from a retrovirus, adenovirus or adeno-associated virus. The retroviral vector particle may be derived from a lentivirus. The lentiviral vector particle may be derived from HIV-1, HIV-2, SIV, FIV, BIV, EIAV, CAEV or visna lentivirus.

Methods for generating lentiviral vectors and in particular the processing of lentiviral vectors, are described in WO 2009/153563.

Another aspect of the invention relates to a cell transduced by the viral vector of the invention.

A “cell transduced by a viral vector particle” is to be understood as a cell, in particular a target cell, into which the nucleic acid carried by the viral vector particle has been transferred.

Use

Another aspect of the invention relates to the viral vector of the invention or a cell or tissue transduced with the viral vector of the invention for use in medicine.

Another aspect of the invention relates to the use of the viral vector of the invention or a cell or tissue transduced with the viral vector of the invention in medicine.

Another aspect of the invention relates to the use of the viral vector of the invention, a production cell of the invention or a cell or tissue transduced with the viral vector of the invention for the preparation of a medicament to deliver a nucleotide of interest to a target site in need of the same.

Such uses of the viral vector or transduced cell of the invention may be for therapeutic or diagnostic purposes, as described herein.

Therapeutic Vectors

Retroviral Therapeutic Vectors

In one embodiment, a retroviral vector of the invention may be used to introduce the three genes that encode three enzymes of the dopamine synthetic pathway to treat Parkinson's disease. The retroviral vector is a non-replicating, self-inactivating minimal lentiviral vector, derived from Human immunodeficiency virus (HIV) or Equine infectious anaemia virus (EIAV) which may be pseudotyped with VSV-G or an alternative viral envelope protein. The genes carried by the retroviral vector may comprise a truncated form of the human tyrosine hydroxylase (TH*) gene (which lacks the N-terminal 160 amino acids involved in feedback regulation of TH), the human aromatic L-amino-acid decarboxylase (AADC), and the human GTP-cyclohydrolase 1 (CH1) gene. The three enzymes may be encoded by the retroviral vector in three separate open reading frames. Alternatively, the retroviral vector may encode a fusion of the TH and CH1 enzymes in a first open reading frame and the AADC enzyme in a second open reading frame. Expression of the genes may be driven by a CMV promoter, and the expression cassette may include one or more IRES elements. The retroviral vector may be administered by direct injection into the striatum of the brain.

In another embodiment, a retroviral vector of the invention may be used as a gene therapy product designed to introduce the corrective MYO7A gene to photoreceptors and supporting retinal pigment epithelial (RPE) cells and thereby attenuate or reverse the deterioration in vision which is associated with Usher 1B Syndrome. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the MYO7A cDNA, which codes for the MYO7A protein (a large gene which is over 100 mb in length). Expression of the large MYO7A gene may be driven by a CMV promoter, a CMV/MYO7A chimeric promoter or an alternative promoter. The retroviral vector may be administered by direct subretinal injection following vitrectomy of the eye.

In another embodiment, a retroviral vector of the invention may be used to introduce the corrective ATP-binding cassette gene, ABCA4 (also known as ABCR), to photoreceptors and thereby attenuate or reverse the pathophysiology which leads to Stargardt disease. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the ABCA4 cDNA, which codes for ABCA4 protein. Expression of the ABCA4 gene may be driven by a CMV promoter, a photoreceptor-specific promoter, such as rhodopsin kinase or an alternative promoter. The retroviral vector may be administered by direct subretinal injection following vitrectomy of the eye.

In another embodiment, a retroviral vector of the invention may be used as a gene therapy product designed to prevent recurrence of aberrant blood vessel growth and/or vascular leakage in the eyes of patients with wet-form age-related macular degeneration (AMD), diabetic macular oedema or retinal vein occlusion, and/or to prevent aberrant blood vessel growth in the eyes of patients with dry-form age-related macular degeneration (AMD). This retroviral vector delivers a gene or genes encoding an anti-angiogenic protein or proteins, such as angiostatin and/or endostatin. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. In one embodiment the retroviral vector expresses human endostatin and angiostatin genes in a bicistronic configuration utilising an internal ribosome entry site (IRES) for delivery to retinal pigment epithelial cells. Expression of the anti-angiogenic gene(s) may be driven by CMV, an RPE-specific promoter such as the vitelliform macular dystrophy 2 (VM D2) promoter (more recently known as the bestrophin promoter) or by an alternative promoter. The retroviral vector may be administered by direct subretinal injection following vitrectomy of the eye.

In another embodiment, a retroviral vector of the invention may be used as a gene therapy product designed to prevent recurrence of aberrant blood vessel growth in oedema in the eyes of patients with wet-form age-related macular degeneration (AMD). This retroviral vector delivers a gene or genes encoding an anti-angiogenic protein or proteins, such as angiostatin and/or endostatin. The retroviral vector is a non-replicating, self-inactivating minimal lentiviral vector derived from Equine infectious anaemia virus (EIAV) which may be pseudotyped with VSV-G or an alternative viral envelope protein. In one embodiment the retroviral vector expresses human endostatin and angiostatin genes in a bicistronic configuration utilising an internal ribosome entry site (IRES) for delivery to retinal pigment epithelial cells. Expression of the anti-angiogenic gene(s) may be driven by CMV, an RPE-specific promoter such as the vitelliform macular dystrophy 2 (VMD2) promoter (more recently known as the bestrophin promoter) or by an alternative promoter. The retroviral vector may be administered by direct subretinal injection following vitrectomy of the eye.

In another embodiment, a retroviral vector of the invention may be used as a gene therapy product designed to prevent corneal graft rejection as a result of neovascularization by delivery of anti-angiogenic gene(s) to the donor cornea prior to grafting. The retroviral vector is a non-replicating, self-inactivating minimal lentiviral vector derived from Human immunodeficiency virus (HIV) or Equine infectious anaemia virus (EIAV) which may be pseudotyped with VSV-G, Ebola or an alternative viral envelope protein. In one embodiment, the retroviral vector will express anti-angiogenic gene(s) such as human endostatin and angiostatin genes in a bicistronic configuration utilizing an internal ribosome entry site (IRES) for ex vivo delivery to corneal grafts. The retroviral vector may be applied to corneal graft tissue ex vivo, and the transduced donor tissue may also be stored prior to transplantation. Expression of the anti-angiogenic gene(s) may be driven by a constitutive promoter such as the CMV promoter; however it is also possible that alternative promoters may be used.

In another embodiment, a retroviral vector of the invention may be used as a gene therapy designed to prevent recurrence of aberrant blood vessel growth and/or vascular leakage in the eyes of patients with wet-form age-related macular degeneration (AMD), diabetic macular oedema or retinal vein occlusion, and/or to prevent aberrant blood vessel growth in the eyes of patients with dry-form age-related macular degeneration (AMD). This retroviral vector delivers a gene encoding a soluble form of fms-like tyrosine kinase. (Soluble Flt-1) The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. Expression of the soluble Flt-1 gene may be driven by CMV, an RPE-specific promoter such as the vitelliform macular dystrophy 2 (VM D2) promoter (more recently known as the bestrophin promoter) or by an alternative promoter. The retroviral vector may be administered by direct subretinal injection following vitrectomy of the eye.

In another embodiment, a retroviral vector of the invention may be used as a gene therapy product designed to prevent recurrence of aberrant blood vessel growth and/or vascular leakage in the eyes of patients with wet-form age-related macular degeneration (AMD), diabetic macular oedema or retinal vein occlusion, and/or to prevent aberrant blood vessel growth in the eyes of patients with dry-form age-related macular degeneration (AMD). This retroviral vector delivers a gene or genes encoding the pigment epithelium-derived factor protein (PEDF). The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. Expression of the PEDF gene may be driven by CMV, an RPE-specific promoter such as the vitelliform macular dystrophy 2 (VMD2) promoter (more recently known as the bestrophin promoter) or by an alternative promoter. The retroviral vector may be administered by direct subretinal injection following vitrectomy of the eye.

In another embodiment, a retroviral vector of the invention may be used as a gene therapy product designed to prevent recurrence of aberrant blood vessel growth and/or vascular leakage in the eyes of patients with wet-form age-related macular degeneration (AMD), diabetic macular oedema or retinal vein occlusion, and/or to prevent aberrant blood vessel growth in the eyes of patients with dry-form age-related macular degeneration (AMD). This retroviral vector delivers a gene or genes encoding an inhibitor of vascular endothelial growth factor (VEGF), such as an anti-VEGF antibody or binding fragment thereof (e.g. aflibercept), a VEGF specific aptamer or a VEGF blocking peptide or polypeptide including, but not limited to, a soluble form of a VEGF receptor and/or an inhibitor of platelet-derived growth factor (PDGF), such as an anti-PDGF antibody or binding fragment thereof, a PDGF specific aptamer or a PDGF blocking peptide or polypeptide including, but not limited to, a soluble form of a PDGF receptor. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. In one embodiment the retroviral vector expresses an inhibitor of VEGF and an inhibitor of PDGF in a bicistronic configuration utilising an internal ribosome entry site (IRES) for delivery to retinal pigment epithelial cells. Expression of the gene(s) may be driven by CMV, an RPE-specific promoter such as the vitelliform macular dystrophy 2 (VMD2) promoter (more recently known as the bestrophin promoter) or by an alternative promoter. The retroviral vector may be administered by direct subretinal injection following vitrectomy of the eye.

In another embodiment, a retroviral vector of the invention may be used to introduce the corrective gene vitelliform macular dystrophy 2 (VMD2) and a cassette encoding a micro-RNA (miRNA) specific for the disease-associated form of VMD2, or the corrective Peripherin 2 encoding RDS gene and a cassette encoding an miRNA specific for the disease-associated form of RDS to retinal pigment epithelial cells and thereby attenuate or reverse the pathophysiology which leads to Best disease or Best vitelliform macular degeneration (BVMD). The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. Expression of the genes may be driven by CMV, an RPE-specific promoter such as the vitelliform macular dystrophy 2 (VMD2) promoter (more recently known as the bestrophin promoter) or by an alternative promoter. The retroviral vector may be administered by direct subretinal injection following vitrectomy of the eye.

In another embodiment, a retroviral vector of the invention may be used to introduce the corrective retinaldehyde binding protein 1 gene, RLBP1, to retinal pigment epithelial cells and thereby attenuate or reverse the pathophysiology which leads to RLBP1-associated retinal dystrophy. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the RLBP1 cDNA, which codes for RLBP1 protein. Expression of the RLBP1 gene may be driven by CMV, an RPE-specific promoter such as the vitelliform macular dystrophy 2 (VMD2) promoter (more recently known as the bestrophin promoter) or by an alternative promoter. The retroviral vector may be administered by direct subretinal injection following vitrectomy of the eye.

In another embodiment, a retroviral vector of the invention may be used as a gene therapy product designed to treat glaucoma. This retroviral vector delivers a gene or genes encoding COX-2 and/or Prostaglandin F2a receptor (FPR) which act to reduce intraocular pressure. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. In one embodiment the retroviral vector expresses COX-2 and Prostaglandin F2a receptor (FPR). genes in a bicistronic configuration utilizing an internal ribosome entry site (IRES) for delivery to the anterior chamber of the eye. Expression of the gene(s) may be driven by CMV or an alternative promoter. The retroviral vector may be administered by transcorneal injection.

In another embodiment, a retroviral vector of the invention may be used to introduce the corrective harmonin gene to attenuate or reverse the pathophysiology which leads to Usher Syndrome 1c. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the harmonin cDNA, which codes for the harmonin protein. Expression of the harmonin gene may be driven by a CMV promoter or an alternative promoter. The retroviral vector may be administered by direct subretinal injection following vitrectomy of the eye.

In another embodiment, a retroviral vector of the invention may be used to introduce the corrective Rab escort protein 1 (REP1) gene to attenuate or reverse the pathophysiology which leads to choroideremia. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the REP1 cDNA, which codes for the REP1 protein. Expression of the REP1 gene may be driven by a CMV promoter or an alternative promoter. The retroviral vector may be administered by direct subretinal injection following vitrectomy of the eye.

In another embodiment, a retroviral vector of the invention may be used to introduce the corrective Cyclic Nucleotide Gated Channel Beta 2 (CNGB2) and/or Cyclic Nucleotide Gated Channel Alpha 3 (CNGA3) genes(s) into the eye to attenuate or reverse the pathophysiology which leads to Achromatopsia. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene(s) carried by the retroviral vector are the CNGB2 and/or CNGA3 gene(s), that code for the CNGB2 and/or CNGA3 proteins. Expression of the gene(s) may be driven by a CMV promoter or an alternative promoter. The retroviral vector may be administered by direct subretinal injection following vitrectomy of the eye.

In another embodiment, a retroviral vector of the invention may be used to introduce the corrective CEP290 gene into the eye to attenuate or reverse the pathophysiology which leads to Leber Congenital Amaurosis (LCA). The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the CEP290 gene, that codes for the centrosomal protein of 290 kDa. Expression of the CEP290 gene may be driven by a CMV promoter or an alternative promoter. The retroviral vector may be administered by direct subretinal injection following vitrectomy of the eye.

In another embodiment, a retroviral vector of the invention may be used to introduce the corrective retinitis pigmentosa GTPase regulator (RPGR) gene into the eye to attenuate or reverse the pathophysiology which leads to x-linked retinitis pigmentosa. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the RPGR cDNA, which codes for the RPGR protein. Expression of the RPGR gene may be driven by a CMV promoter or an alternative promoter. The retroviral vector may be administered by direct subretinal injection following vitrectomy of the eye.

In another embodiment, a retroviral vector of the invention may be used to introduce the corrective retinoschisin 1 (RS1) gene into the eye to attenuate or reverse the pathophysiology which leads to x-linked retinochisis. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the RS1 cDNA, which codes for the RS1 protein. Expression of the RS1 gene may be driven by a CMV promoter or an alternative promoter. The retroviral vector may be administered by direct subretinal injection following vitrectomy of the eye.

In another embodiment, a retroviral vector of the invention may be used to introduce the corrective retinitis pigmentosa 1 (RP1) gene into the eye to attenuate or reverse the pathophysiology which leads to retinitis pigmentosa. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the RP1 cDNA, which codes for the RP1 protein. Expression of the RP1 gene may be driven by a CMV promoter, a photoreceptor-specific promoter, such as rhodopsin kinase or an alternative promoter. The retroviral vector may be administered by direct subretinal injection following vitrectomy of the eye.

In another embodiment, a retroviral vector of the invention may be used to introduce the corrective retinal pigment epithelium-specific 65 kDa protein (RPE65) gene to attenuate or reverse the pathophysiology which leads to Leber congenital amaurosis (LCA) type 2. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the RPE65 cDNA, which codes for the RPE65 protein. Expression of the RPE65 gene may be driven by CMV, an RPE-specific promoter such as the vitelliform macular dystrophy 2 (VM D2) promoter (more recently known as the bestrophin promoter) or by an alternative promoter. The retroviral vector may be administered by direct subretinal injection following vitrectomy of the eye.

In another embodiment, a retroviral vector of the invention may be used to introduce the corrective human proline/arginine-rich end leucine-rich repeat protein (PRELP) gene to attenuate or reverse the pathophysiology which leads to wet-form age-related macular degeneration (AMD), dry-form AMD, diabetic macular oedema or retinal vein occlusion. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene carried by the retroviral vector is the PRELP cDNA, which codes for the PRELP protein. Expression of the PRELP gene may be driven by CMV, an RPE-specific promoter such as the vitelliform macular dystrophy 2 (VM D2) promoter (more recently known as the bestrophin promoter) or by an alternative promoter. The retroviral vector may be administered by direct subretinal injection following vitrectomy of the eye.

In another embodiment, a retroviral vector of the invention may be used to introduce a nucleic acid sequence encoding a synthetic myocilin-specific miRNA into the eye to attenuate or reverse the pathophysiology which leads to juvenile open angle glaucoma by knocking down the expression of myocilin. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. Expression of the synthetic myocilin-specific miRNA may be driven by a CMV promoter or an alternative promoter. The retroviral vector may be administered by direct subretinal injection following vitrectomy of the eye.

In another embodiment, a retroviral vector of the invention may be used to introduce rate-limiting enzyme(s) from the glutathione biosynthesis pathway, glutamate-cysteine ligase (GCL) and/or glutathione synthetase (GSS), and/or a nucleic acid sequence encoding a synthetic gamma-glutamyltransferase (GGT) specific miRNA into the eye to attenuate or reverse the pathophysiology which leads to retinitis pigmentosa by gene augmentation and/or knock-down. The retroviral vector is a non-replicating, self-inactivating minimal lentiviral vector, for example derived from Human immunodeficiency virus (HIV) or Equine infectious anaemia virus (EIAV), which may be pseudotyped with VSV-G or an alternative viral envelope protein. Expression of the GCL and/or GSS gene(s) and/or the synthetic GGT specific miRNA may be driven by a CMV promoter or an alternative promoter. In one embodiment, the retroviral vector may express the gene(s) and/or the synthetic miRNA in a multicistronic configuration utilising one or more internal ribosome entry site (IRES). The retroviral vector may be administered by direct delivery to the anterior chamber of the eye.

In another embodiment, a retroviral vector of the invention may be used as a gene therapy product designed to treat neurodegenerative disorders such as frontotemporal lobe dementia, Alzheimer's disease, Parkinson's disease, Huntington's disease and motor neuron disorders such as Amyotrophic Lateral Sclerosis (ALS). This retroviral vector delivers a gene encoding a VEGF protein; which may be a VEGF-A isoform such as VEGF₁₄₅, VEGF₁₆₅, or VEGF₁₈₉; or may be VEGF-B, VEGF-C, or VEGF-D, such genes having a neuroprotective effect. The retroviral vector is a non-replicating, self-inactivating minimal lentiviral vector derived from Human immunodeficiency virus (HIV) or Equine infectious anaemia virus (EIAV) which may be pseudotyped with Rabies G or VSV-G or an alternative viral envelope protein. Expression of the gene may be driven by CMV or an alternative promoter. The retroviral vector may be administered by direct injection into large muscle groups or by direct injection into the cerebrospinal fluid via intrathecal or intraventricular injection.

In another embodiment, a retroviral vector of the invention may be used as a gene therapy product designed to treat cystic fibrosis. This retroviral vector delivers a gene encoding cystic fibrosis transmembrane conductance regulator (CFTR). The retroviral vector is a non-replicating, self-inactivating minimal lentiviral vector derived from Human immunodeficiency virus (HIV) or Equine infectious anaemia virus (EIAV) which may be pseudotyped with Flu-HA, Sendai virus envelope F or HN, Ebola, baculovirus GP64 or an alternative viral envelope protein. Expression of the gene may be driven by CMV or an alternative promoter. The retroviral vector may be administered intranasally, by use of a nebuliser, or by direct delivery via bronchial alveolar lavage into the lungs.

In another embodiment, a retroviral vector of the invention may be used to introduce the corrective N-Sulfoglucosamine Sulfohydrolase (SGSH) and/or Sulfatase Modifying Factor 1 (SUMF1) gene(s) into the brain to attenuate or reverse the pathophysiology which leads to Sanfilipo syndrome A. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. The gene(s) carried by the retroviral vector are the SGSH cDNA, which codes for the SGSH protein and/or the SUMF1 gene which codes for the SUMF1 protein. Expression of the gene(s) may be driven by a CMV promoter or an alternative promoter. In one embodiment, the retroviral vector may express the SGSH and SUMF1 genes in a bicistronic configuration utilising an internal ribosome entry site (IRES). The retroviral vector may be administered by direct intracerebral injection.

In another embodiment, a retroviral vector of the invention may be used to introduce the corrective acid-alpha glycosidase (GAA) gene into large muscle groups and/or the lungs to attenuate or reverse the pathophysiology which leads to Pompe Disease. This retroviral vector delivers a gene encoding a GAA protein. The retroviral vector is a non-replicating, self-inactivating minimal lentiviral vector derived from Human immunodeficiency virus (HIV) or Equine infectious anaemia virus (EIAV) which may be pseudotyped with Flu-HA, Sendai virus envelope F or HN, Ebola, baculovirus GP64, Rabies G, VSV-G or an alternative viral envelope protein. Expression of the gene may be driven by CMV or an alternative promoter. The retroviral vector may be administered by; (i) direct injection into large muscle groups and/or (ii) intranasally, by use of a nebuliser, or by direct delivery via bronchial alveolar lavage into the lungs.

In another embodiment, a retroviral vector of the invention may be used ex vivo to transduce autologous or allogeneic T cells with a nucleic acid sequence encoding a CD19-specific chimeric antigen receptor (CAR19). These transduced T cells are then infused into a subject to treat cancers and leukaemias expressing CD19. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. Expression of the CAR encoding nucleic acid sequence may be driven by EF1α, CMV or an alternative promoter.

In another embodiment, a retroviral vector of the invention may be used ex vivo to transduce autologous or allogeneic T cells with a nucleic acid sequence encoding a 5T4-specific chimeric antigen receptor (CAR). These transduced T cells then are infused into a subject to treat cancers and leukaemias expressing 5T4. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. Expression of the 5T4 CAR-encoding nucleic acid sequence may be driven by EF1α, CMV or an alternative promoter.

As will be known to those skilled in the art chimeric antigen receptors (CARs) can be produced that are specific for a range of cancer or leukaemia-associated polypeptides. A retroviral vector of the invention may be used ex vivo to transduce autologous or allogeneic T cells with a nucleic acid sequence encoding a chimeric antigen receptor (CAR) specific for any cancer or leukaemia-associated polypeptide. These transduced T cells then are infused into a subject to treat cancers and leukaemias expressing the cancer or leukaemia-associated polypeptide to which the CAR binds. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. Expression of the CAR-encoding nucleic acid sequence may be driven by EF1α, CMV or an alternative promoter. Suitable cancer or leukaemia-associated polypeptides that may be targeted by said CARs include, but are not limited to, the following: mesothelin, folate receptor a, kappa light chain of immunoglobulin, CD30, Carcinoembryonic antigen (CEA), CD138, Ganglioside G2 (GD2), CD33, CD22, Epidermal growth factor receptors (EGFRs) such as EGFR VIII, IL-13Rα2, CD20, ErbBs such as Her2, prostate-specific membrane antigen (PSMA), Lewis Y antigen and fibroblast activation protein (FAB).

In another embodiment, a retroviral vector of the invention may be used ex vivo to transduce autologous or allogeneic T cells with a nucleic acid sequence encoding a T cell receptor (TCR) which is specific for a peptide-MHC expressed on diseased, leukemic or cancerous cells. These transfected T cells then are infused into a subject to treat the disease, cancer or leukaemia that is associated with the expression of the peptide-MHC to which the TCR binds. The retroviral vector is a non-replicating, self-inactivating minimal lentiviral vector derived from Human immunodeficiency virus (HIV) or Equine infectious anaemia virus (EIAV) which may be pseudotyped with VSV-G or an alternative viral envelope protein. Expression of the TCR-encoding nucleic acid sequence may be driven by EF1α, CMV or an alternative promoter. The TCRs that are encoded by the vectors of the invention may be single-chain TCRs (scTCRs) or dimeric TCRs (dTCRs). As will be known to those skilled in the art suitable dTCRs include those described in WO 2003/020763 and suitable scTCRs include those described in WO 1999/018129. In specific aspects of this embodiment the T cells transfected with TCRs may be used to treat AIDs, leukaemia, and cancers including myelomas and sarcomas.

In another embodiment, a retroviral vector of the invention may be used to introduce the gene that encodes the common gamma chain (CD132) to treat x-linked Severe combined immunodeficiency (SCID). The retroviral vector is a non-replicating, self-inactivating minimal lentiviral vector, derived from Equine infectious anaemia virus (EIAV) which may be pseudotyped with VSV-G or an alternative viral envelope protein. Expression of the gene may be driven by a CMV promoter or an alternative promoter. The retroviral vector of the invention may be used ex vivo to transduce bone marrow stem cells. These transduced bone marrow stem cells can then be infused into a subject to treat the disease.

In another embodiment, a retroviral vector of the invention may be used to introduce the gene that encodes adenosine deaminase to treat ADA Severe combined immunodeficiency (SCID). The retroviral vector is a non-replicating, self-inactivating minimal lentiviral vector, derived from derived from Human immunodeficiency virus (HIV) or Equine infectious anaemia virus (EIAV) which may be pseudotyped with VSV-G or an alternative viral envelope protein. Expression of the gene may be driven by a CMV promoter or an alternative promoter. The retroviral vector of the invention may be used ex vivo to transduce bone marrow stem cells. These transduced bone marrow stem cells can then be infused into a subject to treat the disease.

In another embodiment, a retroviral vector of the invention may be used to introduce the gene that encodes the WAS protein to treat Wiskott-Aldrich Syndrome (WAS). The retroviral vector is a non-replicating, self-inactivating minimal lentiviral vector, derived from derived from Human immunodeficiency virus (HIV) or Equine infectious anaemia virus (EIAV) which may be pseudotyped with VSV-G or an alternative viral envelope protein. Expression of the gene may be driven by a CMV promoter or an alternative promoter. The retroviral vector of the invention may be used ex vivo to transduce bone marrow stem cells. These transduced bone marrow stem cells can then be infused into a subject to treat the disease.

In another embodiment, a retroviral vector of the invention may be used to introduce a gene that encodes one of several globins, including wild-type β-globin, wild-type fetal globin, and mutated “anti-sickling” globins to treat Sickle Cell disease or thalassemia. As will be known to those skilled in the art examples of anti-sickling globins include, but are not limited to those described in WO 2014/043131 and WO 1996/009385. The retroviral vector is a non-replicating, self-inactivating minimal lentiviral vector, derived from Human immunodeficiency virus (HIV) or Equine infectious anaemia virus (EIAV) which may be pseudotyped with VSV-G or an alternative viral envelope protein. Expression of the gene may be driven by a CMV promoter or an alternative promoter. The retroviral vector of the invention may be used ex vivo to transduce bone marrow stem cells. These transduced bone marrow stem cells can then be infused into a subject to treat the disease.

In another embodiment, a retroviral vector of the invention may be used to introduce a corrective gene, Factor VIII, to liver, muscle or adipose cells to treat haemophilia A. The retroviral vector is a non-replicating, self-inactivating minimal lentiviral vector derived from the derived from Human immunodeficiency virus (HIV) or Equine infectious anaemia virus (EIAV) which may be pseudotyped with VSV-G or an alternative viral envelope protein. The gene carried by the retroviral vector is Factor VIII. Expression of the Factor VIII gene may be driven by a CMV promoter or an alternative promoter.

In another embodiment, a retroviral vector of the invention may be used to introduce a corrective gene, Factor IX, to liver, muscle or adipose cells to treat haemophilia B. The retroviral vector is a non-replicating, self-inactivating minimal lentiviral vector derived from the derived from Human immunodeficiency virus (HIV) or Equine infectious anaemia virus (EIAV) which may be pseudotyped with VSV-G or an alternative viral envelope protein. The gene carried by the retroviral vector is Factor IX. Expression of the Factor IX gene may be driven by a CMV promoter or an alternative promoter.

In another embodiment, a retroviral vector of the invention may be used to introduce the gene that encodes alpha galactosidase A (α-GAL A) to treat Fabry disease. The retroviral vector is a non-replicating, self-inactivating minimal lentiviral vector, derived from Human immunodeficiency virus (HIV) or Equine infectious anaemia virus (EIAV) which may be pseudotyped with VSV-G or an alternative viral envelope protein. The gene carried by the retroviral vector is the GLA cDNA, which codes for the α-GAL A protein Expression of the gene may be driven by a CMV promoter or an alternative promoter. The retroviral vector of the invention may be used ex vivo to transduce hematopoietic CD34⁺ stem cells. These transduced hematopoietic CD34⁺ stem cells can then be infused into a subject to treat the disease.

In another embodiment, a retroviral vector of the invention may be used to introduce the gene that encodes a deficient enzyme to treat a form of porphyria. The retroviral vector is a non-replicating, self-inactivating minimal lentiviral vector, derived from Human immunodeficiency virus (HIV) or Equine infectious anaemia virus (EIAV) which may be pseudotyped with VSV-G or an alternative viral envelope protein. The gene carried by the retroviral vector is that encoding the deficient enzyme associated with the type of porphyria to be treated selected from the table below. Expression of the gene may be driven by a CMV promoter or an alternative promoter.

| Porphyria type | Deficient enzyme |
|---|---|
| X-linked sideroblastic anemia | δ-aminolevulinate (ALA) |
| (XLSA) | synthase |
| Doss porphyria/ALA dehydratase | δ-aminolevulinate dehydratase |
| deficiency | (ALAD) |
| Acute intermittent porphyria | Hydroxymethylbilane (HMB) |
| (AIP) | synthase |
| Congenital erythropoietic | Uroporphyrinogen (URO) |
| porphyria (CEP) | synthase |
| Porphyria cutanea tarda (PCT) | Uroporphyrinogen (URO) |
| | decarboxylase |
| Hereditary coproporphyria | Coproporphyrinogen (COPRO) |
| (HCP) | oxidase |
| Variegate porphyria (VP) | Protoporphyrinogen (PROTO) |
| | oxidase |
| Erythropoietic protoporphyria | Ferrochelatase |
| (EPP) | |

In another embodiment, a retroviral vector of the invention may be used to introduce the gene that encodes a deficient enzyme to treat a form of mucopolysaccharidosis. The retroviral vector is a non-replicating, self-inactivating minimal lentiviral vector, derived from Human immunodeficiency virus (HIV) or Equine infectious anaemia virus (EIAV) which may be pseudotyped with VSV-G or an alternative viral envelope protein. The gene carried by the retroviral vector is that encoding the deficient enzyme associated with the type of musocpolysaccharidosis to be treated selected from the table below. Expression of the gene may be driven by a CMV promoter or an alternative promoter.

| Mucopolysaccharidosis type | Deficient enzyme |
|---|---|
| Hurler syndrome/Hurler- | α-L-iduronidase |
| Scheie syndrome/Scheie syndrome | |
| Hunter syndrome | Iduronate sulfatase |
| Sanfilippo syndrome A | Heparan sulfamidase |
| Sanfilippo syndrome B | N-acetylglucosaminidase |
| Sanfilippo syndrome C | Heparan-α-glucosaminide |
| | N-acetyltransferase |
| Sanfilippo syndrome D | 3 N-acetylglucosamine |
| | 6-sulfatase |
| Morquio syndrome A | Galactose-6-sulfate sulfatase |
| Morquio syndrome B | β-galactosidase |
| Maroteaux-Lamy syndrome | N-acetylgalactosamine- |
| | 4-sulfatase |
| Sly syndrome | β-glucuronidase |
| Natowicz syndrome | Hyaluronidase |

Production of Retroviral Therapeutic Vectors

Retroviral vectors of the invention may be produced by the transient transfection of HEK293T cells with four plasmids:

(1) the recombinant retroviral vector genome plasmid encoding the required transgene(s) and the nucleic acid sequence of the invention,

(2) the synthetic retroviral gag/pol expression plasmid,

(3) the envelope (env) expression plasmid which may, for example, express VSV-G, and

(4) the RNA-binding protein expression plasmid.

Alternatively, retroviral vectors of the invention, such as HIV, may be produced by the transient transfection of HEK293T cells with five plasmids:

(1) the recombinant HIV vector genome plasmid encoding the required transgene(s), a nucleic acid sequence of the invention, and the RRE sequence,

(2) a synthetic gag/pol expression plasmid,

(3) the envelope (env) expression plasmid which may, for example, express VSV-G,

(4) the RNA-binding protein expression plasmid, and

(5) the REV expression plasmid.

Alternatively, retroviral vectors of the invention, such as HIV, may be produced by the transient transfection of HEK293T cells with at least one modular construct encoding components required for the production of viral vectors and TRAP, wherein the viral genome comprises the nucleic acid sequence of the invention. Suitable modular constructs include, but are not limited to, those described in EP3502260A.

Alternatively a transient transfection system may utilise a cell line which stably expresses TRAP.

Alternatively, retroviral vectors of the invention may be produced by using packaging cells that stably express (1) gag/pol, (2) env and (3) TRAP, and, for HIV vectors, Rev, and wherein a plasmid encoding the recombinant retroviral vector genome encoding the required transgene(s) and nucleic acid sequence of the invention, and for HIV vectors, includes the RRE sequence, is introduced into such cells by transient transfection.

Alternatively, retroviral vectors of the invention may be produced in producer cells that stably express (1) gag/pol, (2) env, (3) TRAP, (4) the recombinant EIAV vector genome encoding the required transgene(s) and the nucleic acid sequence of the invention.

Alternatively, HIV vectors of the invention may be produced in producer cells that stably express (1) gag/pol, (2) env, (3) TRAP, (4) the recombinant HIV vector genome encoding the required transgene(s), a nucleic acid sequence of the invention, and the RRE sequence, and (5) REV.

AAV Therapeutic Vectors

In another embodiment, an AAV vector of the invention may be used to introduce the three genes that encode three enzymes of the dopamine synthetic pathway to treat Parkinson's disease. The genes carried by the AAV vector may comprise a truncated form of the human tyrosine hydroxylase (TH*) gene (which lacks the N-terminal 160 amino acids involved in feedback regulation of TH), the human aromatic L-amino-acid decarboxylase (AADC), and the human GTP-cyclohydrolase 1 (CH1) gene. The three enzymes may be encoded by the AAV vector in three separate open reading frames. Alternatively, the AAV vector may encode a fusion of the TH and CH1 enzymes in a first open reading frame and the AADC enzyme in a second open reading frame. Expression of the genes may be driven by a CMV promoter, and the expression cassette may include one or more IRES elements. The AAV vector may be administered by direct injection into the striatum of the brain.

In another embodiment, an AAV vector of the invention may be used as a gene therapy product designed to introduce the corrective MYO7A gene to photoreceptors and supporting retinal pigment epithelial (RPE) cells and thereby attenuate or reverse the deterioration in vision which is associated with Usher 1B Syndrome. The gene carried by the AAV vector is the MYO7A cDNA, which codes for the MYO7A protein (a large gene which is over 100 mb in length). Expression of the large MYO7A gene may be driven by a CMV promoter, a CMV/MYO7A chimeric promoter or an alternative promoter. The AAV vector may be administered by direct subretinal injection following vitrectomy of the eye.

In another embodiment, an AAV vector of the invention may be used to introduce the corrective ATP-binding cassette gene, ABCA4 (also known as ABCR), to photoreceptors and thereby attenuate or reverse the pathophysiology which leads to Stargardt disease. The gene carried by the AAV vector is the ABCA4 cDNA, which codes for ABCA4 protein. Expression of the ABCA4 gene may be driven by a CMV promoter, a photoreceptor-specific promoter, such as rhodopsin kinase or an alternative promoter. The AAV vector may be administered by direct subretinal injection following vitrectomy of the eye.

In another embodiment, an AAV vector of the invention may be used as a gene therapy product designed to prevent recurrence of aberrant blood vessel growth and/or vascular leakage in the eyes of patients with wet-form age-related macular degeneration (AMD), diabetic macular oedema or retinal vein occlusion, and/or to prevent aberrant blood vessel growth in the eyes of patients with dry-form age-related macular degeneration (AMD). This AAV vector delivers a gene or genes encoding an anti-angiogenic protein or proteins, such as angiostatin and/or endostatin. In one embodiment the AAV vector expresses human endostatin and angiostatin genes in a bicistronic configuration utilising an internal ribosome entry site (IRES) for delivery to retinal pigment epithelial cells. Expression of the anti-angiogenic gene(s) may be driven by CMV, an RPE-specific promoter such as the vitelliform macular dystrophy 2 (VM D2) promoter (more recently known as the bestrophin promoter) or by an alternative promoter. The AAV vector may be administered by direct subretinal injection following vitrectomy of the eye.

In another embodiment, an AAV vector of the invention may be used as a gene therapy product designed to prevent corneal graft rejection as a result of neovascularization by delivery of anti-angiogenic gene(s) to the donor cornea prior to grafting. In one embodiment, the AAV vector will express anti-angiogenic gene(s) such as human endostatin and angiostatin genes in a bicistronic configuration utilizing an internal ribosome entry site (IRES) for ex vivo delivery to corneal grafts. The AAV vector may be applied to corneal graft tissue ex vivo, and the transduced donor tissue may also be stored prior to transplantation. Expression of the anti-angiogenic gene(s) may be driven by a constitutive promoter such as the CMV promoter; however it is also possible that alternative promoters may be used.

In another embodiment, an AAV vector of the invention may be used as a gene therapy designed to prevent recurrence of aberrant blood vessel growth and/or vascular leakage in the eyes of patients with wet-form age-related macular degeneration (AMD), diabetic macular oedema or retinal vein occlusion, and/or to prevent aberrant blood vessel growth in the eyes of patients with dry-form age-related macular degeneration (AMD). This AAV vector delivers a gene encoding a soluble form of fms-like tyrosine kinase. (Soluble Flt-1). Expression of the soluble Flt-1 gene may be driven by CMV, an RPE-specific promoter such as the vitelliform macular dystrophy 2 (VMD2) promoter (more recently known as the bestrophin promoter) or by an alternative promoter. The AAV vector may be administered by direct subretinal injection following vitrectomy of the eye.

In another embodiment, a AAV vector of the invention may be used as a gene therapy product designed to prevent recurrence of aberrant blood vessel growth and/or vascular leakage in the eyes of patients with wet-form age-related macular degeneration (AMD), diabetic macular oedema or retinal vein occlusion, and/or to prevent aberrant blood vessel growth in the eyes of patients with dry-form age-related macular degeneration (AMD). This AAV vector delivers a gene or genes encoding the pigment epithelium-derived factor protein (PEDF). Expression of the PEDF gene may be driven by CMV, an RPE-specific promoter such as the vitelliform macular dystrophy 2 (VMD2) promoter (more recently known as the bestrophin promoter) or by an alternative promoter. The AAV vector may be administered by direct subretinal injection following vitrectomy of the eye.

In another embodiment, an AAV vector of the invention may be used as a gene therapy product designed to prevent recurrence of aberrant blood vessel growth and/or vascular leakage in the eyes of patients with wet-form age-related macular degeneration (AMD), diabetic macular oedema or retinal vein occlusion, and/or to prevent aberrant blood vessel growth in the eyes of patients with dry-form age-related macular degeneration (AMD). This AAV vector delivers a gene or genes encoding an inhibitor of vascular endothelial growth factor (VEGF), such as an anti-VEGF antibody or binding fragment thereof (e.g. aflibercept), a VEGF specific aptamer or a VEGF blocking peptide or polypeptide including, but not limited to, a soluble form of a VEGF receptor and/or an inhibitor of platelet-derived growth factor (PDGF), such as an anti-PDGF antibody or binding fragment thereof, a PDGF specific aptamer or a PDGF blocking peptide or polypeptide including, but not limited to, a soluble form of a PDGF receptor. In one embodiment the AAV vector expresses an inhibitor of VEGF and an inhibitor of PDGF in a bicistronic configuration utilising an internal ribosome entry site (IRES) for delivery to retinal pigment epithelial cells. Expression of the gene(s) may be driven by CMV, an RPE-specific promoter such as the vitelliform macular dystrophy 2 (VMD2) promoter (more recently known as the bestrophin promoter) or by an alternative promoter. The AAV vector may be administered by direct subretinal injection following vitrectomy of the eye.

In another embodiment, an AAV vector of the invention may be used to introduce the corrective gene vitelliform macular dystrophy 2 (VMD2) and a cassette encoding a micro-RNA (miRNA) specific for the disease-associated form of VMD2, or the corrective Peripherin 2 encoding RDS gene and a cassette encoding an miRNA specific for the disease-associated form of RDS to retinal pigment epithelial cells and thereby attenuate or reverse the pathophysiology which leads to Best disease or Best vitelliform macular degeneration (BVMD). Expression of the genes may be driven by CMV, an RPE-specific promoter such as the vitelliform macular dystrophy 2 (VMD2) promoter (more recently known as the bestrophin promoter) or by an alternative promoter. The AAV vector may be administered by direct subretinal injection following vitrectomy of the eye.

In another embodiment, an AAV vector of the invention may be used to introduce the corrective retinaldehyde binding protein 1 gene, RLBP1, to retinal pigment epithelial cells and thereby attenuate or reverse the pathophysiology which leads to RLBP1-associated retinal dystrophy. The gene carried by the AAV vector is the RLBP1 cDNA, which codes for RLBP1 protein. Expression of the RLBP1 gene may be driven by CMV, an RPE-specific promoter such as the vitelliform macular dystrophy 2 (VMD2) promoter (more recently known as the bestrophin promoter) or by an alternative promoter. The AAV vector may be administered by direct subretinal injection following vitrectomy of the eye.

In another embodiment, an AAV vector of the invention may be used as a gene therapy product designed to treat glaucoma. This AAV vector delivers a gene or genes encoding COX-2 and/or Prostaglandin F2a receptor (FPR). In one embodiment the AAV vector expresses COX-2 and Prostaglandin F2a receptor (FPR). genes in a bicistronic configuration utilizing an internal ribosome entry site (IRES) for delivery to the anterior chamber of the eye. Expression of the gene(s) may be driven by CMV or an alternative promoter. The AAV vector may be administered by transcorneal injection.

In another embodiment, an AAV vector of the invention may be used to introduce the corrective harmonin gene to attenuate or reverse the pathophysiology which leads to Usher Syndrome 1c. The gene carried by the AAV vector is the harmonin cDNA, which codes for the harmonin protein. Expression of the harmonin gene may be driven by a CMV promoter or an alternative promoter. The AAV vector may be administered by direct subretinal injection following vitrectomy of the eye.

In another embodiment, an AAV vector of the invention may be used to introduce the corrective Rab escort protein 1 (REP1) gene to attenuate or reverse the pathophysiology which leads to choroideremia. The gene carried by the AAV vector is the REP1 cDNA, which codes for the REP1 protein. Expression of the REP1 gene may be driven by a CMV promoter or an alternative promoter. The AAV vector may be administered by direct subretinal injection following vitrectomy of the eye.

In another embodiment, an AAV vector of the invention may be used to introduce the corrective Cyclic Nucleotide Gated Channel Beta 2 (CNGB2) and/or Cyclic Nucleotide Gated Channel Alpha 3 (CNGA3) genes(s) into the eye to attenuate or reverse the pathophysiology which leads to Achromatopsia. The gene(s) carried by the AAV vector are the CNGB2 and/or CNGA3 gene(s), that code for the CNGB2 and/or CNGA3 proteins. Expression of the gene(s) may be driven by a CMV promoter or an alternative promoter. The AAV vector may be administered by direct subretinal injection following vitrectomy of the eye.

In another embodiment, an AAV vector of the invention may be used to introduce the corrective CEP290 gene into the eye to attenuate or reverse the pathophysiology which leads to Leber Congenital Amaurosis (LCA). The gene carried by the AAV vector is the CEP290 gene, that codes for the centrosomal protein of 290 kDa. Expression of the CEP290 gene may be driven by a CMV promoter or an alternative promoter. The AAV vector may be administered by direct subretinal injection following vitrectomy of the eye.

In another embodiment, an AAV vector of the invention may be used to introduce the corrective retinitis pigmentosa GTPase regulator (RPGR) gene into the eye to attenuate or reverse the pathophysiology which leads to x-linked retinitis pigmentosa. The gene carried by the AAV vector is the RPGR cDNA, which codes for the RPGR protein. Expression of the RPGR gene may be driven by a CMV promoter or an alternative promoter. The AAV vector may be administered by direct subretinal injection following vitrectomy of the eye.

In another embodiment, an AAV vector of the invention may be used to introduce the corrective retinoschisin 1 (RS1) gene into the eye to attenuate or reverse the pathophysiology which leads to x-linked retinochisis. The gene carried by the AAV vector is the RS1 cDNA, which codes for the RS1 protein. Expression of the RS1 gene may be driven by a CMV promoter or an alternative promoter. The AAV vector may be administered by direct subretinal injection following vitrectomy of the eye.

In another embodiment, an AAV vector of the invention may be used to introduce the corrective retinitis pigmentosa 1 (RP1) gene into the eye to attenuate or reverse the pathophysiology which leads to retinitis pigmentosa. The gene carried by the AAV vector is the RP1 cDNA, which codes for the RP1 protein. Expression of the RP1 gene may be driven by a CMV promoter, a photoreceptor-specific promoter, such as rhodopsin kinase or an alternative promoter. The AAV vector may be administered by direct subretinal injection following vitrectomy of the eye.

In another embodiment, an AAV vector of the invention may be used to introduce the corrective retinal pigment epithelium-specific 65 kDa protein (RPE65) gene to attenuate or reverse the pathophysiology which leads to Leber congenital amaurosis (LCA) type 2. The gene carried by the AAV vector is the RPE65 cDNA, which codes for the RPE65 protein. Expression of the RPE65 gene may be driven by CMV, an RPE-specific promoter such as the vitelliform macular dystrophy 2 (VMD2) promoter (more recently known as the bestrophin promoter) or by an alternative promoter. The AAV vector may be administered by direct subretinal injection following vitrectomy of the eye.

In another embodiment, an AAV of the invention may be used to introduce the corrective human proline/arginine-rich end leucine-rich repeat protein (PRELP) gene to attenuate or reverse the pathophysiology which leads to wet-form age-related macular degeneration (AMD), dry-form AMD, diabetic macular oedema or retinal vein occlusion. The gene carried by the AAV vector is the PRELP cDNA, which codes for the PRELP protein. Expression of the PRELP gene may be driven by CMV, an RPE-specific promoter such as the vitelliform macular dystrophy 2 (VM D2) promoter (more recently known as the bestrophin promoter) or by an alternative promoter. The AAV vector may be administered by direct subretinal injection following vitrectomy of the eye.

In another embodiment, an AAV vector of the invention may be used to introduce a nucleic acid sequence encoding a synthetic myocilin-specific miRNA into the eye to attenuate or reverse the pathophysiology which leads to juvenile open angle glaucoma by knocking down the expression of myocilin. Expression of the synthetic myocilin-specific miRNA may be driven by a CMV promoter or an alternative promoter. The AAV vector may be administered by direct subretinal injection following vitrectomy of the eye.

In another embodiment, an AAV vector of the invention may be used to introduce rate-limiting enzyme(s) from the glutathione biosynthesis pathway, glutamate-cysteine ligase (GCL) and/or glutathione synthetase (GSS), and/or a nucleic acid sequence encoding a synthetic gamma-glutamyltransferase (GGT) specific miRNA into the eye to attenuate or reverse the pathophysiology which leads to retinitis pigmentosa by gene augmentation and/or knock-down. Expression of the GCL and/or GSS gene(s) and/or the synthetic GGT specific miRNA may be driven by a CMV promoter or an alternative promoter. In one embodiment, the AAV vector may express the gene(s) and/or the synthetic miRNA in a multicistronic configuration utilising one or more internal ribosome entry site (IRES). The AAV vector may be administered by direct delivery to the anterior chamber of the eye.

In another embodiment, an AAV vector of the invention may be used as a gene therapy product designed to treat neurodegenerative disorders such as frontotemporal lobe dementia, Alzheimer's disease, Parkinson's disease, Huntington's disease and motor neuron disorders such as Amyotrophic Lateral Sclerosis (ALS). This AAV vector delivers a gene encoding a VEGF protein; which may be a VEGF-A isoform such as VEGF₁₄₅, VEGF₁₆₅, or VEGF₁₈₉; or may be VEGF-B, VEGF-C, or VEGF-D, such genes having a neuroprotective effect. Expression of the gene may be driven by CMV or an alternative promoter. The AAV vector may be administered by direct injection into large muscle groups or by direct injection into the cerebrospinal fluid via intrathecal or intraventricular injection.

In another embodiment, an AAV vector of the invention may be used as a gene therapy product designed to treat cystic fibrosis. This AAV vector delivers a gene encoding cystic fibrosis transmembrane conductance regulator (CFTR). Expression of the gene may be driven by CMV or an alternative promoter. The AAV vector may be administered intranasally, by use of a nebuliser, or by direct delivery via bronchial alveolar lavage into the lungs.

In another embodiment, an AAV vector of the invention may be used to introduce the corrective N-Sulfoglucosamine Sulfohydrolase (SGSH) and/or Sulfatase Modifying Factor 1 (SUMF1) gene(s) into the brain to attenuate or reverse the pathophysiology which leads to Sanfilipo syndrome A. The gene(s) carried by the AAV vector are the SGSH cDNA, which codes for the SGSH protein and/or the SUMF1 gene which codes for the SUMF1 protein. Expression of the gene(s) may be driven by a CMV promoter or an alternative promoter. In one embodiment, the AAV vector may express the SGSH and SUMF1 genes in a bicistronic configuration utilising an internal ribosome entry site (IRES). The AAV vector may be administered by direct intracerebral injection.

In another embodiment, an AAV vector of the invention may be used to introduce the corrective acid-alpha glycosidase (GAA) gene into large muscle groups and/or the lungs to attenuate or reverse the pathophysiology which leads to Pompe Disease. This AAV vector delivers a gene encoding a GAA protein. Expression of the gene may be driven by CMV or an alternative promoter. The AAV vector may be administered by; (i) direct injection into large muscle groups and/or (ii) intranasally, by use of a nebuliser, or by direct delivery via bronchial alveolar lavage into the lungs.

In another embodiment, an AAV vector of the invention may be used to introduce a corrective gene, Factor VIII, to liver, muscle or adipose cells to treat haemophilia A. The gene carried by the AAV vector is Factor VIII. Expression of the Factor VIII gene may be driven by a CMV promoter or an alternative promoter.

In another embodiment, an AAV vector of the invention may be used to introduce a corrective gene, Factor IX, to liver, muscle or adipose cells to treat haemophilia B. The gene carried by the AAV vector is Factor IX. Expression of the Factor IX gene may be driven by a CMV promoter or an alternative promoter.

In another embodiment, an AAV vector of the invention may be used to introduce the gene that encodes a deficient enzyme to treat a form of porphyria. The gene carried by the AAV vector is that encoding the deficient enzyme associated with the type of porphyria to be treated selected from the table below. Expression of the gene may be driven by a CMV promoter or an alternative promoter.

| Porphyria type | Deficient enzyme |
|---|---|
| X-linked sideroblastic anemia | δ-aminolevulinate (ALA) |
| (XLSA) | synthase |
| Doss porphyria/ALA dehydratase | δ-aminolevulinate dehydratase |
| deficiency | (ALAD) |
| Acute intermittent porphyria (AIP) | Hydroxymethylbilane (HMB) |
| | synthase |
| Congenital erythropoietic porphyria | Uroporphyrinogen (URO) |
| (CEP) | synthase |
| Porphyria cutanea tarda (PCT) | Uroporphyrinogen (URO) |
| | decarboxylase |
| Hereditary coproporphyria (HCP) | Coproporphyrinogen (COPRO) |
| | oxidase |
| Variegate porphyria (VP) | Protoporphyrinogen (PROTO) |
| | oxidase |
| Erythropoietic protoporphyria (EPP) | Ferrochelatase |

In another embodiment, an AAV vector of the invention may be used to introduce the gene that encodes a deficient enzyme to treat a form of mucopolysaccharidosis. The gene carried by the AAV vector is that encoding the deficient enzyme associated with the type of musocpolysaccharidosis to be treated selected from the table below. Expression of the gene may be driven by a CMV promoter or an alternative promoter.

| Mucopolysaccharidosis type | Deficient enzyme |
|---|---|
| Hurler syndrome/Hurler- | α-L-iduronidase |
| Scheie syndrome/Scheie syndrome | |
| Hunter syndrome | Iduronate sulfatase |
| Sanfilippo syndrome A | Heparan sulfamidase |
| Sanfilippo syndrome B | N-acetylglucosaminidase |
| Sanfilippo syndrome C | Heparan-α-glucosaminide N- |
| | acetyltransferase |
| Sanfilippo syndrome D | 3 N-acetylglucosamine 6-sulfatase |
| Morquio syndrome A | Galactose-6-sulfate sulfatase |
| Morquio syndrome B | β-galactosidase |
| Maroteaux-Lamy syndrome | N-acetylgalactosamine-4-sulfatase |
| Sly syndrome | β-glucuronidase |
| Natowicz syndrome | Hyaluronidase |

In another embodiment, an AAV vector of the invention may be used as a gene therapy product designed to prevent recurrence of aberrant blood vessel growth in oedema in the eyes of patients with wet-form age-related macular degeneration (AMD). This AAV vector delivers a gene or genes encoding an anti-angiogenic protein or proteins, such as angiostatin and/or endostatin. In one embodiment the AAV vector expresses human endostatin and angiostatin genes in a bicistronic configuration utilising an internal ribosome entry site (IRES) for delivery to retinal pigment epithelial cells. Expression of the anti-angiogenic gene(s) may be driven by CMV, an RPE-specific promoter such as the vitelliform macular dystrophy 2 (VM D2) promoter (more recently known as the bestrophin promoter) or by an alternative promoter. The AAV vector may be administered by direct subretinal injection following vitrectomy of the eye.

In another embodiment, an AAV vector of the invention may be used as a gene therapy product designed to treat neurodegenerative disorders such as frontotemporal lobe dementia, Alzheimer's disease, Parkinson's disease, Huntington's disease and motor neuron disorders such as Amyotrophic Lateral Sclerosis (ALS). This AAV vector delivers a gene encoding a VEGF protein; which may be a VEGF-A isoform such as VEGF₁₄₅, VEGF₁₆₅, or VEGF₁₈₉; or may be VEGF-B, VEGF-C, or VEGF-D, such genes having a neuroprotective effect. Expression of the gene may be driven by CMV or an alternative promoter. The AAV vector may be administered by direct injection into the cerebrospinal fluid bathing the spinal cord via intraventricular or intrathecal injection.

Method of Treatment

Another aspect of the invention relates to a method of treatment comprising administering the viral vector of the invention or a cell transduced with the viral vector of the invention to a subject in need of the same.

It is to be appreciated that all references herein to treatment include curative, palliative and prophylactic treatment; although in the context of the present invention references to preventing are more commonly associated with prophylactic treatment. Treatment may also include prevention or slowing of disease progression. The treatment of mammals is particularly preferred. Both human and veterinary treatments are within the scope of the present invention.

In one embodiment, the viral vectors or viral vector particles of the invention may be for use as vaccines. The vaccines may be, for example, human or veterinary virus-based vaccines (e.g. influenza and Newcastle Disease virus vaccines).

The present invention may be of particular use where the vaccine is based on a modified competent virus that harbours a transgene.

As discussed above, avian production cells may be used in the production of viral vectors and viral vector particles for use as vaccines.

Pharmaceutical Compositions

Another aspect of the invention relates to a pharmaceutical composition comprising the viral vector of the invention or a cell or tissue transduced with the viral vector of the invention, in combination with a pharmaceutically acceptable carrier, diluent or excipient.

The present invention provides a pharmaceutical composition for treating an individual by gene therapy, wherein the composition comprises a therapeutically effective amount of a vector. The pharmaceutical composition may be for human or animal usage.

The composition may comprise a pharmaceutically acceptable carrier, diluent, excipient or adjuvant. The choice of pharmaceutical carrier, excipient or diluent can be made with regard to the intended route of administration and standard pharmaceutical practice. The pharmaceutical compositions may comprise, or in addition to, the carrier, excipient or diluent any suitable binder(s), lubricant(s), suspending agent(s), coating agent(s), solubilising agent(s) and other carrier agents that may aid or increase vector entry into the target site (such as for example a lipid delivery system).

Where appropriate, the composition can be administered by any one or more of inhalation; in the form of a suppository or pessary; topically in the form of a lotion, solution, cream, ointment or dusting powder; by use of a skin patch; orally in the form of tablets containing excipients such as starch or lactose, or in capsules or ovules either alone or in admixture with excipients, or in the form of elixirs, solutions or suspensions containing flavouring or colouring agents; or they can be injected parenterally, for example intracavernosally, intravenously, intramuscularly, intracranially, intraoccularly or subcutaneously. For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner.

The vector of the invention may also be used to transduce target cells or target tissue ex vivo prior to transfer of said target cell or tissue into a patient in need of the same. An example of such cell may be autologous T cells and an example of such tissue may be a donor cornea.

Method of Screening

Another aspect of the invention relates to a method of identifying nucleic acid binding sites and/or cognate nucleic acid binding proteins which are capable of interacting such that the translation of a nucleotide of interest is repressed or prevented in a viral vector production cell when operably linked to the nucleic acid binding site, wherein the method comprises analysing the expression of a reporter gene in a cell comprising both the nucleic acid binding site operably linked to the reporter gene, and the nucleic acid binding protein.

The method may allow the identification of new RNA-binding proteins and their corresponding binding sites which are useful in the present invention. The method may also allow the identification of variants of known RNA-binding proteins or binding sites.

In one embodiment, the method allows the identification of binding sites which interact with TRAP.

In another embodiment, the method allows the identification of nucleic acid binding proteins which interact with a binding site that is capable of binding to TRAP.

In one embodiment, the reporter gene encodes a fluorescent protein.

In another embodiment, the reporter gene encodes a positive cell growth selection marker, for example the sh ble gene product enabling cell resistance to Zeocin”.

In another embodiment, the reporter gene encodes a negative cell growth selection marker, for example the HSV thymidine kinase gene product which causes cell death in the presence of Ganciclovir.

An example of screening TRAP-binding sites (tbs) for improved functionality may be as follows:
Synthesise a degenerate DNA library comprising 8-11 repeats of the sequence KAGNN or a total of 8-11 repeats of KAGNN and KAGNNN.
Clone the library within the 5′ UTR of a reporter gene cassette such as GFP (preferably within 12 nucleotides of ORF). The library-linked reporter gene may be optionally cloned into a retroviral vector genome and a retroviral vector library produced.
Stably introduce the library-linked reporter gene cassette into a cell line (this can be achieved by transfection or retroviral vector delivery) and isolate single clones.
Screen clones by parallel transfection using control DNA (e.g. pBlueScript) or TRAP-expressing plasmid DNA. Measure reporter gene expression in both scenarios and identify clones with high, non-repressed reporter gene levels (control) and low, repressed reporter gene levels (TRAP).
Identify tbs sequences by PCR amplification and sequencing of the tbs sequence from target cell genomic DNA from candidate clones.

The invention will now be further described by way of Examples, which are meant to serve to assist one of ordinary skill in the art in carrying out the invention and are not intended in any way to limit the scope of the invention.

Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides for use in the invention to reflect the codon usage of any particular host organism in which the polypeptides for use in the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

Proteins

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds.

Variants, Derivatives, Analogues, Homologues and Fragments

In addition to the specific proteins and nucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

The term “homologue” means an entity having a certain homology with the wild type amino acid sequence and the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95% or 97% or 99% identical to the subject sequence. Although homology can also be considered in terms of similarity, in the context of the present invention it is preferred to express homology in terms of sequence identity.

Homology comparisons can be conducted by eye, or more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is ˜12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Research 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol Lett (1999) 174(2):247-50; FEMS Microbiol Lett (1999) 177(1):187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

All variants, fragments or homologues of the RNA-binding protein of the invention will retain the ability to bind the cognate binding site of the invention such that translation of the NOI is repressed or prevented in a viral vector production cell.

All variants fragments or homologues of the binding site of the invention will retain the ability to bind the cognate RNA-binding protein, such that translation of the NOI is repressed or prevented in a viral vector production cell.

Codon Optimisation

The polynucleotides used in the present invention (including the NOI and/or components of the vector production system) may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of a gene of interest, e.g. a NOI or packaging components in mammalian producer cells, can be achieved. Codon usage tables are known in the art for mammalian cells, as well as for a variety of other organisms.

Codon optimisation of viral vector components has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. In lentiviral vectors codon optimisation also overcomes the Rev/RRE requirement for export, rendering optimised sequences Rev-independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with some exceptions, for example the sequence encompassing the frameshift site of gag-pol (see below).

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the Gag-Pol proteins.

For EIAV the beginning of the overlap has been taken to be nt 1262 (where nucleotide 1 is the A of the gag ATG). The end of the overlap is at 1461 bp. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the Gag-Pol proteins.

In one embodiment, codon optimisation is based on lightly expressed mammalian genes. The third and sometimes the second and third base may be changed.

Due to the degenerate nature of the genetic code, it will be appreciated that numerous gag-pol sequences can be achieved by a skilled worker. Also there are many retroviral variants described which can be used as a starting point for generating a codon-optimised gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at the HIV Databases operated by Los Alamos National Security, LLC at http://hiv-web.lanl.gov. Details of EIAV clones may be found at the National Center for Biotechnology Information (NCBI) database located at http://www.ncbi.nlm.nih.gov.

The strategy for codon-optimised gag-pol sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev-independent. In order to enable the use of anti-rev or RRE factors in the lentiviral vector, however, it would be necessary to render the viral vector generation system totally Rev/RRE-independent. Thus, the genome also needs to be modified. This is achieved by optimising vector genome components. Advantageously, these modifications also lead to the production of a safer system absent of all additional proteins both in the producer and in the transduced cell.

Table 1 shows sequences, wherein K may be T or G, “R” is to be understood as specifying a purine (i.e. A or G) at that position in the sequence, “V” is to be understood as specifying any nucleotide from G, A, or C and “N” is to be understood as specifying any nucleotide at that position in the sequence. For example, this could be G, A, T, C or U. The TRAP binding site (tbs) sequence or 3′ tbs sequence is shown in italics, the multiple cloning site (MCS) is shown underlined, and the Kozak sequence is shown in bold.

TABLE 1

| SEQ | | |
|---|---|---|
| ID | | |
| NO | Description | Sequence |
| 1 | TRAP from Bacillus | MNQKHSSDFVVIKAVEDGVNVIGLTRGTDTKFHHSEK |
| | subtilis | LDKGEVIIAQFTEHTSAIKVRGEALIQTAYGEMKSEKK |
| 2 | TRAP from Aminomonas | MKEGEEAKTSVLSDYVVVKALENGVTVIGLTRGQETK |
| | paucivorans | FAHTEKLDDGEVWIAQFTEHTSAIKVRGASEIHTKHGM |
| | | LFSGRGRNEKG |
| 3 | TRAP from | MNPMTDRSDITGDYVVVKALENGVTIIGLTRGGVTKFH |
| | Desulfotomaculum | HTEKLDKGEIMIAQFTEHTSAIKIRGRAELLTKHGKIRTE |
| | hydrothermale | VDS |
| 4 | TRAP from B. | MYTNSDFVVIKALEDGVNVIGLTRGADTRFHHSEKLDK |
| | stearothermophilus | GEVLIAQFTEHTSAIKVRGKAYIQTRHGVIESEGKK |
| 5 | TRAP from B. | MYTNSDFVVIKALEDGVNVIGLTRGADTRFHHSEKLDK |
| | stearothermophilus | GEVLIAQFTEHTSAIKVRGKAYIQTRHGVIENEGKK |
| | S72N | |
| 6 | TRAP from B. | MNVGDNSNFFVIKAKENGVNVFGMTRGTDTRFHHSE |
| | halodurans | KLDKGEVMIAQFTEHTSAVKIRGKAIIQTSYGTLDTEKD |
| | | E |
| 7 | TRAP from | MVCDNFAFSSAINAEYIVVKALENGVTIMGLTRGKDTK |
| | Carboxydothermus | FHHTEKLDKGEVMVAQFTEHTSAIKIRGKAEIYTKHGVI |
| | hydrogenoformans | KNE |
| 8 | TRAP binding site | GAGUUUAGCGGAGUGGAGAAGAGCGGAGCCGAGC |
| | variant-[KAGNN]₁₁ | CUAGCAGAGACGAGUGGAGCU |
| 9 | TRAP binding site | GAGUUUAGCGGAGUGGAGAAGAGCGGAGCCGAGC |
| | variant-[KAGNN]₁₁ | CUAGCAGAGACGAGAAGAGCU |
| 10 | TRAP binding site | UAGUUUAGUUUAGUUUAGUUUAGUUUAGUU |
| | variant-[KAGNN]₆ | |
| 11 | TRAP binding site | UAGUUUAGUUGAGUUUAGUUGAGUUUAGUU |
| | variant-[KAGNN]₆ | |
| 12 | TRAP binding site | GAGUUUGAGUUGAGUUGAGUUUGAGUUGAGUU |
| | variant-[KAGNN]₆ | |
| 13 | TRAP binding site | UAGUUUGAGUUUAGUUGAGUUUUAGUUGAGUU |
| | variant-[KAGNN]₆ | |
| 14 | TRAP binding site | GAGTTTAGCGGAGTGGAGAAGAGCGGAGCCGAGC |
| | variant-[KAGNN]₁₁ | CTAGCAGAGACGAGTGGAGCT |
| 15 | TRAP binding site | GAGTTTAGCGGAGTGGAGAAGAGCGGAGCCGAGC |
| | variant-[KAGNN]₁₀ | CTAGCAGAGACGAGAA |
| 16 | TRAP binding site | GAGTTTAGCGGAGTGGAGAAGAGCGGAGCCGAGC |
| | variant-[KAGNN]₉ | CTAGCAGAGAC |
| 17 | TRAP binding site | GAGTTTAGCGGAGTGGAGAAGAGCGGAGCCGAGC |
| | variant-[KAGNN]₈ | CTAGCA |
| 18 | TRAP binding site | GAGTTTAGCGGAGTGGAGAAGAGCGGAGCCGAGC |
| | variant-[KAGNN]₇ | C |
| 19 | TRAP binding site | GAGTTTAGCGGAGTGGAGAAGAGCGGAGCC |
| | variant-[KAGNN]₆ | |
| 20 | TRAP binding site | GAGTTTAGCGGAGTGGAGAAAGAGACGGAGCCGA |
| | variant- | GACCTAGCAGAGACGAGAAGAGCT |
| | [KAGNNN]₃[KAGNN]₈ | |
| 21 | TRAP binding site | GAGTTTAGCGGAGTGGAGAAGAGACGGAGCCGAG |
| | variant- | CCTAGCAGAGACGAGAAGAGCT |
| | [KAGNNN]₁[KAGNN]₁₀ | |
| 22 | TRAP binding site | GAGTTTAGCGGAGTGGAGAAGAGCGGAGCCGAGC |
| | variant-[KAGNN]₁₁ | CTAGCAGAGACGAGAAGAGCT |
| 23 | TRAP binding site | GAGTTTAGCGGAGTGGAGAAAGAGCGGAGCCGAG |
| | variant- | CCTAGCA |
| | [KAGNNN]₁[KAGNN]₇ | |
| 24 | TRAP binding site | GAGTTTAGCGGAGTGGAGAAAGAGCGGAGCCGAG |
| | variant- | CC |
| | [KAGNNN]₁[KAGNN]₆ | |
| 25 | L33 Improved leader | CTTTTTCGCAACGGGTTTGCCGCCAGAACACAG |
| 26 | L12 Improved leader | CTTTTTCGCAAC |
| 27 | Extended Kozak | GNNRVVATG |
| | consensus sequence | |
| 28 | Core Kozak consensus | RVVATG |
| | sequence | |
| 29 | 3′ tbs-Kozak junction | [symbol] |
| | variant | |
| 30 | 3′ tbs-Kozak junction | [symbol] |
| | variant | |
| 31 | 3′ tbs-Kozak junction | KA [symbol] ATG |
| | variant | |
| 32 | 3′ tbs-Kozak junction | KAG [symbol] ATG |
| | variant | |
| 33 | 3′ tbs-Kozak junction | [symbol] VVATG |
| | variant | |
| 34 | Optimal 3′ tbs-Kozak | [symbol] G |
| | junction variant | |
| 35 | Optimal 3′ tbs-Kozak | KAGN [symbol] ATG |
| | junction variant | |
| 36 | Optimal 3′ tbs-Kozak | KAGNN [symbol] CATG |
| | junction variant | |
| 37 | 3′ tbs-Kozak junction | KAG [symbol] TG |
| | variant | |
| 38 | Spacer variant | ATAGCAGAGACGGCT |
| 39 | Spacer variant | ATAGCAGAGA |
| 40 | Spacer variant | ATAGC |
| 41 | Spacer variant | ATATCAGAGACGGCTAGCGTATACCA |
| 42 | Spacer variant | ATATCAGAGACGGCT |
| 43 | Spacer variant | AGAGACGGCT |
| 44 | Spacer variant | TACCA |
| 45 | 3′ tbs-MCS-Kozak | GAGCTCTAGAVVATG |
| | variant | |
| 46 | 3′ tbs-MCS-Kozak | GAGCTCGTCGACVATG |
| | variant | |
| 47 | 3′ tbs-MCS-Kozak | GAGCTCGAATTCGAAVVATG |
| | variant | |
| 48 | 3′ tbs-MCS-Kozak | GAGCTCTAGACGTCGACVATG |
| | variant | |
| 49 | 3′ tbs-MCS-Kozak | GAGCTCTAGAATTCGAAVVATG |
| | variant | |
| 50 | 3′ tbs-MCS-Kozak | GAGCTCTAGATATCGATRVVATG |
| | variant | |
| 51 | 3′ tbs-MCS-Kozak | KAGACTAGTACTTAAGCTTRVVATG |
| | variant | |
| 52 | 3′ tbs-MCS-Kozak | GAGCTCTAGACCATG |
| | variant | |
| 53 | 3′ tbs-MCS-Kozak | GAGCTCGTCGACCATG |
| | variant | |
| 54 | 3′ tbs-MCS-Kozak | GAGCTCGAATTCGAACCATG |
| | variant | |
| 55 | 3′ tbs-MCS-Kozak | GAGCTCTAGACGTCGACCATG |
| | variant | |
| 56 | 3′ tbs-MCS-Kozak | GAGCTCTAGAATTCGAACCATG |
| | variant | |
| 57 | 3′ tbs-MCS-Kozak | GAGCTCTAGATATCGATACCATG |
| | variant | |
| 58 | 3′ tbs-MCS-Kozak | KAGACTAGTACTTAAGCTTACCATG |
| | variant | |
| 59 | EMCV M loop | CGTGGTTTTCCTTTGAAAAACACGATGATACC |
| 60 | Optimal (overlapping) | GAGTTTAGCGGAGTGGAGAAGAGCGGAGCCGAGC |
| | tbs-Kozak | CTAGCAGAGACGA [symbol] |
| 61 | Optimal (overlapping) | GAGTTTAGCGGAGTGGAGAAGAGCGGAGCCGAGC |
| | tbs-MCS-Kozak | CTAGCAGAGACGAGAAGAGCTCTAGACCATG |
| 62 | Illustrative nucleic acid | CTTTTTCGCAACGGGTTTGCCGCCAGAACACAGGA |
| | sequence 1 containing | GTTTAGCGGAGTGGAGAAGAGCGGAGCCG |
| | L33 Improved leader, | [symbol] |
| | optimal (overlapping) tbs | |
| | ([KAGNN]₈)-Kozak | |
| | junction | |
| 63 | Illustrative nucleic acid | CTTTTTCGCAACGGGTTTGCCGCCAGAACACAGGA |
| | sequence 2 containing | GTTTAGCGGAGTGGAGAAGAGCGGAGCCGAGCCTA |
| | L33 Improved leader, | GCAGAGACGA [symbol] |
| | optimal (overlapping) tbs | |
| | ([KAGNN]₁₁)-Kozak | |
| | junction | |
| 64 | Illustrative nucleic acid | CTTTTTCGCAACGAGTTTAGCGGAGTGGAGAAGAG |
| | sequence 3 containing | CGGAGCCGAGCCTAGCAGAGACG [symbol] |
| | L12 Improved leader, | |
| | optimal (overlapping) tbs | |
| | ([KAGNN]₁₁)-Kozak | |
| | junction | |
| 65 | Illustrative nucleic acid | CTTTTTCGCAACGGGTTTGCCGCCAGAACACAGGT |
| | sequence 4 for intron- | GTCGTGAAAAGAGTTTAGCGGAGTGGAGAAGAGCG |
| | containing 5′UTRs, | GAGCCGAGCCTAGCAGAGACGAGCCGAGATG |
| | resulting in a spliced | |
| | leader comprising L33, | |
| | optimal (overlapping) tbs | |
| | ([KAGNN]₁₁)-Kozak | |
| | junction | |
| 66 | Illustrative nucleic acid | ATAGCAGAGACGGCTGAGTTTAGCGGAGTGGAGAA |
| | sequence 5 containing | GAGCGGAGCCG [symbol] |
| | improved spacer, optimal | |
| | (overlapping) tbs | |
| | ([KAGNN]₈)-Kozak | |
| | junction | |
| 67 | Illustrative nucleic acid | CTTTTTCGCAACGGGTTTGCCGCCAGAACACAGGA |
| | sequence 6 containing | GTTTAGCGGAGTGGAGAAGAGCGGAGCCGAGCCTA |
| | L33 Improved leader tbs | GCAGAGACGAGAAGAGCTCTAGACCATG |
| | ([KAGNN]₁₁)-MCS- | |
| | Kozak | |
| 68 | Illustrative nucleic acid | ATAGCAGAGACGGCTGAGTTTAGCGGAGTGGAGAA |
| | sequence 7 containing | GAGCGGAGCCGAGCCTAGCAGAGACGAGAAGAGC |
| | improved spacer, tbs | TCTAGACCATG |
| | ([KAGNN]₁₁)-MCS- | |
| | Kozak | |
| 69 | 3′ tbs-Kozak junction | G [symbol] |
| | variant | |
| 70 | 3′ tbs-Kozak junction | G [symbol] TG |
| | variant | |
| 71 | 3′ tbs-Kozak junction | G [symbol] TG |
| | variant | |
| 72 | 3′ tbs-Kozak junction | T [symbol] TG |
| | variant | |
| 73 | 3′ tbs-Kozak junction | [symbol] TG |
| | variant | |
| 74 | 3′ tbs-Kozak junction | [symbol] TG |
| | variant | |
| 75 | 3′ tbs-Kozak junction | [symbol] ATG |
| | variant | |
| 76 | 3′ tbs-Kozak junction | [symbol] ATG |
| | variant | |
| 77 | 3′ tbs-Kozak junction | [symbol] ATG |
| | variant | |
| 78 | 3′ tbs-Kozak junction | [symbol] ATG |
| | variant | |
| 79 | 3′ tbs-Kozak junction | [symbol] ATG |
| | variant | |
| 80 | 3′ tbs-Kozak junction | [symbol] ATG |
| | variant | |
| 81 | 3′ tbs-Kozak junction | [symbol] ATG |
| | variant | |
| 82 | 3′ tbs-Kozak junction | [symbol] ATG |
| | variant | |
| 83 | 3′ tbs-Kozak junction | [symbol] ATG |
| | variant | |
| 84 | 3′ tbs-Kozak junction | [symbol] ATG |
| | variant | |
| 85 | 3′ tbs-Kozak junction | [symbol] ATG |
| | variant | |
| 86 | 3′ tbs-Kozak junction | [symbol] ATG |
| | variant | |
| 87 | 3′ tbs-Kozak junction | [symbol] ATG |
| | variant | |
| 88 | 3′ tbs-Kozak junction | [symbol] ATG |
| | variant | |
| 89 | 3′ tbs-Kozak junction | [symbol] ATG |
| | variant | |
| 90 | 3′ tbs-Kozak junction | [symbol] ATG |
| | variant | |
| 91 | 3′ tbs-Kozak junction | [symbol] ATG |
| | variant | |
| 92 | 3′ tbs-Kozak junction | [symbol] ATG |
| | variant | |
| 93 | L33 Improved leader | CTTTTTCGCAACGGGTTTGCCGCCAGAACACAGGT |
| | derived from a splicing | GTCGTGAAAA |
| | event | |
| 94 | Splice donor region | GGGGCGGCGACTGGTGAGTACGCCAAAAAT |
| | variant | |
| 95 | Splice donor region | GGGGCGGCGACTGCAGACAACGCCAAAAAT |
| | variant | |
| 96 | Major splice donor | TG/GTRAGT |
| | consensus sequence | |
| | variant | |
| 97 | Major splice donor | CTGGT |
| | consensus sequence | |
| | variant | |
| 98 | Mutated splice donor | CAGACA |
| | region variant (MSD- | |
| | 2KO) | |
| 99 | Mutated splice donor | GGCGACTGCAGACAACGCC |
| | region variant (MSD- | |
| | 2KO) | |
| 100 | Mutated splice donor | GTGGAGACT |
| | region variant (MSD- | |
| | 2KOv2) | |
| 101 | Mutated splice donor | GGCGAGTGGAGACTACGCC |
| | region variant (MSD- | |
| | 2KOv2) | |
| 102 | SL2 | GGCGACTGGTGAGTACGCC |
| 103 | Cryptic splice donor | TGAGT |
| | consensus sequence | |
| | variant | |
| 104 | MSD-2KOv2 | GGGGCGGCGAGTGGAGACTACGCCAAAAAT |
| 105 | MSD-2KOm5 | GGGGAAGGCAACAGATAAATATGCCTTAAAAT |
| 106 | Core sequence | /GTGA/GTA |
| 107 | Mutated splice donor | AAGGCAACAGATAAATATGCCTT |
| 108 | 3′ tbs-Kozak junction | [symbol] |
| | variant | |
| 109 | 3′ tbs-Kozak junction | [symbol] ATG |
| | variant | |
| 110 | 3′ tbs-Kozak junction | [symbol] ATG |
| | variant | |
| 111 | 3′ tbs-Kozak junction | [symbol] ATG |
| | variant | |
| 112 | 3′ tbs-Kozak junction | [symbol] ATG |
| | variant | |
| 113 | 3′ tbs-Kozak consensus | [symbol] |
| | sequence | |
| 114 | 3′ tbs-Kozak consensus | [symbol] |
| | sequence | |
| 115 | 3′ tbs-Kozak consensus | [symbol] TG |
| | sequence | |
| 116 | 3′ tbs-Kozak consensus | [symbol] TG |
| | sequence | |
| 117 | Chicken β-Actin/Rabbit | CGGCGGGCGGGAACGTTGCCTTCGCCCCGTGCCC |
| | β-globin chimeric 5′UTR- | CGCTCCGCGCCGCCTCGCGCCGCCCGCCCCGGCT |
| | intron with tbs-kzkV0.G | CTGACTGACCGCGTTACTCCCACAGGTGAGCGGG |
| | variant; exonic sequence | CGGGACGGCCCTTCTCCCTCCGGGCTGTAATTAGC |
| | in bold (spliced together | GCTTGGTTTAATGACGGCTCGTTTCTTTTCTGTGGC |
| | to become 5′UTR | TGCGTGAAAGCCTTAAAGGGCTCCGGGAGGGCCTT |
| | leader), tbskzkV0.G in | TGTGCGGGGGGGAGCGGCTCGGGGGGTGCGTGC |
| | italics | GTGTGTGTGTGCGTGGGGAGCGCCGCGTGCGGCC |
| | | CGCGCTGCCCGGCGGCTGTGAGCGCTGCGGGCGC |
| | | GGCGCGGGGCTTTGTGCGCTCCGCGTGTGCGCGA |
| | | GGGGAGCGCGGGCCGGGGGCGGTGCCCCGCGGT |
| | | GCGGGGGGGCTGCGAGGGGAACAAAGGCTGCGTG |
| | | CGGGGTGTGTGCGTGGGGGGGTGAGCAGGGGGTG |
| | | TGGGCGCGGCGGTCGGGCTGTAACCCCCCCCTGG |
| | | CACCCCCCTCCCCGAGTTGCTGAGCACGGCCCGG |
| | | CTTCGGGTGCGGGGCTCCGTGCGGGGCGTGGCGC |
| | | GGGGCTCGCCGTGCCGGGCGGGGGGTGGCGGCA |
| | | GGTGGGGGTGCCGGGCGGGGCGGGGCCGCCTCG |
| | | GGCCGGGGAGGGCTCGGGGGAGGGGCGCGGCGG |
| | | CCCCGGAGCGCCGGCGGCTGTCGAGGCGCGGCGA |
| | | GCCGCAGCCATTGCCTTTTATGGTAATCGTGCGAGA |
| | | GGGCGCAGGGACTTCCTTTGTCCCAAATCTGGCGG |
| | | AGCCGAAATCTGGGAGGCGCCGCCGCACCCCCTCT |
| | | AGCGGGCGCGGGCGAAGCGGTGCGGCGCCGGCA |
| | | GGAAGGAAATGGGCGGGGAGGGCCTTCGTGCGTC |
| | | GCCGCGCCGCCGTCCCCTTCTCCATCTCCAGCCTC |
| | | GGGGCTGCCGCAGGGGGACGGCTGCCTTCGGGGG |
| | | GGACGGGGCAGGGCGGGGTTCGGCTTCTGGCGTG |
| | | TGACCGGCGGCTTTAGAGCCTCTGCTAACCATGTTC |
| | | ATGCCTTCTTCTTTTTCCTACAGCTCCTGGGCAAA [symbol] |
| | | [symbol] [symbol] |
| | | [symbol] ATG |
| 118 | EF1a 5′UTR-intron with | CTTTTTCGCAACGGGTTTGCCGCCAGAACACAGGT |
| | tbskzkV0.G variant; | AAGTGCCGTGTGTGGTTCCCGCGGGCCTGGCCTCT |
| | exonic sequence in bold | TTACGGGTTATGGCCCTTGCGTGCCTTGAATTACTT |
| | (spliced together to | CCACCTGGCTGCAGTACGTGATTCTTGATCCCGAG |
| | become 5′UTR leader), | CTTCGGGTTGGAAGTGGGTGGGAGAGTTCGTGGCC |
| | tbskzkV0.G in italics | TTGCGCTTAAGGAGCCCCTTCGCCTCGTGCTTGAG |
| | | TTGTGGCCTGGCCTGGGCGCTGGGGCCGCCGCGT |
| | | GCGAATCTGGTGGCACCTTCGCGCCTGTCTCGCTG |
| | | CTTTCGATAAGTCTCTAGCCATTTAAAATTTTTGATG |
| | | ACCTGCTGCGACGCTTTTTTTCTGGCAAGATAGTCT |
| | | TGTAAATGCGGGCCAAGATCAGCACACTGGTATTTC |
| | | GGTTTTTGGGGCCGCGGGCGGCGACGGGGCCCGT |
| | | GCGTCCCAGCGCACATGTTCGGCGAGGCGGGGCC |
| | | TGCGAGCGCGGCCACCGAGAATCGGACGGGGGTA |
| | | GTCTCAAGCTGCCCGGCCTGCTCTGGTGCCTGGCC |
| | | TCGCGCCGCCGTGTATCGCCCCGCCCTGGGCGGC |
| | | AAGGCTGGCCCGGTCGGCACCAGTTGCGTGAGCG |
| | | GAAAGATGGCCGCTTCCCGGCCCTGCTGCAGGGA |
| | | GCACAAAATGGAGGACGCGGCGCTCGGGAGAGCG |
| | | GGCGGGTGAGTCACCCACACAAAGGAAAAGGGCCT |
| | | TTCCGTCCTCAGCCGTCGCTTCATGTGACTCCACG |
| | | GAGTACCGGGCGCCGTCCAGGCACCTCGATTAGTT |
| | | CTCCAGCTTTTGGAGTACGTCGTCTTTAGGTTGGGG |
| | | GGAGGGGTTTTATGCGATGGAGTTTCCCCACACTG |
| | | AGTGGGTGGAGACTGAAGTTAGGCCAGCTTGGCAC |
| | | TTGATGTAATTCTCCTTGGAATTTGCCCTTTTTGAGT |
| | | TTGGATCTTGGTTCATTCTCAAGCCTCAGACAGTGG |
| | | TTCAAAGTTTTTTTCTTCCATTTCAGGTGTCGTGAAAA |
| | | [symbol] [symbol] |
| | | [symbol] G |
| 119 | Spliced sequence | CGGCGGGCGGGAACGTTGCCTTCGCCCCGTGCCC |
| | corresponding to SEQ ID | CGCTCCGCGCCGCCTCGCGCCGCCCGCCCCGGCT |
| | NO: 117; 5′UTR leader | CTGACTGACCGCGTTACTCCCACAGCTCCTGGGCA |
| | sequence in bold, | AA [symbol] |
| | tbskzkV0.G in italics | [symbol] G |
| 120 | Spliced sequence | CTTTTTCGCAACGGGTTTGCCGCCAGAACACAGGT |
| | corresponding to SEQ ID | GTCGTGAAAA [symbol] |
| | NO: 118; 5′UTR leader | [symbol] [symbol] G |
| | sequence in bold, | |
| | tbskzkV0.G in italics | |
| 121 | Chicken β-Actin/Rabbit | CGGCGGGCGGGAACGTTGCCTTCGCCCCGTGCCC |
| | β-globin chimeric 5′UTR- | CGCTCCGCGCCGCCTCGCGCCGCCCGCCCCGGCT |
| | intron (exonic sequence | CTGACTGACCGCGTTACTCCCACAGGTGAGCGGG |
| | in bold (spliced together | CGGGACGGCCCTTCTCCCTCCGGGCTGTAATTAGC |
| | to become 5′UTR | GCTTGGTTTAATGACGGCTCGTTTCTTTTCTGTGGC |
| | leader)) | TGCGTGAAAGCCTTAAAGGGCTCCGGGAGGGCCTT |
| | | TGTGCGGGGGGGAGCGGCTCGGGGGGTGCGTGC |
| | | GTGTGTGTGTGCGTGGGGAGCGCCGCGTGCGGCC |
| | | CGCGCTGCCCGGCGGCTGTGAGCGCTGCGGGCGC |
| | | GGCGCGGGGCTTTGTGCGCTCCGCGTGTGCGCGA |
| | | GGGGAGCGCGGGCCGGGGGCGGTGCCCCGCGGT |
| | | GCGGGGGGGCTGCGAGGGGAACAAAGGCTGCGTG |
| | | CGGGGTGTGTGCGTGGGGGGGTGAGCAGGGGGTG |
| | | TGGGCGCGGCGGTCGGGCTGTAACCCCCCCCTGG |
| | | CACCCCCCTCCCCGAGTTGCTGAGCACGGCCCGG |
| | | CTTCGGGTGCGGGGCTCCGTGCGGGGCGTGGCGC |
| | | GGGGCTCGCCGTGCCGGGCGGGGGGTGGCGGCA |
| | | GGTGGGGGTGCCGGGCGGGGCGGGGCCGCCTCG |
| | | GGCCGGGGAGGGCTCGGGGGAGGGGCGCGGCGG |
| | | CCCCGGAGCGCCGGCGGCTGTCGAGGCGCGGCGA |
| | | GCCGCAGCCATTGCCTTTTATGGTAATCGTGCGAGA |
| | | GGGCGCAGGGACTTCCTTTGTCCCAAATCTGGCGG |
| | | AGCCGAAATCTGGGAGGCGCCGCCGCACCCCCTCT |
| | | AGCGGGCGCGGGCGAAGCGGTGCGGCGCCGGCA |
| | | GGAAGGAAATGGGCGGGGAGGGCCTTCGTGCGTC |
| | | GCCGCGCCGCCGTCCCCTTCTCCATCTCCAGCCTC |
| | | GGGGCTGCCGCAGGGGGACGGCTGCCTTCGGGGG |
| | | GGACGGGGCAGGGCGGGGTTCGGCTTCTGGCGTG |
| | | TGACCGGCGGCTTTAGAGCCTCTGCTAACCATGTTC |
| | | ATGCCTTCTTCTTTTTCCTACAGCTCCTGGGCAAA |
| 122 | EF1a 5′UTR-intron | CTTTTTCGCAACGGGTTTGCCGCCAGAACACAGGT |
| | (exonic sequence in bold | AAGTGCCGTGTGTGGTTCCCGCGGGCCTGGCCTCT |
| | (spliced together to | TTACGGGTTATGGCCCTTGCGTGCCTTGAATTACTT |
| | become 5′UTR leader)) | CCACCTGGCTGCAGTACGTGATTCTTGATCCCGAG |
| | | CTTCGGGTTGGAAGTGGGTGGGAGAGTTCGTGGCC |
| | | TTGCGCTTAAGGAGCCCCTTCGCCTCGTGCTTGAG |
| | | TTGTGGCCTGGCCTGGGCGCTGGGGCCGCCGCGT |
| | | GCGAATCTGGTGGCACCTTCGCGCCTGTCTCGCTG |
| | | CTTTCGATAAGTCTCTAGCCATTTAAAATTTTTGATG |
| | | ACCTGCTGCGACGCTTTTTTTCTGGCAAGATAGTCT |
| | | TGTAAATGCGGGCCAAGATCAGCACACTGGTATTTC |
| | | GGTTTTTGGGGCCGCGGGCGGCGACGGGGCCCGT |
| | | GCGTCCCAGCGCACATGTTCGGCGAGGCGGGGCC |
| | | TGCGAGCGCGGCCACCGAGAATCGGACGGGGGTA |
| | | GTCTCAAGCTGCCCGGCCTGCTCTGGTGCCTGGCC |
| | | TCGCGCCGCCGTGTATCGCCCCGCCCTGGGCGGC |
| | | AAGGCTGGCCCGGTCGGCACCAGTTGCGTGAGCG |
| | | GAAAGATGGCCGCTTCCCGGCCCTGCTGCAGGGA |
| | | GCACAAAATGGAGGACGCGGCGCTCGGGAGAGCG |
| | | GGCGGGTGAGTCACCCACACAAAGGAAAAGGGCCT |
| | | TTCCGTCCTCAGCCGTCGCTTCATGTGACTCCACG |
| | | GAGTACCGGGCGCCGTCCAGGCACCTCGATTAGTT |
| | | CTCCAGCTTTTGGAGTACGTCGTCTTTAGGTTGGGG |
| | | GGAGGGGTTTTATGCGATGGAGTTTCCCCACACTG |
| | | AGTGGGTGGAGACTGAAGTTAGGCCAGCTTGGCAC |
| | | TTGATGTAATTCTCCTTGGAATTTGCCCTTTTTGAGT |
| | | TTGGATCTTGGTTCATTCTCAAGCCTCAGACAGTGG |
| | | TTCAAAGTTTTTTTCTTCCATTTCAGGTGTCGTGAAA |
| | | A |
| 123 | Chicken β-Actin/Rabbit | CGGCGGGCGGGAACGTTGCCTTCGCCCCGTGCCC |
| | β-globin chimeric 5′UTR- | CGCTCCGCGCCGCCTCGCGCCGCCCGCCCCGGCT |
| | intron-tbs consensus | CTGACTGACCGCGTTACTCCCACAGGTGAGCGGG |
| | (exonic sequence in bold | CGGGACGGCCCTTCTCCCTCCGGGCTGTAATTAGC |
| | (spliced together to | GCTTGGTTTAATGACGGCTCGTTTCTTTTCTGTGGC |
| | become 5′UTR leader), | TGCGTGAAAGCCTTAAAGGGCTCCGGGAGGGCCTT |
| | tbs consensus in italics) | TGTGCGGGGGGGAGCGGCTCGGGGGGTGCGTGC |
| | | GTGTGTGTGTGCGTGGGGAGCGCCGCGTGCGGCC |
| | | CGCGCTGCCCGGCGGCTGTGAGCGCTGCGGGCGC |
| | | GGCGCGGGGCTTTGTGCGCTCCGCGTGTGCGCGA |
| | | GGGGAGCGCGGGCCGGGGGCGGTGCCCCGCGGT |
| | | GCGGGGGGGCTGCGAGGGGAACAAAGGCTGCGTG |
| | | CGGGGTGTGTGCGTGGGGGGGTGAGCAGGGGGTG |
| | | TGGGCGCGGCGGTCGGGCTGTAACCCCCCCCTGG |
| | | CACCCCCCTCCCCGAGTTGCTGAGCACGGCCCGG |
| | | CTTCGGGTGCGGGGCTCCGTGCGGGGCGTGGCGC |
| | | GGGGCTCGCCGTGCCGGGCGGGGGGTGGCGGCA |
| | | GGTGGGGGTGCCGGGCGGGGCGGGGCCGCCTCG |
| | | GGCCGGGGAGGGCTCGGGGGAGGGGCGCGGCGG |
| | | CCCCGGAGCGCCGGCGGCTGTCGAGGCGCGGCGA |
| | | GCCGCAGCCATTGCCTTTTATGGTAATCGTGCGAGA |
| | | GGGCGCAGGGACTTCCTTTGTCCCAAATCTGGCGG |
| | | AGCCGAAATCTGGGAGGCGCCGCCGCACCCCCTCT |
| | | AGCGGGCGCGGGCGAAGCGGTGCGGCGCCGGCA |
| | | GGAAGGAAATGGGCGGGGAGGGCCTTCGTGCGTC |
| | | GCCGCGCCGCCGTCCCCTTCTCCATCTCCAGCCTC |
| | | GGGGCTGCCGCAGGGGGACGGCTGCCTTCGGGGG |
| | | GGACGGGGCAGGGCGGGGTTCGGCTTCTGGCGTG |
| | | TGACCGGCGGCTTTAGAGCCTCTGCTAACCATGTTC |
| | | ATGCCTTCTTCTTTTTCCTACAGCTCCTGGGCAAA |
| | | [symbol] [symbol] |
| 124 | EF1a 5′UTR-intron-tbs | CTTTTTCGCAACGGGTTTGCCGCCAGAACACAGGT |
| | consensus (exonic | AAGTGCCGTGTGTGGTTCCCGCGGGCCTGGCCTCT |
| | sequence in bold | TTACGGGTTATGGCCCTTGCGTGCCTTGAATTACTT |
| | (spliced together to | CCACCTGGCTGCAGTACGTGATTCTTGATCCCGAG |
| | become 5′UTR leader), | CTTCGGGTTGGAAGTGGGTGGGAGAGTTCGTGGCC |
| | tbs consensus in italics) | TTGCGCTTAAGGAGCCCCTTCGCCTCGTGCTTGAG |
| | | TTGTGGCCTGGCCTGGGCGCTGGGGCCGCCGCGT |
| | | GCGAATCTGGTGGCACCTTCGCGCCTGTCTCGCTG |
| | | CTTTCGATAAGTCTCTAGCCATTTAAAATTTTTGATG |
| | | ACCTGCTGCGACGCTTTTTTTCTGGCAAGATAGTCT |
| | | TGTAAATGCGGGCCAAGATCAGCACACTGGTATTTC |
| | | GGTTTTTGGGGCCGCGGGCGGCGACGGGGCCCGT |
| | | GCGTCCCAGCGCACATGTTCGGCGAGGCGGGGCC |
| | | TGCGAGCGCGGCCACCGAGAATCGGACGGGGGTA |
| | | GTCTCAAGCTGCCCGGCCTGCTCTGGTGCCTGGCC |
| | | TCGCGCCGCCGTGTATCGCCCCGCCCTGGGCGGC |
| | | AAGGCTGGCCCGGTCGGCACCAGTTGCGTGAGCG |
| | | GAAAGATGGCCGCTTCCCGGCCCTGCTGCAGGGA |
| | | GCACAAAATGGAGGACGCGGCGCTCGGGAGAGCG |
| | | GGCGGGTGAGTCACCCACACAAAGGAAAAGGGCCT |
| | | TTCCGTCCTCAGCCGTCGCTTCATGTGACTCCACG |
| | | GAGTACCGGGCGCCGTCCAGGCACCTCGATTAGTT |
| | | CTCCAGCTTTTGGAGTACGTCGTCTTTAGGTTGGGG |
| | | GGAGGGGTTTTATGCGATGGAGTTTCCCCACACTG |
| | | AGTGGGTGGAGACTGAAGTTAGGCCAGCTTGGCAC |
| | | TTGATGTAATTCTCCTTGGAATTTGCCCTTTTTGAGT |
| | | TTGGATCTTGGTTCATTCTCAAGCCTCAGACAGTGG |
| | | TTCAAAGTTTTTTTCTTCCATTTCAGGTGTCGTGAAA |
| | | A [symbol] |

### EXAMPLES

Experimental Procedures

DNA Cloning

Expression constructs were generated by standard molecular cloning techniques; typically, inserts were generated by re-synthesis (GeneArt) or by short oligo adaptors and inserted into reporter (GFP) plasmids by restriction enzyme digest to generate the indicated constructs. Plasmid DNA was generated by standard transformation and growth of attenuated E. coli strains.

Adherent Cell Culture and Transfection

HEK293T cells were used for transfection in adherent mode—these are typically used in viral vector manufacture, and therefore their use modelled transgene expression/repression in a relevant context. The cells were maintained in complete media (Dulbecco's Modified Eagle Medium (DMEM) (Sigma)) supplemented with 10% heat-inactivated fetal bovine serum (FBS) (Gibco), 2 mM L-glutamine (Sigma) and 1% Non-essential amino acids (NEAA) (Sigma), at 37° C. in 5% CO₂.

HEK293T cells were seeded at 3.5×10⁵ cell per mL of complete media per 0.175 cm² vessel area, and approximately 24 hrs later the cells were transfected using the following mass ratios of plasmids: ‘+TRAP’=34.25 ng/cm² reporter plasmid, 34.25 ng/cm² of pEF1α-TRAP¹ and 68.5 ng/cm² pBluescript; ‘no TRAP’=34.25 ng/cm² reporter plasmid and 102.8 ng/cm² pBluescript. Transfection was mediated by mixing DNA with Lipofectamine 2000CD in FreeStyle serum-free media as per manufacturer's protocol (Life Technologies). Sodium butyrate (Sigma) was added ˜18 hrs later to 10 mM final concentration for 5-6 h, before 10 ml fresh serum-free media replaced the transfection media. Cells were taken for analysis of GFP expression (or luciferase expression were indicated controls were included) approximately 2 days post-transfection.

Suspension Cell Culture and Transfection

HEK293T suspension cells were used for transfection in suspension mode—these are typically used in viral vector manufacture, and therefore their use modelled transgene expression/repression in a relevant context. Cells were grown in Freestyle+0.1% CLC (Gibco) at 37° C. in 5% CO², in a shaking incubator (25 mm orbit set at 190 RPM). Cells were seeded at 8×10⁵ cells per mL in serum-free media and 24 hrs later the cells were transfected using the mass per mL (final culture cvol) ratios of plasmids: ‘+TRAP’=300 ng/mL reporter plasmid, 300 ng/mL of pEF1a-TRAP¹ and 600 ng/mL pBluescript; ‘no TRAP’=300 ng/mL reporter plasmid and 900 ng/mL pBluescript. Transfection was mediated by mixing DNA with Lipofectamine 2000CD in FreeStyle as per manufacturer's protocol (Life Technologies). Sodium butyrate (Sigma) was added ˜18 hrs later to 10 mM final concentration. Cells were taken for analysis of GFP expression (or luciferase expression were indicated controls were included) approximately 2 days post-transfection.

GFP Expression Assay

Transfected cells were prepared for flow cytometry using an Attune-NxT (Thermofisher) and the percentage of GFP expression was measured as well as median fluorescence intensity (MFI). For each experiment, % GFP and MFI values were multiplied to give a relative GFP Expression score. This score was compared to conditions with or without TRAP co-transfection to assess the level of GFP repression.

Luciferase Assay

In replicate cultures, cells were incubated in 100 ul of 1× Passive Lysis Buffer at room temperature added per well. Cells were incubated at room temperature for 45 minutes, and then lysates split into 3× ˜30 ul aliquots and frozen at −80° C. For the Luciferase assay, cell lysates (in PLB) and luciferase assay reagent (LAR) were left to thaw and equilibriate to room temperature before 10 ul of lysates transfected to white 96 well plates. An MLX reader was used to assay luciferase activity using 80 ul LAR/reaction during a 12 second read. Luciferase activity was calculated by using (SUM FLUORESCENT UNITS/READ TIME).

### Example 1: Demonstration of Universally Enhanced Repression by TRAP-Tbs Using Improved Leaders Compared to Native Promoter 5′UTR Leader Sequences

In application of the TRIP system to different promoters (containing different native 5′UTRs of different lengths and composition) it is desirable to be able to simply apply the tbs sequence within a promoter-UTR context to afford efficient repression by TRAP, whilst also maintaining good ‘ON’ levels of expression (without TRAP).

From the outset of this work, it was not known what would be the achievable level of repression mediated by TRAP-tbs, when the tbs is inserted into native UTRs of a variety of constitutive promoters. Ideally, it would be advantageous to be able to supply a single conserved 5′UTR leader sequence together with the tbs when modifying the promoter of choice in order to avoid any of the potential variability in repression levels that might be directed by native 5′UTR sequences. Surprisingly, it was found that the first exon of the EF1a promoter (SEQ ID NO; 25) provides consistently good levels of transgene repression by TRAP compared to 5′UTR leaders comprising native leader sequences, and this leader also provides good ‘ON’ levels of transgene expression in the absence of TRAP (FIG. 3).

The first 33nts of the EF1α exon—herein termed “L33 Improved leader”—was positioned directly upstream of the tbs, and this sequence used to replace the entire 5′UTR of the native leader of the following promoters: RSV, EF1α/EFS, Ubiquitin/UBCs, SV40, human PGK and HSV TK (FIG. 3A). For CMV, the L33 Improved leader was compared to the original 34nt leader used in WO2015/092440, herein termed “original leader”. The tbs was also cloned directly into the native 5′UTRs of these promoters at a heterologous NotI site (see Table I-Panel I/II for details of sequences). These GFP-encoding constructs were individually co-transfected into HEK293T cells+/−a TRAP-expression plasmid, and GFP expression measured two days post-transfection by flow cytometry. A GFP Expression Score (% GFP positive cells×MFI) was generated from flow cytometry data and plotted (FIG. 3B).

The data shows that the L33 Improved leader out-performed the native 5′UTR leaders for all the constitutive promoters tested. The presence of TRAP mediated measureable repression for all the tbs-containing constructs but the level of repression achieved was not as good for native 5′UTR-tbs constructs compared to those containing the L33 Improved leader. Moreover, the ‘ON’ levels of expression (without TRAP) for the L33 Improved leader-containing constructs were generally greater than the native 5′UTR-tbs constructs. The L33 Improved leader performed comparably to the original leader in the CMV promoter, indicating that the greater level of repression by TRAP is achieved in both original leader and L33 Improved leader configurations.

A second Improved leader—herein termed “L12 Improved leader”—was generated by truncating the L33 Improved leader to 12nts (i.e. the first 12 nts of the EF1a Exon 1), and cloned into six constitutive promoter-containing GFP reporter cassettes harboring either the MCS2.1 or MCS4.1 sequences (see below) between the tbs and the Kozak sequence. The reporters were tested for non-repressed or repressed levels of GFP expression by co-transfection of the reporter plasmids with either pBlueScript (No TRAP) or pEF1α-TRAP (TRAP), respectively. Transfected HEK293T cells (suspension, serum-free) were analysed by flow cytometry two days post-transfection, and GFP Expression scores (% GFP×median fluorescence intensity) generated and log-10 transformed (FIG. 8).

The data demonstrate that the L12 Improved leader performed similarly to the L33 Improved leader—allowing full repression by TRAP-tbs, with GFP levels repressed to background levels. Interestingly the ‘ON’ (not repressed/without TRAP) levels of GFP were slightly higher for L12 or L33 depending on the promoter employed. For example, ‘ON’ levels were greater for L12 in EFS constructs but for huPGK the L33 Improved leader gave the great ‘ON’ levels.

This will allow flexibility in being able to choose between either L12 or L33 when considering utilization of the TRIP system with different promoters, so that gene expression levels in the absence of TRAP (i.e. in vector transduced cells) can be maximized, whilst knowing that absolute repression achieved by TRAP-tbs during vector production will be extremely good for either L12 or L33 Improved leaders.

TABLE I
UTR-leader, tbs and MSC variant sequences

| Panel I | UTR-leaders |
|---|---|
| CMV | GTCAGATCCGCTAGCGCTACCGGACTCAGATCTC |
| | [34 nt leader] |
| RSV | GCCATTTGACCATTCACCACATTGGTGTGCACCTC |
| | CAAGGCCAAGATCTTTGTCGATCCTACCATCCACT |
| | CGACACACCCGCCAGCGGCCGC |
| EF1a | CTTTTTCGCAACGGGTTTGCCGCCAGAACACAGGT |
| | GTCGTGAAAACTACCCCTAAAAGCCAAAAGATCTT |
| | TGTCGATCCTACCATCCACTCGACACACCCGCCAG |
| | CGGCCGC |
| UBC | AGCTAGTTCCGTCGCAGCCGGGATTTGGGTCGCGG |
| | TTCTTGTTTGTGGATCGCTGTGATCGTCACTTGGA |
| | CGCAGGGTTCGGGCCTAGGGTAGGCTCTCCTGAAT |
| | CGACAGGCGCCGGACCTCTGGTGAGGGGAGGGATA |
| | AGTGAGGCGTCAGTTTCTTTGGTCGGTTTTATGTA |
| | CCTATCTTCTTAAGTAGCTGAAGCTCCGGTTTTGA |
| | ACTATGCGCTCGGGGTTGGCGAGTGTGTTTTGTGA |
| | AGTTTTTTAGGCACCTTTTGAAATGTAATCATTTG |
| | GGTCAATATGTAATTTTCAGTGTTAGACTTGTAAA |
| | TTGTCCGCTAAATTCTGGCCGTTTTTGGCTTTTTT |
| | GTTAGACAACAGATCTTTGTCGATCCTACCATCCA |
| | CTCGACACACCCGCCAGCGGCCGC |
| SV40 | CTCTGAGCTATTCCAGAAGTAGTGAGGAGGCTTTT |
| | TTGGAGGCCTAGGCTTTTGCAGATCTTTGTCGATC |
| | CTACCATCCACTCGACACACCCGCCAGCGGCCGC |
| huPGK | GTTCCGCATTCTGGCAAGCCTCCGGAGCGCACGTC |
| | GGCAGTCGGCTCCCTCGTTGACCGAATCACCGACC |
| | TCTCTCCCCAGCTGTATTTCCAAAAGATCTTTGTC |
| | GATCCTACCATCCACTCGACACACCCGCCAGCGGC |
| | CGC |
| hsvTK | ACACCGAGCGACCCTGCAGCGACCCGCTTAAGATC |
| | TTTGTCGATCCTACCATCCACTCGACACACCCGCC |
| | AGCGGCCGC |
| Improved | CTTTTTCGCAACGGGTTTGCCGCCAGAACACAG |
| leader L33 | |
| Improved | CTTTTTCGCAAC |
| leader L12 | |
| Panel II | tbs sequence |
| tbs | GAGTTTAGCGGAGTGGAGAAGAGCGGAGCCGAGCC |
| | TAGCAGAGACGAGTGGAGCT |
| tbs consensus | [KAGNN]×11 |
| Panel III | Multicloning site (MSC) variants |
| No MCS | ACAGCCACCATG |
| MCS2.1 | GAGCTCTAG [symbol] CCATG |
| MCS3.1 | GAGCTCGTCG [symbol] CATG |
| MCS3.2 | GAGCTCGAATTCGAACCATG |
| MCS4.1 | GAGCTCTAGACGTCG [symbol] CATG |
| MCS4.2 | GAGCTCTAGAATTCGAACCATG |
| MCS4.3 | GAGCTCTAGATATCGAT [symbol] CCATG |
| MCS4.4 | KAGACTAGTACTTAAGCTTACCATG |

[Panel I] Leaders tested in the 5′UTR-tbs region of different promoters compared to the Improved leaders. The Cytomegalovirus (CMV) promoter was previously used in conjunction with a synthetic leader sequence outlined in WO2015/092440 (original leader), which typically enables the TRAP-tbs complex to repressed transgene expression by >100-fold. The promoters tested were: Rous Sarcoms Virus (RSV), Elongation Factor 1α (EF1α), Ubiquitin C (UBC), Simian Virus 40 (SV40), (human) Phosphoglycerate kinase (huPGK), and Herpes simplex virus (HSV) thymidine kinase (hsvTK). Note that the EF1a and UBC native 5′UTRs result from splicing-out of an intron, which results in a spliced junction and is denoted in the sequence by two bold GG dinucleotides. It should also be noted that since the study is focused on the expression/repression of transgene at the translation level (modulated by TRAP-tbs) the presence of the introns within EF1a and UBC ‘long’ pre-mRNAs are not considered since they are not present in spliced mRNA; the EF1a promoter is therefore directly comparable to the ‘short’ variant (EFS) that lacks the intron, and likewise the UBC is comparable to the ‘short’ UBCs promoter. Further variants of the invention in the context of the EF1a promoter (intron-containing) are described in Example 5. The constitutive promoters evaluated in the study harbored 5′UTR leaders that comprised native leader sequence (underlined), followed by synthetic sequence up to an including a NotI site; the tbs sequence immediately followed the NotI site. The Improved leaders L33 and L12 comprises sequence derived from the first exon of the EF1a promoter. [Panel II] The tbs sequence principally used in the study to compare the impact of UTR leader sequence on transgene (GFP) repression levels by TRAP-tbs, and the semi-degenerate tbs consensus sequence. [Panel III] Sequences comprising the multiple cloning sites sequence evaluated in the study compared to No MCS, which was used in WO2015092440. Underlined sequence comprises the 3′ end of the tbs, italicized sequence comprises restriction enzyme (RE) sites (some overlap the tbs and/or each other to allow for sequence compression in order to minimize the sequence between the tbs and core Kozak sequence), and the core Kozak sequence is in bold.

### Example 2: Identification of Optimal Tbs-MCS-Transgene Configurations

To improve the tractability of the TRIP system—particularly for commercialization within a ‘kit’—it is desirable to be able to have the ability to clone a transgene of interest directly into the expression cassette containing the promoter-5′UTR-tbs sequence via the option of several different restriction enzymes (RE) i.e. a multiple cloning site (MCS) (See FIG. 2A). However, given that 5′UTR leader sequences can modulate the degree of TRAP-mediated repression, and that the close proximity of the tbs to the ATG initiation codon is important, it was not obvious at the outset of this work as to how many and/or what combinations of RE sites could be used whilst maintaining TRAP-mediated repression.

In addition, a further requirement was to ensure that the ‘ON’ levels of the transgene (without TRAP) are high i.e. an efficient core Kozak sequence must be maintained within or between the MCS and the ATG. This required sequence ‘compression’ such that several (overlapping) RE sites could be incorporated in as short a distance as possible from the tbs to the ATG (to retain proximity of tbs to ATG), whilst also maintaining an efficient core Kozak sequence of consensus RVVATG. The design of seven MCS variants cloned between the tbs and the ATG codon of the transgene (in this case GFP) is reported in Table I-Panel III (sequences; SEQ ID NOs: 52-58) and FIG. 4A. Variants MCS2.1 to MCS4.4 incorporate progressively more (overlapping) RE sites between the tbs and the core Kozak sequence, whilst ensuring a relatively short distance (<15nt) between the tbs and the ATG codon. In all cases it was possible to position either a SacI or SpeI site overlapping the 3′terminus of the tbs without disrupting the consensus of the final 1-2 repeats (KAGNN). The MCS variants introduced between 2 to 4 RE sites or 3 to 5 REs sites if the NcoI site is retained in the transgene core Kozak sequence (dependent on a G downstream of ATG); across all the MCS variants a total of 15 RE sites (16 including the NcoI) were tested as part of MCSs in this context, and also in the context of the EFS promoter using the L33 Improved leader described in Example 1 to ensure that any differences observed would not be due to influence of the sequences upstream of the tbs. These GFP-encoding MCS variants (cloned within a scAAV2 vector genome cassette) were individually co-transfected into HEK293T cells+/−a TRAP-expression plasmid, and GFP expression measured two days post-transfection by flow cytometry. A GFP Expression Score (% GFP positive cells×MFI) was generated from flow cytometry data and plotted (FIG. 4B).

The data identifies that all seven MCS variants could be repressed to levels similar to the original reporter construct (driven by stronger CMV promoter), which contained no MCS between the tbs and ATG codon (the tbs and ATG is separated by 9nt in the original reporter). Importantly, six of the seven variants showed better repression by TRAP compared to the original reporter construct, and variants MCS2.1, MCS4.1 and MCS4.4 were repressed to undetectable levels, with virtually no impact on ‘ON’ levels (without TRAP).

To further validate the MCS2.1 and MCS4.1 variants, these sequences were cloned into scAAV2 vector genome reporter constructs encoding the constitutive promoters—CMV, RSV, EFS, UBCs, SV40, huPGK and HSVTK—together with the L33 Improved leader. These GFP-encoding reporter constructs were individually co-transfected into HEK293T cells+/−a TRAP-expression plasmid, and GFP expression measured two days post-transfection by flow cytometry. A GFP Expression Score (% GFP positive cells×MFI) was generated from flow cytometry data and plotted (FIG. 5).

The data demonstrates that the MCS2.1 and MCS4.1 variants display an excellent TRIP repression profile irrespective of the promoter being utilized. The average fold-repression across the set of constructs containing either of the two MCS variants was ˜5000-fold, and for many constructs close to the limit of GFP detection in the assay.

### Example 3: Optimisation of Tbs-Kozak Junction

The testing of the MCS variants in Example 2 demonstrated that the sequence context between the terminus of the tbs and the ATG codon may also play a role in the degree of repression achieved by TRAP. Also, it was hypothesized that improved levels of repression (compared to the original configuration in WO2015092440) might be achieved by ‘hiding’ the Kozak sequence within the 3′ terminus of the tbs (See FIG. 2B). To be able to achieve this, the terminal 1-2 repeat consensus (KAGNN) of the tbs would necessarily need to partially overlap the Kozak sequence.

Four tbs-Kozak variants were designed (see Table II-Panel I) and cloned into a GFP reporter construct as indicated in FIG. 6A. Variants 0, 1 and 2 were designed such that the extended Kozak sequence (conforming to consensus GNNRVVATG) overlapped with the final two tbs repeat sequences, whereas Variant 3 overlapped just the final tbs repeat sequence. These variants, along with the original reporter in which the Kozak sequence is 3 nucleotides downstream of the final tbs repeat sequence (i.e. no overlap), were individually co-transfected into HEK293T cells+/−a TRAP-expression plasmid, and GFP expression measured two days post-transfection by flow cytometry. A GFP Expression Score (% GFP positive cells×MFI) was generated from flow cytometry data and plotted (FIG. 6B).

Two of these tbs-Kozak variants were tested in conjunction with two different promoters (EFS and huPGK) within scAAV2 vector genome expression cassettes, and compared to tbs-containing cassettes that did not have overlapping tbs/Kozak sequences. These GFP reporter vector genome plasmids were individually co-transfected into HEK293T cells+/−a TRAP-expression plasmid, and GFP expression measured two days post-transfection by flow cytometry. A GFP Expression Score (% GFP positive cells×MFI) was generated from flow cytometry data and plotted (FIG. 9). The non-overlapping tbs/Kozak variants (Original and a second variant containing an HpaI site between the tbs and the Kozak) were capable of 50-to-100 fold repression, whereas the new tbs-Kozak variants (tbs_V0 and tbs_V3) were repressed by at least 10-times this (500-to-3500 fold). These tbs-Kozak variants also performed similarly when the L33 or L12 Improved leaders were employed in either EFS or huPGK promoter cassettes. Importantly, the ‘ON’ levels (no TRAP) for the new variants were similar to the non-overlapping tbs/Kozak variants, indicating that the Kozak sequences within the new variants were effective at directing efficient translation.

The data indicates that all of the tbs-Kozak variants were capable of similar levels of ‘ON’ transgene expression, showing that the Kozak sequences directed efficient levels of translation initiation. Variant 1 was poorly repressed by TRAP when compared to the original reporter configuration (10-fold higher levels of GFP expression in the presence of TRAP). However, surprisingly Variants 0, 2 and 3 were repressed to lower levels compared to the original configuration.

TABLE II
Optimal 3′tbs-Kozak junction sequences

| Panel I | Optimal 3′tbs-Kozak junction |
|---|---|
| Original | [kagnn]acaGCCACCATG |
| Variant ‘0’ | \|kaGCC][GAGAT]G |
| Variant ‘1’ | [kagGC][GAGCA]TG |
| Variant ‘2’ | \|kagnG][GAGCC]ATG |
| Variant ‘3’ | [kagnn][GAGAC]CATG |

[Panel I] Variant Kozak sequences designed to overlap the 3′ end of the upstream tbs sequence. The extended Kozak sequence was placed such that the main transgene ATG initiation codon is placed 9 nucleotides downstream of the 3′ terminal KAGNN repeat of the tbs i.e. there is no overlap. In order to position the main transgene ATG initiation codon closer to the upstream tbs, the four variants were designed such that the consensus KAGNN repeat of the 3′ terminal tbs repeats were maintained whilst also maintaining an efficient extended Kozak consensus sequence (herein defined as GNNRVVATG). The KAGNN repeat sequences are within brackets and the Kozak sequences in bold caps.

### Example 4: Identification of an Improved Spacer Sequence Between IRES and Tbs to Enable Greater Fold-Repression of Transgenes in Bi-Cistronic Cassettes

In the original TRIP system reported in WO2015/092440 it was demonstrated for the first time that the TRAP-tbs paradigm could be applied to the repression of IRES-dependent transgenes by insertion of the tbs between the IRES and the ORF; in this configuration a 26 nucleotide spacer was included between the 3′end of the IRES and the tbs.

In the present work, it was hypothesized that the spacer sequence might be optimized to allow for better levels of repression, and so a number of IRES-spacer-tbs-GFP variants were constructed—see Table III-Panel I and FIG. 7A. All these constructs also contained an identical 5′UTR-luciferase sequence comprising the upstream expression cassette, which was used to monitor transfection efficiency in the experiments. The spacer variants were comprised from truncations of either the original 26 nucleotide spacer or a variant of this spacer, and were truncated to 15 or 10 or 5 nucleotides in length. A final variant was made in which no spacer was included. These variants, along with the original 26 nucleotide spacer containing reporter were individually co-transfected into HEK293T cells+/−a TRAP-expression plasmid, and GFP expression measured two days post-transfection by flow cytometry. A GFP Expression Score (% GFP positive cells×MFI) was generated from flow cytometry data and plotted (FIG. 7B). Luciferase activity was also measured to assess transfection efficiency.

The data shows that spacer truncation variants of the original spacer of 15, 10, and 5 nucleotides in length gave lower repressed levels compared to the original spacer, without impacting on ‘ON’ levels (without TRAP). The same was true of the 10 nucleotide spacer based on the variant spacer. Thus, improved spacer sequences had been identified.

Further validation of one of the best spacers (Original, truncated to 15nt) was carried out in the context of positioning the tbs closer to the downstream ATG codon (moved from 9 to 3 nucleotides) and in the context of 11 or 8 repeats in the tbs sequence. All four of these further variants gave improved repression profiles by TRAP compared to the original spacer-containing configuration (FIG. 7C), demonstrating that other the spacer could be utilized in functional but different tbs contexts.

TABLE III
IRES-spacer-Kozak sequences

| Panel I | IRES-Spacer-Kozak sequences |
|---|---|
| Original [26 nt] | CGTGGTTTTCCTTTGAAAAACACGATG |
| | ATACCATAGCAGAGACGGCTAGCGTTA |
| | ACCT-[KAGNN]×7-11-AACGCCACC |
| | ATG |
| Original | CGTGGTTTTCCTTTGAAAAACACGATG |
| [Trunc-15 nt] | ATACCATAGCAGAGACGGCT- |
| | [KAGNN]×7-11-AACGCCACCATG |
| Original | CGTGGTTTTCCTTTGAAAAACACGATG |
| [Trunc-10 nt] | ATACCATAGCAGAGA- |
| | [KAGNN]×7-11-AACGCCACCATG |
| Original | CGTGGTTTTCCTTTGAAAAACACGATG |
| [Trunc-5 nt] | ATACCATAGC-[KAGNN]×7-11-AAC |
| | GCCACCATG |
| No Spacer [0 nt] | CGTGGTTTTCCTTTGAAAAACACGATG |
| | ATACC-[KAGNN]×7-11-AACGCCAC |
| | CATG |
| Variant [26 nt] | CGTGGTTTTCCTTTGAAAAACACGATG |
| | ATACCATATCAGAGACGGCTAGCGTAT |
| | ACCA-[KAGNN]×7-11-AACGCCACC |
| | ATG |
| Variant | CGTGGTTTTCCTTTGAAAAACACGATG |
| [Trunc-15 nt] | ATACCATATCAGAGACGGCT- |
| | [KAGNN]×7-11-AACGCCACCATG |
| Variant | CGTGGTTTTCCTTTGAAAAACACGATG |
| [Trunc-10 nt] | ATACCAGAGACGGCT- |
| | [KAGNN]×7-11-AACGCCACCATG |
| Variant | CGTGGTTTTCCTTTGAAAAACACGATG |
| [Trunc-5 nt] | ATACCTACCA-[KAGNN]×7-11-AAC |
| | GCCACCATG |
| Original | CGTGGTTTTCCTTTGAAAAACACGATG |
| [Trunc-15 nt]- | ATACCATAGCAGAGACGGCT- |
| 3 ntKozak | [KAGNN]×6-10-[GAGAC]CATG |
| Original | CGTGGTTTTCCTTTGAAAAACACGATG |
| [Trunc-15 nt]- | ATACCATAGCAGAGACGGCT- |
| 9 ntKozak | [KAGNN]×7-11-AACGCCACCATG |

[Panel I] Variant spacers used between an IRES and tbs sequence used in the study. The ‘Original’ spacer sequence outlined in WO2015092440 is of 26 nucleotides in length. A variant of this spacer sequence was made (Variant [26nt]). Truncations of both of these spacers was made, and also a variant with no spacer. The Original-[Trunc-15nt] spacer was found to give the best level of repression whilst maintaining good ‘ON’ levels (i.e. in absence of TRAP). The Original-[Trunc-15nt] spacer was then used in combination with either 11×KAGNN repeat or 8×tbsKAGNN repeat tbs sequences, and also with variants in which the distance from the 3′end of the tbs to the downstream transgene ATG initiation codon was reduced from 9nt to 3nt. Italicized sequence indicates the 3′end of the EMCV IRES element, the variant spacer sequences are in bold, the tbs consensus in indicated in normal text, and the downstream transgene extended Kozak sequence is underlined.

### Example 5: Incorporating Improved Overlapping Tbs-Kozak Variants into the Full Length, Intron-Containing EF1a Promoter

The previous Examples indicated that the overlapping tbs-Kozak variants improved repression, compared to non-overlapping tbs/Kozak variants, and this was demonstrated in the context of the EFS promoter (the EF1a promoter truncated by removing its embedded intron). To assess if the tbs-Kozak variants ‘performed’ similarly within the full length EF1a promoter (i.e. post splicing-out of the intron), three tbs-Kozak variants (0, 2 and 3) were cloned into a GFP reporter cassette containing the EF1a promoter (FIG. 10A). After splicing, the 5′UTR contains exon1 (i.e. the L33 Improved leader), a short 12nt sequence comprising the first nucleotides of exon2, and then a tbs-Kozak variant sequence (SEQ ID NO: 60). These GFP reporter plasmids, along with a reporter containing no tbs, were individually co-transfected into suspension, serum-free HEK293T cells+/−a TRAP-expression plasmid, and GFP expression measured two days post-transfection by flow cytometry. A GFP Expression Score (% GFP positive cells×MFI) was generated from flow cytometry data and plotted (FIG. 10B). Further, these tbs-containing GFP expression cassettes were cloned into an HIV-1 based lentiviral vector genome plasmid (in which the MSD and crSD were inactivated, see below), and a similar experiment carried out in suspension, serum-free HEK293T cells, resulting in GFP Expression scores (FIG. 100). The data show that indeed the overlapping tbs-Kozak variants were capable of improved transgene repression compared to the non-overlapping tbs/Kozak variant employed.

### Example 6—MSD-2KO Lentiviral Vectors Produce Less Transgene Protein During Production Due to Ablation of Aberrant Splicing

A further advantage of ablating aberrant splicing during lentiviral vector production is to reduce the amount of transgene-encoding mRNA that leads to transgene protein production.

During the course of this work, we unexpectedly found that transgene-encoding mRNAs were effectively produced from the ‘external’ (CMV) promoter driving the vector genome cassette due to splicing-out from the splicing region of the SL2 (part of the packaging signal) to internal splice acceptor sites (FIG. 11A). The degree to which this occurs depends on the internal sequences between the cppt and the transgene ORF (i.e. the promoter-5′UTR sequence). The use of the EF1a promoter (containing a very strong splice acceptor) in the transgene cassette, results in aberrant splicing from the MSD in over 95% of total transcripts originating from the external promoter (FIG. 12). By comparing total GFP expression in standard or MSD-2KO lentiviral vector production cultures (FIG. 11B), we show that up to 80% of the transgene protein expressed during production originates from the aberrant splice product. We found that combining the MSD-2KO genotype with the TRiP system augmented the reduction in transgene protein produced.

FIG. 11C displays the genetic modification to the SL2 loop of the ‘MSD2KO’ variant of MSD-2KO lentiviral vector genome packaging region, which mutates both the MSD and the cryptic splice donor positioned downstream (the MSD2KO variant has been utilized to generate the data shown in FIG. 11B We also made three other splice donor region mutants: [1] ‘MSD2KOv2’, which also introduced two specific changes within the MSD and cryptic donor sequences, [2] ‘MSD2KOm5’, which replaces the entire SL2 loop with an artificial stem loop and [3] a complete SL2 deletion, thus removing the entire splice donor region (also termed the splicing region). These were independently shown to ablate aberrant splicing activity.

### Example 7: Occlusion of Progressively More of the Core Kozak Sequence by the 3′ Terminal KAGNN Repeat of the Tbs Results in Progressively Greater Transgene Repression by TRAP

In Example 3, a limited number of overlapping tbs-Kozak variants were generated and tested in the context of non-intron-containing promoters EFS and huPGK. These variants were also then tested in the full EF1a promoter, which contains an intron in Example 5, showing that this difficult-to-repress promoter could be repressed by employing overlapping tbs-Kozak variants.

To further exemplify the principle of improved TRAP repression by ‘hiding’ the core Kozak sequence within the 3′ terminal KAGNN repeat of the tbs, a panel of new variants were designed (see Table IV). These were based on all the possible variants encoded by three ‘overlap groups’; KAGatg (where the KAGNN consensus overlaps the ATG of the core Kozak as much as possible, i.e. overlaps by the first two nucleotides of the ATG of the core Kozak), KAGNatg (where the KAGNN consensus overlaps the first nucleotide of the ATG of the core Kozak) and KAGNNatg (where the KAGNN consensus does not overlap the ATG of the core Kozak). (The initial variants tbs-kzkV0, tbs-kzkV1 and tbs-kzkV2 in Examples 3 and 5 fall within these defined groups). Any variants within these groups that generated a ‘GT’ dinucleotide were not generated/evaluated because of the undesirable possibility that these may produce cryptic splice donor sites.

TABLE IV
Overlapping tbs-Kozak variants
generated for further exemplification

| Overlap | tbs-Kozak | Overlapping tbs |
|---|---|---|
| group | variant ID | 3′KAGNN-Kozak variants |
| N/A | Non-overlapping | [KAGNN]×10-[KAGNN] |
| | tbs-Kozak | ACAGCCACCATG |
| KAGatg | tbskzkV0.G | [KAGNN]×10-[ [symbol] ]G |
| | tbskzkV0.T | [KAGNN]×10-[ [symbol] ]G |
| KAGNatg | tbskzkV1.0 | [KAGNN]×10-[G [symbol] ]TG |
| | tbskzkV1.1 | [KAGNN]×10-[G [symbol] ]TG |
| | tbskzkV1.2 | [KAGNN]×10-[G [symbol] ]TG |
| | tbskzkV1.3 | [KAGNN]×10-[T [symbol] ]TG |
| | tbskzkV1.4 | [KAGNN]×10-[T [symbol] ]TG |
| | tbskzkV1.5 | [KAGNN]×10-[T [symbol] ]TG |
| KAGNNatg | tbskzkV2.0 | [KAGNN]×10-[GA [symbol] ]ATG |
| | tbskzkV2.1 | [KAGNN]×10-[GA [symbol] ]ATG |
| | tbskzkV2.2 | [KAGNN]×10-[GA [symbol] ]ATG |
| | tbskzkV2.3 | [KAGNN]×10-[GA [symbol] ]ATG |
| | tbskzkV2.4 | [KAGNN]×10-[GA [symbol] ]ATG |
| | tbskzkV2.5 | [KAGNN]×10-[GA [symbol] ]ATG |
| | tbskzkV2.6 | [KAGNN]×10-[GA [symbol] ]ATG |
| | tbskzkV2.7 | [KAGNN]×10-[GA [symbol] ]ATG |
| | tbskzkV2.8 | [KAGNN]×10-[GA [symbol] ]ATG |
| | tbskzkV2.9 | [KAGNN]×10-[GA [symbol] ]ATG |
| | tbskzkV2.10 | [KAGNN]×10-[GA [symbol] ]ATG |
| | tbskzkV2.11 | [KAGNN]×10-[TA [symbol] ]ATG |
| | tbskzkV2.12 | [KAGNN]×10-[TA [symbol] ]ATG |
| | tbskzkV2.13 | [KAGNN]×10-[TA [symbol] ]ATG |
| | tbskzkV2.14 | [KAGNN]×10-[TA [symbol] ]ATG |
| | tbskzkV2.15 | [KAGNN]×10-[TA [symbol] ]ATG |
| | tbskzkV2.16 | [KAGNN]×10-[TA [symbol] ]ATG |
| | tbskzkV2.17 | [KAGNN]×10-[TA [symbol] ]ATG |
| | tbskzkV2.18 | [KAGNN]×10-[TA [symbol] ]ATG |
| | tbskzkV2.19 | [KAGNN]×10-[TA [symbol] ]ATG |
| | tbskzkV2.20 | [KAGNN]×10-[TA [symbol] ]ATG |
| | tbskzkV2.21 | [KAGNN]×10-[TA [symbol] ]ATG |

All the possible variants representing ‘overlap groups’ pertaining to the consensus of KAGatg or KAGNatg or KAGNNatg were generated, except for those resulting in a ‘GT’ dinucleotide which might generate an unwanted (cryptic) splice donor site. The first 10 KAGNN repeats are presented as a consensus here for clarity but were principally the first 48 nucleotides of SEQ ID NO: 8. The 3′ terminal tbs KAGNN is presented (italicised and bracketed) as encoded in each variant; the core Kozak consensus is in bold and any nucleotides presented as being part of the broader, extended Kozak consensus are underlined.

Generally, most of the variants conformed to the preferred core Kozak consensus of RVVATG, whilst simultaneously being restricted to containing the denoted KAGNN tbs consensus. These variants were cloned into a pEF1α-GFP reporter plasmid, and therefore the 5′UTR contained (after splicing of its intron) the L33 leader (exon 1) plus a short 12nt sequence from exon 2, which was previously shown in Example 5 to be less repressible by TRAP unless an overlapping tbs-Kozak variant was used. Suspension (serum-free) HEK293T cells were transfected with these variants individually with or without a TRAP-expression plasmid under conditions that typically reflected lentiviral vector (LV) transfection/production (e.g. inclusion of sodium butyrate induction), and flow cytometry performed at typical LV harvest times (2 days post-transfection). Global GFP Expression scores were generated (% GFP×MFI; ArbU) for +/−TRAP conditions and then fold-repression values generated and plotted in FIG. 13A. The results demonstrate that the more the core Kozak consensus sequence overlaps the 3′ terminal KAGNN tbs repeat, the better the level of TRAP repression. Statistical analysis (T-Test) of the fold differences of each overlap group demonstrate a significant increase in repression as more of the core Kozak was overlapped with the 3′ terminal KAGNN tbs repeat, with the best repression scores coming from the KAGatg group where ⅔^{rd} of the initiation codon forms part of the KAGNN repeat. All overlap variants produced statistically greater transgene repression by TRAP compared to the non-overlapping tbs variant.

The data were further stratified in FIG. 13B, where non-repressed ‘ON’ transgene levels were displayed from highest to lowest. The two variants from the KAGatg overlap group are highlighted to show that for these two best performing variants in terms of TRAP repression, the GAGatg variant (tbskzkV0.G) gave the highest ‘ON’ levels, presumably because it conforms to the core Kozak consensus of RVVATG, whereas TAGatg (tbskzkV0.T) does not.

All these data support the general principle that overlapping tbs-Kozak variants are more effective in mediating TRAP repression compared to non-overlapping tbs variants, and that preferably the tbs-Kozak overlap conforms to the core Kozak consensus of RVVATG to ensure good ‘ON’ transgene expression in vector-transduced target cells. Thus, the employment of these novel tbs-Kozak variants will enable more efficient transgene repression during viral vector production, potentially leading to increase in viral vector titres if said transgene protein activity is detrimental to viral vector titres and/or activity.

### Example 8: Further Use of an Optimal Overlapping tbs-Kozak Variant to Improve TRAP-Mediated Repression of Common Promoters Harbouring an Intron

In Example 5, the use of overlapping tbs-Kozak variants was shown to improve TRAP-mediated repression when using the full length EF1a promoter, which contains an intron. For similar promoters used widely in viral vector genomes for gene therapy—for example the CAG promoter—the presence of embedded exon/intron sequence means that the degree of TRAP-mediated repression may be affected by sequences within ‘native’ exonic sequences. From the point of view of improving TRAP-mediated repression, it may not be obvious or feasible to alter exonic sequences, especially if these are involved in splicing enhancement (e.g. a splice enhancer element close to the splice donor site). The widely used CAG promoter comprises the CMV enhancer element, the core chicken β-Actin gene promoter-exon1-intron sequence, and the splice acceptor-exonic sequence from the rabbit β-Globin gene. Elsewhere in the invention it was surprisingly found that exon 1 from the EF1a promoter (L33) could be used upstream of all types of tbs variants to improve TRAP-mediated repression, presumably providing good sequence context to support formation a stable TRAP-tbs complex, to enable efficient translation inhibition. The overlapping tbs-Kozak variants were shown to aid TRAP-mediated repression in both EF1a (intron-containing) and several other promoters lacking introns.

In this example (see FIG. 14A), both features were applied to improving TRAP-mediated repression from the CAG promoter by [a] placing the tbskzkV0.G variant (also referred to as Variant ‘0’ in other Examples) within the ‘native’ 5′UTR region of the CAG promoter (SEQ ID NO: 117) and [b], swapping the entire ‘native’ intron-containing 5′UTR region with the EF1a 5′UTR-intron region harbouring the tbskzkV0.G variant (SEQ ID NO: 118). The corresponding spliced sequences are shown as SEQ ID NO: 119 and SEQ ID NO: 120, respectively. In addition, it was shown by exemplification that a promoter typically used without an intron in viral vector genomes—in this case CMV—could be appended with an artificial 5′UTR containing a heterologous intron, expression of which had previously shown to be efficiently repressed by TRAP. Specifically, the 5′UTR with the EF1a 5′UTR-intron region harbouring the tbskzkV0.G variant (SEQ ID NO: 118) was used in the CMV promoter context.

These reporter constructs (encoding GFP) were evaluated for both ‘ON’ expression levels and TRAP-mediated repression in suspension (serum-free) HEK293T cells, modelling a viral vector production scenario. Cells were transfected with GFP reporter plasmid+/−pTRAP, cultures induced with sodium butyrate after transfection (as per typical viral vector production) and cells analysed for GFP expression ˜2 days post-transfection (i.e. at typical viral vector harvest point). GFP Expression scores (% GFP positive×MFI; ArbU) were generated and plotted (FIG. 14B), and TRAP-repression scores displayed. These data indicate that TRAP-mediated expression in typical viral vector production cells can be improved from the CAG promoter using the tbskzkV0.G variant, from 3-fold to 30-fold. Moreover, the data show that the ‘native’ 5′UTR region sequence from different promoters can be replaced with the intron-containing EF1a 5′UTR harbouring the tbsKzkV0.G variant, leading to both substantially improved TRAP-mediated repression (30-40 fold to >100-fold) and maintenance of high gene expression in the absence of TRAP (i.e. modelling expression in viral vector-transduced target cells). Thus, the novel EF1a-5′UTR-intron-tbskzkV0.G sequence may be useful in providing heterologous promoters the known benefits imparted by an intron in target cells (i.e. increased gene expression), whilst also enabling efficient repression of the transgene protein during viral vector production, potentially leading to increase in viral vector titres if said transgene protein activity is detrimental to viral vector titres. This also applies to the use of the EF1α-5′UTR-intron sequence with other overlapping tbs-Kozak sequences.

All publications mentioned in the above specification are herein incorporated by reference. Various modifications and variations of the described aspects of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A nucleic acid sequence comprising a nucleotide of interest and a tryptophan RNA-binding attenuation protein (TRAP) binding site; wherein
(i) said TRAP binding site overlaps the start codon ATG of said nucleotide of interest; and/or
(ii) said nucleic acid sequence also comprises a Kozak sequence, wherein said TRAP binding site overlaps the Kozak sequence.

2. A nucleic acid sequence comprising a nucleotide of interest and a TRAP binding site; wherein
(i) the TRAP binding site comprises a portion of the start codon ATG of said nucleotide of interest or wherein the ATG start codon comprises a portion of the TRAP binding site; and/or
(ii) said nucleic acid sequence also comprises a Kozak sequence, wherein said Kozak sequence comprises a portion of the TRAP binding site.

3. The nucleic acid sequence of claim 1 or 2, wherein the nucleotide of interest is operably linked to the TRAP binding site or the portion thereof.

4. The nucleic acid sequence of any preceding claim, wherein the TRAP binding site or the portion thereof is capable of interacting with tryptophan RNA-binding attenuation protein such that translation of the nucleotide of interest is repressed in a viral vector production cell.

5. The nucleic acid sequence of any preceding claim, wherein the nucleotide of interest is translated in a target cell which lacks the tryptophan RNA-binding attenuation protein.

6. The nucleic acid sequence of any preceding claim, wherein the TRAP binding site or the portion thereof comprises multiple repeats of the sequence KAGN2-3.

7. The nucleic acid sequence of any preceding claim, wherein the TRAP binding site or the portion thereof comprises multiple repeats of the sequence KAGN2.

8. The nucleic acid sequence of any preceding claim, wherein the TRAP binding site or the portion thereof comprises at least 6 repeats of the sequence KAGN2.

9. The nucleic acid sequence of any preceding claim, wherein the TRAP binding site or the portion thereof comprises at least 8 repeats of the sequence KAGN2-3.

10. The nucleic acid sequence of claim 9, wherein the number of KAGNNN repeats is 1 or less.

11. The nucleic acid sequence of any preceding claim, wherein the TRAP binding site or the portion thereof comprises at least 8-11 repeats of the sequence KAGN2.

12. The nucleic acid sequence of any preceding claim, wherein the TRAP binding site or the portion thereof comprises 11 repeats of the sequence KAGN2-3, wherein the number of KAGNNN repeats is 3 or less.

13. The nucleic acid sequence of any preceding claim wherein the Kozak sequence and/or start codon overlaps the 3′ terminal of the TRAP binding site or of the portion thereof.

14. The nucleic acid sequence of claim 13 wherein the Kozak sequence and/or start codon overlaps the 3′ terminal KAGNN repeat of the TRAP binding site or of the portion thereof.

15. The nucleic acid sequence of any preceding claim, wherein said Kozak sequence comprises the sequence RNNATG (SEQ ID NO:125) or RVVATG (SEQ ID NO:28).

16. The nucleic acid sequence of any preceding claim, wherein said overlapping Kozak sequence and/or start codon and TRAP binding site or portion thereof comprises any one of SEQ ID NOs: 29-33.

17. The nucleic acid sequence of any preceding claim wherein said nucleic acid sequence comprises any one of SEQ ID NOs: 34-37, 69-92 or 108-112, preferably SEQ ID: 114).

18. The nucleic acid sequence of claim 17, wherein said nucleic acid sequence comprises one of SEQ ID NO: 34 or SEQ ID NO: 35.

19. The nucleic acid sequence of any preceding claim, wherein the distance between the transcription start site/end of promoter to start of the TRAP binding site or of the portion thereof is 1 to 33 nucleotides in length.

20. The nucleic acid sequence of any preceding claim, wherein the distance between the transcription start site/end of promoter to start of the TRAP binding site or of the portion thereof is 1 to 12 nucleotides in length.

21. The nucleic acid sequence of any preceding claim, wherein the TRAP binding site or the portion thereof lacks a type II restriction enzyme site, preferably a SapI restriction enzyme site.

22. The nucleic acid sequence of any preceding claim, wherein said nucleic acid sequence comprises a 5′ leader sequence upstream of the TRAP binding site or the portion thereof.

23. The nucleic acid sequence of claim 22, wherein said leader sequence comprises a sequence derived from the non-coding EF1α exon 1 region.

24. The nucleic acid sequence of claim 23, wherein said leader sequence comprises a sequence as defined in SEQ ID NO:25 or SEQ ID NO:26.

25. The nucleic acid sequence of any preceding claim, wherein said sequence comprises an internal ribosome entry site (IRES).

26. The nucleic acid sequence of claim 25, wherein said sequence comprises a spacer sequence between an internal ribosome entry site (IRES) and the TRAP binding site or the portion thereof.

27. The nucleic acid sequence of claim 26, wherein said spacer is between 0 and 30 nucleotides in length.

28. The nucleic acid sequence of claim 27, wherein said spacer is 15 nucleotides in length.

29. The nucleic acid sequence of any one of claims 26 to 28, wherein the spacer is 3 or 9 nucleotides from the 3′ end of the TRAP binding site or the portion thereof and the downstream initiation codon of the nucleotide of interest.

30. The nucleic acid sequence of any one of claims 26 to 29, wherein the spacer comprises a sequence as defined in any one of SEQ ID NOs:38-44, preferably the spacer comprises a sequence as defined in SEQ ID NO:39.

31. The nucleic acid sequence of any preceding claim, wherein the nucleotide of interest gives rise to a therapeutic effect.

32. The nucleic acid sequence of any preceding claim, wherein the nucleic acid sequence further comprises an RRE sequence or functional substitute thereof.

33. The nucleic acid sequence of any preceding claim, wherein said nucleic acid sequence is a vector transgene expression cassette.

34. The nucleic acid sequence of any preceding claim, wherein the 3′ terminal KAGNN repeat of the TRAP binding site or of the portion thereof overlaps at least the first nucleotide of the start codon ATG.

35. The nucleic acid sequence of claim 34, wherein the 3′ terminal KAGNN repeat of the TRAP binding site or of the portion thereof overlaps the first two nucleotides of the start codon ATG

36. The nucleic acid sequence of claim 34, wherein the 3′ terminal KAGNN repeat of the TRAP binding site or of the portion thereof overlaps the first nucleotide of the start codon ATG within a core Kozak sequence

37. The nucleic acid sequence of any one of claims 34 to 36, wherein the nucleic acid sequence comprises a sequence as defined in SEQ ID NO: 114 or SEQ ID NO: 116.

38. A viral vector comprising the nucleic acid sequence of any one of claims 1 to 37 or 67 to 92.

39. The viral vector of claim 38, wherein the viral vector comprises more than one nucleotide of interest and wherein at least one nucleotide of interest is operably linked to a TRAP binding site or a portion thereof as defined in any of claims 1 to 12.

40. The viral vector of claim 38 or claim 39, wherein the viral vector is derived from a retrovirus, adenovirus, adeno-associated virus, herpes simplex virus, vaccinia virus or baculovirus.

41. The viral vector of claim 40, wherein the viral vector is derived from a lentivirus, preferably wherein the viral vector is derived from HIV-1, HIV-2, SIV, FIV, BIV, EIAV, CAEV or visna lentivirus.

42. A viral vector production system comprising a set of nucleic acid sequences encoding the components required for production of the viral vector, wherein the RNA genome of the viral vector comprises the nucleic acid sequence of any of claims 1 to 37 or 67 to 92.

43. The viral vector production system of claim 42, wherein the viral vector is derived from a retrovirus, adenovirus or adeno-associated virus, preferably wherein the viral vector is a retroviral vector and the viral vector production system comprises nucleic acid sequences encoding Gag and Pol proteins, the tryptophan RNA-binding attenuation protein, and Env protein, or functional substitutes thereof.

44. The viral vector production system of claim 43, wherein the viral vector production system further comprises a nucleic acid sequence encoding rev or a functional substitute thereof.

45. The viral vector production system of any of claims 42 to 44, wherein the viral vector is derived from a lentivirus, preferably wherein the viral vector is derived from HIV-1, HIV-2, SIV, FIV, BIV, EIAV, CAEV or visna lentivirus.

46. A DNA construct for use in the viral vector production system of any of claims 42 to 45 comprising the nucleic acid sequence of any of claims 1 to 37 or 67 to 92.

47. A DNA construct for use in the viral vector production system of any of claims 42 to 45 comprising a nucleic acid sequence encoding the tryptophan-RNA binding attenuation protein.

48. A set of DNA constructs for use in the viral vector production system of any of claims 42 to 45 comprising the DNA construct of claim 46 or claim 47, a DNA construct encoding Gag and Pol proteins, and a DNA construct encoding Env protein, or functional substitutes thereof, preferably wherein the set of DNA constructs further comprises a DNA construct encoding a rev sequence or a functional substitute thereof.

49. A viral vector production cell comprising the nucleic acid sequence of any of claims 1 to 37 or 67 to 92, the viral vector production system of any of claims 42 to 45 or the DNA constructs of any of claims 446 to 48.

50. The viral vector production cell of claim 49, wherein the cell is transiently transfected with a vector encoding a tryptophan-RNA binding attenuation protein.

51. The viral vector production cell of claim 49, wherein the cell stably expresses a tryptophan-RNA binding attenuation protein.

52. A process for producing viral vectors comprising introducing the nucleic acid sequence of any of claims 1 to 37 or 67 to 92, the viral vector production system of any of claims 42 to 45 or the DNA constructs of any of claims 46 to 48 into a viral vector production cell and culturing the production cell under conditions suitable for the production of the viral vectors.

53. A viral vector produced by the viral vector production system of any of claims 42 to 45, using the viral vector production cell of any of claims 49 to 51 or by the process of claim 52.

54. The viral vector of claim 53 which comprises the nucleic acid sequence of any of claims 1 to 37 or 67 to 92.

55. The viral vector of claim 53 or claim 54 which is derived from a retrovirus, adenovirus or adeno-associated virus.

56. The viral vector of claim 55 which is derived from a lentivirus.

57. The viral vector of claim 56 which is derived from HIV-1, HIV-2, SIV, FIV, BIV, EIAV, CAEV or visna lentivirus.

58. A cell transduced by the viral vector of any one of claims 38 to 41 or 53 to 57.

59. The viral vector of any of claims 38 to 41 or 53 to 57 or the cell of claim 58 for use in medicine.

60. Use of the viral vector of any of claims 38 to 41 or 53 to 57 or the cell of claim 58 for the preparation of a medicament to deliver a nucleotide of interest to a target site in need of the same.

61. A method of treatment comprising administering the viral vector of any of claims 38 to 41 or 53 to 57 or the cell of claim 58 to a subject in need of the same.

62. A pharmaceutical composition comprising the viral vector of any of claims 38 to 41 or 53 to 57 or the cell of claim 58 in combination with a pharmaceutically acceptable carrier, diluent or excipient.

63. A method of identifying nucleic acid binding sites and/or nucleic acid binding proteins which are capable of interacting such that the translation of a nucleotide of interest is repressed in a viral vector production cell when operably linked to the nucleic acid binding site, wherein the method comprises analysing the expression of a reporter gene in a cell comprising both the nucleic acid binding site operably linked to the reporter gene and the nucleic acid binding protein.

64. The method of claim 63, wherein the reporter gene encodes a fluorescent protein.

65. A method of repressing translation of a nucleotide of interest (NOI) in a viral vector production cell, the method comprising introducing into the viral vector production cell the nucleic acid sequence as defined any one of claims 1 to 37 or 67 to 92, and a nucleic acid sequence encoding a tryptophan-RNA binding attenuation protein (TRAP), wherein the TRAP binds to the TRAP binding site, or the portion thereof, thereby repressing translation of the NOI.

66. A method of increasing viral vector titers in a eukaryotic vector production cell, the method comprising introducing into the eukaryotic vector production cell the viral vector production system of any one of claims 42 to 45 and a nucleic acid sequence encoding a tryptophan-RNA binding attenuation protein (TRAP), wherein the TRAP binds to the TRAP binding site, or the portion thereof, and represses translation of the NOI, thereby increasing viral vector titres relative to a viral vector having no TRAP binding site.

67. A nucleic acid sequence comprising a nucleotide of interest, a binding site for tryptophan RNA-binding attenuation protein (TRAP), a multiple cloning site and a Kozak sequence, wherein said multiple cloning site is overlapping with or located downstream to the 3′ KAGN₂₋₃ repeat of the TRAP binding site and upstream of the Kozak sequence.

68. The nucleic acid sequence of claim 67, wherein said nucleic acid sequence comprises any one of SEQ ID NOs: 45-58.

69. The nucleic acid sequence of claim 68, wherein said nucleic acid sequence comprises any one of SEQ ID NOs: 52-58.

70. The nucleic acid sequence of claim 69, wherein said nucleic acid sequence comprises any one of SEQ ID NO: 52, SEQ ID NO:55 or SEQ ID NO: 58.

71. The nucleic acid sequence of any one of claim 1-37 or 67-70, wherein the nucleic acid sequence further comprises a promoter-5′ UTR region.

72. The nucleic acid sequence of claim 71, wherein the TRAP binding site or portion thereof and Kozak sequence or the TRAP binding site, multiple cloning site and Kozak sequence is located within the 5′ UTR of the promoter-5′ UTR region.

73. The nucleic acid sequence of claim 71 or claim 72, wherein the promoter-5′ UTR region further comprises an intron, preferably wherein the intron is upstream of the TRAP binding site or portion thereof.

74. The nucleic acid sequence of any one of claims 71 to 73, wherein the promoter-5′ UTR region is an engineered promoter comprising a heterologous intron within the 5′ UTR.

75. A nucleic acid sequence encoding the RNA genome of a viral vector, wherein the RNA genome of the viral vector comprises a nucleic acid sequence according to any one of claims 1 to 37 or 67 to 74.

76. The nucleic acid sequence of any of claims 1 to 37 or 67 to 74, wherein said nucleic acid sequence is comprised within an RNA genome of a viral vector.

77. The nucleic acid sequence of any one of claims 1 to 37 or 67 to 74, wherein said nucleic acid sequence is operably linked to a nucleotide sequence encoding the RNA genome of a viral vector.

78. The nucleic acid sequence of any one of claims 75 to 77 or the viral vector production system of any one of claims 43 to 45, wherein the major splice donor site in the RNA genome of the viral vector is inactivated.

79. The nucleic acid sequence or viral vector production system of claim 78, wherein the major splice donor site and the cryptic splice donor site 3′ to the major splice donor site in the RNA genome of the viral vector are inactivated, preferably wherein the cryptic splice donor site is the first cryptic splice donor site 3′ to the major splice donor site.

80. The nucleic acid sequence or viral vector production system of claim 75, wherein said cryptic splice donor site is within 6 nucleotides of the major splice donor site.

81. The nucleic acid sequence or viral vector production system of any one of claims 78 to 80, wherein the major splice donor site and cryptic splice donor site are mutated or deleted.

82. The nucleic acid sequence or viral vector production system of any one of claims 78 to 81, wherein the nucleotide sequence encoding the RNA genome of the viral vector prior to inactivation of the splice sites comprises a sequence as set forth in any of SEQ ID NOs: 94, 96, 97, 102, 103 and/or 106.

83. The nucleic acid sequence or viral vector production system of any one of claims 78 to 82, wherein the nucleotide sequence encoding the RNA genome of the viral vector comprises a sequence with a mutation or deletion relative to the sequence as set forth in any of SEQ ID NOs: 94, 96, 97, 102, 103 and/or 106.

84. The nucleic acid sequence or viral vector production system of any one of claims 78 to 83, wherein the nucleotide sequence encoding the RNA genome of the viral vector comprises an inactivated major splice donor site which would otherwise have a cleavage site between nucleotides corresponding to nucleotides 13 and 14 of SEQ ID NO:94.

85. The nucleic acid sequence or viral vector production system of any one of claims 78 to 84, wherein the nucleotide sequence of the major splice donor site prior to inactivation comprises the sequence as set forth in SEQ ID NO: 97.

86. The nucleic acid sequence or viral vector production system of any one of claims 79 to 85, wherein the nucleotide sequence of the cryptic splice donor site prior to inactivation comprises the sequence as set forth in SEQ ID NO: 103.

87. The nucleic acid sequence or viral vector production system of any one of claims 79 to 86, wherein the nucleotide sequence encoding the RNA genome of the viral vector comprises an inactivated cryptic splice donor site which would otherwise have a cleavage site between nucleotides corresponding to nucleotides 17 and 18 of SEQ ID NO:94.

88. The nucleic acid sequence or viral vector production system of any one of claims 78 to 87, wherein the nucleotide sequence encoding the RNA genome of the viral vector comprises a sequence as set forth in any of SEQ ID NOs: 95, 98, 99, 100, 101, 104, 105 and/or 107.

89. The nucleic acid sequence or viral vector production system of any one of claims 78 to 88, wherein the nucleotide sequence encoding the RNA genome of the viral vector does not comprise a sequence as set forth in SEQ ID NO:102.

90. The nucleic acid sequence or viral vector production system of any one of claims 78 to 89, wherein the splicing activity from the major splice donor site and cryptic splice donor site of the RNA genome of the viral vector is suppressed or ablated.

91. The nucleic acid sequence or viral vector production system of any one of claims 78 to 90, wherein the splicing activity from the major splice donor site and cryptic splice donor site of the RNA genome of the viral vector is suppressed or ablated in transfected cells or in transduced cells.

92. The nucleic acid sequence or viral vector production system of any one of claims 67 to 91, wherein the viral vector is derived from a lentivirus.


SEQUENCE LISTING (198 sequences)

SEQ ID NO: 1 - protein, 75 aa, Bacillus subtilis
MNQKHSSDFVVIKAVEDGVNVIGLTRGTDTKFHHSEKLDKGEVIIAQFTEHTSAIKVRGEALIQTAYGEMKSEKK

SEQ ID NO: 2 - protein, 86 aa, Aminomonas paucivorans
MKEGEEAKTSVLSDYVVVKALENGVTVIGLTRGQETKFAHTEKLDDGEVWIAQFTEHTSAIKVRGASEIHTKHGMLFSGRGRNEKG

SEQ ID NO: 3 - protein, 81 aa, Desulfotomaculum hydrothermale
MNPMTDRSDITGDYVVVKALENGVTIIGLTRGGVTKFHHTEKLDKGEIMIAQFTEHTSAIKIRGRAELLTKHGKIRTEVDS

SEQ ID NO: 4 - protein, 74 aa, Bacillus stearothermophilus
MYTNSDFVVIKALEDGVNVIGLTRGADTRFHHSEKLDKGEVLIAQFTEHTSAIKVRGKAYIQTRHGVIESEGKK

SEQ ID NO: 5 - protein, 74 aa, Bacillus stearothermophilus
MYTNSDFVVIKALEDGVNVIGLTRGADTRFHHSEKLDKGEVLIAQFTEHTSAIKVRGKAYIQTRHGVIENEGKK

SEQ ID NO: 6 - protein, 76 aa, Bacillus halodurans
MNVGDNSNFFVIKAKENGVNVFGMTRGTDTRFHHSEKLDKGEVMIAQFTEHTSAVKIRGKAIIQTSYGTLDTEKDE

SEQ ID NO: 7 - protein, 80 aa, Carboxydothermus hydrogenoformans
MVCDNFAFSSAINAEYIVVKALENGVTIMGLTRGKDTKFHHTEKLDKGEVMVAQFTEHTSAIKIRGKAEIYTKHGVIKNE

SEQ ID NO: 8 - RNA, 55 nt, Artificial Sequence
  TRAP binding site variant
gaguuuagcggaguggagaagagcggagccgagccuagcagagacgaguggagcu

SEQ ID NO: 9 - RNA, 55 nt, Artificial Sequence
  TRAP binding site variant
gaguuuagcggaguggagaagagcggagccgagccuagcagagacgagaagagcu

SEQ ID NO: 10 - RNA, 30 nt, Artificial Sequence
  TRAP binding site variant
uaguuuaguuuaguuuaguuuaguuuaguu

SEQ ID NO: 11 - RNA, 30 nt, Artificial Sequence
  TRAP binding site variant
uaguuuaguugaguuuaguugaguuuaguu

SEQ ID NO: 12 - RNA, 32 nt, Artificial Sequence
  TRAP binding site variant
gaguuugaguugaguugaguuugaguugaguu

SEQ ID NO: 13 - RNA, 32 nt, Artificial Sequence
  TRAP binding site variant
uaguuugaguuuaguugaguuuuaguugaguu

SEQ ID NO: 14 - DNA, 55 bp, Artificial Sequence
  TRAP binding site variant
gagtttagcggagtggagaagagcggagccgagcctagcagagacgagtggagct

SEQ ID NO: 15 - DNA, 50 bp, Artificial Sequence
  TRAP binding site variant
gagtttagcggagtggagaagagcggagccgagcctagcagagacgagaa

SEQ ID NO: 16 - DNA, 45 bp, Artificial Sequence
  TRAP binding site variant
gagtttagcggagtggagaagagcggagccgagcctagcagagac

SEQ ID NO: 17 - DNA, 40 bp, Artificial Sequence
  TRAP binding site variant
gagtttagcggagtggagaagagcggagccgagcctagca

SEQ ID NO: 18 - DNA, 35 bp, Artificial Sequence
  TRAP binding site variant
gagtttagcggagtggagaagagcggagccgagcc

SEQ ID NO: 19 - DNA, 30 bp, Artificial Sequence
  TRAP binding site variant
gagtttagcggagtggagaagagcggagcc

SEQ ID NO: 20 - DNA, 58 bp, Artificial Sequence
  TRAP binding site variant
gagtttagcggagtggagaaagagacggagccgagacctagcagagacgagaagagct

SEQ ID NO: 21 - DNA, 56 bp, Artificial Sequence
  TRAP binding site variant
gagtttagcggagtggagaagagacggagccgagcctagcagagacgagaagagct

SEQ ID NO: 22 - DNA, 55 bp, Artificial Sequence
  TRAP binding site variant
gagtttagcggagtggagaagagcggagccgagcctagcagagacgagaagagct

SEQ ID NO: 23 - DNA, 41 bp, Artificial Sequence
  TRAP binding site variant
gagtttagcggagtggagaaagagcggagccgagcctagca

SEQ ID NO: 24 - DNA, 36 bp, Artificial Sequence
  TRAP binding site variant
gagtttagcggagtggagaaagagcggagccgagcc

SEQ ID NO: 25 - DNA, 33 bp, Artificial Sequence
  L33 Improved leader sequence
ctttttcgcaacgggtttgccgccagaacacag

SEQ ID NO: 26 - DNA, 12 bp, Artificial Sequence
  L12 Improved leader sequence
ctttttcgcaac

SEQ ID NO: 27 - DNA, 10 bp, Artificial Sequence
  extended Kozak consensus sequence
  misc_feature (2)..(3): n is a, c, g, or t
gnnrvvatgg

SEQ ID NO: 28 - DNA, 6 bp, Artificial Sequence
  core Kozak consensus sequence
rvvatg

SEQ ID NO: 29 - DNA, 6 bp, Artificial Sequence
  3′ tbs-Kozak junction variant
gagatg

SEQ ID NO: 30 - DNA, 7 bp, Artificial Sequence
  3′ tbs-Kozak junction variant
kagvatg

SEQ ID NO: 31 - DNA, 8 bp, Artificial Sequence
  3′ tbs-Kozak junction variant
kagvvatg

SEQ ID NO: 32 - DNA, 9 bp, Artificial Sequence
  3′ tbs-Kozak junction variant
kagrvvatg

SEQ ID NO: 33 - DNA, 10 bp, Artificial Sequence
  3′ tbs-Kozak junction variant
  misc_feature (4)..(4): n is a, c, g, or t
kagnrvvatg

SEQ ID NO: 34 - DNA, 11 bp, Artificial Sequence
  optimal 3′ tbs-Kozak junction variant
kagccgagatg

SEQ ID NO: 35 - DNA, 13 bp, Artificial Sequence
  optimal 3′ tbs-Kozak junction variant
  misc_feature (4)..(4): n is a, c, g, or t
kagnggagccatg

SEQ ID NO: 36 - DNA, 14 bp, Artificial Sequence
  optimal 3′ tbs-Kozak junction variant
  misc_feature (4)..(5): n is a, c, g, or t
kagnngagaccatg

SEQ ID NO: 37 - DNA, 12 bp, Artificial Sequence
  3′ tbs-Kozak junction variant
kaggcgagcatg

SEQ ID NO: 38 - DNA, 15 bp, Artificial Sequence
  spacer variant
atagcagagacggct

SEQ ID NO: 39 - DNA, 10 bp, Artificial Sequence
  spacer variant
atagcagaga

SEQ ID NO: 40 - DNA, 5 bp, Artificial Sequence
  spacer variant
atagc

SEQ ID NO: 41 - DNA, 26 bp, Artificial Sequence
  spacer variant
atatcagagacggctagcgtatacca

SEQ ID NO: 42 - DNA, 15 bp, Artificial Sequence
  spacer variant
atatcagagacggct

SEQ ID NO: 43 - DNA, 10 bp, Artificial Sequence
  spacer variant
agagacggct

SEQ ID NO: 44 - DNA, 5 bp, Artificial Sequence
  spacer variant
tacca

SEQ ID NO: 45 - DNA, 15 bp, Artificial Sequence
  3′ tbs-MCS-Kozak variant
gagctctagavvatg

SEQ ID NO: 46 - DNA, 16 bp, Artificial Sequence
  3′ tbs-MCS-Kozak variant
gagctcgtcgacvatg

SEQ ID NO: 47 - DNA, 20 bp, Artificial Sequence
  3′ tbs-MCS-Kozak variant
gagctcgaattcgaavvatg

SEQ ID NO: 48 - DNA, 21 bp, Artificial Sequence
  3′ tbs-MCS-Kozak variant
gagctctagacgtcgacvatg

SEQ ID NO: 49 - DNA, 22 bp, Artificial Sequence
  3′ tbs-MCS-Kozak variant
gagctctagaattcgaavvatg

SEQ ID NO: 50 - DNA, 23 bp, Artificial Sequence
  3′ tbs-MCS-Kozak variant
gagctctagatatcgatrvvatg

SEQ ID NO: 51 - DNA, 25 bp, Artificial Sequence
  3′ tbs-MCS-Kozak variant
kagactagtacttaagcttrvvatg

SEQ ID NO: 52 - DNA, 15 bp, Artificial Sequence
  3′ tbs-MCS-Kozak variant
gagctctagaccatg

SEQ ID NO: 53 - DNA, 16 bp, Artificial Sequence
  3′ tbs-MCS-Kozak variant
gagctcgtcgaccatg

SEQ ID NO: 54 - DNA, 20 bp, Artificial Sequence
  3′ tbs-MCS-Kozak variant
gagctcgaattcgaaccatg

SEQ ID NO: 55 - DNA, 21 bp, Artificial Sequence
  3′ tbs-MCS-Kozak variant
gagctctagacgtcgaccatg

SEQ ID NO: 56 - DNA, 22 bp, Artificial Sequence
  3′ tbs-MCS-Kozak variant
gagctctagaattcgaaccatg

SEQ ID NO: 57 - DNA, 23 bp, Artificial Sequence
  3′ tbs-MCS-Kozak variant
gagctctagatatcgataccatg

SEQ ID NO: 58 - DNA, 25 bp, Artificial Sequence
  3′ tbs-MCS-Kozak variant
kagactagtacttaagcttaccatg

SEQ ID NO: 59 - DNA, 32 bp, Encephalomyocarditis virus
cgtggttttcctttgaaaaacacgatgatacc

SEQ ID NO: 60 - DNA, 56 bp, Artificial Sequence
  optimal (overlapping) tbs-Kozak
gagtttagcggagtggagaagagcggagccgagcctagcagagacgagccgagatg

SEQ ID NO: 61 - DNA, 65 bp, Artificial Sequence
  optimal (overlapping) tbs-MCS-Kozak
gagtttagcggagtggagaagagcggagccgagcctagcagagacgagaagagctctagaccatg

SEQ ID NO: 62 - DNA, 74 bp, Artificial Sequence
  illustrative nucleic acid sequence 1 containing L33 Improved leader, optimal (overlapping) tbs ([KAGNN]8)-Kozak junction
ctttttcgcaacgggtttgccgccagaacacaggagtttagcggagtggagaagagcggagccgagccgagatg

SEQ ID NO: 63 - DNA, 89 bp, Artificial Sequence
  illustrative nucleic acid sequence 2 containing L33 Improved leader, optimal (overlapping) tbs ([KAGNN]11)-Kozak junction
ctttttcgcaacgggtttgccgccagaacacaggagtttagcggagtggagaagagcggagccgagcctagcagagacgagccgagatg

SEQ ID NO: 64 - DNA, 68 bp, Artificial Sequence
  illustrative nucleic acid sequence 3 containing L12 Improved leader, optimal (overlapping) tbs ([KAGNN]11)-Kozak junction
ctttttcgcaacgagtttagcggagtggagaagagcggagccgagcctagcagagacgagccgagatg

SEQ ID NO: 65 - DNA, 101 bp, Artificial Sequence
  illustrative nucleic acid sequence 4 for intron-containing 5′UTRs, resulting in a spliced leader comprising L33, optimal (overlapping) tbs ([KAGNN]11)-Kozak junction
ctttttcgcaacgggtttgccgccagaacacaggtgtcgtgaaaagagtttagcggagtggagaagagcggagccgagcctagcagagacgagccgagatg

SEQ ID NO: 66 - DNA, 56 bp, Artificial Sequence
  illustrative nucleic acid sequence 5 containing improved spacer, optimal (overlapping) tbs ([KAGNN]8)-Kozak junction
atagcagagacggctgagtttagcggagtggagaagagcggagccgagccgagatg

SEQ ID NO: 67 - DNA, 98 bp, Artificial Sequence
  illustrative nucleic acid sequence 6 containing L33 Improved leader tbs ([KAGNN]11)-MCS-Kozak
ctttttcgcaacgggtttgccgccagaacacaggagtttagcggagtggagaagagcggagccgagcctagcagagacgagaagagctctagaccatg

SEQ ID NO: 68 - DNA, 80 bp, Artificial Sequence
  illustrative nucleic acid sequence 7 containing improved spacer, tbs ([KAGNN]11)-MCS-Kozak
atagcagagacggctgagtttagcggagtggagaagagcggagccgagcctagcagagacgagaagagctctagaccatg

SEQ ID NO: 69 - DNA, 7 bp, Artificial Sequence
  3′ tbs-Kozak junction variant
gagaatg

SEQ ID NO: 70 - DNA, 7 bp, Artificial Sequence
  3′ tbs-Kozak junction variant
gagcatg

SEQ ID NO: 71 - DNA, 7 bp, Artificial Sequence
  3′ tbs-Kozak junction variant
gaggatg

SEQ ID NO: 72 - DNA, 7 bp, Artificial Sequence
  3′ tbs-Kozak junction variant
tagaatg

SEQ ID NO: 73 - DNA, 7 bp, Artificial Sequence
  3′ tbs-Kozak junction variant
tagcatg

SEQ ID NO: 74 - DNA, 7 bp, Artificial Sequence
  3′ tbs-Kozak junction variant
taggatg

SEQ ID NO: 75 - DNA, 8 bp, Artificial Sequence
  3′ tbs-Kozak junction variant
gagaaatg

SEQ ID NO: 76 - DNA, 8 bp, Artificial Sequence
  3′ tbs-Kozak junction variant
gagacatg

SEQ ID NO: 77 - DNA, 8 bp, Artificial Sequence
  3′ tbs-Kozak junction variant
gagagatg

SEQ ID NO: 78 - DNA, 8 bp, Artificial Sequence
  3′ tbs-Kozak junction variant
gagcaatg

SEQ ID NO: 79 - DNA, 8 bp, Artificial Sequence
  3′ tbs-Kozak junction variant
gagccatg

SEQ ID NO: 80 - DNA, 8 bp, Artificial Sequence
  3′ tbs-Kozak junction variant
gagcgatg

SEQ ID NO: 81 - DNA, 8 bp, Artificial Sequence
  3′ tbs-Kozak junction variant
gaggaatg

SEQ ID NO: 82 - DNA, 8 bp, Artificial Sequence
  3′ tbs-Kozak junction variant
gaggcatg

SEQ ID NO: 83 - DNA, 8 bp, Artificial Sequence
  3′ tbs-Kozak junction variant
gagggatg

SEQ ID NO: 84 - DNA, 8 bp, Artificial Sequence
  3′ tbs-Kozak junction variant
tagaaatg

SEQ ID NO: 85 - DNA, 8 bp, Artificial Sequence
  3′ tbs-Kozak junction variant
tagacatg

SEQ ID NO: 86 - DNA, 8 bp, Artificial Sequence
  3′ tbs-Kozak junction variant
tagagatg

SEQ ID NO: 87 - DNA, 8 bp, Artificial Sequence
  3′ tbs-Kozak junction variant
tagcaatg

SEQ ID NO: 88 - DNA, 8 bp, Artificial Sequence
  3′ tbs-Kozak junction variant
tagccatg

SEQ ID NO: 89 - DNA, 8 bp, Artificial Sequence
  3′ tbs-Kozak junction variant
tagcgatg

SEQ ID NO: 90 - DNA, 8 bp, Artificial Sequence
  3′ tbs-Kozak junction variant
taggaatg

SEQ ID NO: 91 - DNA, 8 bp, Artificial Sequence
  3′ tbs-Kozak junction variant
taggcatg

SEQ ID NO: 92 - DNA, 8 bp, Artificial Sequence
  3′ tbs-Kozak junction variant
tagggatg

SEQ ID NO: 93 - DNA, 45 bp, Artificial Sequence
  L33 improved leader derived from a splicing event
ctttttcgcaacgggtttgccgccagaacacaggtgtcgtgaaaa

SEQ ID NO: 94 - DNA, 30 bp, Artificial Sequence
  splice donor region variant
ggggcggcgactggtgagtacgccaaaaat

SEQ ID NO: 95 - DNA, 30 bp, Artificial Sequence
  splice donor region variant
ggggcggcgactgcagacaacgccaaaaat

SEQ ID NO: 96 - DNA, 8 bp, Artificial Sequence
  major splice donor consensus sequence variant
tggtragt

SEQ ID NO: 97 - DNA, 5 bp, Artificial Sequence
  major splice donor consensus sequence variant
ctggt

SEQ ID NO: 98 - DNA, 6 bp, Artificial Sequence
  mutated splice donor region variant (MSD-2KO)
cagaca

SEQ ID NO: 99 - DNA, 19 bp, Artificial Sequence
  mutated splice donor region variant (MSD-2KO)
ggcgactgcagacaacgcc

SEQ ID NO: 100 - DNA, 9 bp, Artificial Sequence
  mutated splice donor region variant (MSD-2KOv2)
gtggagact

SEQ ID NO: 101 - DNA, 19 bp, Artificial Sequence
  mutated splice donor region variant (MSD-2KOv2)
ggcgagtggagactacgcc

SEQ ID NO: 102 - DNA, 19 bp, Artificial Sequence
  splice donor region SL2
ggcgactggtgagtacgcc

SEQ ID NO: 103 - DNA, 5 bp, Artificial Sequence
  cryptic splice donor consensus sequence variant
tgagt

SEQ ID NO: 104 - DNA, 30 bp, Artificial Sequence
  mutated splice donor (MSD) region, MSD-2KOv2
ggggcggcgagtggagactacgccaaaaat

SEQ ID NO: 105 - DNA, 32 bp, Artificial Sequence
  mutated splice donor (MSD) region, MSD-2KOm5
ggggaaggcaacagataaatatgccttaaaat

SEQ ID NO: 106 - DNA, 7 bp, Artificial Sequence
  core sequence
gtgagta

SEQ ID NO: 107 - DNA, 23 bp, Artificial Sequence
  mutated splice donor
aaggcaacagataaatatgcctt

SEQ ID NO: 108 - DNA, 6 bp, Artificial Sequence
  3′ tbs-Kozak junction variant
tagatg

SEQ ID NO: 109 - DNA, 8 bp, Artificial Sequence
  3′ tbs-Kozak junction variant
gagatatg

SEQ ID NO: 110 - DNA, 8 bp, Artificial Sequence
  3′ tbs-Kozak junction variant
gagctatg

SEQ ID NO: 111 - DNA, 8 bp, Artificial Sequence
  3′ tbs-Kozak junction variant
tagatatg

SEQ ID NO: 112 - DNA, 8 bp, Artificial Sequence
  3′ tbs-Kozak junction variant
tagctatg

SEQ ID NO: 113 - DNA, 6 bp, Artificial Sequence
  3′ tbs-Kozak junction variant
  misc_feature (4)..(5): n is a, c, g, or t
kagnng

SEQ ID NO: 114 - DNA, 6 bp, Artificial Sequence
  3′ tbs-Kozak junction variant
kagatg

SEQ ID NO: 115 - DNA, 7 bp, Artificial Sequence
  3′ tbs-Kozak consensus sequence
  misc_feature (4)..(5): n is a, c, g, or t
kagnntg

SEQ ID NO: 116 - DNA, 7 bp, Artificial Sequence
  3′ tbs-Kozak consensus sequence
  misc_feature (4)..(4): n is a, c, g, or t
kagnatg

SEQ ID NO: 117 - DNA, 1082 bp, Artificial Sequence
  Chicken beta-Actin / Rabbit beta-globin chimeric 5′UTR-intron with tbs-kzkV0.G variant
cggcgggcgggaacgttgccttcgccccgtgccccgctccgcgccgcctcgcgccgcccgccccggctctgactgaccgcgttactcccacaggtgagcgggcgggacggcccttctccctccgggctgtaattagcgcttggtttaatgacggctcgtttcttttctgtggctgcgtgaaagccttaaagggctccgggagggcctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcgggccgggggcggtgccccgcggtgcgggggggctgcgaggggaacaaaggctgcgtgcggggtgtgtgcgtgggggggtgagcagggggtgtgggcgcggcggtcgggctgtaacccccccctggcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctttagagcctctgctaaccatgttcatgccttcttctttttcctacagctcctgggcaaagagtttagcggagtggagaagagcggagccgagcctagcagagacgagccgagatg

SEQ ID NO: 118 - DNA, 1040 bp, Artificial Sequence
  EF1a 5′UTR-intron with tbskzkV0.G variant
ctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagagtttagcggagtggagaagagcggagccgagcctagcagagacgagccgagatg

SEQ ID NO: 119 - DNA, 161 bp, Artificial Sequence
  spliced sequence corresponding to SEQ ID NO 117
cggcgggcgggaacgttgccttcgccccgtgccccgctccgcgccgcctcgcgccgcccgccccggctctgactgaccgcgttactcccacagctcctgggcaaagagtttagcggagtggagaagagcggagccgagcctagcagagacgagccgagatg

SEQ ID NO: 120 - DNA, 101 bp, Artificial Sequence; identical to SEQ ID NO: 65
  spliced sequence corresponding to SEQ ID NO 118

SEQ ID NO: 121 - DNA, 1026 bp, Artificial Sequence
  Chicken beta-Actin / Rabbit beta-globin chimeric 5′UTR-intron
cggcgggcgggaacgttgccttcgccccgtgccccgctccgcgccgcctcgcgccgcccgccccggctctgactgaccgcgttactcccacaggtgagcgggcgggacggcccttctccctccgggctgtaattagcgcttggtttaatgacggctcgtttcttttctgtggctgcgtgaaagccttaaagggctccgggagggcctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcgggccgggggcggtgccccgcggtgcgggggggctgcgaggggaacaaaggctgcgtgcggggtgtgtgcgtgggggggtgagcagggggtgtgggcgcggcggtcgggctgtaacccccccctggcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctttagagcctctgctaaccatgttcatgccttcttctttttcctacagctcctgggcaaa

SEQ ID NO: 122 - DNA, 984 bp, Artificial Sequence
  EF1a 5′UTR-intron
ctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaa

SEQ ID NO: 123 - DNA, 1030 bp, Artificial Sequence
  Chicken beta-Actin / Rabbit beta-globin chimeric 5′UTR-intron-tbs consensus
  misc_feature (1027)..(1030): kagn(2-3) may be repeated 10-11 times
  misc_feature (1030)..(1030): n may be repeated 2-3 times
cggcgggcgggaacgttgccttcgccccgtgccccgctccgcgccgcctcgcgccgcccgccccggctctgactgaccgcgttactcccacaggtgagcgggcgggacggcccttctccctccgggctgtaattagcgcttggtttaatgacggctcgtttcttttctgtggctgcgtgaaagccttaaagggctccgggagggcctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcgggccgggggcggtgccccgcggtgcgggggggctgcgaggggaacaaaggctgcgtgcggggtgtgtgcgtgggggggtgagcagggggtgtgggcgcggcggtcgggctgtaacccccccctggcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctttagagcctctgctaaccatgttcatgccttcttctttttcctacagctcctgggcaaakagn

SEQ ID NO: 124 - DNA, 988 bp, Artificial Sequence
  EF1a 5′UTR-intron-tbs consensus
  misc_feature (985)..(988): kagn(2-3) may be repeated 10-11 times
  misc_feature (988)..(988): n may be repeated 2-3 times
ctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaakagn

SEQ ID NO: 125 - DNA, 6 bp, Artificial Sequence
  Kozak sequence
  misc_feature (2)..(3): n is a, c, g, t or u
rnnatg

SEQ ID NO: 126 - DNA, 12 bp, Artificial Sequence
  original tbs-Kozak region sequence
acagccaccatg

SEQ ID NO: 127 - DNA, 17 bp, Artificial Sequence
  HpaI variant tbs-Kozak region sequence
gagttaacgccaccatg

SEQ ID NO: 128 - DNA, 13 bp, Artificial Sequence
  full Kozak consensus sequence for DNA
gccgccrccatgg

SEQ ID NO: 129 - RNA, 13 nt, Artificial Sequence
  full Kozak consensus sequence for RNA
gccgccrccaugg

SEQ ID NO: 130 - RNA, 10 nt, Artificial Sequence
  extended Kozak consensus sequence
  misc_feature (2)..(3): n is a, c, g, or u
gnnrvvaugg

SEQ ID NO: 131 - DNA, 153 bp, Artificial Sequence
  main U1 snRNA sequence [clover leaf] (nt 410-562) of the U1_256 sequence
gcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg

SEQ ID NO: 132 - DNA, 34 bp, Artificial Sequence
  UTR-leader sequence (CMV)
gtcagatccgctagcgctaccggactcagatctc

SEQ ID NO: 133 - DNA, 92 bp, Artificial Sequence
  UTR-leader sequence (RSV)
gccatttgaccattcaccacattggtgtgcacctccaaggccaagatctttgtcgatcctaccatccactcgacacacccgccagcggccgc

SEQ ID NO: 134 - DNA, 91 bp, Artificial Sequence
  UTR-leader sequence (EF1a)
ctttttcgcaacgggtttgccgccagaacacaggtgtcgtgaaaactacccctaaaagccaaaagatctttgtcgatcctaccatccactc

SEQ ID NO: 135 - DNA, 405 bp, Artificial Sequence
  UTR-leader sequence (UBC)
agctagttccgtcgcagccgggatttgggtcgcggttcttgtttgtggatcgctgtgatcgtcacttggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagacttgtaaattgtccgctaaattctggccgtttttggcttttttgttagacaacagatcttgatcctaccatccactcgacacacccgccagcggccgc

SEQ ID NO: 136 - DNA, 104 bp, Artificial Sequence
  UTR-leader sequence (SV40)
ctctgagctattccagaagtagtgaggaggcttttttggaggcctaggcttttgcagatctttgtcgatcctaccatccactcgacacacccgccagcggccgc

SEQ ID NO: 137 - DNA, 143 bp, Artificial Sequence
  UTR-leader sequence (huPGK)
gttccgcattctggcaagcctccggagcgcacgtcggcagtcggctccctcgttgaccgaatcaccgacctctctccccagctgtatttccaaaagatctttgtcgatcctaccatccactcgacacacccgccagcggccgc

SEQ ID NO: 138 - DNA, 79 bp, Artificial Sequence
  UTR-leader sequence (hsvTK)
acaccgagcgaccctgcagcgacccgcttaagatctttgtcgatcctaccatccactcgacacacccgccagcggccgc

SEQ ID NO: 139 - DNA, 33 bp, Artificial Sequence; identical to SEQ ID NO: 25
  UTR-leader sequence (Improved leader L33)

SEQ ID NO: 140 - DNA, 12 bp, Artificial Sequence
  UTR-leader sequence (Improved leader L12)
ctttttcgcaac

SEQ ID NO: 141 - DNA, 55 bp, Artificial Sequence; identical to SEQ ID NO: 14
  tbs sequence

SEQ ID NO: 142 - DNA, 55 bp, Artificial Sequence
  tbs consensus sequence
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
  misc_feature (54)..(55): n is a, c, g, or t
kagnnkagnnkagnnkagnnkagnnkagnnkagnnkagnnkagnnkagnnkagnn

SEQ ID NO: 143 - DNA, 12 bp, Artificial Sequence
  multiple cloning site (MCS) sequence variant, No MCS
acagccaccatg

SEQ ID NO: 144 - DNA, 15 bp, Artificial Sequence
  MCS sequence variant, MCS2.1
gagctctagaccatg

SEQ ID NO: 145 - DNA, 16 bp, Artificial Sequence
  MCS sequence variant, MCS3.1
gagctcgtcgaccatg

SEQ ID NO: 146 - DNA, 20 bp, Artificial Sequence
  MCS sequence variant, MCS3.2
gagctcgaattcgaaccatg

SEQ ID NO: 147 - DNA, 21 bp, Artificial Sequence
  MCS sequence variant, MCS4.1
gagctctagacgtcgaccatg

SEQ ID NO: 148 - DNA, 22 bp, Artificial Sequence
  MCS sequence variant, MCS4.2
gagctctagaattcgaaccatg

SEQ ID NO: 149 - DNA, 23 bp, Artificial Sequence
  MCS sequence variant, MCS4.3
gagctctagatatcgataccatg

SEQ ID NO: 150 - DNA, 25 bp, Artificial Sequence
  MCS sequence variant, MCS4.4
kagactagtacttaagcttaccatg

SEQ ID NO: 151 - DNA, 17 bp, Artificial Sequence
  3′tbs-Kozak junction sequence, original
  misc_feature (4)..(5): n is a, c, g, or t
kagnnacagccaccatg

SEQ ID NO: 152 - DNA, 11 bp, Artificial Sequence
  3′tbs-Kozak junction sequence, variant ′0′
kagccgagatg

SEQ ID NO: 153 - DNA, 12 bp, Artificial Sequence
  3′tbs-Kozak junction sequence, variant ′1′
kaggcgagcatg

SEQ ID NO: 154 - DNA, 13 bp, Artificial Sequence
  3′tbs-Kozak junction sequence, variant ′2′
  misc_feature (4)..(4): n is a, c, g, or t
kagnggagccatg

SEQ ID NO: 155 - DNA, 14 bp, Artificial Sequence
  3′tbs-Kozak junction sequence, variant ′3′
  misc_feature (4)..(5): n is a, c, g, or t
kagnngagaccatg

SEQ ID NO: 156 - DNA, 105 bp, Artificial Sequence
  IRES-spacer-Kozak variant sequence, Original [26nt]
  misc_feature (59)..(63): kagnn may be repeated 7-11 times
  misc_feature (67)..(68): n is a, c, g, or t
  misc_feature (72)..(73): n is a, c, g, or t
  misc_feature (77)..(78): n is a, c, g, or t
  misc_feature (82)..(83): n is a, c, g, or t
  misc_feature (87)..(88): n is a, c, g, or t
  misc_feature (92)..(93): n is a, c, g, or t
cgtggttttcctttgaaaaacacgatgataccatagcagagacggctagcgttaacctkagnnkagnnkagnnkagnnkagnnkagnnkagnnaacgccaccatg

SEQ ID NO: 157 - DNA, 94 bp, Artificial Sequence
  IRES-spacer-Kozak variant sequence, Original [Trunc-15nt]
  misc_feature (48)..(52): kagnn may be repeated 7-11 times
  misc_feature (56)..(57): n is a, c, g, or t
  misc_feature (61)..(62): n is a, c, g, or t
  misc_feature (66)..(67): n is a, c, g, or t
  misc_feature (71)..(72): n is a, c, g, or t
  misc_feature (76)..(77): n is a, c, g, or t
  misc_feature (81)..(82): n is a, c, g, or t
cgtggttttcctttgaaaaacacgatgataccatagcagagacggctkagnnkagnnkagnnkagnnkagnnkagnnkagnnaacgccaccatg

SEQ ID NO: 158 - DNA, 89 bp, Artificial Sequence
  IRES-spacer-Kozak variant sequence, Original [Trunc-10nt]
  misc_feature (43)..(47): kagnn may be repeated 7-11 times
  misc_feature (51)..(52): n is a, c, g, or t
  misc_feature (56)..(57): n is a, c, g, or t
  misc_feature (61)..(62): n is a, c, g, or t
  misc_feature (66)..(67): n is a, c, g, or t
  misc_feature (71)..(72): n is a, c, g, or t
  misc_feature (76)..(77): n is a, c, g, or t
cgtggttttcctttgaaaaacacgatgataccatagcagagakagnnkagnnkagnnkagnnkagnnkagnnkagnnaacgccaccatg

SEQ ID NO: 159 - DNA, 84 bp, Artificial Sequence
  IRES-spacer-Kozak variant sequence, Original [Trunc-5nt]
  misc_feature (38)..(42): kagnn may be repeated 7-11 times
  misc_feature (46)..(47): n is a, c, g, or t
  misc_feature (51)..(52): n is a, c, g, or t
  misc_feature (56)..(57): n is a, c, g, or t
  misc_feature (61)..(62): n is a, c, g, or t
  misc_feature (66)..(67): n is a, c, g, or t
  misc_feature (71)..(72): n is a, c, g, or t
cgtggttttcctttgaaaaacacgatgataccatagckagnnkagnnkagnnkagnnkagnnkagnnkagnnaacgccaccatg

SEQ ID NO: 160 - DNA, 79 bp, Artificial Sequence
  IRES-spacer-Kozak variant sequence, No Spacer [0nt]
  misc_feature (33)..(37): kagnn may be repeated 7-11 times
  misc_feature (41)..(42): n is a, c, g, or t
  misc_feature (46)..(47): n is a, c, g, or t
  misc_feature (51)..(52): n is a, c, g, or t
  misc_feature (56)..(57): n is a, c, g, or t
  misc_feature (61)..(62): n is a, c, g, or t
  misc_feature (66)..(67): n is a, c, g, or t
cgtggttttcctttgaaaaacacgatgatacckagnnkagnnkagnnkagnnkagnnkagnnkagnnaacgccaccatg

SEQ ID NO: 161 - DNA, 105 bp, Artificial Sequence
  IRES-spacer-Kozak variant sequence, Variant [26nt]
  misc_feature (59)..(63): kagnn may be repeated 7-11 times
  misc_feature (67)..(68): n is a, c, g, or t
  misc_feature (72)..(73): n is a, c, g, or t
  misc_feature (77)..(78): n is a, c, g, or t
  misc_feature (82)..(83): n is a, c, g, or t
  misc_feature (87)..(88): n is a, c, g, or t
  misc_feature (92)..(93): n is a, c, g, or t
cgtggttttcctttgaaaaacacgatgataccatatcagagacggctagcgtataccakagnnkagnnkagnnkagnnkagnnkagnnkagnnaacgccaccatg

SEQ ID NO: 162 - DNA, 94 bp, Artificial Sequence
  IRES-spacer-Kozak variant sequence, Variant [Trunc-15nt]
  misc_feature (48)..(52): kagnn may be repeated 7-11 times
  misc_feature (56)..(57): n is a, c, g, or t
  misc_feature (61)..(62): n is a, c, g, or t
  misc_feature (66)..(67): n is a, c, g, or t
  misc_feature (71)..(72): n is a, c, g, or t
  misc_feature (76)..(77): n is a, c, g, or t
  misc_feature (81)..(82): n is a, c, g, or t
cgtggttttcctttgaaaaacacgatgataccatatcagagacggctkagnnkagnnkagnnkagnnkagnnkagnnkagnnaacgccaccatg

SEQ ID NO: 163 - DNA, 89 bp, Artificial Sequence
  IRES-spacer-Kozak variant sequence, Variant [Trunc-10nt]
  misc_feature (43)..(47): kagnn may be repeated 7-11 times
  misc_feature (51)..(52): n is a, c, g, or t
  misc_feature (56)..(57): n is a, c, g, or t
  misc_feature (61)..(62): n is a, c, g, or t
  misc_feature (66)..(67): n is a, c, g, or t
  misc_feature (71)..(72): n is a, c, g, or t
  misc_feature (76)..(77): n is a, c, g, or t
cgtggttttcctttgaaaaacacgatgataccagagacggctkagnnkagnnkagnnkagnnkagnnkagnnkagnnaacgccaccatg

SEQ ID NO: 164 - DNA, 84 bp, Artificial Sequence
  IRES-spacer-Kozak variant sequence, Variant [Trunc-5nt]
  misc_feature (38)..(42): kagnn may be repeated 7-11 times
  misc_feature (46)..(47): n is a, c, g, or t
  misc_feature (51)..(52): n is a, c, g, or t
  misc_feature (56)..(57): n is a, c, g, or t
  misc_feature (61)..(62): n is a, c, g, or t
  misc_feature (66)..(67): n is a, c, g, or t
  misc_feature (71)..(72): n is a, c, g, or t
cgtggttttcctttgaaaaacacgatgatacctaccakagnnkagnnkagnnkagnnkagnnkagnnkagnnaacgccaccatg

SEQ ID NO: 165 - DNA, 86 bp, Artificial Sequence
  IRES-spacer-Kozak variant sequence, Original [Trunc-15nt]-3ntKozak
  misc_feature (48)..(52): kagnn may be repeated 6-10 times
  misc_feature (56)..(57): n is a, c, g, or t
  misc_feature (61)..(62): n is a, c, g, or t
  misc_feature (66)..(67): n is a, c, g, or t
  misc_feature (71)..(72): n is a, c, g, or t
  misc_feature (76)..(77): n is a, c, g, or t
cgtggttttcctttgaaaaacacgatgataccatagcagagacggctkagnnkagnnkagnnkagnnkagnnkagnngagaccatg

SEQ ID NO: 166 - DNA, 94 bp, Artificial Sequence; identical to SEQ ID NO: 157
  IRES-spacer-Kozak variant sequence, Original [Trunc-15nt]-9ntKozak
  misc_feature (48)..(52): kagnn may be repeated 7-11 times
  misc_feature (56)..(57): n is a, c, g, or t
  misc_feature (61)..(62): n is a, c, g, or t
  misc_feature (66)..(67): n is a, c, g, or t
  misc_feature (71)..(72): n is a, c, g, or t
  misc_feature (76)..(77): n is a, c, g, or t
  misc_feature (81)..(82): n is a, c, g, or t

SEQ ID NO: 167 - DNA, 67 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
  misc_feature (54)..(55): n is a, c, g, or t
kagnnkagnnkagnnkagnnkagnnkagnnkagnnkagnnkagnnkagnnkagnnacagccaccatg

SEQ ID NO: 168 - DNA, 56 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagatg

SEQ ID NO: 169 - DNA, 56 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagatg

SEQ ID NO: 170 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagcatg

SEQ ID NO: 171 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagaatg

SEQ ID NO: 172 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngaggatg

SEQ ID NO: 173 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagaatg

SEQ ID NO: 174 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagcatg

SEQ ID NO: 175 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntaggatg

SEQ ID NO: 176 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagccatg

SEQ ID NO: 177 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagaaatg

SEQ ID NO: 178 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagagatg

SEQ ID NO: 179 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagacatg

SEQ ID NO: 180 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagatatg

SEQ ID NO: 181 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagcaatg

SEQ ID NO: 182 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagcgatg

SEQ ID NO: 183 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagctatg

SEQ ID NO: 184 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngaggaatg

SEQ ID NO: 185 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagggatg

SEQ ID NO: 186 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngaggcatg

SEQ ID NO: 187 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagaaatg

SEQ ID NO: 188 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagagatg

SEQ ID NO: 189 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagacatg

SEQ ID NO: 190 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagatatg

SEQ ID NO: 191 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagcaatg

SEQ ID NO: 192 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagcgatg

SEQ ID NO: 193 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagccatg

SEQ ID NO: 194 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagctatg

SEQ ID NO: 195 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntaggaatg

SEQ ID NO: 196 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagggatg

SEQ ID NO: 197 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntaggcatg

SEQ ID NO: 198 - DNA, 13 bp, Artificial Sequence
  mutated splice donor (MSD) region, deltaSL2
ggggcgcaaaaat